(19) United States
(12) Patent Application Publication
(10) Pub. No.: US 2010/0055133 A1
(43) Pub. Date: Mar. 4, 2010

(54) PHARMACEUTICAL COMPOSITIONS

(75) Inventors: Andrew John Duffield, Berkhamsted (GB); Graham Jackson, Christ Church (GB); Steven E. Frisbee, Reston, VA (US)

Correspondence Address: SCHWEGMAN, LUNDBERG & WOESSNER, P.A., P.O. BOX 2938, MINNEAPOLIS, MN, 55402, US

(73) Assignee: Biovail Laboratories International (Barbados) S.R.L, Christ Church (BB)

(21) Appl. No.: 12/540,144

(22) Filed: Aug. 12, 2009

(30) Foreign Application Priority Data
Aug. 12, 2008 (GB) GB 0814695.3

(51) Int. Cl. A61K 39/08; A61K 31/435; A61K 31/5513; A61P 25/00

(52) U.S. Cl. 424/239.1; 514/294; 514/221

(57) ABSTRACT

The present invention provides for a pharmaceutical composition that includes tetrabenazine and a release-retarding agent; and a method of treating a hyperkinetic movement disorder (e.g., Huntington's disease, chorea associated with Huntington's disease, hemiballismus, senile chorea, tic disorders, tardive dyskinesia, myoclonlus, dystoniia and/or Tourette's syndrome). The method includes administering an effective amount of the pharmaceutical composition, for a period of time effective to treat the hyperkinetic movement disorder.

### RELATED APPLICATIONS

This application claims priority from U.K. Patent Application No. GB 0814695.3, filed Aug. 12, 2008; which is incorporated herein by reference.

### BACKGROUND

Tetrabenazine (chemical name: 1,3,4,6,7,11b-hexahydro-9,10-dimethoxy-3-(2-methylpropyl)-2H-benzo(a)quinolizin-2-one) has been in use as a pharmaceutical drug since the late 1950s. Initially developed as an anti-psychotic, tetrabenazine is currently used in the symptomatic treatment of hyperkinetic movement disorders such as Huntington's disease, hemiballismus, senile chorea, tic, tardive dyskinesia, myoclonus, dystonia and Tourette's syndrome, see for example Ondo et al., Am. J. Psychiatry. (1999) August; 156(8):1279-81 and Jankovic et al., Neurology (1997) February; 48(2):358-62.

The chemical structure of tetrabenazine is as shown below.

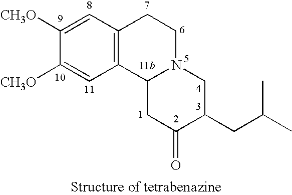

The compound has chiral centers at the 3 and 11b carbon atoms and hence can, theoretically, exist in a total of four isomeric forms, as shown below.

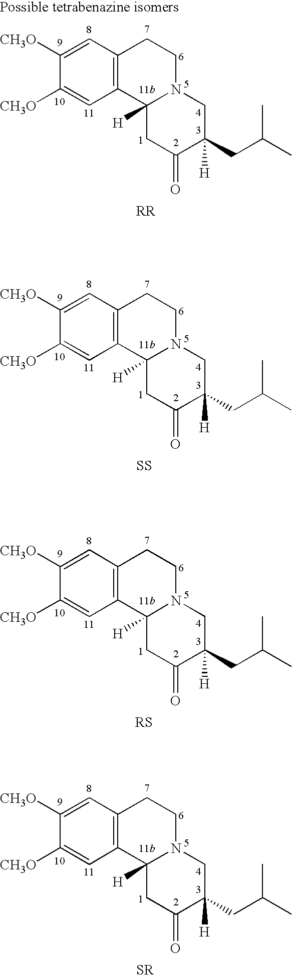

Commercially available tetrabenazine is a racemic mixture of the RR and SS isomers.

Tetrabenazine has somewhat poor and variable bioavailability. It is extensively metabolised by first-pass metabolism, and little or no unchanged tetrabenazine is typically detected in the urine. The major metabolite is dihydrotetrabenazine (chemical name: 2-hydroxy-3-(2-methylpropyl)-1,3,4,6,7,11b-hexahydro-9,10-dimethoxy-benzo(a)quinolizine) which is formed by reduction of the 2-keto group in tetrabenazine, and is believed to be primarily responsible for the activity of the drug (see Melvar et al., Drug Metab. Disp, 15, 250-255 (1987) and J. Pharm. Sci., 76, No. 6, 461-465 (1987)).

The preparation of tetrabenazine and of its salts, in particular the hydrochloride, is described in GB 789 789. The preparation of α-dihydrotetrabenazine and its salts, in particular the hydrochloride, is described in GB 800 969. The preparation of (±)-α-dihydrotetrabenazine is described by Brossi (Helv. Chim. Acta., 41:249-251 (1958)). The preparation of (+)-α-dihydrotetrabenazine is described by Kilbourn (Eur. J Pharmacol, 278:249-251 (1995)). The preparation of 3,11b cis isomers of dihydrotetrabenazine is described in WO 2005/077946.

Tetrabenazine is an effective and safe drug for the treatment of a variety of hyperkinetic movement disorders and, in contrast to typical neuroleptics, has not been demonstrated to cause tardive dyskinesia. Nevertheless, tetrabenazine does exhibit a number of dose-related side effects including causing depression, Parkinsonism, drowsiness, nervousness or anxiety, insomnia and, in rare cases, neuroleptic malignant syndrome.

Formulating drugs as controlled-release formulations can sometimes reduce the side effects of drugs by smoothing out the Cmax value and can also provide simplified once-a-day administration.

Tetrabenazine is soluble at acid pH (as found in the stomach) but the solubility decreases dramatically at the higher pH values found lower down the gastrointestinal (GI) tract. Comparative Example 1 as herein described illustrates that tetrabenazine is practically insoluble in the pH range of 3-12 and slightly soluble at pH 2 (as found in the stomach). Immediate-release formulation tablets including tetrabenazine which are currently available are designed to disintegrate in the stomach leading to dissolution and absorption of tetrabenazine in the stomach.

Immediate-release formulations require that a drug is administered in a high dose at a given time only to have to repeat that dose several hours or days later. This is inconvenient to the patient and can result in damaging side effects. In contrast, controlled-release formulations enable drugs to be delivered to the patient continually for prolonged the periods and in a controlled fashion.

However, the ambient pH increases moving down the GI tract. For example, the pH in the duodenum is about 6 and increases to about 7-8 in the ileum and decreases slightly in the colon to 5-7. At these pH levels tetrabenazine is practically insoluble.

Therefore, it would be expected that by formulating tetrabenazine as a controlled-release formulation, so preventing the drug from being released in the stomach and delaying release until the drug reaches regions of the GI tract where it is less soluble, the bioavailability of tetrabenazine would be significantly reduced.

The use of hydroxypropylmethylcellulose as a carrier in an extended release formulation of felodipine has been described in several publications; see for example (I) Abrahammsson et al., Pharmaceutical Research, Vol. 10, No. 5, 1993, pp 709-714; (2) Vuong et al., Poster: The Effect of In-Vitro Dissolution Parameters on the Release Rate of a Low Dose, Low Solubility Drug from Extended Release Hypromellose Matrix Formulations”; Controlled Release Society 2006; and (3) Wingstrand et al, International Journal of Pharmaceutics, 60 (1990), 151-156. Felodipine is a non-basic dihydropyridine derivative which is understood to be generally insoluble in aqueous media, including acidic media. Tetrabenazine by contrast is a basic compound which, whilst poorly soluble or insoluble in the pH range 3-12, is soluble to a significantly greater extent at stomach pH.

US 2005/0064034 (Andrx Pharmaceuticals) discloses controlled release formulations for poorly soluble drugs wherein a formulation contains several different granular preparations having different drug release properties. Hydroxypropylmethylcellulose is one of several polymers disclosed in US 2005/0064034. This document contains no reference to tetrabenazine or any compounds of similar structure to tetrabenazine. The only drug substances for which specific examples of formulations are disclosed are metronidazole and clarithromycin.

### SUMMARY

The present invention provides for a pharmaceutical composition that includes tetrabenazine and a release-retarding agent. The present invention also provides for a method of treating a hyperkinetic movement disorder. The method includes administering an effective amount of the pharmaceutical composition, for a period of time effective to treat the hyperkinetic movement disorder.

The invention further provides a pharmaceutical composition for use in treating a hyperkinetic movement disorder wherein the composition includes tetrabenazine and a release-retarding agent. The invention also provides the use of tetrabenazine and a release-retarding agent for the manufacture of a pharmaceutical composition for treating a hyperkinetic movement disorder.

Particular aspects and embodiments of the invention as are set forth below and in the claims appended hereto.

### DESCRIPTION OF THE FIGURES

FIG. 1 graphically illustrates the plasma concentration of alpha-dihydrotetrabenazine (ng/ml) over time after administering to subjects (n=13) the 50 mg tetrabenazine tablets made and tested as described in Examples 1 and 2. The subjects received the tablets during fasting (“fast”; ▪ symbols) or after consuming a meal (“fed”; symbols). The area under the curve (AUC) was determined for the fed and fast subjects and the ratio of the fed to fast AUC₀₋ₜ was 144.71% (where t is the last timepoint of blood sampling that had detectable drug), whereas the ratio of the fed to fast AUC_{0-∞} is 138.55%. The Cmax fed:fast ratio was 238.71%. As illustrated the tetrabenazine tablets release larger amounts of tetrabenazine when the subject has consumed food.

FIG. 2 graphically illustrates the plasma concentration of beta-dihydrotetrabenazine (ng/ml) over time after administering to subjects (n=13) the 50 mg tetrabenazine tablets made and tested as described in Examples 1 and 15. The subjects received the tablets during fasting (“fast”; ▪ symbols) or after consuming a meal (“fed”; symbols). The area under the curve (AUC) was determined for the fed and fast subjects and the ratio of the fed to fast AUC₀₋ₜ was 153.44% (where t is the last timepoint of blood sampling that had detectable drug), whereas the ratio of the fed to fast AUC_{0-∞} is 133.45%. The Cinax fed:fast ratio was 263.46%. As illustrated the tetrabenazine tablets release larger amounts of tetrabenazine when the subject has consumed food.

FIG. 3 graphically illustrates the plasma concentration of alpha-dihydrotetrabenazine (ng/ml) over time after administering to subjects (n=13) the 50 mg tetrabenazine tablets made and tested as described in Examples 1 and 15, compared to the plasma concentration of alpha-dihydrotetrabenazine (ng/ml) in fasting subjects after administering an immediate release tetrabenazine formulation (Nitoman®; ▴ symbols). The subjects received the Example 1 tablets during fasting (“fast”; ▪ symbols) or after consuming a meal (“fed”; symbols). The area under the curve (AUC) was determined for the fed and fast subjects. For subjects receiving the formulation described in Example 1, the fed to fast AUC₀₋ₜ was 102.67% (where t is the last timepoint of blood sampling that had detectable drug), while the ratio of the fed to fast AUC_{0-∞} is 102.20% and the Cmax fed:fast ratio was 73.03%. For fasting subjects receiving the immediate release formulation (Nitoman®; ▴ symbols), the fed to fast AUC₀₋ₜ was 67.177% (where t is the last timepoinlt of blood sampling that had detectable drug), while the ratio of the fed to fast AUC_{0-∞} is 70.91% and the Cmax fed:fast ratio was 25.30%. As illustrated the tetrabenazine tablets release larger amounts of tetrabenazine when the subject has consumed food.

FIG. 4 graphically illustrates the plasma concentration of beta-dihydrotetrabenazine (ng/ml) over time after administering to subjects (n=13) the 50 mg tetrabenazine tablets made and tested as described in Examples 1 and 15, compared to the plasma concentration of alpha-dihydrotetrabenazine (ng/ml) in fasting subjects after administering an immediate release tetrabenazine formulation (Nitoman®; ▴ symbols). The subjects received the Example 1 tablets during fasting (“fast”; ▪ symbols) or after consuming a meal (“fed”; symbols). The area under the curve (AUC) was determined for the fed and fast subjects. For subjects receiving the formulation described in Example 1, the fed to fast AUC₀₋ₜ was 94.50% (where t is the last timepoint of blood sampling that had detectable drug), while the ratio of the fed to fast AUC_{0-∞} is 93.89% and the Cmax fed:fast ratio was 69.29%. For fasting subjects receiving the immediate release formulation (Nitoman®), the fed to fast AUC₀₋ₜ was 58.27% (where t is the last timepoint of blood sampling that had detectable drug), while the ratio of the fed to fast AUC_{0-∞} is 68.82% and the Cmax fed:fast ratio was 21.24%. As illustrated the tetrabenazine tablets release larger amounts of tetrabeniazinie when the subject has consumed food.

FIG. 5 graphically illustrates the dissolution profile for the tetrabenazine formulation described in Example 32 when stirred at 50 rpm (♦), 75 rpm () and 100 rpm (▴). The percent tetrabenazine dissolved is shown on the y-axis with the time (hours) shown on the x-axis. Dissolution was performed in 0.1M HCl using paddles and sinkers, with 45 μm in-line large surface filter tips and a 15 ml pull volume. The results shown for each line are the mean of 12 tests.

FIG. 6 graphically illustrates the dissolution profile for the tetrabenazine formulation described in Example 32 in different dissolution media, including 0.1 N HCl (♦), pH 4.5 acetate buffer (▪), water at pH 5.1 (▴) and pH 6.8 phosphate buffer (). The percent tetrabenazine dissolved is shown on the y-axis with the time (hours) shown on the x-axis. Dissolution was performed using paddles and sinkers, with 45 μm in-line large surface filter tips and a 15 ml pull volume.

### DESCRIPTION

Certain embodiments of the present invention relate to a tetrabenazine composition. The tetrabenazine composition includes a safe and pharmaceutically effective amount of tetrabenazine and a release-retarding agent. In such embodiments, the composition provides for fewer incidences of hyperkinetic movement (e.g., chorea associated with Huntington's disease or tics associated with Tourette's syndrome) and/or less severe hyperkinetic movement.

Certain embodiments of the present invention relate to methods of reducing incidences of hyperkinetic movement and/or methods of reducing the severity of hyperkinetic movement. The methods include administering a safe and pharmaceutically effective amount of the tetrabenazine composition to a subject in need of tetrabenazine administration.

Certain embodiments of the present invention relate to methods of treating a condition. The method includes administering a safe and pharmaceutically effective amount of the tetrabenazine composition to a subject in need of tetrabenazine administration. In such embodiments, the tetrabenazine composition provides for fewer incidences of hyperkinetic movement and/or reduces the severity of hyperkinetic movement.

Certain embodiments of the present invention relate to a method of treating a subject at risk of hyperkinetic movement. The method include administering to the subject a safe and effective amount of the tetrabenazine composition.

As shown herein below, it is demonstrated that a safe and pharmaceutically effective amount of a tetrabenazine composition that includes tetrabenazine and a release-retarding agent has a propensity to treat hyperkinetic movement and/or to reduce the severity of hyperkinetic movement. This allows one to reduce the incidences of hyperkinetic movement, to reduce the severity of such hyperkinetic movement, to treat subjects who would otherwise not be candidates for tetrabenazine therapy because of the adverse effects associated with tetrabenazine administration, and/or to treat a subject with lower doses of tetrabenazine than would be possible and safe with a formulation containing an equivalent molar amount of tetrabenazine.

Certain embodiments relate to compositions that include a release-retarding agent, tetrabenazine:

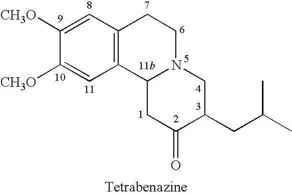

and pharmaceutically acceptable carriers, excipients and/or diluents.

In certain embodiments of the present invention, the tetrabenazine can be in the form of its anhydrous, hydrated, and solvated forms; in the form of prodrugs or metabolites; and in the form of individually optically active isomers of tetrabenazine, such as for example the RR, SS, RS, SR and any mixture thereof, for example, the racemic mixture of the RR and SS isomers.

As discussed infra and generally known in the art, appropriate dissolution medium and appropriate conditions for assaying the dissolution characteristics of pharmaceutical dosage forms such as tablets are well known in the art and are contained in the United States Pharmacopoeia and its European or Japanese counterparts, and include by way of example dissolution in USP Type 1 apparatus (Rotating Basket Method) in 900 ml water; 0.1 N HCl; 0.1N HCl+0.1% Cetrimide; USP buffer pH 1.5; Acetate buffer pH 4.5; Phosphate Buffer pH 6.5; or Phosphate Buffer pH 7.4 at 75 RPM at 37 degrees C±0.5 degrees C. Additionally, other examples of appropriate dissolution media include USP-3 media and USP-3 dissolution conditions e.g,, SGF pH 1.2; Acetate buffer pH 4.5 and Phosphate Buffer pH 6.8.

Certain embodiments of the present invention contemplate the use of tetrabenazine, to produce once-daily administrable tablets or other dosage forms that are bioequivalent to Xenazine® (tetrabenazine) tablets, as defined by FDA criteria when administered once daily to a subject in need thereof. In particular, at least one of the Tmax, Cmax or AUC profile of certain embodiments of the present invention is within 80-125% of Xenazine® when administered once daily to a subject in need thereof.

Certain embodiments of the present invention contemplate the use of 10 mg, 12.5 mg, 15 mg, 20 mg, 25 mg, 30 mg and/or 50 mg of tetrabenazine, to produce once-daily administrable tablets or other dosage forms that are bioequivalent to Xenazine® (tetrabenazine) tablets, as defined by FDA criteria when administered once daily to a subject in need thereof. In particular at least one of the Tmax, Cmax, or AUC profile of certain embodiments of the present invention is within 80-125% of Xenazine® when administered once daily to a subject in need thereof. In certain embodiments, these tetrabenazine formulations can have a significant food effect.

Certain embodiments of the present invention relate to a once daily tetrabenazine composition. The once daily tetrabenazine composition includes a safe and pharmaceutically effective amount of tetrabenazine and a release-retarding agent. In such embodiments, the composition provides for fewer incidences of hyperkinetic movement (e.g., chorea associated with Huntington's disease or tics associated with Tourette's syndrome) and/or less severe hyperkinetic movement. In further specific embodiments of the present invention, the once daily tetrabenazine composition can include 10 mg, 12.5 mg, 15 mg, 20 mg, 25 mg, 30 mg or 50 mg of tetrabenazine.

Certain embodiments of the present invention relate to methods of reducing incidences of hyperkinetic movement and/or methods of reducing the severity of hyperkinetic movement. The methods include administering a safe and pharmaceutically effective amount of the once daily tetrabenazine composition to a subject in need of tetrabenazine administration. In further specific embodiments of the present invention, the once daily tetrabenazine composition can include 10 mg, 12.5 mg, 15 mg, 20 mg, 25 mg, 30 mg or 50 mg of tetrabenazine.

Certain embodiments of the present invention relate to methods of treating a condition. The method includes administering, once a day, a safe and pharmaceutically effective amount of the once daily tetrabenazine composition to a subject in need of tetrabenazine administration. In such embodiments, the tetrabenazine composition provides for fewer incidences of hyperkinetic movement and/or reduces the severity of hyperkinetic movement. In further specific embodiments of the present invention, the once daily tetrabenazine composition can include 10 mg, 12.5 mg, 15 mg, 20 mg, 25 mg, 30 mg or 50 mg of tetrabenazine.

Certain embodiments of the present invention relate to a method of treating a subject at risk of hyperkinetic movement. The method include administering, once daily, to the subject a safe and effective amount of the once daily tetrabenazine composition. In further specific embodiments of the present invention, the once daily tetrabenazine composition can include 10 mg, 12.5 mg, 15 mg, 20 mg, 25 mg, 30 mg or 50 mg of tetrabenazine.

Certain embodiments of the present invention include modified-release formulations, controlled-release formulations, extended release formulations, prolonged-release formulations, delayed release formulations, enhanced absorption formulations, pulsatile release formulations, gastro-retention formulations using floatable microparticles, and/or sustained-re lease formulations.

Certain embodiments of the present invention include immediate release formulations. In such embodiments, the tetrabenazine formulations include tetrabenazine and a release-retarding agent. The tetrabenazine formulations may act as immediate release formulations when administered within about 1 hour, before or after, of ingesting food (e.g., a high-fat food or a high-fat beverage).

In a particular implementation of certain embodiments of the present invention, the tetrabenazine composition includes multiparticulates.

Certain embodiments of the present invention include controlled release matrix tablet formulations.

In a more particular implementation of certain embodiments of the invention, the pharmaceutical composition includes a safe and effective amount of tetrabenazine and a release-retarding agent.

In certain embodiments of the present invention, the pharmaceutical composition is an oral unit dosage form. Such an oral unit dosage form can contain a variety of tetrabenazine doses, for example, a range of doses from about 1 mg to about 100 mg, or from about 3 mg to about 75 mg tetrabenazine. In further embodiments of the present invention, the unit dosage form: (i) contains about 10 mg of tetrabenazine, or (ii) contains about 12.5 mg of tetrabenazine, or (iii) contains about 15 mg of tetrabenazine, or (iv) contains about 20 mg of tetrabenazine, or (v) contains about 25 mg of tetrabenazine, or (vi) contains about 30 mg of tetrabenazine, or (vii) contains about 50 mg of tetrabenazine.

In certain embodiments of the present invention, the tetrabenazine is the sole therapeutic agent. In other embodiments, the oral unit dosage form contains tetrabensazine and an additional therapeutic agent, for example, amantadine, pimozide, haloperidol and/or clonidine.

In certain embodiments of the present invention, the pharmaceutical composition is a tablet, powder, capsule, sachet, troche or lozenge.

In certain embodiments of the present invention, the pharmaceutical composition further includes at least one of a diluent, disintegrant, glidant and lubricant.

In certain embodiments of the present invention, the diluent is a sugar. In a further embodiment of the present invention, the sugar is lactose. In a further embodiment of the present invention, the diluent is included in an amount of about 15% (w/w) to about 60% (w/w) of the composition. In a further embodiment of the present invention, the diluent is included in an amount of about 30% (w/w) to about 40% (w/w) of the composition.

In certain embodiments of the present invention, the disintegrant is starch. In a further embodiment of the present invention, the disintegrant is included in an amount of about 7.5% (w/w) to about 45% (w/w) of the composition. In a further embodiment of the present invention, the disintegrant is included in an amount of about 15% (w/w) to about 30% (w/w) of the composition.

In certain embodiments of the present invention, the glidant is talc and/or colloidal silicon dioxide. In a further embodiment of the present invention, the glidant is included in an amount of about 0.5% (w/w) to about 3% (w/w) of the composition. In a further embodiment of the present invention, the glidant is included in an amount of about 1% (w/w) to about 2% (w/w) of the composition.

In certain embodiments of the present invention, the lubricant is magnesium stearate. In a further embodiment of the present invention, the lubricant is included in an amount of about 0.05 (w/w) to about 3% (w/w) of the composition. In a further embodiment of the present invention, the lubricant is included in an amount of about 0.1 (w/w) to about 2% (w/w) of the composition.

In certain embodiments of the present invention, the tetrabenazine is included in an amount of about 5% (w/w) to about 20% (w/w) of the composition.

In certain embodiments of the present invention, the pharmaceutical composition exhibits a food effect.

In certain embodiments of the present invention, the release-retarding agent includes an agent selected from a cellulose derivative, a polyoxyalkylene block co-polymer, and mixtures thereof.

In certain embodiments of the present invention: (i) the release-retarding agent includes a cellulose derivative; or (ii) the release-retarding agent is a cellulose derivative. In further embodiments of the present invention, the release-retarding agent includes hydroxypropyl methyl cellulose (HPMC). In further embodiments of the present invention, the release-retarding agent is included in an amount of about 10% (w/w) to about 60% (w/w) of the composition. In further embodiments of the present invention, the release-retarding agent is included in an amount of about 20% (w/w) to about 40% (w/w) of the composition.

In a more particular implementation of certain embodiments of the invention, a safe and effective amount of the pharmaceutical composition is administered to a subject for a period of time effective to treat a hyperkinetic movement disorder.

In certain embodiments of the present invention, the hyperkinetic movement disorder includes at least one of chorea associated with Huntington's disease, Huntington's disease, hyperkinetic movement, hemiballismus, senile chorea, tic disorders, tardive dyskinesia, myoclonus, dystonia and Tourette's syndrome.

In certain embodiments of the present invention, the pharmaceutical composition is administered within about 1 hour of ingesting food. In further embodiments of the present invention, the pharmaceutical composition is administered within about 1 hour before ingesting food. In further embodiments of the present invention, the pharmaceutical composition is administered within about 1 hour after ingesting food.

In certain embodiments of the present invention, the pharmaceutical composition is administered within about 1 hour of ingesting a high-fat food or a high-fat beverage. In further embodiments of the present invention, the pharmaceutical composition is administered within about 1 hour before ingesting a high-fat food or a high-fat beverage. In further embodiments of the present invention, the pharmaceutical composition is administered within about 1 hour after ingesting a high-fat food or a high-fat beverage.

In certain embodiments of the present invention, the Fed/Fast ratio of the systemic exposure (AUC) of each of the active metabolites alpha- and beta-dihydrotetrabenazine is at least about 140%.

In certain embodiments of the present invention, the Fed/Fast ratio of the peak concentration (Cmax) of each of the active metabolites alpha- and beta-dihydrotetrabenazine is at least about 220%.

In certain embodiments of the present invention, the Cmax of each of the active metabolites alpha- and beta-dihydrotetrabenazine in the blood is obtained between about 3 hours and about 6 hours after administration of the composition.

In certain embodiments of the present invention, the pharmaceutical composition is administered about once a day (q.d.).

In certain embodiments of the present invention, the pharmaceutical composition is administered about twice a day (b.i.d.).

In certain embodiments of the present invention, a safe and effective amount of the pharmaceutical composition is administered to a subject for a period of time effective to treat a hyperkinetic movement disorder; wherein the method of treating the hyperkinetic movement disorder in a patient in need thereof reduces the incidence of hyperkinetic movement in the patient.

In certain embodiments of the present invention, a safe and effective amount of the pharmaceutical composition is administered to a subject for a period of time effective to treat a hyperkinetic movement disorder; wherein the method of treating the hyperkinetic movement disorder in a patient in need thereof reduces the severity of hyperkinetic movement in the patient.

In certain embodiments of the present invention, a safe and effective amount of the pharmaceutical composition is administered to a subject for a period of time effective to treat a hyperkinetic movement disorder; wherein the patient experiences a lower incidence of adverse effects.

In certain embodiments of the present invention, a safe and effective amount of the pharmaceutical composition is administered to a subject for a period of time effective to treat a hyperkinetic movement disorder; wherein the patient experiences a lower severity of adverse effects.

In further embodiments of the present invention, the adverse effects include at least one of akathisia, depression, suicidal thoughts, suicidal behavior (suicidality), dizziness, drowsiness, sedation, somnolence, insomnia, fatigue, nervousness, anxiety, nausea and Parkinsonism.

Definitions

The following definitions are provided in order to more specifically describe the invention. Otherwise all terms are to be accorded their ordinary meaning as they would be construed by one of ordinary skill in the art, i.e. pharmaceutical drug formulations.

The term “incidences of hyperkinetic movement” as used herein is defined to mean the number of minor motor abnormalities (e.g., unintentionally initiated, uncompleted and/or uncontrollable movements) as determined by behavioral observations of unintentional movements of any part of the body, or by using the unified Huntington's disease rating scale which provides an overall rating system based on motor, behavioral, cognitive, and functional assessments.

The term “reducing incidences of hyperkinetic movement” or “fewer incidences of hyperkinetic movement,” as used herein, is defined to mean that the administration of compositions of the present invention containing tetrabenazine results in fewer incidences of hyperkinetic movement.

In specific embodiments of the invention, the reduction of incidences of hyperkinetic movement refers to the incidences of hyperkinetic movement upon administration of a composition of the present invention, as compared to the administration of a composition containing an equivalent molar amount of tetrabenazine, when exposed to identical conditions and after identical periods of time.

In further specific embodiments of the invention, the reduction of incidences of hyperkinetic movement refers to the incidences of hyperkinetic movement upon administration of a composition of the present invention, as compared to the administration of Xenazine® (tetrabenazine) tablets containing an equivalent molar amount of tetrabenazine, when exposed to identical conditions and after identical periods of time.

The term “severity of hyperkinetic movement” as used herein is defined to mean the degree of minor motor abnormalities (e.g., unintentionally initiated, uncompleted and/or uncontrollable movements) as determined by behavioral observations of unintentional movements of any part of the body, or by using the unified Huntington's disease rating scale which provides an overall rating system based on motor, behavioral, cognitive, and functional assessments.

The term “reducing severity of hyperkinetic movement” or “lower severity of hyperkinetic movement,” as used herein, is defined to mean that the administration of compositions of the present invention containing tetrabenazine results in less severe hyperkinetic movement.

In specific embodiments of the invention, the reduction of severity of hyperkinetic movement refers to the severity or degree of hyperkinetic movement upon administration of a composition of the present invention, as compared to the administration of a composition containing an equivalent molar amount of tetrabenazine, when exposed to identical conditions and after identical periods of time.

In further specific embodiments of the invention, the reduction of severity of hyperkinetic movement refers to the severity or degree of hyperkinetic movement upon administration of a composition of the present invention, as compared to the administration of Xenazine® (tetrabenazine) tablets containing an equivalent molar amount of tetrabenazine, when exposed to identical conditions and after identical periods of time.

The terms “adverse effects associated with tetrabenazine” or “side effects of tetrabenazine” as used herein are used interchangeably, and mean the adverse drug reactions resulting from the administration of tetrabenazine or a mixture of tetrabenazine with one or more other drugs, non-limiting examples of which include, e.g., akathisia, depression, suicidal thoughts, suicidal behavior (suicidality), dizziness, drowsiness, sedation, somnolence, insomnia, fatigue, nervousness, anxiety, nausea and Parkinsonism.

The term “depression” as used herein refers to any nervous system disorder and/or mental condition characterized by, but not limited to, the following symptoms: depressed mood, anhedonia, feelings of intense sadness and despair, mental slowing, loss of concentration, pessimistic worry, agitation, self-deprecation, disturbed sleep patterns (e.g. insomnia, loss of REM sleep, or hypersomnia), anorexia, changes in appetite and weight loss or weight gain, Psychomotor agitation, decreased energy, decreased libido, and changes in hormonal circadian rhythms, withdrawal, altered daily rhythms of mood, activity, temperature and neuroendocrine function, and combinations thereof. Non-limiting examples of “depression” include major depressive disorder, bipolar depressed mood disorder, adjustment mood disorder, and post-partum mood disorder.

The term “condition” as used herein when referring to the administration of tetrabenazine, means a condition, disease or disorder which can be treated with tetrabenazine. Non-limiting examples of which include Huntington's disease, hyperkinetic movement, hemiballismus, senile chorea, tic disorders, tardive dyskinesia, myoclonus, dystonia and Tourette's syndrome.

The terms “treatment,” “treating” or “treat” as used herein when referring to a condition, and as understood in the art, are defined to mean an approach for obtaining beneficial or desired results, including clinical results. Beneficial or desired clinical results can include, but are not limited to, alleviation of one or more symptoms of the condition, diminishment of extent of disease or condition, stabilized (i.e. not worsening) state of disease or condition, preventing spread of disease, delay or slowing of disease progression, palliation of the disease state, and remission (whether partial or total), whether detectable or undetectable. “Treatment” can also mean prolonging survival of a subject as compared to the expected survival of the subject if not receiving treatment.

The terms “at risk,” “patient at risk,” and “a subject at risk of hyperkinetic movement” refers to those subjects that either through existing illness, prior medical illness, past history of seizures, prior exposure, testing, dosing or other administration of tetrabenazine are known to have a greater propensity to have hyperkinetic movement, compared to a subject who does not exhibit hyperkinetic movement under the same or similar conditions and/or a subject who based on a clinical evaluation of the subject's health, other medications and/or treatments is expected to have a greater propensity to have hyperkinetic movement.

The term “palliating” as used herein when referring to a condition means that the extent and/or undesirable clinical manifestations of a condition or disease state are lessened and/or time course of the progression is slowed or lengthened, as compared to not treating the condition.

The terms “subject” or “patient” as used herein are used interchangeably and mean all members of the animal kingdom (e.g. humans).

The term “subject in need of” as used herein when referring to tetrabenazine administration, means a subject having a condition that can be treated with tetrabenazine.

The term “effective amount” or “pharmaceutically effective amount” as used herein are used interchangeably, and are defined to mean the amount or quantity of the active drug (e.g. tetrabenazine) or polymorph or isomer thereof, which is sufficient to elicit an appreciable biological response when administered to a patient. It will be appreciated that the precise therapeutic dose will depend on the age and condition of the patient and the nature of the condition to be treated and will be at the ultimate discretion of the attendant physician.

The term “pharmaceutically acceptable” as used herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The term “dissolution profile” or “release profile” as used herein are used interchangeably in this application, and are defined to mean a quality control test conducted according to instructions found in the United States Pharmacopoeia (“USP”), i.e. using a USP apparatus design with a dissolution medium as found in the USP. Dissolution tests in-vitro measure the rate and extent of dissolution of the active drug in an aqueous dissolution medium. The dissolution rate or in-vitro release rates of drug from the modified release dosage forms of the present invention can be measured using one of many USP apparatus designs and dissolution media; non-limiting examples of which include a USP Type 1 apparatus design or USP Type 2 apparatus design, with a dissolution medium selected from water; 0.1N HCl; 0.1N HCl with added Sodium Chloride (e.g. 15.7 g NaCl/Litre); 0.1N HCl with added 0.1% Cetrimide; USP Buffer pH 1.5; Acetate Buffer pH 4.5; Phosphate Buffer pH 6.5; Phosphate Buffer pH 6.8; and Phosphate Buffer pH 7.4. The terms “% released” and “% dissolved”, when referring to a dissolution profile, are used interchangeably in this application and are defined to mean the extent (%) of active drug released in an aqueous dissolution medium (in vitro).

The terms “active,” “active agent,” “active pharmaceutical agent,” “active drug” or “drug” as used herein are used interchangeably and are defined to mean any active pharmaceutical ingredient (“API”), including its pharmaceutically acceptable salts (non-limiting examples of which include the hydrochloride salts, the hydrobromide salts, the hydroiodide salts, and the saccharinate salts), as well as the anhydrous, hydrated, and solvated forms, polymorphs, prodrugs, metabolites, and the individually optically active enantiomers of the API. The active drug includes the molecule or ion and the appended portions of the molecule that cause the drug to be an ester or salt of the molecule.

The term “moiety” as used herein is defined to mean the molecule or ion, excluding those appended portions of the molecule that cause the drug to be an ester or salt of the molecule, responsible for the physiological or pharmacological action of the drug substance.

The terms “formulation” or “composition” as used herein are used interchangeably and refer to the drug in combination with pharmaceutically acceptable carriers and additional inert ingredients. The formulation can be administrable by a variety of means.

The term “dosage form” as used herein is defined to mean a pharmaceutical preparation or system in which a dose of at least one active drug is included. For example, a dosage form can include at least one modified release dosage form, at least one osmotic dosage form, at least one erosion modified release dosage form, at least one dissolution modified release dosage form, at least one diffusion modified release dosage form, at least one modified release matrix core, at least one modified release matrix core coated with at least one modified release coat, at least one enteric coated dosage form, at least one dosage form surrounded by at least one osmotic subcoat, capsules, minitablets, caplets, uncoated microparticles, microparticles coated with at least one modified release coat, or any combination thereof.

The term “medicament” as used herein refers to oral and non-oral dosage forms, including but not limited to, all modified release dosage forms, osmosis controlled release systems, erosion controlled release systems, dissolution controlled release systems, diffusion controlled release systems, matrix tablets, enteric coated tablets, single and double coated tablets (including the extended release and enhanced absorption tablets as described herein), capsules, minitablets, caplets, coated beads, granules, spheroids, pellets, microparticles, suspensions, topicals such as transdermal and transmucosal compositions and delivery systems (containing or not containing matrices), injectables, and inhalable compositions.

“Modified release dosage forms” as used herein is defined (e.g. as by the United States Pharmacopoeia “USP”) as those whose drug release characteristics of time course and/or location are chosen to accomplish therapeutic or convenience objectives not offered by conventional immediate release dosage forms. The rate of release of the active drug from a modified release dosage form is controlled by features of the dosage form and/or in combination with physiologic or environmental conditions rather than by physiologic or environmental conditions alone. The modified release dosage forms of certain embodiments can be contrasted with conventional immediate release dosage forms which typically produce large maximum/minimum plasma drug concentrations (Cmax/Cmin) due to rapid absorption of the drug into the body (i.e., in-vivo, relative to the drug's therapeutic index; i.e., the ratio of the maximum drug concentration needed to produce and maintain a desirable pharmacological response). In conventional immediate release dosage forms, the drug content is released into the gastrointestinal tract within a short period of time, and plasma drug levels peak shortly after dosing. The design of conventional immediate release dosage forms is generally based on getting the fastest possible rate of drug release, and therefore absorbed, often at the risk of creating undesirable dose related side effects. The modified release dosage forms of certain embodiments of the invention, on the other hand, improve the therapeutic value of the active drug by reducing the ratio of the maximum/minimum plasma drug concentration (Cmax/Cmin) while maintaining drug plasma levels within the therapeutic window. The modified release dosage forms of certain embodiments attempt to deliver therapeutically effective amounts of tetrabenazine as a once-daily dose so that the ratio Cmax/Cmin in the plasma at steady state is less than the therapeutic index, and to maintain drug levels at constant effective levels to provide a therapeutic benefit over a period of time (e.g. 24-hour period). The modified release dosage forms of certain embodiments of the invention, therefore, avoid large peak-to-trough fluctuations normally seen with conventional or immediate release dosage forms and can provide a substantially flat serum concentration curve throughout the therapeutic period. Modified-release dosage forms can be designed to provide a quick increase in the plasma concentration of the tetrabenazine which remains substantially constant within the therapeutic range of tetrabenazine for a period of time (e.g. 24-hour period). Alternatively, modified-release dosage forms can be designed to provide a quick increase in the plasma concentration of the drug, which although may not remain constant, declines at a rate such that the plasma concentration remains within the therapeutic range for a period of time (e.g. 24-hour period). The modified release dosage forms of certain embodiments of the invention can be constructed in many forms known to one of ordinary skill in the drug delivery arts and described in the prior art. The USP considers that the terms controlled release, prolonged release and sustained release are interchangeable. Accordingly, the terms “modified-release”, controlled-release”, “control-releasing”, “rate-controlled release”, “extended release”, “prolonged-release”, and “sustained-release” are used interchangeably herein. For the discussion herein, the definition of the term “modified-release” encompasses the scope of the definitions for the terms “extended release”, “enhanced-absorption”, “controlled release”, “sustained release” and “delayed release”.

“Controlled release dosage forms”, “control-releasing dosage forms”, “rate-controlled release dosage forms”, or dosage forms which exhibit a “controlled release” of the tetrabenazine, as used herein are used interchangeably in this application and are defined to mean dosage forms which release the tetrabenazine in a controlled manner per unit time in-vivo. For example, controlled release dosage forms can be administered once daily, and release the tetrabenazine at a controlled rate and provide plasma concentrations of the drug that remain controlled with time within the therapeutic range of tetrabenazine over a 24-hour period. The rate of release of the tetrabenazine from a controlled release dosage form is controlled by features of the dosage form and/or in combination with physiologic or environmental conditions rather than by physiologic or environmental conditions alone. The controlled release dosage forms of certain embodiments of the invention can be contrasted to immediate release dosage forms which typically produce large maximum/minimum plasma drug concentrations (Cmax/Cmin) due to rapid absorption of the drug into the body i.e., in-vivo, relative to the drug's therapeutic index i.e., the ratio of the maximum drug concentration needed to produce and maintain a desirable pharmacological response. In immediate release dosage forms, the drug content is released into the gastrointestinal tract within a short period of time, and plasma drug levels peak shortly after dosing. The design of immediate release dosage forms is generally based on getting the fastest possible rate of drug release, and therefore absorbed, often at the risk of creating undesirable dose related side effects. The controlled release dosage forms of certain embodiments of the invention, on the other hand, improve the therapeutic value of the active drug by reducing the ratio of the maximum/minimum plasma drug concentration (Cmax/Cmin) while maintaining drug plasma levels within the therapeutic window. The controlled release dosage forms of certain embodiments of the invention attempt to deliver therapeutically effective amounts of tetrabenazine as a dose administered at least once-daily so that the ratio Cmax/Cmin in the plasma at steady state is less than the therapeutic index, and to maintain drug levels at constant effective levels to provide therapeutic benefit over a period of time (e.g. a 24-hour period). The controlled release dosage forms of certain embodiments of the invention, therefore, avoid large peak-to-trough fluctuations normally seen with immediate release dosage forms and provide a substantially flat serum concentration curve throughout the therapeutic period. The controlled release dosage forms of certain embodiments of the invention can be constructed in many forms known to one of ordinary skill in the drug delivery arts and described in the prior art such as for example, osmotic dosage forms, multiparticulate dosage forms, and gastric retention dosage forms.

“Sustained-release dosage forms” or dosage forms which exhibit a “sustained-release” of tetrabenazine as used herein is defined to mean dosage forms administered at least once-daily that provide a release of tetrabenazine sufficient to provide a therapeutic dose soon after administration, and then a gradual release over a period of time such that the sustained-release dosage form provides a therapeutic benefit over a period of time (e.g. a 12-hour or 24-hour period).

“Extended-release dosage forms” or dosage forms which exhibit an “extended release” of tetrabenazine as used herein is defined to mean dosage forms administered at least once-daily that release the tetrabenazine slowly, so that plasma concentrations of the tetrabenazine are maintained at a therapeutic level for an extended period of time such that the extended release dosage form provides therapeutic benefit over a period of time (e.g. 24-hour period).

“Delayed-release dosage forms” or dosage forms which exhibit a “delayed release” of tetrabenazine as used herein is defined to mean dosage forms administered at least once-daily that do not effectively release drug immediately following administration but at a later time. Delayed-release dosage forms provide a time delay prior to the commencement of drug-absorption. This time delay is referred to as “lag time” and should not be confused with “onset time” which represents latency, that is, the time required for the drug to reach minimum effective concentration.

“Enhanced absorption dosage forms” or dosage forms which exhibit an “enhanced absorption” of the active drug as used herein is defined to mean dosage forms that when exposed to like conditions, will show higher release and/or more absorption of the tetrabenazine as compared to other dosage forms with the same or higher amount of tetrabenazine. The same therapeutic effect can be achieved with less tetrabenazine in the enhanced absorption dosage form as compared to other dosage forms.

The term “microparticle”, as used herein refers to a drug formulation in discrete particulate form, and is interchangeable with the terms “microspheres”, “spherical particles”, “microcapsules”, “particles”, “multiparticulates”, “granules”, “spheroids”, beads” and “pellets”.

The term “tablet” as used herein refers to a single dosage form, i.e. the single entity containing the active pharmaceutical agent that is administered to the subject. The tern “tablet” also includes a tablet that may be the combination of one or more “minitablets”.

The term “orally disintegrating tablet” (ODT) is a drug dosage form formulated and designed to be dissolved on the tongue withing about 30 seconds, rather than swallowed hole. The ODT serves as an alternative dosage form for patients who experience dysphagia (difficulty in swallowing).

The term “controlled release matrix” as used herein is defined to mean a dosage form in which the tetrabenazine, is dispersed within a matrix, which matrix can be either insoluble, soluble, or a combination thereof. Controlled release matrix dosage forms of the insoluble type are also referred to as “insoluble polymer matrices”, “swellable matrices”, or “lipid matrices” depending on the components that make up the matrix. Controlled release matrix dosage forms of the soluble type are also referred to as “hydrophilic colloid matrices”, “erodible matrices”, or “reservoir systems”. Controlled release matrix dosage forms of the invention refer to dosage forms including an insoluble matrix, a soluble matrix or a combination of insoluble and soluble matrices in which the rate of release is slower than that of an uncoated non-matrix conventional or immediate release dosage forms or uncoated “normal release matrix” dosage forms. Controlled release matrix dosage forms can be coated with a “control-releasing coat” to further slow the release of the tetrabenazine from the controlled release matrix dosage form. Such coated controlled release matrix dosage forms can exhibit “modified-release”, controlled-release”, “sustained-release”, “extended-release”, “prolonged-release”, “delayed-release” or combinations thereof of the active drug.

The term “normal release matrix” as used herein is defined to mean dosage forms in which the tetrabenazine, is dispersed within a matrix, which matrix can be either insoluble, soluble, or combinations thereof but constructed such that the release of the active drug mimics the release rate of an uncoated non-matrix conventional or immediate release dosage form including the drug. The release rate from normal release matrix dosage forms can be slowed down or modified in conjunction with a controlled release coat.

The terms “osmotic dosage form”, “osmotic delivery device”, “modified release osmotic dosage form” or “controlled release osmotic dosage form” as used herein are used interchangeably in this application, and are defined to mean dosage forms which dispense the tetrabenazine, all or in part as a result of the presence of an osmotic agent in the dosage form driving solvent (e.g. water, dissolution media, gastric fluid, intestinal fluid, or mixtures thereof) into the core of the dosage form, which subsequently facilitates the release of drug from the core.

The term “osmosis” as used herein refers to the flow of a solvent through a selectively-permeable membrane (e.g. controlled release coat) from a region of high solvent potential to a region of low solvent potential. The selectively-permeable membrane is permeable to the solvent, but not to the solute, resulting in a pressure gradient across the membrane. Non-limiting examples of selectively-permeable membranes include semipermeable membranes, and microporous, asymmetric membranes (which can be permeable, semipermeable, perforated. or unperforated) and can deliver the active drug(s) by osmotic pumping, diffusion or the combined mechanisms of diffusion and osmotic pumping. Thus, in principle, osmosis controlled release of the active drug(s) involves osmotic transport of an aqueous media into the osmotic dosage form followed by dissolution of the active drug(s) and the subsequent transport of the saturated solution of the active drug by osmotic pumping of the solution through at least one passageway in the selectively-permeable membrane and/or by diffusion through the selectively-permeable membrane.

The term “osmotic pressure gradient” as used herein is defined to mean the difference in hydrostatic pressure produced by a solution in a space divided by a selectively-permeable membrane due to a differential in the concentrations of solute.

The terms “osmotic agent”, “osmagent”, “osmotically effective solute”, “osmotic enhancer” “osmotically effective compounds”, “osmotic solutes”, “osmopolymer” and “osmotic fluid imbibing agents” as used herein are used interchangeably, and define any material that is soluble (i.e. can be partially or totally solubilized) or swellable in a solvent (e.g. water) that enters the composition, and which exhibits an osmotic pressure gradient across the selectively-permeable membrane (e.g. controlled release coat), thus increasing the hydrostatic pressure inside the osmotic dosage form.

The terms “controlled release coat”, “control releasing coat”, “modified release coat” and “rate-controlling coat” as used herein are used interchangeably in this application, and are defined to mean a functional coat which includes at least one modified release polymer. Non-limiting examples of modified release polymers include pH independent polymers, pH dependent polymers (such as for example enteric or reverse enteric types), soluble polymers, insoluble polymers, lipids, lipidic materials, and mixtures thereof. When applied onto a dosage form, the controlled release coat can modify (e.g. slow) the rate of release of the active drug. For example, the controlled release coat can be designed such that when the coat is applied onto a dosage form, the dosage form in conjunction with the controlled release coat, exhibits a “modified-release,” “controlled-release,” “sustained-release,” “extended-release” and/or “delayed-release” profile. Combinations thereof are permissible. The controlled release coat can optionally include additional materials that can alter the functionality of the controlled release coat. The term “modified release” is interchangeable with the terms “controlled release,” “control releasing” and “rate controlling.” The term “coat” is interchangeable with the term “coating.”

The term “enteric coat” as used herein is defined to mean a coating or barrier applied to a dosage form that can control the location in the digestive system where the active drug(s) is absorbed. For example, an enteric coating can be used to: (i) protect the drug from the destructive action of the enzymes or low pH environment of the stomach; (ii) prevent nausea or bleeding associated with the irritation of the gastric mucosa by the drug; and/or (iii) deliver the drug in an undiluted form in the intestine. Based on these criteria, in certain embodiments, the enteric coated dosage form can be regarded as a type of delayed release dosage form. They differ from sustained release dosage forms in that with sustained release dosage forms, the drug release is extended over a period of time to maintain therapeutic blood levels and to decrease the incidence of side effects caused by a rapid release; whereas, with enteric coatings, the primary objective is to confine the release of the drug to a predetermined region of the gastrointestinal tract. Enteric coatings work by presenting a surface that is substantially stable at acidic pH, but breaks down at higher pH to allow release of the drug in the intestine.

The term “reverse enteric coat” as used herein is defined to mean a coating or barrier applied to a dosage form that can control the location in the digestive system where the active drug(s) is absorbed. Reverse enteric coatings work by presenting a surface that is substantially stable at a pH above 5, but breaks down at a pH up to about 5, to allow release of the drug in gastric juices. As such, the drug is soluble, swellable and/or permeable in digestive fluids (e.g., pH of about 5), and is substantially insoluble and/or stable at a pH higher than 5.

The term “enteric polymer” as used herein is defined to mean a polymeric substance that when used in an enteric coat formulation, is substantially insoluble and/or substantially stable under acidic conditions exhibiting a pH of less than about 5 and which are substantially soluble or can decompose under conditions exhibiting a pH of about 5 or more. Non-limiting examples of such enteric polymers include carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butyrate phthalate, polyvinyl acetal phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate and mixtures thereof. Enteric polymers can be used individually or in combination with other hydrophobic or hydrophilic polymers in an enteric coat, a normal release matrix core, a controlled release matrix core, and/or in a controlled release coat. Enteric polymers can be combined with other pharmaceutically acceptable excipients to either facilitate processing of a coat including the enteric polymer or to alter the functionality of the coat.

The term “functional coat” as used herein is defined to mean a coating that affects the rate of release in-vitro or in-vivo of the active drug(s).

The term “non-functional coat” as used herein is defined to mean a coating that does not substantially affect the rate of release in-vitro or in-vivo of the active drug, but can enhance the chemical, biological, physical stability characteristics, or the physical appearance of the modified release dosage form.

The term “core” as used herein is defined to mean a solid vehicle in which at least one active drug is uniformly or non-uniformly dispersed. The core can be formed by methods and materials well known in the art, such as for example by compressing, fusing, or extruding the active drug together with at least one pharmaceutically acceptable excipient. The core can be manufactured into, for example, a homogenous or non-homogenous unitary core, a multiparticle, or a plurality of microparticles compressed into a unitary core. Non-limiting examples of cores include microparticle cores, matrix cores, and osmotic cores. The core(s) can be coated with at least one functional coat and/or non-functional coat.

The terms “modified release matrix core”, “controlled release matrix core” or “matrix core” when referring to a controlled release matrix dosage form, as used herein are used interchangeably, and are defined to mean a core in which at least one active drug is dispersed within a matrix which controls or delays the release of the active drug over a 24-hour period so as to allow a composition including the modified release matrix core to be administered as a once-a-day composition. The release rate of the active drug from the modified release matrix core can be modified by the porosity and tortuosity of the matrix, (i.e. its pore structure). The addition of pore-forming hydrophilic salts, solutes, or wicking agents can influence the release rate, as can the manipulation of processing parameters. For example, the compression force used in the manufacture of the modified release matrix core can alter the porosity of the matrix core and hence the rate of release of the active drug. It will be understood by one of ordinary skill in the art of drug delivery that a more rigid matrix will be less porous and hence release the active drug more slowly compared to a less rigid modified release matrix core. The modified release matrix core can include insoluble or inert matrix dosage forms, swellable matrix dosage forms, swellable and erodable matrix dosage form, hydrophobic matrix dosage forms, hydrophilic matrix dosage forms, erodable matrix dosage forms, reservoir dosage forms, or any combination thereof. The modified release matrix core can include at least one insoluble matrix, at least one swellable matrix, at least one swellable and erodable matrix, at least one hydrophobic matrix, at least one hydrophilic matrix, at least one erodable matrix, or a combination thereof in which the rate of release is slower than that of uncoated immediate-release dosage forms. Modified release matrix cores can be coated with at least one controlled release coat to further slow the release of the active drug from the modified release matrix core. Such coated modified release matrix cores can exhibit modified-release, controlled-release, sustained-release, extended-release, prolonged-release, bi-phasic release, delayed-release or combinations thereof of the active drug. Modified release matrix cores can also be coated with a non-functional soluble coat.

The term “plasticizer” as used herein includes any compounds capable of plasticizing or softening a polymer or a binder used in the present invention. The use of plasticizers is optional, and can be included in the dosage form to modify the properties and characteristics of the polymers used in the coat(s) or core of the dosage form for convenient processing during manufacture of the coat(s) and/or the core of the dosage form. Once the coat(s) and/or core have been manufactured, certain plasticizers can function to increase the hydrophilicity of the coat(s) and/or the core of the dosage form in the environment of use. During manufacture of the coat(s) and/or core, the plasticizer can lower the melting temperature or glass transition temperature (softening point temperature) of the polymer or binder. Plasticizers can be included with a polymer and lower its glass transition temperature or softening point. Plasticizers also can reduce the viscosity of a polymer. Plasticizers can impart some particularly advantageous physical properties to the dosage forms of the invention.

The terms “pore former”, “pore forming agent”, and “pore forming additive” as used herein are used interchangeably in this application, and are defined to mean an excipient that can be added to a coating (e.g. the controlled release coat), wherein upon exposure to fluids in the environment of use, the pore former dissolves or leaches from the coating to form pores, channels or paths in the coating, that can fill with the environmental fluid and allow the fluid to enter the core and dissolve the active drug, and modify the release characteristics of the formulation. The pore formers can be inorganic or organic, and include materials that can be dissolved, extracted or leached from the coating in the environment of use.

The term “steady state” as used herein means that the blood plasma concentration curve for a given drug does not substantially fluctuate after repeated doses to dose of the formulation.

“AUC” as used herein means area under the plasma concentration-time curve, as calculated by the trapezoidal rule over a time interval (e.g. complete 24-hour interval); and signifies the extent of the absorption of a drug.

“Cmax” as used herein means the highest plasma concentration of the drug attained within the dosing interval (e.g., 24 hours).

“Cmin” as used herein means the minimum plasma concentration of the drug attained within the dosing interval (e.g. 24 hours).

“Cavg” as used herein means the plasma concentration of the drug within the dosing interval (e.g. 24-hours), and is calculated as AUC/dosing interval.

“Tmax” as used herein means the time period which elapses after administration of the dosage form at which the plasma concentration of the drug attains the highest plasma concentration of drug attained within the dosing interval (e.g. 24 hours).

The term “bioequivalence” as used herein is defined as there being about a 90% or greater probability that the bioavailability (AUC) of the active drug as determined by standard methods is from about 80% to about 125% of the second orally administrable dosage form including the same dose of the active drug and that there is about 90% or greater probability that the maximum blood plasma concentration (Cmax) of the active drug as measured by standard methods is from about 80% to about 125% of the second orally administrable dosage form. For example, the reader is referred to the final version of the guidance approved by the US Food and Drug Administration at the time of filing of this patent application i.e., the March 2003 Guidance for Industry Bioavailability and Bioequivalence Studies for Orally Administered Drug Products General Considerations, U.S. Department of Health and Human Services, Food and Drug Administration, Center for Drug Evaluation and Research (CDER), for a detailed discussion on bioequivalence.

The terms “a”, “an” or “at least one” as used herein are used interchangeably in this application, and are defined to mean “one” or “one or more”.

The numerical parameters set forth in the following specification and attached claims that are modified by the term “about”, are approximations that can vary depending upon the technological properties of the particular case. For example, the term “about” can mean within an acceptable error range (e.g. standard deviations) for the particular value as determined by one of ordinary skill in the art, which will depend in part on how the value is measured or determined, e.g., the limitations of the measurement system. At the very least, and not as an attempt to limit the application of the doctrine of equivalents to the scope of the claims, each numerical parameter modified by the term “about” should at least be construed in light of the number of reported significant digits and by applying ordinary rounding techniques. The terms “about” and “approximately” as used herein are used interchangeably.

Other terms are defined as they appear in the following description and should be construed in the context with which they appear.

The present invention encompasses compositions containing safe and pharmaceutically effective levels of the tetrabenazine, that can be used for the treatment of a condition in subjects that can benefit from tetrabenazine administration, wherein the compositions containing safe and pharmaceutically effective levels of the tetrabenazine that unexpectedly provide for the reduction of incidences of and/or the reduction in severity of hyperkinetic movement.

Certain compositions containing tetrabenazine contain from about 5 mg to about 50 mg of tetrabenazine. The range of tetrabenazine of from about 5 mg to about 50 mg includes, for example all values and ranges there between, for example, 5 mg, 7.5 mg, 10 mg, 12.5 mg, 15 mg, 20 mg, 25 mg, 30 mg, 35 mg, 40 mg, 45 mg and 50 mg. For example, certain embodiments include a composition which includes 5 mg, 7.5 mg, 10 mg, 12.5 mg, 15 mg, 20 mg, 25 mg, 30 mg, 35 mg, 40 mg, 45 mg and 50 mg of tetrabenazine per unit dose.

The present invention encompasses orally administered dosage forms containing tetrabenazine. The dosages can be conveniently presented in unit dosage form and prepared by any of the methods well-known in the art of pharmacy. A “solid dosage form” as used herein, means a dosage form that is neither liquid nor gaseous. Dosage forms include solid dosage forms, such as tablets, powders, microparticles, capsules, suppositories, sachets, troches, patches and lozenges as well as liquid suspensions and elixirs. Capsule dosages contain the solid composition within a capsule that can be made of gelatin or other conventional encapsulating material.

The modified release dosage forms contemplated in the present invention can be multiparticulate or monolithic. For example, those skilled in the pharmaceutical art and the design of medicaments are aware of modified release matrices conventionally used in oral pharmaceutical compositions adopted for modified release and means for their preparation

A modified release formulation containing tetrabenazine according to the present invention can be coated with one or more functional or non-functional coatings. Non-limiting examples of functional coatings include controlled release polymeric coatings, enteric polymeric coatings, and the like. Non-functional coatings are coatings that do not substantially affect drug release, but which affect other properties; such as the enhancement of the chemical, biological or physical stability characteristics, or the enhancement of the physical appearance of the formulation.

In at least one embodiment of the present invention a tetrabenazine composition includes a controlled release polymeric coating that includes an acrylic polymer. Suitable acrylic polymers include but are not limited to acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polyacrylamide, poly(methacrylic acid anhydride), glycidyl methacrylate copolymers and mixtures thereof.

In at least one embodiment polymerizable quaternary ammonium compounds are employed in the controlled release coat, of which non-limiting examples include quaternized aminoalkyl esters and aminoalkyl amides of acrylic acid and methacrylic acid, for example β-methacryl-oxyethyl-trimethyl-ammonium methosulfate, β-acryloxy-propyl-trimethyl-ammonium chloride, trimethylaminomethyl-methacrylamide methosulfate and mixtures thereof. The quaternary ammonium atom can also be part of a heterocycle, as in methacryloxyethylmethyl-morpholiniom chloride or the corresponding piperidinium salt, or it can be joined to an acrylic acid group or a methacrylic acid group by way of a group containing hetero atoms, such as a polyglycol ether group. Further suitable polymerizable quaternary ammonium compounds include quaternized vinyl-substituted nitrogen heterocycles such as methyl-vinyl pyridinium salts, vinyl esters of quaternized amino carboxylic acids, styryltrialkyl ammonium salts, and mixtures thereof. Other polymerizable quaternary ammonium compounds useful in the present invention include acryl- and methacryl-oxyethyltrimethyl-ammonium chloride and methosulfate, benzyldimethylammoniumethyl-methacrylate chloride, diethylmethylammoniumethyl-acrylate and -methacrylate methosulfate, N-trimethylammoniumpropylmethacrylamide chloride, N-trimethlylammonium-2,2-dimethylpropyl-1-methacrylate chloride and mixtures thereof.

In at least one embodiment the acrylic polymer of the controlled release coat is comprised of one or more ammonio methacrylate copolymers. Ammonio methacrylate copolymers (such as those sold under the Trade Mark EUDRAGIT® RS and RL) are described in National Formulary (NF) XVII as fully polymerized copolymers of acrylic and methacrylic acid esters with a low content of quaternary ammonium groups. Two or more ammonio methacrylate copolymers having differing physical properties can be incorporated in the controlled release coat of certain embodiments. For example, it is known that by changing the molar ratio of the quaternary ammonium groups to the neutral (meth)acrylic esters, the permeability properties of the resultant coating can be modified.

In certain other embodiments of the present invention, the controlled release coat further includes a polymer whose permeability is pH dependent, Such as anionic polymers synthesized from methacrylic acid and methacrylic acid methyl ester. Such polymers are commercially available, e.g., from Rohm Pharma GmbH under the trade name EUDRAGIT® L and EUDRAGIT®) S. The ratio of free carboxyl groups to the esters is known to be 1:1 in EUDRAGIT® L and 1:2 in EUDRAGIT® S. EUDRAGIT® L is insoluble in acids and pure water, but becomes increasingly permeable above pH 5.0. EUDRAGIT® S is similar, except that it becomes increasingly permeable above pH 7. The hydrophobic acrylic polymer coatings can also include a polymer which is cationic in character based on dimethylaminoethyl methacrylate and neutral methacrylic acid esters (such as EUDRAGIT® E, commercially available from Rohm Pharma). The hydrophobic acrylic polymer coatings of certain embodiments of the present invention can further include a neutral copolymer based on poly (meth)acrylates, such as EUDRAGIT® NE (NE=neutral ester), commercially available from Rohm Pharma. EUDRAGIT® NE 30D lacquer films are insoluble in water and digestive fluids, but permeable and swellable.

In at least one other-embodiment of the invention, the controlled release coat includes a dispersion of poly (ethylacrylate, methyl methacrylate) 2:1 (KOLLICOAT® EMM 30 D, BASF).

In at least one other embodiment of the invention, the controlled release coat includes a polyvinyl acetate stabilized with polyvinylpyrrolidone and sodium lauryl sulfate such as KOLLICOAT® SR30D (BASF). The dissolution profile can be altered by changing the relative amounts of different acrylic resin lacquers included in the coating. Also, by changing the molar ratio of polymerizable permeability-enhancing agent (e.g., the quaternary ammonium compounds) to the neutral (meth)acrylic esters, the permeability properties (and thus the dissolution profile) of the resultant coating can be modified.

In at least one embodiment of the invention the controlled release coat includes ethylcellulose, which can be used as a dry polymer (such as ETHOCEL®, Dow Corning) solubilized in organic solvent prior to use, or as an aqueous dispersion. One suitable commercially-available aqueous dispersion of ethylcellulose is AQUACOAT® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the AQUACOAT® can be intimately mixed with a suitable plasticizer prior to use. Another suitable aqueous dispersion of ethylcellulose is commercially available as SURELEASE® (Colorcon, Inc., West Point, Pa., U.S.A.). This product can be prepared by incorporating plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

Other examples of polymers that can be used in the controlled release coat include cellulose acetate phthalate, cellulose acetate trimaletate, hydroxy propyl methylcellulose phthalate, polyvinyl acetate phthalate, polyvinyl alcohol phthalate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, ethyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, pullulan, collagen, casein, agar, gum arabic, sodium carboxymethyl cellulose, (swellable hydrophilic polymers) poly(hydroxyalkyl methacrylate) (molecular weight from about 5 k to about 5000 k), polyvinylpyrrolidone (molecular weight from about 10 k to about 360 k), anionic and cationic hydrogels, zein, polyamides, polyvinyl alcohol having a low acetate residual, a swellable mixture of agar and carboxymethyl cellulose, copolymers of maleic anhydride and styrene, ethylene, propylene or isobutylene, pectin (molecular weight from about 30 k to about 300 k), polysaccharides such as agar, acacia, karaya, tragacanth, algins and guar, polyacrylamides, POLYOX® polyethylene oxides (molecular weight from about 100 k to about 5000 k), AQUAKEEP® acrylate polymers, diesters of polyglucan, crosslinked polyvinyl alcohol and poly N-vinyl-2-pyrrolidone, hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, methyl ethyl cellulose, ethylhydroxy ethylcellulose, cellulose acetate, cellulose butyrate; cellulose propionate, gelatin, starch, maltodextrin, pullulan, polyvinyl pyrrolidone, polyvinyl alcohol, polyvinyl acetate, glycerol fatty acid esters, polyacrylamide, polyacrylic acid, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures thereof.

In at least one embodiment of the invention the dosage forms are coated with polymers in order to facilitate mucoadhesion within the gastrointestinal tract. Non-limiting examples of polymers that can be used for mucoadhesion include carboxymethylcellulose, polyacrylic acid, CARBOPOL™, POLYCARBOPHIL™, gelatin, other natural or synthetic polymers, and mixtures thereof.

In addition to the modified release dosage forms described herein, other modified release technologies known to those skilled in the art can be used in order to achieve the modified release formulations of certain embodiments of the present invention. Such formulations can be manufactured as a modified release oral formulation, for example, in a suitable tablet or multiparticulate formulation known to those skilled in the art. In either case, the modified release dosage form can optionally include a controlled release carrier which is incorporated into a matrix along with the drug, or which is applied as a controlled release coating.

Tablets

In certain embodiments of the present invention, there is provided a modified-release tablet having a core including tetrabenazine, and conventional excipients, wherein the composition including the tetrabenazine provides for the reduction of incidences of and/or severity of hyperkinetic movement. The core can be surrounded by a controlled release coat which can control the release of tetrabenazine.

Extended Release (XR) Tablets

In certain embodiments of the present invention, there is provided an extended-release (XR) tablet having a core including tetrabenazine and conventional excipients, wherein the tetrabenazine provides for the reduction of incidences of and/or severity of hyperkinetic movement. The core can be surrounded by a controlled release coat, which controls the release of tetrabenazine. The tablet optionally can include one or more additional functional or non-functional coats surrounding the core or controlled release coat.

The XR Core

The core of the extended-release tablet includes an effective amount of tetrabenazine, a binder, and a lubricant; and can contain other conventional inert excipients. The amount of the tetrabenazine present in the XR core can vary in an amount from about 5% to about 99% by weight ofthe tablet dry weight, including all values and ranges therebetween.

A binder (also sometimes called adhesive) can be added to a drug-filler mixture to increase the mechanical strength of the granules and tablets during formation. Binders can be added to the formulation in different ways: (1) as a dry powder, which is mixed with other ingredients before wet agglomeration, (2) as a solution, which is used as agglomeration liquid during wet agglomeration, and is referred to as a solution binder, and (3) as a dry powder, which is mixed with the other ingredients before compaction. In this form the binder is referred to as a dry binder. Solution binders are a common way of incorporating a binder into granules. In certain embodiments, the binder used in the XR tablets is in the form of a solution binder. Non-limiting examples of binders useful for the core include hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats. hydrocarbons. normal waxes, stearic acid, stearyl alcohol, hydrophobic and hydrophilic polymers having hydrocarbon backbones, and mixtures thereof. Specific examples of water-soluble polymer binders include modified starch, gelatin, polyvinylpyrrolidone, cellulose derivatives (such as for example hydroxypropyl methylcellulose (HPMC) and hydroxypropyl cellulose (HPC)), polyvinyl alcohol and mixtures thereof. The amount of binder present can vary from about 0.5% to about 25% by weight of the tablet dry weight, including all values and ranges therebetween. For example, in certain embodiments the binder is present in an amount of from about 0.5% to about 15% by weight of the tablet dry weight; in other embodiments from about 1% to about 6% by weight of the tablet dry weight; and in still other embodiments at about 3% by weight of the tablet dry weight. For example, in certain embodiments of the 174 mg, 348 mg and 522 mg dose tablets, the binder is present in an amount of from about 1% to about 6% by weight of each dry core weight, and in other embodiments at about 3% by weight of each dry core weight. In at least one embodiment of the 522 mg dose tablet, the binder is present in an amount of about 4% by weight of dry core weight. In at least one embodiment of the invention the binder is polyvinyl alcohol.

Lubricants can be added to pharmaceutical formulations to decrease any friction that occurs between the solid and the die wall during tablet manufacturing. High friction during tabletting can cause a series of problems, including inadequate tablet quality (capping or even fragmentation of tablets during ejection, and vertical scratches on tablet edges) and may even stop production. Accordingly, lubricants are added to tablet formulations of certain embodiments of the XR tablet formulation described herein. Non-limiting examples of lubricants useful for the core include glyceryl behenate, stearic acid, hydrogenated vegetable oils (such as hydrogenated cottonseed oil (STERPTEX®), hydrogenated soybean oil (STEROTEX® HM) and hydrogenated soybean oil & castor wax (STERPTEX® K), stearyl alcohol, leucine, polyethylene glycol (MW 1450, suitably 4000, and higher), magnesium stearate, glyceryl monostearate, stearic acid, polyethylene glycol, ethylene oxide polymers (for example, available under the registered trademark CARBOWAX® from Union Carbide, Inc., Danbury, Conn.), sodium lauryl sulfate, magnesium lauryl sulfate, sodium oleate, sodium stearyl fumarate, DL-leucine, colloidal silica, mixtures thereof and others as known in the art. In at least one embodiment of the present invention, the lubricant is glyceryl behenate (for example, COMPRITOL® 888). The amount of lubricant present can vary from about 0.1% to about 6% by weight of the tablet dry weight, including all values and ranges therebetween. For example, in certain embodiments the amount of lubricant present is from about 2% to about 3% by weight of the tablet dry weight; and in other embodiments the amount of lubricant present is at about 3% by weight of the tablet dry weight. In certain embodiments of the 174 mg, 348 mg and 522 mg dose XR tablets of the invention, the lubricant is present in an amount of about 3% by weight of the tablet dry weight, or from about 1% to about 6% by weight of the dry core weight. For example, in certain embodiments the lubricant is present in an amount of about 3% by weight of the dry core weight for the 174 mg, 348 mg and 522 mg dose XR tablets. In at least one embodiment of the 522 mg dose tablet, the lubricant is present in an amount of about 4% by weight of dry core weight.

At this stage, the XR core formulation of certain embodiments of the present invention, is an uncoated immediate release formulation resulting in about 100% dissolution of the tetrabenazine within about 1 hour. In at least one embodiment the XR core is a normal release matrix formulation. In certain embodiments the core includes an effective pharmaceutical amount of tetrabenazine, a binder (e.g. polyvinyl alcohol), and a lubricant (e.g. glycetyl behenate). Additional inert excipients consistent with the objects of the invention can also be added to the core formulation. The additional inert excipients can be added to facilitate the preparation and/or improve patient acceptability of the final extended-release dosage form as described herein. The additional inert excipients are well known to the skilled artisan and can be found in the relevant literature, for example in the Handbook of Pharmaceutical Excipients. Non-limiting examples of such excipients include spray dried lactose, sorbitol, mannitol, and any cellulose derivative.

In certain embodiments the core of the tetrabenazine composition (e.g. core of an XR tablet) can be made according to any one of the methods described herein.

In at least one embodiment of the invention, the granules to be compressed to form the core of the tetrabenazine XR tablet of the invention described herein, are manufactured by the wet granulation process. Wet granulation involves agitation of a powder (the active drug) by convention in the presence of a liquid (the solution binder) followed by drying. For forming the granules, which are to be eventually compressed into the tablet cores, the tetrabenazine is first granulated, for example, with a solution binder, in a granulator, for example using a fluidized bed granulator (e.g. a fluidized bed granulator manufactured by Glatt (Germany) or Aeromatic (Switzerland)). The binder (e.g. polyvinyl alcohol) is first dissolved or dispersed in a suitable solvent (e.g. water). The solution binder is then top sprayed onto the drug in a granulator (e.g. a fluidized bed granulator). Alternatively, granulation can also be performed in a conventional or high shear mixer. If necessary, the additional inert excipients (e.g. a filler) can be mixed with the tetrabenazine prior to the granulation step.

The granules formed are subsequently dried and then sieved prior to blending the granules with the lubricant. In certain embodiments, the dried granules are sieved through a 1.4 mm mesh screen. The sieved granules are then blended with the lubricant, and if necessary, any other additional inert excipients, which can improve processing of the extended-release tablets of the invention. Blending of the granules with the lubricant, and if necessary, any additional inert excipients, such as for example a glidant, can be performed in a V-blender or any other suitable blending apparatus. Glidants can improve the flowability of the powder. This for example, can be helpful during tablet production at high production speeds and during direct compaction. However, because the requirement for adequate flow is high, a glidant is often also added to a granulation before tabletting. The blended granules are subsequently pressed into tablets and are hereinafter referred to as tablet cores. Tablet cores can be obtained by the use of standard techniques and equipment well known to the skilled artisan. For example, the XR tablet cores can be obtained by a rotary press (also referred to as a multi-station press) fitted with suitable punches.

The granules can also be manufactured by using other processes known to the skilled artisan. Examples of other granule manufacturing processes include dry granulation (e.g. slugging, roller compaction), direct compression, extrusion, spheronization, melt granulation, and rotary granulation.

An example of the granulation process for the XR cores (60 kg batch) is as follows: A Fluid Bed Processor is used for granulation in order to agglomerate the particles of the materials to obtain a uniform particle size for the final blend. The granulating solution is prepared by dissolving the binder (e.g. polyvinyl alcohol) in hot purified water while mixing. The percent solids content can be adjusted to obtain a viscosity to control the build up (agglomeration size) of the material. A lower viscosity leads to smaller particles, and a higher viscosity leads to larger particles. In addition, the application rate (e.g. from about 150 gm/min to about 250 gm/min; or about 200 gm/min), position of Spray gun (e.g. center position) and nozzle size (e.g. from about 0.5 mm to about 2 mm; or about 1 mm) and atomization pressure (e.g. from 20 psi to about 40 psi; or about 30 psi) contribute further to control particle size. The active material is fluidized and heated (e.g. from about 35° C. to about 45° C.; or about 40° C.) prior to start of solution application. During the spray cycle, the bed temperature (e.g. from about 35° C. to about 45° C.; or about 40° C.) is kept at a constant temperature to avoid over-wetting. Once all the required binder solution is applied, the material is further dried to the targeted LOD value (i.e. loss on drying) (e.g. below about 1%) prior to unloading. The amount of binder (e.g. polyvinyl alcohol) is from about 2% to about 6%, and in some cases about 3%; and the solution concentration is from about 3% to about 7%, and in some cases about 4.5%. The time of agglomeration process for the 60 kg batch is from about 45 minutes to about 220 minlutes, and in some cases about 150 minutes. Once the granulate is dry, material is passed through a 1.4 and 2.00 mill screen to remove any oversized particles. The oversize particles are passed through the mill to reduce oversize particles. Oversized particles are generally not present in an amount to exceed about 5% of total yield. The screened and milled materials are placed into a shell blender (e.g. V-Blender, Bin blender) and the lubricant (e.g. glyceryl behenate) is added. The lubricant is screened and added to the granules and blended at the predetermined number of revolutions or time (e.g. mix time of about 5 min to about 15 min, and in some cases about 10 min). The percent lubricant is from about 0.5% to about 4%, and in some cases about 2%. The level of lubrication is established for sufficient coverage of either larger or smaller particle size distribution. Additional characteristics include bulk density (e.g. from about 0.3 gm/ml to about 0.8 gm/ml, and in some cases about 0.5 gm/ml), and moisture content (e.g. not more than about 1%). Particle size and flow of final blend are factors in obtaining uniform fill of cavities on a rotary press. The flow and top rotation speed of the press are adjusted (dependant on the type/size of press) so as to not jeopardize the weight uniformity of individual tablets. The product blend is passed through a hopper into a feed frame to fill the die cavities passing under the feed frame. Weight adjustments are made to keep the weight within the specified range, and adjustments made to the pressure settings to obtain the required hardness. Some components monitored for the tablets are tablet thickness and friability (e.g. less than about 0.5%). Suitable thickness (related to overall surface area) and lower friability help reduce core damage and loss of active during coating. Tablet samples are removed at predetermined intervals to monitor specifications.

Coatings

The tablet cores can be coated for administration to a subject. In at least one embodiment of the invention, the tablet cores are coated with a controlled release coating (“XR Controlled Release Coat”) that can provide an extended release of tetrabenazine. In at least one other embodiment, the tablet cores are coated with an aqueous controlled release coating that includes an aqueous dispersion of a neutral ester copolymer without any functional groups (“AQ Controlled Release Coat”).

Prophetic examples of controlled release coat formulations are provided below. It should be understood that the constituents and/or proportions of the constituents in these coatings as well as the amounts thereof can be varied in order to achieve formulations possessing different release characteristics. In all instances wherein prophetic examples are provided these compositions are intended to be exemplary and it should be understood that the specific procedures, constituents, amounts thereof and the like can be varied in order to obtain a composition possessing desired properties.

In at least one embodiment the controlled release coat is a coating formulation that provides a delayed release of the active drug(s) from the tablet core. In such embodiments the coating formulation to be applied to the core can include:

| | |
|---|---|
| EUDRAGIT ® L12.5 | about 50% by weight of coating suspension |
| Triethyl citrate | about 0.63% by weight of coating suspension |
| Talc | about 1.25% by weight of coating suspension |
| Isopropyl alcohol | about 48.12% by weight of coating suspension |
| Solids total = | about 8.1% |
| Polymer content of | about 6.3% |
| suspension = | |

In certain embodiments the controlled release coating of the tetrabenazine dosage form (e.g. controlled release coat of an XR tablet) can be made according to any one of the methods described herein.

Preparation of the controlled release coating formulation of such embodiments (e.g. controlled release coat that can provide a delayed release of the active drug) can be as follows: Talc and triethyl citrate are homogenized in the solvent by means of a homogenizer for approximately 10 minutes. The suspension is poured directly into the EUDRAGIT® L12.5 dispersion and stirred gently to avoid sedimentation. The coating is sprayed onto tablets until approximately 5 mg/cm2 of EUDRAGIT® L has been applied to the tablet core.

In at least one embodiment the controlled release coat can provide a sustained release of the active drug from the tablet core. The coating formulation can include:

| | |
|---|---|
| EUDRAGIT ® RL 12.5 | about 10% by weight of coating suspension |
| EUDRAGIT ® RS 12.5 | about 30% by weight of coating suspension |
| Dibutyl sebacate | about 0.5% by weight of coating suspension |
| Talc | about 3.5 g by weight of coating suspension |
| Magnesium stearate | about 1% by weight of coating suspension |
| Acetone | about 27.5% by weight of coating suspension |
| Isopropyl alcohol | about 27.5% by weight of coating suspension |
| Solids total = | about 10% |
| Polymer content of | about 5% |
| suspension = | |

Preparation of the controlled release coating formulation of such embodiments (i.e. controlled release coat that can provide a sustained release of the active drug) can be as follows: Dibutyl sebacate, talc and magnesium stearate are mixed and finely dispersed together with the diluents acetone and isopropyl alcohol. The suspension is then combined with the EUDRAGIT® polymer dispersions. The coating is sprayed onto the core until approximately 10 mg/cm2 of polymer has been applied to the core.

In at least one embodiment the controlled release coat is a polymer blend coating possessing pH dependent polymer (e.g. EUDRAGIT® L30D55) in combination with a sustained release polymer (e.g. AQUACOAT®). Such a coating formulation can include:

| | |
|---|---|
| AQUACOAT ® | about 21% by weight of coating suspension |
| (ethylcellulose 30%) | |
| EUDRAGIT ® L30 D 55 | about 21% by weight of coating suspension |
| Triethyl citrate | about 3% by weight of coating suspension |
| Water | about 55% by weight of coating suspension |
| Solids total = | about 15.6% |
| Polymer content of | about 12.6% |
| suspension = | |

Application of the polymer blend coating can be as follows: Coating applied to a 10 mg/cm2 application of polymer to the drug core.

In at least one embodiment the controlled release coat is a drug coating containing at least one other drug (e.g. Citalopram) on top of a core containing a release-retarding agent. The coating formulation can include:

| | |
|---|---|
| KOLLIDON ® VA64 | about 2.5% by weight of drug coating suspension |
| (Vinylpyrrolidone- | |
| vinyl acetate | |
| copolymer) | |
| KLUCEL ™ EF | about 2.5% by weight of drug coating suspension |
| (Hydroxypropyl- | |
| cellulose) | |
| Citalopram | about 2% by weight of drug coating suspension |
| Talc | about 3% by weight of drug coating suspension |
| 2-propanol | about 90% by weight of drug coating suspension |
| Solids total = | about 10% |
| Polymer content of | about 5% |
| suspension = | |

Application of the drug coating formulation can be as follows: Drug coating is sprayed onto tablets until the desired amount of other drug (e.g. Citalopram) is applied.

A top-coat can subsequently be applied as a cosmetic coating and also to prevent tablet sticking.

The top-coat formulation applied to the drug coated core can include:

| | |
|---|---|
| KOLLIDON ® VA64 | about 2.5% by weight of |
| (Vinylpyrrolidone-vinyl acetate copolymer) | top-coat suspension |
| KLUCEL ™ EF | about 2.5% by weight of |
| (Hydroxypropylcellulose) | top-coat suspension |
| Talc | about 2.5% by weight of |
| | top-coat suspension |
| Isopropyl alcohol | about 92.5% by weight of |
| | top-coat suspension |
| Solids total = | about 7.5% |
| Polymer content of suspension = | about 5% |

Application of the top-coating formulation can be as follows: Coating is applied to about a 2% weight gain (expressed as % of drug coated tablet core)

The Extended Release (XR) Controlled Release Coat

The XR controlled release coat is a semi-permeable coat including a water-insoluble, water-permeable film-forming polymer, a water-soluble polymer, and optionally a plasticizer.

Non-limiting examples of water-insoluble, water-permeable film-forming polymers useful for the XR controlled release coat of certain embodiments include cellulose ethers, cellulose esters, polyvinyl alcohol and mixtures thereof. In certain embodiments the water-insoluble, water-permeable film forming polymers can be the ethyl celluloses, and can be selected from the following non-limiting examples: ethyl cellulose grades PR100, PR45, PR20, PR10 and PR7 (ETHOCEL®, Dow), and any combination thereof. In at least one embodiment of the invention, ethyl cellulose grade PR 100 is the water-insoluble, water-permeable film-forming polymer. In certain embodiments the amount of the water-insoluble water-permeable film-forming polymer can vary from about 1% to about 12% by weight of the tablet dry weight, including all values and ranges therebetween. For example, in certain embodiments the amount of the water-insoluble water-permeable film-forming polymer is present in an amount from about 5% to about 10%, and in other embodiments from about 6% to about 8% by weight of the tablet dry weight. In certain embodiments of the 174 mg dose modified-release tablets of the invention, the amount of water-insoluble water permeable film-forming polymer is from about 3% to about 8% by weight of the tablet dry weight, and in other embodiments from about 6% to about 7% by weight of the tablet dry weight. With respect to the controlled release coat itself, the amount of water-insoluble water-permeable film-froming polymer in certain embodiments of the 174 mg dose tablet can be from about 35% to about 60% by weight of the controlled release coat dry weight, including all values and ranges therebetween; and in certain embodiments from about 40% to about 50% by weight of the controlled release coat dry weight. In certain embodiments of the 348 mg dose modified-release tablet of the invention, the amount of water-insoluble water-permeable film-forming polymer can be from about 2% to about 5% by weight of the tablet dry weight, and in other embodiments from about 3% to about 4% by weight of the tablet dry weight. With respect to the controlled release coat itself, the water-insoluble water-permeable film-forming polymer in certain embodiments of the 348 mg dose tablet is present in an amount of about 40% by weight of the controlled release coat dry weight. In certain embodiments of the 522 mg dose modified-release tablet of the invention, the amount of water-insoluble water-permeable film-forming polymer can be from about 0.5% to about 10% by weight of the tablet dry weight, and in other embodiments from about 1% to about 6% by weight of the tablet dry weight. With respect to the controlled release coat itself, the water-insoluble water-per-meable film-forming polymer in certain embodiments of the 522 mg dose tablet is present in an amount of about 37% by weight ofthe controlled release coat dry weight.

Non-limiting examples of water-soluble polymers useful for the XR controlled release coat include polyvinylpyrrolidone, hydroxypropyl methylcellulose, hydroxypropyl cellulose and mixtures thereof. In at least one embodiment the water-soluble polymer is polyvinylpyrrolidone (POVIDONE® USP). The amount of water-soluble polymer can vary from about 1.5% to about 10% by weight of the tablet dry weight, including all values and ranges therebetween. For example, in certain embodiments the amount of water-soluble polymer is from about 3% to about 8%, and in other embodiments at about 4% by weight of the tablet dry weight. With respect to the controlled release coat itself, in certain embodiments the amount of water-soluble polymer present is from about 25% to about 55% by weight of the controlled release coat dry weight. For certain embodiments of the 174 mg dose of the extended release tablet of the invention, the amount of water-soluble polymer is from about 3% to about 5% by weight of the tablet dry weight, and from about 25% to about 50% by weight of the controlled release coat dry weight, including all values and ranges therebetween. For certain embodiments of the 348 mg dose of the extended release tablet of the invention, the amount of water-soluble polymer present is fiom about 2% to about 5% of the tablet dry weight and from about 40% to about 50% by weight of the controlled release coat dry weight, including all values and ranges tlierebetweenl. For certain embodiments of the 522 mg dose of the extended release tablet of the invention, the amount of water-soluble polymer present is from about 2% to about 5% of the tablet dry weight and from about 40% to about 50% by weight of the controlled release coat dry weight, including all values and ranges therebetween.

In certain embodiments, the XR controlled release coat further includes a plasticizer. The use of plasticizers is optional, and they can be added to film coating formulations to modify the physical properties of a polymer to make it more usable during manufacturing. Plasticizers can be high boiling point organic solvents used to impart flexibility to otherwise hard or brittle polymeric materials. Plasticizers generally cause a reduction in the cohesive intermolecular forces along the polymer chains resulting in various changes in polymer properties including a reduction in tensile strength, and increase in elongation and a reduction in the glass transition or softening temperature of the polymer. The amount and choice of the plasticizer can affect the hardness of a tablet and can even affect its dissolution or disintegration characteristics, as well as its physical and chemical stability. Certain plasticizers can increase the elasticity and/or pliability of a coat, thereby decreasing the coat's brittleness. Once the dosage form is manufactured, certain plasticizers can function to increase the hydrophilicity of the coat(s) and/or the core of the dosage form in the environment of use (in-vitro or in-vivo). Non-limiting examples of plasticizers that can be used in the controlled release coat described herein include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, polyols (e.g. polyethylene glycol) of various molecular weights, and mixtures thereof. It is contemplated and within the scope of the invention, that a combination of plasticizers can be used in the present formulation. In at least one embodiment of the invention, the plastizer is polyethylene glycol 4000, dibutyl sebacate or a mixture thereof. The amount of plasticizer for the controlled release coat can vary in an amount of from about 0.5% to about 4% by weight of the tablet dry weight, including all values and ranges therebetween. For example, in certain embodiments the plasticizer is present in an amount of from about 2% to about 3% by weight of the tablet dry weight. For certain embodiments of the 174 mg dose extended-release tablet of the invention, the amount of plasticizer present in the controlled release coat is from about 1% to about 4% by weight of the tablet dry weight. For certain embodiments of the 348 mg dose extended release tablet of the invention, the amount of plasticizer present is from about 0.5% to about 4% by weight of the tablet dry weight. For certain embodiments of the 522 mg dose extended release tablet of the invention, the amount of plasticizer present is from about 0.5% to about 4% by weight of the tablet dry weight. In certain embodiments of the 174 mg, 348 ing and 522 mg dosage forms, the plasticizer is present in an amount of from about 6% to about 30% by weight of the controlled release coat dry weight, including all values and ranges therebetween. For example, in certain embodiments the plasticizer is present in an amount of about 12% by weight of the controlled release coat dry weight.

The ratio of water-insoluble water-permeable film forming polymer:plasticizer:water-soluble polymer for the XR controlled release coat of certain embodiments of the invention described herein can vary from about 3:1:4 to about 5:1:2, including all values and ranges therebetween. For example, in certain embodiments the ratio of water-insoluble water-permeable film forming polymer:plasticizer:water-soluble polymer for the XR controlled release coat is about 4:1:3. For certain other embodiments of the XR tablet the ratio of the water-insoluble water-permeable film-forming polymer:plasticizer:water-soluble polymer in the XR controlled release coat is from about 7:2:6 to about 19:5:18, including all values and ranges therebetween. In at least one embodiment the ratio of water-insoluble water-permeable film forming polymer:plasticizer:water-soluble polymer for the XR controlled release coat is about 13:4:12. In at least one embodiment of the 522 mg dosage form, the ratio of water-insoluble water-permeable film forming polymer:plasticizer:water-soluble polymer for the XR controlled release coat is about 13:6:16.

In certain embodiments the XR controlled release coat of the tetrabenazine tablet can be made according to any one of the methods described herein.

Preparation and application of the XR controlled release coat can be as follows. The water-insoluble water-permeable film-forming polymer (e.g. ethylcellulose), and the plasticizer (e.g. polyethylene glycol 4000), are dissolved in an organic solvent (e.g. a mixture of ethyl alcohol). In the manufacture of embodiments that do not require a plasticizer, the water-insoluble water-permeable film-forming polymer can be dissolved in the organic solvent without the plasticizer. The water-soluble polymer (e.g. polyvinyl pyrrolidone) is next added until a homogenous mixture is achieved. The resulting controlled release coat solution is then sprayed onto the tablet cores using a tablet coater, fluidized bed apparatus or any other suitable coating apparatus known in the art until the desired weight gain is achieved. The tablet cores coated with the controlled release coat are subsequently dried.

An example of the coating process for the XR controlled release coat is as follows: The XR controlled release coat solution is prepared by dissolving the water insoluble polymer (e.g. ethylcellulose) and water soluble polymer (e.g. polyvinylpyrrolidone) and an ethyl alcohol mixture while mixing and is followed with the addition of the plasticizer(s) (e.g. mixture of polyethylene glycol 4000 and dibutyl sebacate). Once completely dissolved, the solution is homogenized to obtain a uniform mixture of appropriate viscosity. This procedure helps obtain a complex mix of a water permeable film to control the release of the active drug. The composition of the solution can be formulated to contain various levels of the water insoluble polymer and water soluble polymer and a mix of the plasticizer(s). The release function is further controlled by the film thickness applied and measured as weight gain of solids in the coating required. Tablets are coated in a perforated coating pan with control of pan speed (e.g. from about 8 rpm to about 14 rpm, and in some cases about 12 rpm), spray rate (e.g. from about 150 gm/min to about 250 gm/min, and in some cases about 200 gm/min), atomization pressure (e.g. from about 15 psi to about 25 psi, and in some cases about 20 psi), supply volume (from about 800 to about 1000 cubic ft/min, and in some cases about 900 cubic ft/min), and air temperature (e.g. from about 50° C. to about 60° C., and in some cases about 55° C.), monitored through a bed temperature and/or outlet temperature of from about 38° C. to about 42° C., and in some cases about 40° C. On completion of the coating cycle, tablets are dried and unloaded into bulk containers. The printing process includes the transfer of a print image from a print plate covered with edible black ink and transferred via a print roll or print pad onto the surface of the tablets. The printed tablets are transferred through a drying element prior to discharging into bulk containers. Samples for final testing are taken throughout the printing process.

The skilled artisan will appreciate that controlling the permeability can control the release of the tetrabenazine and/or the amount of coating applied to the tablet cores. The permeability of the XR controlled release coat can be altered by varying the ratio of the water-insoluble, water-permeable film-forming polymer:plasticizer:water-soluble polymer and/or the quantity of coating applied to the tablet core. A more extended release can be obtained with a higher amount of water-insoluble, water-permeable film forming polymer. The addition of other excipients to the tablet core can also alter the permeability of the controlled release coat. For example, if it is desired that the tablet core further include an expanding agent, the amount of plasticizer in the controlled release coat could be increased to make the coat more pliable, as the pressure exerted on a less pliable coat by the expanding agent could rupture the coat. Further, the proportion of the water-insoluble water-permeable film forming polymer and water-soluble polymer can also be altered depending on whether a faster or slower dissolution and/or release profile is desired.

Depending on the dissolution or in-vivo release profile desired, the weight gained after coating the tablet core with the XR controlled release coat typically can vary from about 3% to about 30% of the weight of the dry tablet core. For a 174 mg dose extended release tablet according to certain embodiments, the weight gain can typically vary from about 10% to about 17% of the weight of the dry tablet core. For example in the 174 mg tablet of certain embodiments, the weight gain is about 14% of the weight of the dry tablet core. For the 348 mg dose extended release tablet of certain embodiments, the weight gain can vary from about 7% to about 10% of the weight of the dry tablet core. For example in the 348 mg tablet of certain embodiments, the weight gain is about 9% of the weight of the dry tablet core. For the 522 mg dose extended release tablet of certain embodiments, the weight gain can vary from about 5% to about 15% of the weight of the dry tablet core. For example in the 522 mng tablet of certain embodiments, the weight gain is about 8.5% of the weight of the dry tablet core.

The XR tablet of certain embodiments of the invention provides an extended-release of the tetrabenazine. In at least one embodiment no pore forming agent is present in the XR coating formulation. An extended release tetrabenazine formulation is provided in certain embodiments such that after about 2 hours, not more than about 20% of the tetrabenazine content is released. For example, in certain embodiments, from about 2% to about 18%, fiom about 4% to about 8%, or about 5% of the tetrabenazine content is released after about 2 hours. After about 4 hours, from about 15% to about 45% of the tetrabenazine content is released. For example, in certain embodiments from about 21% to about 37%, from about 28% to about 34%, or about 32% of the tetrabenazine content is released after about 4 hours. After about 8 hours, about 40% to about 90% of the tetrabenazinie content is released. For example, in certain embodiments from about 60% to about 85%, from about 68% to about 74%, or about 74% of the tetrabenazine content is released after about 8 hours. After about 16 hours not less than about 80% of the tetrabenazine content is released. For example, in certain embodiments not less than about 93%, not less than about 96%, or not less than about 99% of the tetrabenazine content is released after about 16 hours.

Also, extended release tablets are provided in certain embodiments wherein after about 2 hours not more than about 40% (e.g., about 33%) of the tetrabenazine is released; after about 4 hours from about 40 to about 75% of the tetrabenazine is released (e.g., about 59%); after about 8 hours at least about 75% of the tetrabenazine is released (e.g.,. about 91%); and after about 16 hours at least about 85% of the tetrabenazine is released (e.g., about 97%). In all instances herein when actual or prophetic dissolution profiles are provided this means that the medicament possesses such a profile in at least one dissolution medium under prescribed conditions such as are identified herein and are well known to those skilled in the art. Such dissolution media, dissolution conditions and apparatus for use therein are disclosed in the United States Pharmacopoeia (USP) and European and Japanese counterparts thereof. Additionally, specific examples thereofare provided in this application.

Controlled Release Matrix

In other embodiments of the present invention, a controlled release matrix is provided from which the kinetics of drug release from the matrix core are dependent at least in part upon the diffusion and/or erosion properties of excipients within the composition. In this embodiment controlled release matrices contain an effective amount of tetrabenazine and at least one pharmaceutically acceptable excipient. The amount of the tetrabenazine present in the controlled release matrix can vary in an amount of from about 40% to about 90% by weight of the matrix tablet dry weight. For example, in certain embodiments tetrabenazinle is present in an amount from about 60% to about 80%, and in other embodiment at about 70% by weight of the matrix tablet dry weight. The controlled release matrix can be multiparticulate or uniparticulate, and can be coated with at least one functional or non-functional coating, or an immediate release coating containing another drug. Functional coatings include by way of example controlled release polymeric coatings, enteric polymeric coatings, and the like. Non-functional coatings are coatings that do not affect drug release but which affect other properties (e.g., they can enhance the chemical, biological, or the physical appearance of the controlled release formulation). Those skilled in the pharmaceutical art and the design of medicaments are well aware of controlled release matrices conventionally used in oral pharmaceutical compositions adopted for controlled release and means for their preparation.

Suitable excipient materials for use in such controlled release matrices include, by way of example, release-resistant or controlled release materials such as hydrophobic polymers, hydrophilic polymers, lipophilic materials and mixtures thereof. Non-limiting examples of hydrophobic, or lipophilic components include glyceryl monostearate, mixtures of glyceryl monostearate and glyceryl monopalmitate (MYVAPLEX™, Eastman Fine Chemical Company), glycerylmonooleate, a mixture of mono, di and tri-glycerides (ATMUL™ 84S), glycerylmonolaulate, paraffin, white wax, long chain carboxylic acids, long chain carboxylic acid esters, long chain carboxylic acid alcohols, and mixtures thereof. The long chain carboxylic acids can contain from about 6 to about 30 carbon atoms; in certain embodiments at least about 12 carbon atoms, and in other embodiments from about 12 to about 22 carbon atoms. In some embodiments this carbon chain is fully saturated and unbranched, while others contain one or more double bonds. In at least one embodiment the long chain carboxylic acids contain about 3-carbon rings or hydroxyl groups. Non-limiting examples of saturated straight chain acids include n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid, melissic acid and mixtures thereof. Also useful are unsaturated monoolefinic straight chain monocarboxylic acids. Non-limiting examples of these include oleic acid, gadoleic acid, erucic acid and mixtures thereof. Also useful are unsaturated (polyolefinic) straight chain monocaboxyic acids. Non-limiting examples of these include linoleic acid, linolenic acid, arachidonic acid, behenolic acid and mixtures thereof. Useful branched acids include, for example, diacetyl tartaric acid. Non-limiting examples of long chain carboxylic acid esters include glyceryl monostearates; glyceryl monopalmitates; mixtures of glyceryl monostearate and glyceryl monopalmnitate (MYVAPLEX™ 600, Eastman Fine Chemical Company); glyceryl monolinoleate; glyceryl monooleate; mixtures of glyceryl monopalmitate, glyceryl monostearate glyceryl monooleate and glyceryl monolinoleate (MYVEROL™ 18-92, Eastman Fine Chemical Company); glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate (MYVEROL™ 18-99, Eastman Fine Chemical Company); acetylated glycerides such as distilled acetylated monoglycerides (MYVACET™ 5-07, 7-07 and 9-45, Eastman Fine Chemical Company); mixtures of propylene glycol monoesters, distilled monoglycerides, sodium stearoyl lactylate and silicon dioxide (MYVATEX™ TL, Eastman Fine Chemical Company); mixtures of propylene glycol monoesters, distilled monoglycerides, sodium stearoyl lactylate and silicon dioxide (MYVATEX™ TL, Eastman Fine Chemical Company) d-alpha tocopherol polyethylene glycol 1000 succinate (Vitamin E TPGS, Eastman Chemical Company); mixtures of mono- and diglyceride esters such as ATMUL™ (Humko Chemical Division of Witco Chemical); calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate. sorbitan monooleate; other sorbitan esters of long chain carboxylic acids; succinylated monoglycerides; stealyl monoglyceryl citrate; stearyl heptanoate; cetyl esters of waxes, cetearyl octanoate; C10-C30 cholesterol/lavosterol esters; sucrose long chain carboxylic acid esters; and mixtures thereof.

The alcohols useful as excipient materials for controlled release matrices can include the hydroxyl forms of the carboxylic acids exemplified above and also cetearyl alcohol.

In addition, waxes can be useful alone or in combination with the materials listed above, as excipient materials for the controlled release matrix embodiments of the present invention. Non-limiting examples of these include white wax, paraffin, microcrystalline wax, carnauba wax, and mixtures thereof.

The lipophilic agent can be present in an amount of from about 5% to about 90% by weight of the controlled release matrix dosage form. For example, in certain embodiments the lipophilic agent is present in an amount of from about 10% to about 85%, and in other embodiments from about 30% to about 60% by weight of the controlled release matrix dosage form.

Non-limiting examples of hydrophilic polymers that can be used in certain embodiments of the controlled release matrix dosage form include hydroxypropylmethylcellulose (HPMC), hydroxypropylcellulose (HPC), hydroxyethylcellulose (HEC), carboxymethylcellulose (CMC) or other cellulose ethers, polyoxyethylene, alginic acid, acrylic acid derivatives such as polyacrylic acid, CARBOPOL™ (B. F. Goodrich, Cleveland, Ohio), polymethacrylate polymer such as EUDRAGIT® RL, RS, R, S, NE and E (Rhome Pharma, Darmstadt, Germany), acrylic acid polymer, methacrylic acid polymer, hydroyethyl methacrylic acid (HEMA) polymer, hydroxymethyl methacrylic acid (HMMA) polymer, polyvinyl alcohols and mixtures thereof.

The hydrophilic polymer can be present in an amount of from about 10% to about 90% by weight of the controlled release matrix dosage form. For example, in certain embodiments the hydrophilic polymer is present in an amount of from about 20% to about 75%, and in other embodiments from about 30% to about 60% by weight of the controlled release matrix dosage form.

In at least one embodiment, the controlled release matrix dosage form includes hydroxypropylmethylcellulose (HPMC). HPMC is an anhydroglucose in which some of the hydroxyl groups are substituted with methyl groups to form methyl ether moieties, and others are substituted with hydroxypropyl groups or with methoxypropyl groups to form hydroxypropyl ether or methoxypropyl ether moieties. Non-limiting examples of hydroxypropyl methylcelluloses that are commercially available include METHOCEL® E (USP type 2910). METHOCEL® F (USP type 2906), METHOCEL® J (USP type 1828), METHOCEL® K (USP type 2201), and METHOCEL® 310 Series, products of The Dow Chemical Company, Midland, Mich., USA. The average degree of methoxyl substitution in these products can range from about 1.3 to about 1.9 (of the three positions on each unit of the cellulose polymer that are available for substitution) while the average degree of hydroxypropyl substitution per unit expressed in molar terms can range from about 0.13 to about 0.82. The dosage form can include the different HPMC grades having different viscosities. The size of a HPMC polymer is expressed not as molecular weight but instead in terms of its viscosity as about a 2% solution by weight in water. Different HPMC grades can be combined to achieve the desired viscosity characteristics. For example, the at least one pharmaceutically acceptable polymer can include two HPMC polymers such as for example METHOCEL® K3 LV (which has a viscosity of about 3 cps) and METHOCEL® K100M CR (which has a viscosity of about 100,000 cps). In addition, the polymer can include two hydroxypropylcellulose forms such as KLUCEL® LF and KLUCEL® EF. In addition, the at least one polymer can include a mixture of a KLUCEL® and a METHOCEL®.

In at least one embodiment the controlled release matrix dosage form includes a polyethylene oxide (PEO). PEO is a linear polymer of unsubstituted ethylene oxide. In certain embodiments poly(ethylene oxide) polymers having viscosity-average molecular weights of about 100,000 Daltons and higher are used. Non-limiting examples of poly(ethylene oxide)s that are commercially available include: POLYOX® NF, grade WSR Coagulant, molecular weight 5 million; POLYOX® grade WSR 301, molecular weight 4 million; POLYOX® grade WSR 303, molecular weight 7 million; POLYOX® grade WSR N-60K, molecular weight 2 million; and mixtures thereof. These particular polymers are products of Dow Chemical Company, Midland, Mich., USA. Other examples of polyethylene oxides exist and can likewise be used. The required molecular weight for the PEO can be obtained by mixing PEO of differing molecular weights that are available commercially.

In at least one embodiment of the controlled release matrix dosage form, PEO and HPMC are combined within the same controlled release matrix. In certain embodiments, the poly(ethylene oxide)s have molecular weights ranging from about 2,000,000 to about 10,000,000 Da. For example, in at least one embodiment the polyethylene oxides have molecular weights ranging from about 4,000,000 to about 7,000,000 Da. In certain embodiments the HPMC polymers have a viscosity within the range of about 4,000 centipoises to about 200,000 centipoises. For example, in at least one embodiment the HPMC polymers have a viscosity of from about 50,000 centipoises to about 200,000 centipoises, and in other embodiments from about 80,000 centipoises to about 120,000 centipoises. The relative amounts of PEO and HPMC within the controlled release matrix can vary within the scope of the invention. In at least one embodiment the PEO:HPMC weight ratio is from about 1:3 to about 3: 1. For example, in certain embodiments the PEO:HPMC weight ratio is from about 1:2 to about 2:1. As for the total amount of polymer relative to the entire matrix, this can vary as well and can depend on the desired drug loading. In at least one embodiment the total amount of polymer in the matrix can constitute from about 15% to about 90% by weight of the matrix dosage form. For example, in certain embodiments the total amount of polymer in the matrix is from about 20% to about 75%, in other embodiments from about 30% to about 60%, and in still other embodiments from about 10% to about 20% by weight of the matrix dosage form.

In at least one embodiment of the invention the controlled release matrix dosage forn includes a hydrophobic polymer such as ethylcellulose. The viscosity of ethylcellulose can be selected in order to influence of rate the drug release. In certain embodiments the ethylcellulose has a viscosity from about 7 to about 100 cP (when measured as a 5% solution at 25° C. in an Ubbelolide viscometer, using a 80:20 toluene:ethanol solvent.) In certain embodiments the hydrophobic polymer can constitute from about 10% to about 90% by weight of the matrix dosage form. For example, in at least one embodiment the hydrophobic polymer constitutes from about 20% to about 75%, and in other embodiments from about 30% to about 60% by weight of the matrix dosage form.

In at least one embodiment of the invention the controlled release matrix dosage form includes at least one binder. In certain embodiments the binder is water-insoluble. Examples of binders include hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Non-limiting examples of water-soluble polymer binders include modified starch, gelatin, polyvinylpyrrolidone, cellulose derivatives (such as for example hydroxypropyl methylcellulose (HPMC) and hydroxypropyl cellulose (HPC)), polyvinyl alcohol and mixtures thereof. In at least one embodiment, the binder can be present in an amount of from about 0.1% to about 20% by weight of the matrix dosage form. For example, in certain embodiments the binder is present in an amount of from about 0.5% to about 15%, and in other embodiments from about 2% to about 10% by weight of the matrix dosage form.

In at least one embodiment of the invention the controlled release matrix dosage form includes at least one lubricant. Non-limiting examples of lubricants include stearic acid, hydrogenated vegetable oils (such as hydrogenated cottonseed oil (Sterotex®), hydrogenated soybean oil (STEROTEX® HM) and hydrogenated soybean oil & castor wax (STEROTEX® K)) stearyl alcohol, leucine, polyethylene glycol (MW 1450, suitably 4000, and higher), magnesium stearate, glycetyl monostearate, stearic acid, glycerylbehenate, polyethylene glycol, ethylene oxide polymers (for example, available under the registered trademark CARBOWAX®D from Union Carbide, Inc., Danbury, Conn.), sodium lauryl sulfate, magnesium lauryl sulfate, sodium oleate, sodium stearyl fumarate, DL-leucine, colloidal silica, and mixtures thereof. The lubricant can be present in an amount of from about 0 to about 4% by weight of the compressed uncoated matrix. For example, in certain embodiments the lubricant is present in an amount of from about 0% to about 2.5% by weight of the compressed, uncoated matrix.

In at least one embodiment of the invention the controlled release matrix dosage form includes a plasticizer. Non-limiting examples of plasticizers include dibutyl sebacate, diethyl phthalate, triethyl citrate, tributyl citrate, triacetin, citric acid esters such as triethyl citrate NF XVI, tributyl citrate, dibutyl phthalate, 1,2-propylene glycol, polyethylene glycols, propylene glycol, diethyl phthalate, castor oil, acetylated monoglycerides, phthalate esters, and mixtures thereof. In at least one embodiment, the plasticizer can be present in an amount of from about 1% to about 70% by weight of the controlled release polymer in the matrix dosage form. For example, in certain embodiments the plasticizer is present in an amount of fiom about 5% to about 50%, and in other embodiments from about 10% to about 40% by weight of the controlled release polymer in the matrix dosage form.

In at least one embodiment of the invention the controlled release matrix dosage form includes at least one diluent, non-limiting examples of which include dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol, sucralfate, calcium hydroxyl-apatite, calcium phosphates, fatty acid salts such as magnesium stearate, and mixtures thereof. In certain embodiments the diluent can be added in an amount so that the combination of the diluent and the active substance includes up to about 60%, and in other embodiments up to about 50%, by weight of the composition.

In at least one embodiment of the invention the controlled release matrix dosage form includes a solubilizer. The solubilizer can act to increase the instantaneous solubility of the tetrabenazine. The solubilizer can be selected from hydrophilic surfactants or lipophilic surfactants or mixtures thereof. The surfactants can be anionic, nonionic, cationic, and zwitterionic surfactants. The hydrophilic non-ionic surfactants can be selected from the group comprised of, but not limited to: polyethylene glycol sorbitan fatty acid esters and hydrophilic transesterification products of a polyol with at least one member of the group from triglycerides, vegetable oils, and hydrogenated vegetable oils such as glycerol, ethylene glycol, polyethylene glycol, sorbitol, propylene glycol, pentaerythritol, or a saccharide, d-α-tocopheryl polyethylene glycol 1000 succinate. The ionic surfactants can be selected from the group comprised of, but not limited to: alkylammonum salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates ; fatty acid salts; sodium docusate; acyl lactylates; mono-and di-acetylated tartaric acid esters of mono-and di-glycerides; succinylated mono-and di-glycerides; citric acid esters of mono-and di-glycerides; and mixtures thereof. The lipophilic surfactants can be selected from the group comprised of, but not limited to: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono-and di-glycerides; hydrophobic transesterification products of a polyol with at least one member of the group from glycerides, vegetable oils, hydrogenated vegetable oils, fatty acids and sterols; oil-soluble vitamins/vitamin derivatives; PEG sorbitan fatty acid esters, PEG glycerol fatty acid esters, polyglycerized fatty acid, polyoxyethylene-polyoxypropylene block copolymers, sorbitan fatty acid esters; and mixtures thereof. In at least one embodiment the solubilizer can be selected from: PEG-20-glyceryl stearate (CAPMUL® by Abitec), PEG-40 hydrogenated castor oil (CREMOPHOR RH 40® by BASF), PEG 6 corn oil (LABRAFIL® by Gattefosse), lauryl macrogol-32 glyceride (GELUCIRE44/14® by Gattefosse) stearoyl macrogol glyceride (GELUCIRE50/13® by Gattefosse), polyglyceryl-10 mono dioleate (CAPROL® PEG860 by Abitec), propylene glycol oleate (LUTROL® by BASF), Propylene glycol dioctanoate (CAPTEX® by Abitec), Propylene glycol caprylate/caprate (LABRAFAC® by Gattefosse), Glyceryl monooleate (PECEOL® by Gattefrosse), Glycerol monolinoleate (MAISINE® by Gattefrosse), Glycerol monostearate (CAPMUL® by Abitec), PEG-20 sorbitan monolaurate (TWEEN20® by ICI), PEG-4 lauryl ether (BRIJ30® by ICI), Sucrose distearate (SUCROESTER7® by Gattefosse), Sucrose monopalmitate (SUCROESTER15® by Gattefosse), polyoxyethylene-polyoxypropylene block copolymer (LUTROL® series BASF), polyethylene glycol 660 hydroxystearate, (SOLUTOL® by BASF), Sodium lauryl sulfate, Sodium dodecyl sulphate, Dioctyl suphosuccinate, L-hydroxypropyl cellulose, hydroxylethylcellulose, hydroxyl propylcellulose, Propylene glycol alginate, sodium taurocholate, sodium glycocholate, sodium deoxycholate, betains, polyethylene glycol (CARBOWAX® by DOW), d-α-tocopheryl polyethylene glycol 1000 succinate, (Vitamin E TPGS® by Eastman), and mixtures thereof. In at least one other embodiment the solubilizer can be selected from PEG-40 hydrogenated castor oil (CREMOPHOR RH 40® by BASF), lauryl macrogol-32 glyceride (GELUCIRE44/14® by Gattefosse) stearoyl macrogol glyceride (GELUCIRE 50/13® by Gattefosse), PEG-20 sorbitan monolaurate (TWEEN 20® by ICI), PEG-4 lauryl ether (BRIJ30® by ICI), polyoxyethylene-polyoxypropylene block copolymer (LUTROL® series BASF), Sodium lauryl sulphate, Sodium dodecyl sulphate, polyethylene glycol (CARBOWAX(®) by DOW), and mixtures thereof.

In at least one embodiment of the invention the controlled release matrix dosage form includes a swelling enhancer. Swelling enhancers are members of a category of excipients that swell rapidly to a large extent resulting in an increase in the size of the tablet. At lower concentrations, these excipients can be used as superdisintegrants; however at concentrations above 5% w/w these agents can function as swelling enhancers and help increase the size of the matrix dosage form. According to certain embodiments of the matrix dosage forms of the invention, examples of swelling enhancers include but are not limited to: low-substituted hydroxypropyl cellulose, microcrystalline cellulose, cross-linked sodium or calcium carboxymethyl cellulose, cellulose fiber, cross-linked polyvinyl pyrrolidone, cross-linked polyacrylic acid, cross-linked Amberlite resin, alginates, colloidal magnesium-aluminum silicate, corn starch granules, rice starch granules, potato starch granules, pregelatinised starch, sodium carboxymethyl starch and mixtures thereof. In at least one embodiment of the matrix dosage forms, the swelling enhancer is cross-linked polyvinyl pyrrolidone. The content of the swelling enhancer can be from about 5% to about 90% by weight of the matrix dosage form. For example, in certain embodiments the swelling enhancer is present in an amount of from about 10% to about 70%, and in other embodiments from about 15% to about 50% by weight of the matrix dosage form.

In at least one embodiment of the invention the controlled release matrix dosage form includes additives for allowing water to penetrate into the core of the preparation (hereinafter referred to as “hydrophilic base”). In certain embodiments, the amount of water required to dissolve 1 g of the hydrophilic base is not more than about 5 ml, and in other embodiments is not more than about 4 ml at the temperature of about 20° C.±5° C. The higher the solubility of the hydrophilic base in water, the more effective is the base in allowing water into the core of the preparation. The hydrophilic base includes, inter alia, hydrophilic polymers Such as polyethylene glycol (PEG); (e.g. PEG400, PEG 1500, PEG4000, PEG6000 and PEG20000, produced by Nippon Oils and Fats Co.) and polyvinylpyrrolidone (PVP); (e.g. PVP K30, of BASF), sugar alcohols such as D-sorbitol, xylitol, or the like, sugars such as sucrose, anhydrous maltose, D-fructose, dextran (e.g. dextran 40), glucose or the like, surfactants such as polyoxyethylene-hydrogenated castor oil (HCO; e.g. CREMOPHOR™ RH40 produced by BASF, HCO-40 and HCO-60 produced by Nikko Chemicals Co.), polyoxyethylene-polyoxypropylene glycol (e.g. Pluronic F68 produced by Asahi Denka Kogyo K.K.), polyoxyethylene-sorbitan high molecular fatty acid ester (TWEEN™; e.g. TWEEN™ 80 produced by Kanto Kagaku K.K.), or the like; salts such as sodium chloride, magnesium chloride., or the like; organic acids such as citric acid, tartaric acid., or the like; amino acids such as glycine, .β-alanine, lysine hydrochloride, or the like; and amino sugars such as meglumine. In at least one embodiment the hydrophilic base is PEG6000, PVP, D-sorbitol, or mixtures thereof.

In another embodiment of the invention the controlled release matrix dosage form includes at least one disintegrant. Non-limiting examples of disintegrants for use in the matrix dosage form include croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and mixtures thereof. In at least one embodiment the disintegrant is selected from cross-linked polyvinylpyrrolidone (e.g. KOLLIDON® CL), cross-linked sodium carboxymethylcellulose (e.g. AC-DI-SOL™), starch or starch derivatives such as sodium starch glycolate (e.g. EXPLOTAB®), or combinations with starch (e.g. PRIMOJEL™), swellable ion-exchange resins, such as AMBERLITE™ IRP 88, formaldehyde-casein (e.g. ESMA SPRENG™), and mixtures thereof. In at least one embodiment the disintegrant is sodium starch glycolate. The disintegrant can be present in certain embodiments in an amount of from about 0% to about 20% of the total weight of the matrix.

The controlled release matrices of the present invention can further contain one or more pharmaceutically acceptable excipients such as granulating aids or agents, colorants, flavorants, pH adjusters, anti-adherents, glidants and like excipients conventionally used in pharmaceutical compositions.

Multiparticles within a Tablet Matrix

The formulations of erodible tablet matrices of the present invention can assume the form of contained microparticles within the tablet body. In at least one embodiment the formulation includes microparticles distributed within the matrix tablet blend and compressed as a controlled release single unit. As this tablet swells and erodes, the multiparticles are hydrated and released from the dosage form in a controlled fashion over time to sustain the drug release. The multiparticles can be of any composition that promotes or controls drug release; for example immediate release, enhanced absorption, controlled release, pulsatile release, or extended release. Conventional methods can be used for containing the microparticles within the tablet in this manner.

In at least one embodiment of the invention including water swellable polymers formulated into the matrix, the release kinetics of the tetrabenazine from the matrix are dependent upon the relative magnitude of the rate of polymer swelling at the moving rubbery/glassy front and the rate of polymer erosion at the swollen polymer/dissolution medium front. The release kinetics for the release of the tetrabenazine from the matrix can be approximated by the following equation:

Mt/MT=ktn

where t is time,

Mt is the amount of the pharmaceutical agent which has been released at time t,

MT is the total amount of the pharmaceutical agent contained in the matrix,

k is a constant, and

n is the release kinetics exponent

This equation is valid so long as n remains nearly constant. When n is equal to one, the release of the pharmaceutical agent from the matrix has zero-order kinetics. The amount of pharmaceutical agent released is then directly proportional to the time.

Where the swelling process of the polymer chosen for the excipient is the primary process controlling the drug release (compared to erosion of the swollen polymer), non-zero order release kinetics can result. Generally, these release kinetics dictate a value of n approaching 0.5, leading to square-root Fickian-type release kinetics.

In at least one embodiment of the invention, polymers are selected for inclusion into the formulation to achieve zero order kinetics. The release kinetics of the matrix can also be dictated by the pharmaceutical agent itself. A drug which is highly soluble can tend to be released faster than drugs which have low solubility. Where a drug has high solubility, polymer swelling and erosion takes place rapidly to maintain zero order release kinetics. If the swelling and erosion take place too slowly, the swelling process of the polymer is the primary process controlling the drug release (since the drug will diffuse from the swollen polymer before the polymer erodes). In this situation, non-zero order release kinetics can result. As a result, the administration of a highly soluble pharmaceutical agent requires a relatively rapidly swelling and eroding excipient. To use such a material to produce a matrix which will last for 24 hours can require a large matrix. To overcome this difficulty, a doughnut-shaped matrix with a hole though the middle can be used with a less rapidly swelling and eroding polymer. With such a matrix, the surface area of the matrix increases as the matrix erodes. This exposes more polymer, resulting in more polymer swelling and erosion as the matrix shrinks in size. This type of matrix can also be used with very highly soluble pharmaceutical agents to maintain zero order release kinetics.

In at least one other embodiment of the invention, zero order drug release kinetics can be achieved by controlling the surface area of the matrix dosage form that is exposed to erosion. When water is allowed to diffuse into a polymer matrix composition zero order release is obtained when the release rate is governed or controlled by erosion of a constant surface area per time unit. In order to ensure that the erosion of the polymer matrix composition is the predominant release mechanism, it is helpful to provide a polymer matrix composition which has properties that ensures that the diffusion rate of water into the polymer matrix composition substantially corresponds to the dissolution rate of the polymer matrix composition into the aqueous medium. Thus, by adjusting the nature and amount of constituents in the polymer matrix composition a zero order release mechanism can be achieved. The compositions employed are coated in such a manner that at least one surface is exposed to the aqueous medium and this surface has a substantially constant or controlled surface area during erosion. In the present context controlled surface area relates to a predetermined surface area typically predicted from the shape of the coat of the unit dosage system. It may have a simple uniform cylindrical shape or the cylindrical form can have one or more tapered ends in order to decrease (or increase) the initial release period. Accordingly, these embodiments provide a method for controlling the release of tetrabenazine into an aqueous medium by erosion of at least one surface of a pharmaceutical composition including tetrabenazine.

The coating platform includes a polymeric material insoluble in water and optionally insoluble in biodegradable biological liquids, and able to maintain its impermeability characteristics at least until the complete transfer of the tetrabenazine contained in the deposit-core. It is applied to a part of the external deposit-core surface chosen such as to suitably direct and quantitatively regulate the release of the tetrabenazine. In this respect, as the support-platform is impermeable to water, the polymeric material of the deposit-core in certain embodiments can swell only in that portion of the deposit not coated with the platform.

The support-platform can be obtained by compressing prechosen polymeric materials onto the deposit-core, by immersing the deposit-core in a solution of said polymeric materials in normal organic solvents, or by spraying said solutions. Polymeric materials usable for preparing the support-platform can be chosen from the class including acrylates, celluloses and derivatives such as ethylcellulose, cellulose acetate-propionate, polyethylenes and methacrylates and copolymers of acrylic acid, polyvinylalcohols and mixtures thereof. This platform can have a thickness of from about 2 mm (for example, if applied by compression) to about 10 microns (for example, if applied by spraying or immersion), and includes from about 10% to about 90% of the total surface of the system.

A factor in controlling the release of the tetrabeniazine is the intensity and duration of the swelling force developed by the swellable polymeric materials contained in the deposit-core on contact with aqueous fluids. In this respect, the energy for activating, executing and regulating the release of the tetrabenazine can be determined by the swelling force developed in the deposit-core when this comes into contact with water or with biological liquids. Said force has an intensity and duration which can vary in relation to the type and quantity of the polymeric materials used in formulating the deposit, and it lies between limits having a maximum value which occurs in the case of a deposit mainly containing the swellable polymer, and a minimum value which occurs in the case of a deposit mainly containing the gellable polymer. Said swellable polymer can be present in an amount of from about 5% to about 80% by weight, and said gellable polymer present in an amount of from about 10% to about 90% by weight, with respect to the mixture forming the deposit-core.

A further control factor is the geometry of the support-platform, which limits the swelling of the deposit and directs the emission of material from it. Within the scope of these embodiments it is possible to conceive many systems for the controlled release of tetrabenazine, which base their operation on the swelling force and differ from each other by the type of support-platform used.

In another embodiment of the present invention, a swellable matrix dosage form is provided in which the tetrabenazine is dispersed in a polymeric matrix that is water-swellable rather than merely hydrophilic, that has an erosion rate that is substantially slower than its swelling rate, and that releases the tetrabenazine primarily by diffusion. The rate of diffusion of the tetrabenazine out of the swellable matrix can be slowed by increasing the drug particle size, by the choice of polymer used in the matrix, and/or by the choice of molecular weight of the polymer. The swellable matrix is comprised of a relatively high molecular weight polymer that swells upon ingestion. In at least one embodiment the swellable matrix swells upon ingestion to a size that is at least twice its unswelled volume, and that promotes gastric retention during the fed mode. Upon swelling, the swellable matrix can also convert over a prolonged period of time from a glassy polymer to a polymer that is rubbery in consistency, or from a crystalline polymer to a rubbery one. The penetrating fluid then causes release of the tetrabenazine in a gradual and prolonged manner by the process of solution diffusion, i.e., dissolution of the tetrabenazine in the penetrating fluid and diffusion of the dissolved tetrabenazine back out of the swellable matrix. The swellable matrix itself is solid prior to administration and, once administered, remains undissolved in (i.e., is not eroded by) the gastric fluid for a period of time sufficient to permit the majority of the tetrabenazine to be released by the solution diffusion process during the fed mode. The rate-limiting factor in the release of the tetrabenazine from the swellable matrix is therefore controlled diffusion of the tetrabenazine from the swellable matrix rather than erosion, dissolving or chemical decomposition of the swellable matrix.

As such, the swelling of the polymeric matrix can achieve at least the following objectives: (i) renders the matrix sufficiently large to cause retention in the stomach during the fed mode; (ii) localizes the release of the drug to the stomach and small intestine so that the drug will have its full effect without colonic degradation, inactivation, or loss of bioavailability; (iii) retards the rate of diffusion of the drug long enough to provide multi-hour, controlled delivery of the drug into the stomach.

The tetrabenazine in the swellable matrix can be present in an effective amount of from about 0.1% to about 99% by weight of the matrix. For example, in certain embodiments tetrabenazine is present in the swellable matrix in an amount of from about 0.1% to about 90%, in other embodiments from about 5% to about 90%, in still other embodiments from about 10% to about 80%, and in even still other embodiments from about 25% to about 80% by weight of the swellable matrix.

The water-swellable polymer formling the swellable matrix in accordance with these embodiments of the present invention can be any polymer that is non-toxic, that swells in a dimensionally unrestricted manner upon imbibition of water, and that provides for a modified release of the tetrabenazine. Non-limiting examples of polymers suitable for use in the swellable matrix include cellulose polymers and their derivatives, such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethylenieimine), polyurethane hydrogels, and crosslinked polyacrylic acids and their derivatives, and mixtures thereof. Further examples include copolymers of the polymers listed in the preceding sentence, including block copolymers and grafted polymers. Specific examples of copolymers include PLURONIC® and TECTONIC®, which are polyethylene oxide-polypropylene oxide block copolymers available from BASF Corporation, Chemicals Div., Wyandotte, Mich., USA.

The terms “cellulose” and “cellulosic”, as used within this section regarding the swellable matrix embodiments of the present invention, can denote a linear polymer of anhydroglucose. Non-limiting examples of cellulosic polymers include alkyl-substituted cellulosic polymers that ultimately dissolve in the gastrointestinal (GI) tract in a predictably delayed manner. In certain embodiments the alkyl-substituted cellulose derivatives are those substituted with alkyl groups of 1 to 3 carbon atoms each. Non-limiting examples include methylcellulose, hydroxymethyl-cellulose, hydroxyethylcellulose, hydroxypropylcelluose, hydroxypropylmethylcellulose, carboxymethylcellulose, and mixtures thereof. In terms of their viscosities, one class of alkyl-substituted celluloses includes those whose viscosity is within the range of about 100 to about 110,000 centipoises as a 2% aqueous solution at 20° C. Another class includes those whose viscosity is within the range of about 1,000 to about 4,000 centipoises as a 1% aqueous solution at 20° C. In certain embodiments the alkyl-substituted celluloses are hydroxyethylcellulose and hydroxypropylmethylcellulose. In at least one embodiment the hydroxyethylcellulose is NATRASOL® 250HX NF (National Formulary), available from Aqualon Company, Wilmington, Del., USA.

Polyalkylene oxides that can be used in certain embodiments of the swellable matrices include those having the properties described above for alkyl-substituted cellulose polymers. In at least one embodiment the polyalkylene oxide is poly(ethylene oxide), which term is used herein to denote a linear polymer of unsubstituted ethylene oxide. In at least one embodiment the poly(ethylene oxide) polymers have molecular weights of about 4,000,000 and higher. For example, in certain embodiment the poly(ethylene oxide) polymers have molecular weights within the range of about 4,500,000 to about 10,000,000, and in other embodiments have molecular weights within the range of about 5,000,000 to about 8,000,000. In certain embodiments the poly(ethylene oxide)s are those with a weight-average molecular weight within the range of about 1×105to about 1×107, and in other embodiments within the range of about 9×105 to about 8×106. Poly(ethylene oxide)s are often characterized by their viscosity in solution. For example, in certain embodiments the poly(ethylene oxide)s have a viscosity range of about 50 to about 2,000,000 centipoises for a 2% aqueous solution at 20° C. In at least one embodiment the poly(ethylene oxide) is one or more of POLYOX® NF, grade WSR Coagulant, molecular weight 5 million, and grade WSR 303, molecular weight 7 million, both products of Union Carbide Chemicals and Plastics Company Inc. of Danbury, Conn., USA. Mixtures thereof are operable.

Polysaccharide gums, both natural and modified (semi-synthetic) can be used in the swellable matrix embodiments of the present invention. Non-limiting examples include dextran, xanthan gum, gellan gum, welan gum, rhamsan gum, and mixtures thereof. In at least one embodiment the polysaccharide gum is xanthan gum.

Crosslinked polyacrylic acids that can be used in the swellable matrices of the present invention include those whose properties are the same as those described above for alkyl-substituted cellulose and polyalkylene oxide polymers. In certain embodiments the crosslinked polyacrylic acids are those with a viscosity ranging from about 4,000 to about 40,000 centipoises for a 1% aqueous solution at 25° C. Non-limiting examples of suitable crosslinked polyacrylic acids include CARBOPOL® NF grades 971P, 974P and 934P (BFGoodrich Co., Specialty Polymers and Chemicals Div., Cleveland, Ohio, USA). Further examples of suitable crosslinked polyacrylic acids include polymers known as WATER LOCK®, which are starch/acrylates/acrylamide copolymers available from Grain Processing Corporation, Muscatine, Iowa, USA.

The hydrophilicity and water swellability of these polymers can cause the drug-containing swellable matrices to swell in size in the gastric cavity due to ingress of water in order to achieve a size that can be retained in the stomach when introduced during the fed mode. These qualities also cause the swellable matrices to become slippery, which provides resistance to peristalsis and further promotes their retention in the stomach. The release rate of drug from the swellable matrix is primarily dependent upon the rate of water imbibition and the rate at which the drug dissolves and diffuses from the swollen polymer, which in turn is related to the drug concentration in the swellable matrix. Also, because these polymers dissolve very slowly in gastric fluid, the swellable matrix maintains its physical integrity over at least a substantial period of time, for example in many cases at least about 90% and in certain embodiments over about 100% of the dosing period. The particles will then slowly dissolve or decompose. Complete dissolution or decomposition may not occur until about 24 hours or more after the intended dosing period ceases, although in most cases, complete dissolution or decomposition will occur within about 10 to about 24 hours after the dosing period.

The amount of polymer relative to the drug can vary, depending on the drug release rate desired and on the polymer, its molecular weight, and excipients that may be present in the formulation. The amount of polymer will typically be sufficient to retain at least about 40% of the drug within the swellable matrix about one hour after ingestion (or immersion in the gastric fluid). In certain embodiments, the amount of polymer is such that at least about 50% of the drug remains in the matrix about one hour after ingestion; in other embodiments at least about 60%, and in still other embodiments at least about 80% ofthe drug remains in the swellable matrix about one hour after ingestion. In certain embodiments the drug will be substantially all released from the swellable matrix within about 10 hours; and in other embodiments within about 8 hours, after ingestion, and the polymeric matrix will remain substantially intact until all of the drug is released. In other embodiments the amount of polymer will be such that after about 2 hours no more than about 40% is released; after about 4 hours from about 40% to about 75% is released; after about 8 hours at least about 75% is released, and after about 16 hours at least about 85% is released. The term “substantially intact” is used herein to denote a polymeric matrix in which the polymer portion substantially retains its size and shape without deterioration due to becoming solubilized in the gastric fluid or due to breakage into fragments or small particles.

In other exemplary embodiments the swellable matrix after about 2 hours will release no more than about 40% of the tetrabeniazinie, after about 4 hours from about 40% to about 75%, after about 8 hours at least about 75%, and after about 16 hours at least about 85% of the tetrabenazine.

The water-swellable polymers of the swellable matrices can be used individually or in combination. Certain combinations will often provide a more controlled release of the drug than their components when used individually. Examples include cellulose-based polymers combined with gums, such as hydroxyethyl cellulose or hydroxypropyl cellulose combined with xanthan gum. Another example is poly(ethylene oxide) combined with xanthan gum.

The benefits of certain embodiments of this invention can be achieved over a wide range of drug loadings and polymer levels, with the weight ratio of drug to polymer ranging in general from about 0.01:99.99 to about 80:20, including all values and ranges therebetween. For example, in certain embodiments the drug loadings (expressed in terms of the weight percent of drug relative to total of drug and polymer) are within the range of about 15% to about 80%; in other embodimenits within the range of about 30% to about 80%; and in still other embodiments within the range of about 30% to about 70%. In at least one embodiment the drug loading is within the range of about 0.01% to about 80%, and in at least one other embodiment from about 15% to about 80%. In at least one embodiment the weight ratio of tetrabenazine to polymer in the swellable matrix is from about 15:85 to about 80:20.

The formulations of the swellable matrices of the present invention can assume the form of microparticles, tablets, or microparticles retained in capsules. In at least one embodiment the formulation includes microparticles consolidated into a packed mass for ingestion, even though the packed mass will separate into individual particles after ingestion. Conventional methods can be used for consolidating the microparticles in this manner. For example, the microparticles can be placed in gelatin capsules known in the art as “hard-filled” capsules and “soft-elastic” capsules. The compositions of these capsules and procedures for filling them are known among those skilled in drug formulations and manufacture. The encapsulating material should be highly soluble so that the particles are freed and rapidly dispersed in the stomach after the capsule is ingested.

In certain embodiments of the swellable matrices of the present invention, the formulation contains an additional amount of tetrabenazine applied as a quickly dissolving coating on the outside of the microparticle or tablet. This coating is referred to as a “loading dose” and it is included for immediate release into the recipient's bloodstream upon ingestion of the formulation without first undergoing the diffusion process that the remainder of the drug in the formulation must pass before it is released. The “loading dose” can be high enough to quickly raise the blood concentration of the drug but not high enough to produce the transient overdosing that is characteristic of immediate release dosage forms that are not formulated in accordance with this invention.

In at least one embodiment of the swellable matrices of the present invention, the dosage form is a size 0 gelatin capsule containing either two or three pellets of drug-impregnated polymer. For two-pellet capsules, the pellets are cylindrically shaped, about 6.6 mm or about 6.7 mm in diameter (or more generally, from about 6.5 mm to about 7 mm in diameter) and about 9.5 mm or about 10.25 mm in length (or more generally, from about 9 mm to about 12 mm in length). For three-pellet capsules, the pellets are again cylindrically shaped, about 6.6 mm in diameter and about 7 mm in length. For a size 00 gelatin capsule with two pellets, the pellets are cylindrical, about 7.5 mm in diameter and about 11.25 mm in length. For a size 00 gelatin capsule with three pellets, the pellets are cylindrical, about 7.5 mm in diameter and about 7.5 mm in length. In at least one other embodiment, the dosage form is a single, elongated tablet, with dimensions of about 18 mm to about 22 mm in length, from about 6.5 mm to about 10 mm in width, and from about 5 mm to about 7.5 mm in height. In at least one other embodiment, the dosage form is a single, elongated tablet, with dimensions of from about 18 mm to about 22 mm in length, from about 6.5 mm to about 7.8 mm in width, and from about 6.2 mm to about 7.5 mm in height. In at least one embodiment the dimensions are about 20 mm in length, about 6.7 mm in width, and about 6.4 mm in height. These are merely examples; the shapes and sizes can be varied considerably.

In certain embodiments the tetrabenazine-containing matrix can be made according to any one of the methods described herein.

The particulate drug/polymer mixture or drug-impregnated swellable polymer matrix of certain embodiments can be prepared by various conventional mixing, comminution and fabrication techniques readily apparent to those skilled in the chemistry of drug formulations. Examples of such techniques include: (I) Direct compression, using appropriate punches and dies, such as those available from Elizabeth Carbide Die Company, Inc., McKeesport, Pa., USA; the punches and dies are fitted to a suitable rotary tableting press, such as the Elizabeth-Hata single-sided Hata Auto Press machine, with either 15, 18 or 22 stations, and available from Elizabeth-Hata International, Inc., North Huntington. Pa., USA; (2) Injection or compression molding using suitable molds fitted to a compression unit, such as those available from Cincinnati Milacron, Plastics Machinery Division, Batavia, Ohio, USA.; (3) Granulation followed by compression; and (4) Extrusion in the form of a paste, into a mold or to an extrudate to be cut into lengths.

In regards to the swellable matrices of certain embodiments of the present invention, when microparticles are made by direct compression, the addition of lubricants can be helpful and, in certain embodiments, helpful to promote powder flow and to prevent capping of the microparticle (breaking off of a portion of the particle) when the pressure is relieved. Non-limiting examples of suitable lubricants include magnesium stearate (in a concentration of from about 0.25% to about 3% by weight, and in certain embodiments less than about 1% by weight, in the powder mix), and hydrogenated vegetable oil (in certain embodiments hydrogenated and refined triglycerides of stearic and palmitic acids at from about 1% to about 5% by weight, for example in at least one embodiment at about 2% by weight). Additional excipients can be added to enhance powder flowability and reduce adherence.

Certain embodiments of the swellable matrices of the present invention can find utility when administered to a subject who is in the digestive state (also referred to as the postprandial or “fed” mode). The postprandial mode is distinguishable from the interdigestive (or “fasting”) mode by their distinct patterns of gastroduodenal motor activity, which determine the gastric retention or gastric transit time of the stomach contents.

The controlled release matrices of certain embodiments of the present invention can be manufactured by methods known in the art. An example of a method of manufacturing controlled release matrices is melt-extrusion of a mixture containing the tetrabenazine, hydrophobic polymer(s), hydrophilic polymer(s), and optionally a binder, plasticizer, and other excipient(s) as described above. Other examples of methods of manufacturing controlled release matrices include wet granulation, dry granulation (e.g. slugging, roller compaction), direct compression, melt granulation, and rotary granulation.

Additionally, controlled release particles which can be compressed or placed in capsules can be produced by combining the tetrabenazine and a hydrophobic fusible component and/or a diluent, optionally with a release modifying agent including a water soluble fusible material or a particulate soluble or insoluble organic or inorganic material. Examples of potential hydrophobic fusible components include hydrophobic materials such as natural or synthetic waxes or oils (e.g., hydrogenated vegetable oil, hydrogenated castor oil, microcrystalline wax, Beeswax, carnauba wax and glyceyl monostearate). In at least one embodiment the hydrophobic fusible component has a melting point from about 35° C. to about 140° C. Examples of release modifying agents include polyethylene glycol and particulate materials such as dicalcium phosphate and lactose.

In certain embodiments, controlled release matrices can be produced by mechanically working a mixture of tetrabenazine, a hydrophobic fusible component, and optionally a release component including a water soluble fusible material or a particulate soluble or insoluble organic or inorganic material under mixing conditions that yield aglomerates, breaking down the agglomerates to produce controlled release seeds having desired release properties; and optionally adding more carrier or diluent and repeating the mixing steps until controlled release seeds having desired release properties are obtained. These particles also can be size separated (e.g. by sieving and encapsulated in capsules or compressed into a matrix).

The amount of the hydrophobic fusible material used in the foregoing methods can range from about 10% to about 90% by weight. Mixers useful in such methods are known and include conventional high-speed mixers with stainless steel interiors. For example, a mixture can be processed until a bed temperature of about 40° C. or higher is realized, and the mixture achieves a cohesive granular texture including desired particle sizes.

As noted if the mixture contains agglomerates, they can be broken down using conventional methods to produce a mixture of powder and particles of the desired size which, can be size-separated using a sieve, screen or mesh of the appropriate size. This material can be returned to a high-speed mixer and further processed as desired until the hydrophobic fusible materials begin to soften/melt, and optionally additional hydrophobic material can be added and mixing continued until particles having a desired size range are obtained. Still further, particles containing tetrabenazine can be produced by melt processing as known in the art and combined into capsules or compressed into matrices.

These particles can be combined with one or more excipients such as diluents, lubricants, binding agents, flow aids, disintegrating agents, surface acting agents, water soluble materials, colorants, and the like.

In addition, the controlled release matrices can optionally be coated with one or more functional or non-functional coatings using well-known coating methods. Examples of coatings can include the XR controlled release coat and the EA matrix coating described herein, which can further control the release of the tetrabenazine.

In at least one embodiment, the controlled release matrices can each be coated with at least one taste-masking coating. The taste-masking coating can mask the taste of the tetrabenazine in the matrices. In at least one embodiment the taste-masking coating formulations contain polymeric ingredients. It is contemplated that other excipients consistent with the objects of the present invention can also be used in the taste-masking coating.

In at least one embodiment of the matrix dosage form, the taste-masking coating includes a polymer such as ethylcellulose, which can be used as a dry polymer (such as ETHOCEL®, Dow Corning) solubilized in organic solvent prior to use, or as an aqueous dispersion. One commercially-available aqueous dispersion of ethylcellulose is AQUACOAT(® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the Aquacoat is intimately mixed with a suitable plasticizer prior to use. Another aqueous dispersion of ethylcellulose is commercially available as SURELEASE® (Colorcon, Inc., West Point, Pa., U.S.A.). This product can be prepared by incorporating plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

In other embodiments of the matrix dosage form, polymethacrylate acrylic polymers can be employed as taste masking polymers. In at least one embodiment, the taste masking coating is an acrylic resin lacquer used in the form of an aqueous dispersion, such as that which is commercially available from Rohm Pharma under the trade name EUDRAGIT® or from BASF under the trade name KOLLICOAT®. In further embodiments, the acrylic coating includes a mixture of two acrylic resin lacquers commercially available from Rohm Pharma under the trade names EUDRAGIT® RL and EUDRAGIT® RS, respectively. EUDRAGIT® RL and EUDRAGIT® RS are copolymers of acrylic and methacrylic esters with a low content of quaternary ammonium groups, the molar ratio of ammonium groups to the remaining neutral (meth)acrylic esters being 1:20 in EUDRAGIT® RL and 1:40 in EUDRAGIT® RS. The mean molecular weight is 150,000. The code designations RL (high permeability) and RS (low permeability) refer to the permeability properties of these agents. EUDRAGIT® RL/RS mixtures are insoluble in water and in digestive fluids. However, coatings formed from the same are swellable and permeable in aqueous solutions and digestive fluids. EUDRAGIT® RL/RS dispersions or solutions of the certain embodiments can be mixed together in any desired ratio in order to ultimately obtain a taste masking coating having a desirable drug dissolution profile. Controlled release formulations of certain embodiments can be obtained, for example, from a retardant coating derived from 100% EUDRAGIT® RL; 50% EUDRAGIT® RL with 50% EUDRAGIT® RS; and 10% EUDRAGIT® RL with 90% EUDRAGIT® RS.

In other embodiments of the matrix dosage form, the taste masking polymer can be an acrylic polymer which is cationic in character based on dimethylaminoethyl methacrylate and neutral methacrylic acid esters (such as EUDRAGIT® E, commercially available from Rohm Pharma). The hydrophobic acrylic polymer coatings of the present invention can further include a neutral copolymer based on poly (meth)acrylates, such as EUDRAGIT® NE (NE=neutral ester), commercially available from Rohm Pharma. EUDRAGIT® NE 30D lacquer films are insoluble in water and digestive fluids, but permeable and swellable.

In other embodiments of the matrix dosage form, the taste masking polymer is a dispersion of poly (ethylacrylate, methyl methacrylate) 2:1 (KOLLICOAT® EMM 30 D, BASF).

In other embodiments of the matrix dosage form, the taste masking polymer can be a polyvinyl acetate stabilized with polyvinylpyrrolidone and sodium lauryl sulfate such as KOLLICOAT® SR30D (BASF).

Other taste masking polymers that can be used in the matrix dosage forms include hydroxypropylcellulose (HPC); hydroxypropylmethylcellulose (HPMC); hydroxyethylcellulose; gelatin; gelatin/acacia; gelatin/acacia/vinvylmethylether maleic anhydride; gelatin/acacia/ethylenemaleic anhydride; carboxymethyl cellulose; polyvinvylalcohol; nitrocellulose; polyvinylalcohol-polyethylene glycol graft-copolymers; shellac; wax and mixtures thereof.

The taste-masking coatings can be applied to the matrices from one or more organic or aqueous solvent solutions or suspensions. In at least one embodiment of the matrix dosage forms the organic solvents that can be used to apply the taste-masking coatings include one or more of acetone, lower alcohols such as ethanol, isopropanol and alcohol/water mixtures, chlorinated hydrocarbons, and the like. Devices used to coat the matrices of certain embodiments with a taste-masking coating include those conventionally used in pharmaceutical processing, such as fluidized bed coating devices. The controlled release coatings applied to the matrices can contain ingredients other than the cellulosic polymers. One or more colorants, flavorants, sweeteners, can also be used in the taste-masking coating.

In some embodiments of the matrix dosage forms, a pore former can be included into the taste masking coat in order to influence the rate of release of tetrabenazine from the matrix. In other embodiments, a pore former is not included in the taste masking coat. The pore formers can be inorganic or organic, and may be particulate in nature and include materials that can be dissolved, extracted or leached from the coating in the environment of use. Upon exposure to fluids in the environment of use, the pore-formers can for example be dissolved, and channels and pores are formed that fill with the environmental fluid.

For example, the pore-formers of certain embodiments of the matrix dosage forms can include one or more water-soluble hydrophilic polymers in order to modify the release characteristics of the formulation. Examples of suitable hydrophilic polymers that can be used as pore-former-s include hydroxypropylmetlhylcellulose, cellulose ethers and protein-derived materials of these polymers, the cellulose ethers, such as hydroxyalkylcelluloses. carboxyalkylcelluloses and mixtures thereof. Also, synthetic water-soluble polymers can be used, examples of which include polyvinylpyrrolidonle, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, sorbitol and mixtures thereof. In at least one embodiment, the hydrophilic polymer includes hydroxypropyl-methylcellulose.

Other non-limiting examples of pore-formers that can be used in the taste masking coat include alkali metal salts such as lithium carbonate, sodium chloride, sodium bromide, potassium chloride, potassium sulfate, potassium phosphate, sodium acetate, sodium citrate and mixtures thereof. The pore-forming solids can also be polymers which are soluble in the environment of use, such as CARBOWAX™ and CARBOPOL™. In addition, the pore-formers embrace diols, polyols, polyhydric alcohols, polyalkylene glycols, polyglycols, poly(a-w)alkylenediols and mixtures thereof. Other pore-formers which can be useful in the formulations of certain embodiments of the present invention include starch, modified starch, and starch derivatives, gums, including but not limited to xanthan gum, alginic acid, other alginates, benitonite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambda-carrageenan, gum karaya, biosynthetic gum, and mixtures thereof. Other pore-formers include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In general, the amount of pore-former included in the taste masking coatings of certain embodiments of the matrix dosage forms can be from about 0.1% to about 80%, by weight, relative to the combined weight of polymer and pore-forimier. The percentage of pore former as it relates to the dry weight of the taste-masking polymer, can have an influence on the drug release properties of the coated matrix. In at least one embodiment that uses water soluble pore formers such as hydroxypropylmethylcellulose, a taste masking polymer: pore former dry weight ratio of from about 10:1 to about 1:1 can be present. In certain embodiments the taste masking polymer: pore former dry weight ratio is from about 8:1 to about 1.5:1; and in other embodiments from about 6:1 to about 2:1. In at least one embodiment using EUDRAGIT® NE30D as the taste masking polymer and a hydroxypropylmethylcellulose (approx 5cps viscosity (in a 2% aqueous solution)) such as METHOCEL® E5, PHARMACOAT® 606G as the water soluble pore former, a taste masking polymer: pore former dry weight ratio of about 2:1 is present.

Colorants that can be used in the taste-masking coating of certain embodiments of the matrix dosage forms include food, drug and cosmetic colors (FD&C), drug and cosmetic colors (D&C) or external drug and cosmetic colors (Ext. D&C). These colors are dyes, lakes, and certain natural and derived colorants. Useful lakes include dyes absorbed on aluminum hydroxide or other suitable carriers.

Flavorants that can be used in the taste-masking coating of certain embodiments of the matrix dosage forms include natural and synthetic flavoring liquids. An illustrative list of such flavorants includes volatile oils, synthetic flavor oils, flavoring aromatics, oils, liquids, oleoresins and extracts derived from plants, leaves, flowers, fruits, stems and combinations thereof. A non-limiting representative list of these includes citric oils, such as lemon, orange, grape, lime and grapefruit, and fruit essences, including apple, pear, peach, grape, strawberry, raspberry, cherry, plum, pineapple, apricot, or other fruit flavors. Other useful flavorants include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

Sweeteners that can be used in the taste-masking coating of certain embodiments of the matrix dosage forms include glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Steva Rebaudiana (Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners can be used alone or in any combination thereof.

The matrix taste masking coat can also include one or more pharmaceutically acceptable excipients such as lubricants, emulsifiers, anti-foaming agents, plasticizers, solvents and the like.

Lubricants can be included to help reduce friction of coated matrices during manufacturing. The lubricants that can be used in the taste masking coat of certain embodiments of the present invention include but are not limited to adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (i.e. COMPRITOL™), STEAR-O-WET™, MYVATEX™ TL and mixtures thereof. In at least one embodiment, the lubricant is selected from magnesium stearate, talc and a mixture thereof. The lubricant can be present in an amount of from about 1% to about 100% by weight of the polymer dry weight in the taste masking coat. For example, ih certain embodiments wherein the taste masking polymer is EUDRAGIT® NE30D or EUDRAGIT® NE40D (Rohm America LLC) together with a hydrophilic pore former, the lubricant is present in an amount of from about 1% to about 30% by weight of the polymer dry weight; in other embodiments from about 2% to about 20%; and in still other embodiments at about 10% by weight of the matrix taste masking coat dry weight. In another embodiment where the taste masking polymer is ethylcellulose (ETHOCEL™ PR100, PR45, PR20, PR10 or PR7 polymer, or a mixture thereof), the lubricant can be present in an amount of from about 10% to about 100% by weight of the matrix taste-masking coat dry weight; in another embodiment from about 20% to about 80%; and in still another embodiments at about 50% by weight of the matrix taste masking coat dry weight. In other embodiments, the taste masking coat does not include a pore former.

Emulsifying agent(s) (also called emulsifiers or emulgents) can be included in the matrix taste masking coat to facilitate actual emulsification during manufacture of the coat, and also to ensure emulsion stability during the shelf-life of the product. Emulsifying agents useful for the matrix taste masking coat composition of certain embodiments include, but are not limited to naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Combinations of emulsifying agents are operable. In at least one embodiment, the emulsifying agent is TWEEN™ 80. The emulsifying agent(s) can be present in an amount of from about 0.01% to about 5% by weight of the matrix taste masking polymer dry weight. For example, in certain embodiments the emulsifying agent is present in an amount of from about 0.05% to about 3%; in other embodiments from about 0.08% to about 1.5%, and in still other embodiments at about 0.1% by weight of the matrix taste masking polymer dry weight.

Anti-foaming agent(s) can be included in the matrix taste masking coat to reduce fi-othing or foaming during manufacture of the coat. Anti-foaming agents useful for the coat composition include, but are not limited to simethicone, polyglycol, silicon oil, and mixtures thereof. In at least one embodiment the anti-foaming agent is Simethicone C. The anti-foaming agent can be present in an amount of from about 0.1% to about 10% of the matrix taste masking coat weight. For example, in certain embodiments the anti-foaming agent is present in an amount of from about 0.2% to about 5%; in other embodiments from about 0.3% to about 1%, and in still other embodiments at about 0.6% by weight of the matrix taste masking polymer dry weight.

Plasticizer(s) can be included in the matrix taste masking coat to provide increased flexibility and durability during manufacturing. Plasticizers that can be used in the matrix taste masking coat of certain embodiments include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, and mixtures thereof. The plasticizer can be present in an amount of from about 1% to about 80% of the taste masking polymer dry weight. For example, in certain embodiments the plasticizer is present in an amount of from about 5% to about 50%, in other embodiments from about 10% to about 40%, and in still other embodiments at about 20% of the taste masking polymer dry weight.

In some embodiments mixtures of plasticizers are provided, e.g., a mixture of PEG 4000 and Dibutyl Sebacate (DBS).

The taste-masking coating can be present in an amount of from about 1% to about 90% by weight of the matrix, depending upon the choice of polymer, the ratio of polymer :pore former, and the total surface area of the matrix formulation. Since a certain thickness of taste masking coating has to be achieved in order to achieve effective taste masking, the amount of taste masking polymer coating used during manufacture is related to the total surface area of the batch of uncoated matrices that requires a coating. For example, the taste masking polymer surface area coverage can range from about 0.5 mg/cm² to about 20 mg/cm². For example, in certain embodiments the surface area coverage of the taste masking polymer is from about 0.6 mg/cm² to about 10 mg/cm², and in other embodiments is from about 1 mg/cm² to about 5 mg/cm². In at least one embodiment of the invention, EUDRAGIT® E is employed as the taste masking polymer at a surface area coverage of about 4 mg/cm2.

In the absence of an accurate determination of total surface area of a matrix, the amount of taste masking polymer to be applied can be expressed as a percentage of the uncoated matrix. For example, in certain embodiments the taste-masking coating is present in an amount of from about 5% to about 60%; in other embodiments from about 10% to about 40%; and in still other embodiments from about 15% to about 35% by weight of the matrix. In at least one embodiment the taste-masking coating is present in an amount of about 30% by weight of the matrix.

Prophetic examples of matrix tablet formulations are described below. It should be understood that these examples are intended to be exemplary and that the specific constituents, amounts thereof, and formulation methods may be varied therefiom in order to achieve different release characteristics:

In at least one embodiment, the controlled matrices include:

| | |
|---|---|
| Tetrabenazine | about 30.0% by weight of the matrix |
| Hydroxypropylmethylcellulose E50 | about 10.0% by weight of the matrix |
| Hydroxypropylmethylcellulose | about 30.0% by weight of the matrix |
| K15M | |
| Calcium phosphate dehydrate | about 9.5% by weight of the matrix |
| ATMUL ™ 84S | about 20.0% by weight of the matrix |
| (mono/di/tri glycerides) | |
| Magnesium stearate | about 0.5% by weight of the matrix |

Preparation of the matrix formulation can be as follows: Combine the drug, a portion of each HPMC, calcium phosphate and Atmul 84S in a planetary mixer and dry mix for 15 minutes. Add a solution of the remainder of the HPMC in water to the mixer while mixing, until a wet mass is obtained. Pass the wet material through a screen to make the resultant granules of uniform size (to achieve uniform drying) and dry in an oven at about 40° C. for about 24 hours. Mill the dried granules through a Fitzpatrick Mill, knives forward, and collect the material in a mixer. Add the magnesium stearate and mix for about 5 minutes. The resultant mixture is tableted on a suitable tablet press.

In at least one embodiment, the controlled release matrices include a deposit-core and support-platform. Preparation of the deposit-core can be as follows: Deposit-cores can be prepared using the following materials in the stated quantities:

| | |
|---|---|
| Tetrabenazine | about 45.0 g |
| hydroxypropyl methylcellulose | about 35.0 g |
| (METHOCEL ® K 100M-Colorcon) | |
| mannitol | about 10.0 g |
| ethylcellulose (high viscosity-BDH) | about 3.75 g |
| 3.75 g magnesium stearate | about 1.0 g |
| 5:1 ethanol-chloroform mixture | about 75.0 ml |

The tetrabenazine is mixed intimately with the mannitol and hydroxypropyl methylcellulose in a suitable mixer. The solution of ethylcellulose in ethanol-chloroform is prepared separately, and is used for wetting the previously obtained powder mixture. The resultant homogeneous mass is forced through an 800 micron screen and then dried to obtain a granulate which is passed through a 420 micron screen. The homogeneous granulate obtained is mixed with the magnesium stearate and then compressed using concave punches of diameter 7 mm (radius of curvature 9 mm) using a pressure of about 3000 kg/cm² to obtain cylindrical deposit-cores with convex bases.

Application of the support-platform can be as follows: The support-platform can be applied by coating one or both the convex bases of the deposit-core with a solution of about 15 g low-permeability acrylic-methacrylic copolymer (EUDRAGIT® RS Rohm Pharma) in methylene chloride of a quantity to make up to 100 ml. Thereafter about 0.3 ml of said solution is applied to each base to be covered, taking care to protect the lateral core surface. The system is then dried with tepid air. The quantity of polymeric material deposited is sufficient to keep the structure intact during transfer.

In at least one embodiment, the matrix formulation is a polyethylene oxide (PEO) based tablet matrix formulation including:

| | |
|---|---|
| Tetrabenazine | about 50% |
| PEO WSR Coagulant | about 15% |
| (polyethylene oxide) | |
| METHOCEL ® K100M | about 15% |
| (hydroxypropylmethyl cellulose) | |
| Avicel PH101 | about 19% |
| (microcrystalline cellulose) | |
| Magnesium Stearate | about 1% |

Preparation of the PEO based tablet matrix formulation can be as follows: Excipients dry blended in an appropriate mixer and compressed into tablets using conventional apparatus.

Multiparticulates

In certain embodiments of the present invention, a multiparticulate system is provided which contains multiple microparticles each containing an effective amount of tetrabenazine and at least one pharmaceutically acceptable excipient. The multiparticulates can be contained within a capsule, or can be compressed into a matrix or tablet, that upon ingestion disintegrate into multiple units (e.g. pellets), wherein the sub-units or pellets possess the desired controlled release properties of the dosage form. The multiparticulates or the multiple unit dosage forms can be surrounded by one or more coatings. Examples of such coatings include polymeric controlled release coatings, delayed release coatings, enteric coatings, immediate release coatings, taste-masking coatings, extended release coatings, and non-functional coatings.

The tetrabenazine in the microparticles of certain embodiments can be present in an effective amount of from about 0.1% to about 99% by weight of the microparticles. For example, il certain embodiments tetrabenazine is present in the microparticles in an amount of from about 0.1% to about 90%, in other embodiments from about 5% to about 90%, in still other embodiments from about 10% to about 80%, and in even still other embodiments from about 20% to about 75% by weight of the microparticle. In certain embodiments wherein the microparticles are manufactured using a splieronization process, the tetrabenazine can be present in the microparticles in an amount of from about 0.1% to about 60%; in other such embodiments from about 5% to about 50%; and in still other such embodiments from about 10% to about 40% by weight of the microparticle. In at least one embodiment wherein the microparticles are manufactured using a spheronization process, the tetrabenazine is present in the microparticle in an amount of about 30% by weight of the microparticle. In certain embodiments wherein the microparticles are manufactured using a drug layering on bead process, the tetrabenazine can be present in the microparticles in an amount of from about 0.1% to about 60%; in other such embodiments from about 5% to about 50%. and in still other such embodiments from about 10% to about 40% by weight of the microparticle. In at least one embodiment wherein the micropat-ticles are manufactured using a drug layering on bead process, the tetrabenazine is present hi the microparticle in an amount of about 25% by weight of the mnicroparticle.

In addition to the tetrabenazine, the microparticles of the present invention also include at least one pharmaceutically acceptable excipient. Excipients can be added to facilitate in the preparation, patient acceptability and functioning of the dosage form as a drug delivery system. Examples of possible excipients include spheronization aids, solubility enhancers, disintegrating agents, diluents, lubricants, binders, fillers, glidants, suspending agents, emulsifying agents, anti-foaming agents, flavoring agents, coloring agents, chemical stabilizers, pH modifiers, and mixtures thereof. Depending on the intended main function, excipients to be used in formulating compositions are subcategorized into different groups. However, one excipient can affect the properties of a composition in a series of ways, and many excipients used in compositions can thus be described as being multifunctional.

The microparticles of certain embodiments of the present invention can be manufactured using standard techniques known to one of skill in the art. In certain embodiments the microparticles can be made according to any one of the methods described herein. Useful microparticles include drug-layered microparticles and drug-containing microparticles.

Drug-Containing Microparticles

Microparticles containing drug in the core can be prepared by a number of different procedures. For example: In a spray drying process, an aqueous solution of core material and hot solution of polymer is atomized into hot air, the water then evaporates, and the dry solid is separated in the form of pellets, for example by air suspension. A spray-drying process can produce hollow pellets when the liquid evaporates at a rate that is faster than the diffusion of the dissolved substances back into the droplet interior, or if due to capillary action the dissolved substance migrates out with the liquid to the droplet surface, leaving behind a void. Another example is a spray congealing process, where a slurry of drug material that is insoluble in a molten mass is spray congealed to obtain discrete particles of the insoluble materials coated with the congealed substance. A further example is a fluidized bed based granulation/pelletization process, where a dry drug is suspended in a stream of hot air to form a constantly agitated fluidized bed. An amount of binder or granulating liquid is then introduced in a finely dispersed form to cause pelletization.

The drug-containing microparticles of certain embodiments of the present invention can also be made by, for example, a spheronization process. One method of manufacturing the drug-containing microparticles is the applicant's proprietary CEFORM™ (Centrifugally Extruded & Formed Microspheres/Microparticles) technology, which is the simultaneous use of flash heat and centrifugal force, using proprietary designed equipment, to convert dry powder systems into microparticles of uniform size and shape. The production of microparticles containing an active drug using this CEFORM™ technology is known. This patent deals with the use of LIQUIFLASH® processing to spheronize compositions containing one or more active drugs to form LIQUIFLASH® microparticles.

With the CEFORM™ technology, the processing of the drug-containing microparticles of the present invention is carried out in a continuous fashion, whereby a pre-blend of drug and excipients is fed into a spinning “microsphere head”, also termed as a “spheronizing head”. The microsphere head, which is a multi-aperture production unit, spins on its axis and is heated by electrical power. The drug and excipient(s) pre-blend is fed into the center of the head with an automated feeder. The material moves, via centrifugal force, to the outer rim where the heaters, located in the rim of the head, heat the material. Microparticles are formed when the molten material exits the head, which are then cooled by convection as they fall to the bottom of the microparticle chamber. The product is then collected and stored in suitable product containers. Careful selection of the types and levels of excipient(s) control microparticle properties such as sphericity, surface morphology, and dissolution rate. One advantage of such a process is that the microparticles are produced and collected from a dry feedstock without the use of any solvents.

There are at least two approaches that can be used to produce drug-containing microparticles using the CEFORM process: (i) the encapsulation approach and (ii) the co-melt approach. In the encapsulation approach, the process is conducted below the melting point of the drug. Therefore, the excipients are designed to melt and entrain the drug particles on passing through the apertures to form microparticles. The resulting microparticles contain the drug, in its native state, essentially enveloped by or as an intimate matrix with the resolidified excipients. In the co-melt approach, the process is conducted above the melting point of the drug. In this case, the drug and the excipients melt or become fluid simultaneously upon exposure to the heat. The molten mixture exits the head and forms microparticles, which cool as they fall to the bottom of the collection bin where they are collected.

In at least one embodiment the microparticles are manufactured using the encapsulation approach. In the encapsulation approach the excipient(s) which are chosen have a lower melting point than the drug with which they will be combined. Therefore the spheronizing process can be performed at lower temperatures. than the melting point of the drug. As a result, this can reduce the risk of polymeric interconversion, which can occur when using processing temperatures close to the melting point.

In a prophetic example of certain embodiments of the present invention, the manufacturing process for the microparticles can hypothetically be as follows: Spheronization aid is screened through a 425 micron (μm) screen. In at least one embodiment, the spheronization aid is distilled glyceryl monostearate (i.e. DMG-03VF). About 50% of the spheronization aid is added to a bowl in a high shear mixer. In at least one embodiment, the bowl is a 6 liter bowl and the high shear mixer is a Diosna P1-6 high speed mixer granulator. The active drug is then added to the bowl of the mixer, and then the remainder of the spheronization aid is added. The material is then blended in the mixer for a time from about 1 minute to about 30 minutes; in certain embodiments from about 3 minutes to about 10 minutes; and in at least one embodiment at about 6 minutes. The mixer motor speed is from about 50 rpm to about 2000 rpm; in certain embodiments from about 200 rpm to about 500 rpm; and in at least one embodiment at about 300 rpm. The chopper motor speed is from about 50 rpm to about 2000 rpm; in certain embodiments from about 200 rpm to about 500 rpm; and in at least one embodiment at about 400 rpm. The blended material is then spheronized in a CEFORM™ spheronizing head. The spheronizing head speed is from about 5 Hz to about 60 Hz; in certain embodiments from about 10 Hz to about 30 Hz; and in at least one embodiment at about 15 Hz. In at least one embodiment the CEFORM™ spheronizing head is a 5 inch head. The spheronizing head temperature is maintained at a temperature from about 70° C. to about 110° C.; in certain embodiments from about 80° C. to about 105° C.; and in at least one embodiment at about 95° C. The microparticles obtained from the spinning process are then screened through a screen that is from about 150 μm to about 800 μm.

For microparticles manufactured using a spheronization process such as the CEFORM™ process, the microparticles include, in addition to the tetrabenazine, at least one spheronization aid. Spheronization aids can assist the drug-containing mix to form robust durable spherical particles. Some examples of materials useful as spheronization aids include, but are not limited to glyceryl monostearate, glyceryl behenate, glyceryl dibehenate, glyceryl palmitostearate, hydrogenated oils such as hydrogenated castor oil marketed under the name CUTINA™ HR, fatty acid salts such as magnesium or calcium stearate, polyols such as mannitol, sorbitol, xylitol, stearic acid, palmitic acid, sodium lauryl sulfate, polyoxyethylene ethers, esterified polyoxyethylenes such as PEG-32 distearate, PEG-150 distearate, cetostearyl alcohol, waxes (e.g. carnauba wax, white wax, paraffin wax) and wax-like materials. Certain thermo-plastic or thermo-softening polymers can also function as spheronization aids. Some non-limiting examples of such thermo-plastic or thermo-softening polymers include Povidone, cellulose ethers and polyvinylalcohols. Combinations of spheronization aids can be used. In at least one embodiment, the spheronization aid is glyceryl monostearate (i.e. DMG-03VF). The spheronization aid can be present in an amount of from about 0.1% to about 99% by weight of the microparticle. For example, in certain embodiments the spheronization aid is present in an amount of from about 5% to about 90%; in other embodiments from about 10% to about 80%; in still other embodiments from about 15% to about 70%; and in even still other embodiments from about 20% to about 60% by weight of the microparticle. In at least one embodiment the spheronization aid is present in an amount of about 50% by weight of the micropar-ticle. In at least one othel embodiment, the microparticles include about 50% (w/w) oftetrabenazine and about 50% (w/w) of the spheronizationi aid.

In certain embodiments, each micropar-ticle can also include at least one solubility enhancer. Solubility enhancers can act as splieronizilng aids and be used as the sole excipient with the tetrabenazine. Solubility enhancers can be surfactatits. Certain embodiments of the invention include a solubility enhancer that is a hydrophilic sulfactanit. Hydrophiilic surfactanits can be used to provide any of several advantageous characteristics to the compositions, including: increased solubility of the tetrabenazine in the microparticle; improved dissolution of tetrabenazine; improved solubilization of the tetrabenazine upon dissolution; enhanced absorption and/or bioavailability of the tetrabenazine. The hydrophilic surfactant can be a single hydrophilic surfactant or a mixture of hydrophilic surfactants, and can be ionic or non-ionic.

Likewise, various other embodiments of the invention include a lipophilic component, which can be a lipophilic surfactant. including a mixture of lipophilic surfactants, a triglyceride, or a mixture thereof. The lipophilic surfactant can provide any of the advantageous characteristics listed above for hydrophilic surfactants, as well as further enhancing the function of the surfactants. These various embodiments are described in more detail below.

As is well known in the art, the terms “hydrophilic” and “lipophilic” are relative terms. To function as a surfactant, a compound includes polar or charged hydrophilic moieties as well as non-polar hydrophobic (lipophilic) moieties; i.e., a surfactant compound is amphiphilic. An empirical parameter commonly used to characterize the relative hydrophilicity and lipophilicity of non-ionic amphiphilic compounds is the hydrophilic-lipophilic balance (the “HLB” value). Surfactants with lower HLB values are more lipophilic, and have greater solubility in oils, whereas surfactants with higher HLB values are more hydrophilic, and have greater solubility in aqueous solutions.

Using HLB values as a rough guide, hydrophilic surfactants can generally be considered to be those compounds having an HLB value greater than about 10, as well as anionic, cationic, or zwitterionic compounds for which the HLB scale is not generally applicable. Similarly, lipophilic surfactants can be compounds having an HLB value less than about 10.

It should be appreciated that the HLB value of a surfactant is merely a rough guide generally used to enable formulation of industrial, pharmaceutical and cosmetic emulsions. For many surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or lipophilicity for use in the present invention, as described herein.

Solubility enhancers can be any surfactant suitable for use in pharmaceutical compositions. Suitable surfactants can be anionic, cationic, zwitterionic or non-ionic.

Refined, distilled or fractionated surfactants, purified fractions thereof, or re-esterified fractions, are within the scope of the invention.

Although polyethylene glycol (PEG) itself does not function as a surfactant, a variety of PEG-fatty acid esters have useful surfactant properties. Polyethylene glycol (PEG) fatty acid diesters are also suitable for use as surfactants in the compositions of the present invention. In general, mixtures of surfactants are also useful in the present invention, including mixtures of two or more commercial surfactant products. Several PEG-fatty acid esters are marketed commercially as mixtures or mono- and diesters.

A large number of surfactants of different degrees of lipophilicity or hydrophilicity can be prepared by reaction of alcohols or polyalcohols with a variety of natural and/or hydrogenated oils. In certain embodiments, the oils used are castor oil or hydrogenated castor oil or an edible vegetable oil such as corn oil, olive oil, peanut oil, palm kernel oil, apricot kernel oil, or almond oil. Examples of alcohols include glycerol, propylene glycol, ethylene glycol, polyethylene glycol, sorbitol, and pentaerythritol. Polyglycerol esters of fatty acids are also suitable surfactants for the present invention. Esters of propylene glycol and fatty acids are suitable surfactants for use in the present invention. In general, mixtures of surfactants are also suitable for use in the present invention. In particular, mixtures of propylene glycol fatty acid esters and glycerol fatty acid esters are suitable and are commercially available.

Another class of surfactants is the class of mono- and diglycerides. These surfactants are generally lipophilic. Sterols and derivatives of sterols are suitable surfactants for use in the present invention. These surfactants can be hydrophilic or lipophilic. A variety of PEG-sorbitan fatty acid esters are available and are suitable for use as surfactants in the present invention. In general, these surfactants are hydrophilic, although several lipophilic surfactants of this class can be used. Ethers of polyethylene glycol and alkyl alcohols are suitable surfactants for use in the present invention. Esters of sugars are suitable surfactants for use in the present invention. Several hydrophilic PEG-alkyl phenol surfactants are available, and are suitable for use in the present invention. Sorbitan esters of fatty acids are suitable surfactants for use in the present invention.

Esters of lower alcohols (C2 to C4) and fatty acids (C8 to C18) are suitable surfactants for use in the present invention. Ionic surfactants, including cationic, anionic and zwitterionic surfactants, are suitable hydrophilic surfactants for use in the present invention. In certain embodiments, the surfactant is an anionic surfactant such as a fatty acid salt, a bile salt, or a combination thereof. In other embodiments the surfactant is a cationic surfactant such as a carnitine. Examples of ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Ionizable surfactants, when present in their unionized (neutral, non-salt) form, are lipophilic surfactants suitable for use in the compositions of the present invention. Particular examples of such surfactants include free fatty acids, particularly C6-C22 fatty acids, and bile acids. Derivatives of oil-soluble vitamins, such as vitamins A, D, E, K, etc., are also useful surfactants for the compositions of the present invention. An example of such a derivative is tocopheryl PEG-1000 succinate (TPGS, available from Eastman).

In certain embodiments, surfactants or mixtures of surfactants that solidify at ambient room temperature are used. In other embodiments, surfactants or mixtures of surfactants that solidify at ambient room temperature in combination with particular lipophilic components, such as triglycerides, or with addition of appropriate additives, such as viscosity modifiers, binders, thickeners, and the like, are used.

Examples of non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxpropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; polyethoxylated fat-soluble vitamins or derivatives; and mixtures thereof.

In certain embodiments, the non-ionic hydrophilic surfactant is selected from the group including polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; polyoxyethylene hydrogenated vegetable oils, and mixtures thereof. The glyceride can be a monoglyceride, diglyceride, triglyceride, or a mixture thereof.

In certain other embodiments, the surfactants used are non-ionic hydrophilic surfactants that are reaction mixtures of polyols and fatty acids, glycerides, vegetable oils, hydrogenated vegetable oils or sterols. These reaction mixtures are largely composed of the transesterification products of the reaction, along with often complex mixtures of other reaction products. The polyol can be glycerol, ethylene glycol, polyethylene glycol, sorbitol, propylene glycol, pentaerythritol, a saccharide. or a mixture thereof.

The hydrophilic surfactant can also be, or include as a component, an ionic surfactant. Examples of ionic surfactants include alkyl ammonium salts; bile acids and salts, analogues, and derivatives thereof; fusidic acid and derivatives thereof; fatty acid derivatives of amino acids, oligopeptides, and polypeptides; glyceride derivatives of amino acids, oligopeptides, and polypeptides; acyl lactylates; mono-,diacetylated tartaric acid esters of mono-,diglycerides; succinylated monoglycerides; citric acid esters of mono-,diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

In certain embodiments the ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono-,diacetylated tartaric acid esters of mono-,diglycerides; succinylated monoglycerides; citric acid esters of mono-diglycerides; carnitines; and mixtures thereof.

Examples of ionic surfactants include lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

In certain embodiments, ionic surfactants used include lecithin, lysolecithin, phosphatidylcholine, phosphatidylethanolanmine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof. In at least one embodiment, the ionic surfactant is selected from lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

Examples of lipophilic surfactants include alcohols; polyoxyethylenle alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylenle sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; and mixtures thereof.

As with the hydrophilic surfactants, lipophilic surfactants can be reaction mixtures of polyols and fatty acids, glycerides, vegetable oils, hydrogenated vegetable oils, and sterols.

In certain embodiments, the lipophilic surfactants include one or more selected from the group including fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, sterols, and mixtures thereof.

In certain other embodiments, the lipophilic surfactants include one or more selected from the group including lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof. Among the glycerol fatty acid esters, the esters can be mono- or diglycerides, or mixtures of mono- and diglycerides, where the fatty acid moiety is a C6 to C22 fatty acid.

Other embodiments include lipophilic surfactants which are the reaction mixture of polyols and fatty acids, glycerides, vegetable oils, hydrogenated vegetable oils, and sterols. Examples of polyols are polyethylene glycol, sorbitol, propylene glycol, pentaerythritol, and mixtures thereof.

Combinations of solubility enhancers (i.e. surfactants) can be used. Examples of macrogol fatty acid esters useful as solubility enhancers include GELUCIRE® 50/13 and GELUCIRE® 44/14. In at least one embodiment the solubility enhancer is GELUCIRE® 50/13. The solubility enhancer can be present in an amount of from about 0.1% to about 99% by weight of the microparticle. For example, in certain embodiments, the solubility enhancer is present in an amount of from about 1% to about 80%; in other embodiments from about 10% to about 60%; in still other embodiments from about 15% to about 45% by weight of the microparticle. In at least one embodiment the solubility enhancer is present in an amount of about 35% by weight ofthe microparticle.

It is contemplated that in some embodiments, one or more other pharmaceutically acceptable excipients consistent with the objects of the present invention can be used in the microparticles, such as a lubricant, a binder, a pH modifier, a filler and/or a glidant.

The process for manufacturing the drug-containing microparticles of certain embodiments of the present invention by spheronization are not limited to the CEFORM™ technology, and any other technology resulting in the formation of the microparticles consistent with the objects of the present invention can also be used. For example, microparticles of certain embodiments of the invention can also be manufactured by extrusion/spheronization, granulation or pelletization.

Extrusion/spheronization is a multi-step process used to make uniformly sized spherical particles. The technique offers the ability to incorporate active ingredients without producing excessively large particles. The main steps in the process are:

Dry-mixing of ingredients to achieve a homogenous powder dispersion;

Wet massing using for example a high-shear wet granulator to form rod

shaped particles of uniform diameter;

Extrusion to form rod-shaped particles of uniform diameter:

Spheronization to round off the rods into spherical particles;

Screening to achieve the desired narrow particle size distribution.

The mixing vessel used for dry-mixing can be of any size and shape compatible with the size of the formulation to be produced. For example, commercially available mixing devices such as planetary mixers, high shear mixers, or twin cone blenders can be used. If relatively small quantities of formulation are to be prepared, a simple mortar and pestle can be sufficient to mix the ingredients. The type of mixing vessel would be apparent to one skilled in the pharmaceutical art. The moistened mass formed by wet-massing in conventional granulation equipment is extruded through a perforated mesh in order to produce cylindrical filaments. The port of the meshes can determine the diameter of the filaments. A port ranging from about 0.2 mm to about 3 mm can be used in this process. In at least one embodiment utilizing this process, the port ranges from about 0.4 mm to about 2 mm. The extrusion can be carried out using screw, double screw, “sieve and basket” kind, “roll extruder”, “ram extruder” extruders or any other pharmaceutically acceptable means to produce cylindrical filaments. In certain embodiments utilizing this extrusion/spheronization process, a double screw coaxial extruder is used. The spheronization device includes a hollow cylinder with a horizontal rotating plate. The filaments are broken in short segments which are transformed in spherical or quasi-spherical particles on the upper surface of the rotating plate at a velocity ranging from about 200 rpm to about 2,000 rpm. The particles can be dried in any pharmaceutically acceptable way, such as for example by air drying in a static condition. The particles are used as they are or they are coated to obtain granules to use in tablets, capsules, packets or other pharmaceutical formulations.

A prophetic example of an extrusion/spheronization formulation including tetrabenazine can be as follows: In this example, the tetrabenazine can be present in an amount of from about 1% to about 70% w/w. In certain embodiments within this example, the tetrabenazine is present in an amount of from about 10% to about 40% w/w; in other embodiments from about 10% to about 20%; and in still other embodiments about 10% w/w. In this example, the filler can be present in an amount of from about 0% to about 90% w/w. In certain embodiments of thi s example, the filler is present in an amount of from about 10% to about 70%; and in other embodiments at about 50% w/w. In this example, the microcrystalline cellulose can be present in an anount of frof about 10% to about 90% w/w. In certain embodiments of this example, the microcrystalline cellulose is present in an amount of from about 10% to about 80%; and in other embodiments from about 20% to about 60% w/w. In this example, the bindei can be present in an amount of from about 0% to about 10% w/w. In certain embodiments of this example, the binder is present in an amount of from about 1% to about 8%; and in other eebodiments from about 2% to about 4% w/w. In this example, water can be present in an amount of from about 10% to about 80% w/w. In certain embodiments of this example, water is present in an amount of from about 15% to about 70%; and in other embodiments from about 20% to about 50% w/w. Suitable fillers that can be used in this example include but are not limited to calcium phosphate dibasic, tricalcium phosphate, calcium carbonate, starcc (such as corn, maize, potato and rice starches), modified starches (such as carboxymetliyl starch, etc.), microcrystalline cellulose, sucrose, dextrose, maltodextrinis, lactose, and fructose. Suitable lubricants that can be used in this example include but are not limited to metal stearates (such as calcium, magnesium on zinc stearates), stearic acid, hydrogenated vegetable oils, talc, starch, light mineral oil, sodium benzoate, sodium chloride, sodium lauryl sulfate, magnesium lauryl sulfate, sodium stearyl fumarate, glyceryl behenate and polyethylene glycol (such as CARBOWAX™ 4000 and 6000). Suitable antiadheaents in this example include but are not limited to colloidal silicon dioxide. Suitable binders in this example include but are not limited to ethyl cellulose, a polymethacrylate polymer, polyvinylalcohol, polyvinyl pyrrolidone, polyvinylpyrrolidone-vinylacetate copolymer (e.g. KOLLIDON® VA64) hydroxyethylcellulose, low molecular weight hydroxypropylmethylcellulose (e.g. viscosity of about 1-50 cps at about 20° C.; about 2-12 cps at about 20° C.; or about 4-6 cps at about 20° C.), hydroxypropylcellulose polymethacrylates, and mixtures thereof.

The drug-containing microparticles formed by extrusion/spheronization in this prophetic example can be produced using cross-linked amphiphilic polymers by the following steps: (a) the mixing of one or more cross-linked amphiphilic polymers with tetrabenazine and optionally other pharmaceutical excipients in order to obtain a uniform mixture in the form of dry powder to which a suitable amount of liquid is added to obtain a pasty consistency; (b) the extrusion of the mixture obtained from step (a) through a perforated mesh in order to obtain cylindrical filaments having desired length and diameter; (c) the spheronization of the filaments in order to obtain a product in the form of spherical multiparticulates; (d) the drying of the product; and (e) the optional depositing of a drug on the surface of the microparticles. “Cross-linked amphiphilic polymer” refers in this example to polymers showing characteristics of swellability in the whole pH range of aqueous solutions and also in solvents or solvent mixtures having different polarity characteristics. The polymers can be cross-linked either physically through the interpenetration of the macromolecular meshes, or chemically, thus showing points of link among the macromolecular chains. Non-limiting examples of such polymers include cross-linked polyvinyl pyrrolidone, sodium carboxymethylcellulose, sodium glycolate starch and dextrans. Optional excipients include dispersing, emulsifying, wetting agents and coloring agents. The expression “uniform mixture” in this example means that the components of the mixture are uniformly dispersed in the formulation by a mixing process which assures the uniform distribution of each component. A reasonable mixing time can range from about 1 to about 60 minutes using one of the mixing equipments conventionally used for the dry mixing of the powders (e.g. “V”, fixed body, rotating body, sigma mixers). The term “liquid” in this example means any liquid substance or mix (solution or emulsion) of liquids of normal pharmaceutical use able to moisten the powder mix, as for example water, aqueous solutions having different pH, organic solvents of normal pharmaceutical use (e.g. alcohols, chlorinated solvents), and oils. Among the oils and surfactants which can be used in this example are: natural oils, either saturated or unsaturated (olive, peanut, soybean, corn, coconut, palm, sesame and similar oils); semisynthetic and synthetic mono-, di- and triglycerides containing saturated and/or unsaturated fatty acids and their polyhydroxyethylated derivatives (caprico-caprilic triglycerides [MYGLIOL™, CAPTEX™, LABRAFAC™, LIPO™], saturated or unsaturated polyhydroxylated triglycerides of various kind [LABRAFIL™, LABRAFAC™“Hydro, GELUCIRE™]); liquid waxes (isopropyl myristate, isopropyl-caprinate, -caprylate, -laurate, -palmitate, -stearate); fatty acids esters (ethyl oleate, oleyl oleate); silicone oils; polyethylene glycols (PEG 200, PEG 400, PEG 600, PEG 1000, and so on): polyglycolic glycerides (for example LABRASOL™); polyglycols (propylene glycol, tetraglycol, and ethoxydiglycol (TRANSCUTOL™), sorbitan-esters of fatty acids (for example SPAN®, ARLACEL®, BRIJ®), polyoxyethylenesorbitan esters of fatty acids (for example TWEEN®, CAPMUL®, LIPOSORB®), polypropylene oxide-polyethylene oxide (Poloxamer) copolymers, polyethylene glycol esters (PEG)-glycerol (LABRASOL®, LABRAFIL®), PEG esters and long chain aliphatic acids or alcohols (for example CREMOPHOR®), polyglycerid esters (PLUROL®), saccharide, fatty acid esters (sucro-esters), and mixtures thereof. Moreover, anionic surfactants (for example sodium lauryl sulfate, sodium stearate, sodium oleate) or cationic surfactants (for example tricetol), can be used as well as lecithins, phospholipids and their semi-synthetic or synthetic derivatives. Also tetrabenazine and/or excipients can be dissolved, dispersed and/or emulsified in such liquids.

In a particular embodiment formed by an extrusion/spheronization process from the prophetic example described above, the moistening liquid includes an oil/surfactant system wherein the tetrabenazine optionally emulsified with an aqueous phase is dissolved or dispersed. The amount of liquid with respect to the solid used in the preparation of the mixture can range from about 1% to about 80% by weight. As a prophetic example ofthis embodiment, a mixture of tetrabenazine and KOLLIDON™ CL in a ratio equal to about 1:3 by weight is co-milled obtaining the mixture in the form of powder having about 100% ofgranLulonietry lower than about 50 microns. The mixture is moistened using a liquid demineralized water containing KOLLIDON™ 25 (polyvinyl pyrrolidone, BASF) in a solution 3% w/w. The extrusion is carried out forcing the moistened mass through a threader having diameter of the holes equal to about 1 mm. The operative parameters in this prophetic example can be as follows: powder flow rate: about 4.5 kg/h; liquid flow rate: about 4.1 kg/h; torsional stress: about 27%; head temperature: about 46° C.; and screw rotation velocity: about 140 rpm. The extrusion filaments are then processed in a spheronizator adjusted at a velocity equal to about 1,000 rpm for about 2 minutes. The obtained microparticles are then dried in a fluid bed for about 2 hours to a maximum temperature equal to about 59° C. At the end of the drying the product is discharged and is mechanically screened separating the fraction ranging from about 0.7 mm to about 1.2 mm.

Another prophetic example of a drug-containing microparticle embodiment of the invention formed by an extrusion/spheronization process, uses a charged resin, the steps of which can include: (a) adding the charged resin, tetrabenazine and other excipients, to a mixing vessel; (b) mixing the ingredients to obtain a uniform mixture; (c) adding a granulating solution—a liquid capable of wetting the dry mixture. Liquids resulting in conversion of the dry powder mixture into a wet granulation that supports subsequent extrusion and spheronization (marumerization) are included. Typically, water or aqueous solutions are employed. Alcohols, typically ethanol or isopropanol, can be included with the granulating water to enhance the workability of the granulation. In another embodiment of this invention, one or more of the components of the formulation is first dissolved in water and this solution is used to produce the wet granulation. An active ingredient or an excipient which is present at very low concentration can initially be dissolved or suspended in the granulating solvent to assure more uniform distribution throughout the formulation. (d) granulating the mixture until a uniform granulation results; (e) extruding the wet granulation through a screen to produce strands of granulation; (f) spheronizing the strands of granulation to produce spherical multiparticulates; and (g) collecting and drying the spherical multiparticulates. By “charged resin” is meant in this example to mean a polymer with ionizable functional groups that becomes useful in the embodiment of this invention. This broadly encompasses any polymer that upon ionization, is capable of producing cationic or anionic polymeric chains and which support spheronization. Typically from about 10% to about 70% by weight of the spherical multiparticulate is charged resin. Non limiting examples of these charged resins include sodium polystyrene sulfonate which is sold under the trade name AMBERLITE IRP-69™ by Rohm and Haas, Co., Philadelphia, Pa.; the chloride salt of cholestyramine resin USP, sold as AMBERLITE IRP-276™ by Rohm and Haas, Co., Philadelphia, Pa.; the acid form of methacrylic acid-divinyl benzene, sold as AMBERLITE IRP-64™ by Rohm and Haas Co., Philadelphia, Pa.; carboxypolymethylenes sold under the trade names CARBOPOL™ 974P and CARBOPOL™ 934P by B. F. Goodrich, Inc., Brecksville, Ohio, and sodium polyacrylate, sold under the trade name AQUAKEEP™ J-550 by Seitetsu Kagaku, Japan. In order for the resin to maintain the desired degree of ionization, agents which produce an acidic or basic environment during granulation and spheronization can be included within the formulation. Among the groups of compounds that can exert this effect are acids, bases, and the salts of acids and bases such as adipic acid, citric acid, fumaric acid, tartaric acid, succinic acid, sodium carbonate, sodium bicarbonate, sodium citrate, sodium acetate, sodium phosphates, potassium phosphates, ammonium phosphate, magnesium oxide, magnesium hydroxide, sodium tartate, and tromethamine. Certain compounds can be added to the granulation to provide the proper degree of hydration of the charged resin, medicament and excipients. These hydrating agents include sugars such as lactose, sucrose, mannitol, sorbitol, pentaerythritol, glucose and dextrose. Polymers such as polyethylene glycol as well as surfactants and other organic and inorganic salts can also be used to modulate polymer hydration.

In another prophetic example, multiparticulates containing tetrabenazine can be obtained as follows:

| Component | Percent w/w |
|---|---|
| Tetrabenazine | about 8.7 |
| Citric Acid | about 8.7 |
| Sodium dodecyl sulfate | about 21.7 |
| Sodium Chloride | about 17.4 |
| Povidone 29-32K | about 8.7 |
| AMBERLITE IRP-69 | about 34.8 |
| Butylated Hydroxyanisol | about 0.0002 |

In this prophetic example, approximately 5.75 kg of the above formulation is mixed in a planetary mixer for about 15 minutes. The butylated hydroxyanisol is dissolved in about 60 cc of ethanol and water is added to bring the final solution to a volume of about 133 cc. This solution is added to the planetary mixer over about a two (2) minute period. The mixer is then granulated with seven aliquots of about 250 cc of water added over about a fifteen minute period. The granulation thus formed is extruded through a 1.0 mm screen and aliquots spheronized by marumerization at approximately 1200 rpm for approximately 10 minutes each. The spherical multiparticulates formed are then dried at about 50° C. for about 24 hours.

Another embodiment of this invention involves the production of drug containing microparticles in the form of ‘pearls’. Pearls can be manufactured by mixing tetrabenazine with one or more pharmaceutical excipients in molten form; the melt is forced to pass through a nozzle which is subjected to a vibration; the pearls formed are allowed to fall in a tower countercurrentwise to a gas; and the solid pearls are collected in the bottom of the tower. In this example, the quantity of tetrabenazine can vary from about 4% to about 85% by weight; and in certain embodiments from about 30% to about 50% by weight. The additives which enable the crystallization of the supercooled product to be induced in this example can be chosen from the following: fatty alcohols such as: cetyl alcohol, stearyl alcohol, fatty acids such as: stearic acid, palmitic acid, glycerol esters such as: glycerol palmitostearate, the glycerol stearate marketed under the mark PRECIROL™, the glycerol behenate marketed under the mark COMPRITOL™, hydrogenated oils such as: hydrogenated castor oil marketed under the mark CUTINA™ HR, fatty acid salts such as: magnesium or calcium stearate, polyols such as: mannitol, sorbitol, xylitol, waxes such as: white wax, carnauba wax, paraffin wax, polyoxyethylene glycols of high molecular weight, and esterified polyoxyethylenes such as: PEG-32 distearate, and PEG-150 distearate. To these crystallization additives it can be desirable in this example to add polymers which are soluble or dispersible in the melt, and which provide a controlled and adjustable dissolution of the pearls when they are used, examples of which include: cellulose derivatives (hydroxypropyl cellulose, hydroxypropyl methyl cellulose, hydroxyethyl cellulose, ethyl cellulose, carboxymethyl cellulose), acrylic resins (marketed under the mark EUDRAGIT®), polyvinyl acetates (marketed under the mark RHODOPAS®), polyalkylene (ethylene propylene), polylactic, maleic anhydride and silicone resins. In addition, inorganic additives can be added to accelerate the solidification of the active substances, examples of which include: silicas, inorganic oxides such as titanium or iron oxide, phosphates, carbonates, clays, and talc. In addition, a surface-active agent can be added to improve the dispersion of the active substance in the crystallization additive, examples of which include: sorbitol esters, the polyoxyethylene polysorbates marketed under the mark TWEEN®, and glycols such as glycerine or propylene glycol. The process for the preparation of pearls include preparing a melt of the tetrabenazine with one or more excipients. This melt can be prepared by separately melting the various constituents and then mixing them or by melting the mixture of the constituents, possible insoluble compounds being added at the end of the melting so as to obtain a homogeneous mass. The nature of the constituents of the melt is chosen by the person skilled in the art, which is considered as a function of the compatibility of the constituents, the viscosity of the mixture of constituents, the nozzle diameter, the hydrophilicity of the active substance, the surface tension of the active substance, the particle size of the insoluble additives, the flow rate of the nozzle, the temperature of the tower, its height and, above all, the size of the desired pearls, the proportion of tetrabenazine to be included therein and the desired release time of the active substance.

Alternative procedures other than extrusion or spheronization for manufacturing drug-containing microparticles can include wet granulation, solvent granulation and melt granulation. All of these techniques involve the addition of an inactive binder to aggregate smaller particles into larger granules. For example, wet granulation and solvent granulation involve the addition of a liquid binder which aggregates the active materials and excipients into granules. After granulation, the liquid can be removed by a separate drying step. Melt granulation is similar to wet granulation, but uses a low melting point solid material as a binder. The solid binder in melt granulation is melted and acts as a liquid binder thereby aggregating the powdered active material and excipients into granules. The binder thereby, can be incorporated into the granules when the granules cool.

Certain embodiments of the present invention include microparticles manufactured by a process for producing granules by rotomelt granulation that includes mixing tetrabenazine and a powdered excipient material that has a higher melting point than tetrabenazine in a zone wherein both powdered materials are maintained in a fluidized state by a rising stream of gas in an apparatus having a rapidly rotating horizontal-disk located within a vertical vessel having a bottom surface; wherein said rapidly rotating disk is located on the bottom surface of the vertical vessel wherein said gas is at a temperature sufficient to cause the tetrabenazine to at least partially melt thereby causing said powdered materials to aggregate and form granules. Other embodiments of the present invention include microparticles manufactured by a process for producing granules by rotomelt granulation including mixing powdered binder material and tetrabenazine wherein the tetrabenazine has a higher melting point than the powdered binder material in a zone wherein both powdered materials are maintained in a fluidized state by a rising stream of gas in an apparatus having a rapidly rotating horizontal-disk located within a vertical vessel having a bottom surface; and wherein said rapidly rotating disk is located on the bottom surface of the vertical vessel wherein said gas is at a temperature sufficient to cause the powdered binder material to at least partially melt thereby causing said powdered materials to aggregate and form granules.

In rotomelt granulation, one of the feed powders must have a lower melting point than the other powder in order to serve as a binder. The feed powders are introduced into a vertical vessel with rotatable horizontal-disk located in the bottom of the vessel. The powder is maintained in fluidized state by at least one stream of filtered air being circulated from the bottom of the vertical vessel through one or more inlets. The rotatable horizontal disk is then rotated while the air supplied to fluidize the powder is maintained at a temperature sufficient to soften or melt the lower melting point powder. The temperature to which the binder must be heated to soften can be empirically determined by observing the formation of granules at various temperatures for various binders. It is presently believed that temperatures from about 3° C. to about 5° C. below the melting point or melting range provides sufficient softening to result in granule formation. The lower melting point powder then acts as a binding agent to promote the aggregation of powder particles into granules. Suitable powders for use in rotomelt granulation have a diameter size in the range of from about 5 microns to about 150 microns; and in certain embodiments have a diameter size in the range of about 35 microns to about 80 microns. The temperature which the components will be exposed to depends on the binder employed to aggregate the powders. Generally, the melting point of the binder is above about 30° C.; and in certain embodiments is below about 100° C.

The powders used in these microparticles manufactured by rotomelt granulation can be formed into granules by at least two alternative granulation mechanisms. The first mechanism for granule formation utilizes a larger particulate binder and a smaller particulate powder. The temperature during the rotomelt granulation is then elevated only to the point where the external surface of the binder particles become tacky. As the second powdered material of a smaller size is contacted with the tacky surface it forms a microlayer on the surface of the binder particle. This granulation mechanism results in granules which have size distribution similar to the original binder particles employed. Alternatively, the rotomelt granulation can be conducted at a temperature at which the binder acts as a cement bridging the gaps between the unmelted particles (this is referred to as agglomeration). This mechanism results in the formation of granules where the components are intermingled. For each binder used the mechanism can be controlled primarily by the temperature at which the rotomelt granulation is performed. Those skilled in the art will appreciate that the granules formed can be observed by electron microscopy to determine the type of granulation process occurring. If one particular type of granule is desired, the process conditions or starting materials can be varied to produce the desired granules.

Other embodiments of this invention involve the combined granulation and coating of tetrabenazine into microparticles in which the drug is at least partly located within the microparticle core but capable of immediate release. To do this, the tetrabenazine and a granular disintegrant are first dry-mixed; the powder obtained is then granulated, in the presence of a mixture of excipients including at least one binder capable of binding the particles together to give grains; the grains thus formed are then coated by spraying with a suspension including at least one coating agent and a membrane disintegrant; and then the coated granules obtained are dried. The distinction between the actual granulation and coating steps is relatively theoretical, insofar as, even though the primary function of the binder used in the granulation step is to bind together the particles, it nevertheless already partially coats the grains formed. Similarly, even though the primary function of the coating agent used in the actual coating step is to complete the final coating of each of the grains, it may, however, arbitrarily bind other coated grains by a mechanism of granular agglomeration. The binder and the coating agent are chosen from the group including cellulose polymers and acrylic polymers. However, even though the binder and the coating agent are chosen from the same group of compounds, they nevertheless differ from each other in their function as previously mentioned. Among the cellulose polymers that can be advantageously chosen are ethylcellulose, hydroxypropylcellulose (HPC), carboxymethylcellulose (CMC) and hydroxypropylmethylcellulose (HPMC), or mixtures thereof. Among the acrylic polymers that can be advantageously chosen are the ammonio-methacrylate copolymer (EUDRAGIT® RL or RS), the polyacrylate (EUDRAGIT® NE) and the methacrylic acid copolymer (EUDRAGIT® L or S), EUDRAGIT® being a registered trademark of Rohm. In at least one embodiment, the binder is of the same nature as the coating agent. To further accelerate the release of the tetrabenazine, the coating suspension also includes a permeabilizer which, on account of its intrinsic solubility properties, causes perforation of the membrane coating, thus allowing the tetrabenazine to be released. Non-limiting examples of permeabilizers include povidone and its derivatives, polyethylene glycol, silica, polyols and low-viscosity cellulose polymers. Polymers of the type such as hypromellose, whose viscosity is equal to about 6 centipoises, are used, for example, as low-viscosity cellulose polymer. In at least one embodiment, the dry-mixing of initial powder and the granulation, coating and drying steps are performed in a fluidized bed. In this case, the initial powder mixture is first fluidized before being granulated by spraying said powder with the excipient mixture including at least the binder, the grains obtained then being coated by spraying with the coating suspension, the coated granules formed finally being dried in the fluidized bed. In at least one embodiment, the mixture of excipients used during the granulation step and the coating suspension used during the coating step form a single mixture. In this case, the granulation step can be distinguished from the spraying step by varying different parameters, such as the rate of spraying of the mixture and the atomization pressure of said mixture. Thus, only some of the mixture of excipients is used during the granulation step, while the other portion can be used during the coating step. Thus, the rate of spraying of the coating suspension is higher during the granulation step than during the coating step, whereas the atomization pressure of the coating suspension is lower during the granulation step than during the coating step. In practice, at the laboratory scale in a fluidized-bed device, for example of the type such as Glatt GPCGI, during the granulation step, the rate of spraying of the coating suspension is from about 10 grams/minute to about 25 grains/minute, and the atomization pressure is from about 1 bar to about 1.8 bar. During the coating step, the rate of spraying of the coating suspension is from about 5 grams/minute to about 15 grams/minute, while the atomization pressure is from about 1.5 bar to about 2.5 bar.

In at least one embodiment, from about 10% to about 20% oftthe mixture of excipients is sprayed during the granulation step, the remainder being sprayed during the coating step.

As a prophetic example of these embodiments that involve the combined granulation and coating of tetrabenazine into microparticles in which the drug is at least partly located within the microparticle core but capable of immediate release, the microparticles can be manufactured according to the following process: A granulation solution is first prepared by dissolving about 48 g of ethylcellulose in about 273 g of ethyl alcohol. A coating suspension is then prepared by mixing about 97 g of ethylcellulose, about 28.5 g of polyethylene glycol 6000, about 26 g of sodium croscarmellose, about 10 g of precipitated silica and about 27.5 g of aspartam in about 1900 g of ethyl alcohol, until a homogeneous suspension is obtained. The powder mixture consisting of about 700 grams of tetrabenazine and about 35 grams of Acdisol is then fluidized. The granulation is then started by spraying the granulation solution for about 15 to about 20 minutes at a spraying rate of about 25 grams/minute and a suspension atomization pressure of about 0.8 bar. The actual coating is then performed by spraying the coating suspension for about 1 hour 30 minutes at a spraying rate of about 15 to about 20 grams/minute and a suspension spraying pressure of about 1.5 bar.

Another embodiment of the invention for coating the tetrabenazine material, thereby forming a drug-containing microparticle, involves the formation of coated microcrystals that can subsequently be incorporated into a tablet. Through selection of the appropriate polymer the microcrystals can possess diversified features such as gastroresistance, gastrorelease, gastroretention, pulsatile release, and controlled release due to the fact that the said coated or non-coated microcrystals and microgranules preserve, after having been shaped in the form of a multiparticulate tablet, their initial properties amongst which are included masking of taste, gastroresistance, gastrorelease, gastroretention, pulsatile release, and controlled release of the tetrabenazine. In certain embodiments of this example, the following non-limiting list of polymers can be selected for coating of the tetrabenazine in conventional fluidized based coating equipment: ethylcellulose (EC); hydroxypropylcellulose (HPC); hydroxypropylmethylcellulose (HPMC); gelatin; gelatin/acacia; gelatin/acacia/vinylmethylether maleic anhydride; gelatin/acacia/ethylenemaleic anhydride; carboxymethyl cellulose; polyvinvylalcohol; cellulose acetate phthalate; nitrocellulose; shellac; wax; polymethacrylate polymers such as EUDRAGIT® RS; EUDRAGIT® RL or combinations of both, EUDRAGIT® E and EUDRAGIT® NE30D; KOLLICOAT™ SR30D; and mixtures thereof.

Drug-Layered Microparticles

The drug-layered microparticles of certain embodiments can be made by coating an inert particle or core, such as a non-pareil sphere (e.g. sugar sphere), with the tetrabenazine and a polymeric binder. In certain embodiments of the drug-layered microparticles, the inert cores include water-insoluble materials such as cellulose spheres or silicon dioxide. In other embodiments, the inert cores include water-soluble materials such as starch, salt, pH modifiers, solubilizers, or sugar spheres. The inert cores can have a diameter ranging from about 100 microns to about 2000 microns. For example, in certain embodiments the diameter of the inert cores range from about 100 microns to about 2000 microns. In at least one embodiment, the inert cores are Sugar Spheres NF, containing not less than about 62.5% and not more than about 91.5% of sucrose. In at least one embodiment the inert cores have substantially consistent bulk density, low friability, and low dust generation properties. In at least one embodiment, the inert cores are coated with an osmotic sub-coat including an osmotic agent and a polymeric binding agent. Further, the inert cores can initially be coated with a seal-coat to provide a more consistent core surface and to minimize any osmotic effects. The seal-coat layer can be applied to the core prior to the application of the drug, polymeric binder, and any polymeric film layers. In at least one embodiment, the seal-coat layer does not substantially modify the release of the tetrabenazine. Examples of suitable sealants that can be used in the seal-coat include permeable or soluble agents such as hydroxypropyl methylcellulose, hydroxypropyl cellulose, ethylcellulose, a polymethacrylate polymer, hydroxypropyl ethylcellulose, xanthan gum, and mixtures thereof. In at least one embodiment the sealant used in the seal-coat is hydroxypropyl methylcellulose. Other agents can be added to improve the processability of the sealant. Examples of such agents include talc, colloidal silica, polyvinyl alcohol, titanium dioxide, micronized silica, fumed silica, glycerol monostearate, magnesium trisilicate, magnesium stearate, and mixtures thereof. The seal-coat layer can be applied from solution (e.g. aqueous) or suspension using a fluidized bed coater (e.g. Wurster coating), or in a pan coating system. Examples of such seal-coats coatings are commercially available such as those sold tinder the trademarks OPADRY® White Y-1-7000 and OPADRY® OY/B/28920 White, each of which is available from Colorcon Limited, England.

The binding agent of these drug-layered embodiments is used to adhere the tetrabenazine layer to the inert core or seal-coat of the core. In certain embodiments, the binding agent is water soluble, possesses sufficiently high adhesivity in order to adhere the tetrabenazine layer to the inert core, and possesses an appropriate viscosity to provide substantial adhesion between the inert core and the tetrabenazine. In other embodiments the binding agent is water-insoluble. In at least one embodiment the binding agent is ethyl cellulose, a polymethacrylate polymer, polyvinylalcohol, polyvinyl pyrrolidone, polyvinylpyriolidone-vinylacetate copolymer (such as KOLLIDON® VA64), hydroxyethylcellulose, low molecular weight hydroxypropylmethylcellulose (e.g. viscosity of about 1-50 cps at about 20° C; about 2-12 cps at about 20° C; or about 4-6 cps at about 20° C), hydroxypropylcellouse, polymethacrylates, or mixtures thereof. For example, in certain embodiments the composition of the binder for tetrabenazine is from about 1% to about 35% w/w; in other embodiments firom about 2% to about 15% w/w; and in still other embodiments from about 3% to about 12% w/w, expressed as a percentage of the total weight of the core.

Solvents can be used to apply the tetrabenazine to the inert core, examples of which include lower alcohols such as ethanol, isopropanol and alcohol/water mixtures, acetone and chlorinated hydrocarbons.

The drug-layered microparticles can be prepared by forming a suspension or solution of the binder and the tetrabenazine and then layering the suspension or solution on to the inert or sub-coated core using any of the layering techniques known in the art, such as fluidized bed coating or pan coating. This can be affected in a single coating or the process can be carried out in multiple layers, optionally with intervening drying/evaporation steps. This process can be conducted so as to produce microparticles containing a desired amount of tetrabenazine and achieve the desired dosage and release thereof upon in-vivo administration.

In certain embodiments, the drug-layered microparticles can be manufactured using for example, the procedure in the following hypothetical experiment: tetrabenazine (about 2.8 kg) and hydroxypropyl methylcellulose (METHOCEL® E5) (about 0.40 kg) is dissolved in a mixture of water and isopropyl alcohol. The active drug solution can then be sprayed onto sugar spheres 30/35 (about 8.06 kg) in a fluidized bed processor with a Wurster insert. The active core microparticles can then be dried in a fluidized bed processor until the loss on drying is below about 1%. The tetrabeniazinie microparticles can then be passed through a 16 mesh screen and a 30 mesh screen and microparticles can be collected that are smaller than 16 mesh and larger than 30 mesh.

In other embodiments, drug-layered microparticles containing pH modifier can be manufactured using for example, the procedure in the following hypothetical experiment: tetrabenazine (about 2.8 kg), hydroxypropyl methylcellulose (METHOCEL® E5) (about 0.35 kg), and fumaric acid (about 0.20 kg) is dissolved in a mixture of water and isopropyl alcohol. The active drug solution can then be sprayed onto sugar spheres 30/35 (about 8.06 kg) in a fluidized bed processor with a Wurster insert. The active core microparticles can then be dried in a fluidized bed processor until the loss on drying is below about 1%. The tetrabenazine microparticles can then be passed through a 16 mesh screen and a 30 mesh screen and microparticles can be collected that are smaller than 16 mesh and larger than 30 mesh.

Microparticle Taste-Masking Coatings

The microparticles of the present invention can each be coated with at least one taste-masking coating. The taste-masking coating can mask the taste of the active drug in the microparticles. In at least one embodiment the taste-masking coating formulations contain polymeric ingredients. It is contemplated that other excipients consistent with the objects of the present invention can also be used in the taste-masking coating.

In at least one embodiment, the taste-masking coating includes a polymer such as ethylcellulose, which can be used as a dry polymer (such as ETHOCEL®, Dow Corning) solubilized in organic solvent prior to use, or as an aqueous dispersion. One commercially-available aqueous dispersion of ethylcellulose is AQUACOAT® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the AQUACOAT® is intimately mixed with a suitable plasticizer prior to use. Another aqueous dispersion of ethylcellulose is commercially available as SURELEASE® (Colorcon, Inc., West Point, Pa., U.S.A.). This product can be prepared by incorporating plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

In other embodiments, polymethacrylate acrylic polymers can be employed as taste masking polymers. In at least one embodiment, the taste masking coating is an acrylic resin lacquer used in the form of an aqueous dispersion, such as that which is commercially available from Rohm Pharma under the trade name EUDRAGIT® or from BASF under the trade name KOLLICOAT®R. In further embodiments, the acrylic coating includes a mixture of two acrylic resin lacquers commercially available from Rohm Pharma under the trade names EUDRAGIT® RL and EUDRAGIT® RS, respectively.

EUDRAGIT® RL and EUDRAGIT® RS are copolymers of acrylic and methacrylic esters with a low content of quaternary ammonium groups, the molar ratio of ammonium groups to the remaining neutral (meth)acrylic esters being 1:20 in EUDRAGIT® RL and 1:40 in EUDRAGIT® RS. The mean molecular weight is 150,000. The code designations RL (high permeability) and RS (low permeability) refer to the permeability properties of these agents. EUDRAGIT® RL/RS mixtures are insoluble in water and in digestive fluids. However, coatings formed from the same are swellable and permeable in aqueous solutions and digestive fluids. EUDRAGIT® RL/RS dispersions or solutions of certain embodiments can be mixed together in any desired ratio in order to ultimately obtain a taste masking coating having a desirable drug dissolution profile. In certain embodiments formulations can be obtained, for example, from a coating derived from 100% EUDRAGIT® RL; 50% EUDRAGIT® RL with 50% EUDRAGIT® RS; and 10% EUDRAGIT® RL with 90% EUDRAGIT® RS.

In other embodiments, the taste masking polymer can be an acrylic polymer which is cationic in character based on d imethiylamiiinoetilyl methacrylate and neutral inethacrylic acid esters (such as EUDRAGIT® E, commercially available from Rohm Pharma). The hydrophobic acrylic polymer coatings of the present invention can further include a neutral copolymer based on poly (meth)acrylates, such as EUDRAGIT® NE (NE=neutral ester), commercially available from Rohm Pharma. EUDRAGIT® NE 30D lacquer films are insoluble in water and digestive fluids, but permeable and swellable.

In other embodiments, the taste masking polymer is a dispersion of poly (ethylacrylate, methyl methacrylate) 2:1 (KOLLICOAT® EMM 30 D, BASF).

In other embodiments, the taste masking polymer can be a polyvinyl acetate stabilized with polyvinylpyrrolidone and sodium lauryl sulfate such as KOLLICOAT® SR30D (BASF).

Other taste masking polymers include hydroxypropylcellulose (HPC); hydroxypropylmethylcellulose (HPMC); hydroxyethylcellulose; gelatin; gelatin/acacia; gelatin/acacia/vinvylmethylether maleic anhydride; gelatin/acacia/ethylenemaleic anhydride; carboxymethyl cellulose; polyvinvylalcohol; nitrocellulose; polyvinylalcohol-polyethylene glycol graft-copolymers; shellac; wax and mixtures thereof.

The taste-masking coatings can be applied to the microparticles from one or more organic or aqueous solvent solutions or suspensions. In at least one embodiment the organic solvents that can be used to apply the taste-masking coatings include one or more of acetone, lower alcohols such as ethanol, isopropanol and alcohol/water mixtures, chlorinated hydrocarbons, and the like. Devices used to coat the microparticles of the invention with a taste-masking coating include those conventionally used in pharmaceutical processing, such as fluidized bed coating devices. The coatings applied to the microparticles can contain ingredients other than the functional polymers. One or more colorants, flavorants, sweeteners, can also be used in the taste-masking coating.

In some embodiments a pore former can be included into the taste masking coat in order to influence the rate of release of tetrabenazine from the microparticle. In other embodiments, a pore former is not included in the taste masking coat. The pore formers can be inorganic or organic, and include materials such as particulate materials that can be dissolved, extracted or leached from the coating in the environment of use. Upon exposure to fluids in the environment of use, the pore-formers can for example be dissolved, and channels and pores are formed that fill with the environmental fluid.

For example, the pore-formers of certain embodiments can include one or more water-soluble hydrophilic polymers in order to modify the release characteristics of the formulation. Examples of suitable hydrophilic polymers used as pore-formers include hydroxypropylmetlhylcellulose, cellulose ethers and protein-derived materials of these polymers, the cellulose ethers, such as hydroxyalkylcelluloses and carboxyalkylcelluloses. Also, synthetic water-soluble polymers can be used, examples of which include polyvinylpyrrolidone, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, sorbitol and mixtures thereof. In at least one embodiment, the hydrophilic polymer includes hydroxypropyl-methylcellulose.

Other non-limiting examples of pore-formers that can be used in certain embodiments containing a taste masking coat include alkali metal salts such as lithium carbonate, sodium chloride, sodium bromide, potassium chloride, potassium sulfate, potassium phosphate, sodium acetate, sodium citrate and mixtures thereof. The pore-forming solids can also be polymers which are soluble in the environment of use, such as CARBOWAX™, and CARBOPOL™. In addition, the pore-formers embrace diols, polyols, polyhydric alcohols, polyalkylene glycols, polyglycols, poly(a-w)alkylenediols and mixtures thereof. Other pore-formers which can be useful in the formulations of certain embodiments of the present invention include starch, modified starch, and starch derivatives, gums, including but not limited to xanthan gum, alginic acid, other alginates, benitonite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambdacarrageenan, gum karaya, biosynthetic gum, and mixtures thereof Other pore-formers include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidenle), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In general, the amount of pore-former included in the taste masking coatings of certain embodiments of the present invention can be from about 0.1% to about 80%, by weight, relative to the combined weight of polymer and pore-former. The percentage of pore former as it relates to the dry weight of the taste-masking polymer, can have an influence on the drug release properties of the coated microparticle. In at least one embodiment that uses water soluble pore formers such as hydroxypropylmethylcellulose, a taste masking polymer: pore former dry weight ratio of from about 10:1 to about 1:1 can be present. In certain embodiments the taste masking polymer: pore former dry weight ratio is from about 8:1 to about 1.5:1: and in other embodiments from about 6:1 to about 2:1. In at least one embodiment using EUDRAGIT® NE30D as the taste masking polymer and a hydroxypropylmethlylcellulose (approx 5 cps viscosity (in a 2% aqueous solution)) such as METHOCEL® E5, Pharmacoat 606G as the water soluble pore former, a taste masking polymer: pore former dry weight ratio of about 2:1 is present.

Colorants that can be used in the taste-masking coating include food, drug and cosmetic colors (FD&C), drug and cosmetic colors (D&C) or external drug and cosmetic colors (Ext. D&C). These colors are dyes, lakes, and certain natural and derived colorants. Useful lakes include dyes absorbed on aluminum hydroxide or other suitable carriers.

Flavorants that can be used in the taste-masking coating include natural and synthetic flavoring liquids. An illustrative list of such flavorants includes volatile oils, synthetic flavor oils, flavoring aromatics, oils, liquids, oleoresins and extracts derived from plants, leaves, flowers, fruits, stems and combinations thereof. A non-limiting representative list of these includes citric oils. such as lemon, orange, grape, lime and grapefruit, and fruit essences, including apple, pear, peach, grape, strawberry, raspberry, cherry, plum, pineapple, apricot, or other fruit flavors. Other useful flavorants include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin), and mixtures thereof.

Sweeteners that can be used in the taste-masking coating include glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Steva Rebaudiana (Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners can be used alone or in any combination thereof.

The microparticle taste masking coat can also include one or more pharmaceutically acceptable excipients such as lubricants, emulsifiers, anti-foaming agents, plasticizers, solvents and the like.

Lubricants can be included to help reduce friction of coated microparticles during manufacturing. The lubricants that can be used in the taste masking coat of the present invention include but are not limited to adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (i.e. COMPRITOL™), STEAR-O-WET™, MYVATEX™ TL and mixtures thereof. In at least one embodiment, the lubricant is selected from magnesium stearate. talc and a mixture thereof. Combinations of these lubricants are operable. The lubricant can each be present in an amount of from about 1% to about 100% by weight of the polymer dry weight in the taste masking coat. For example, in certain embodiments wherein the taste masking polymer is EUDRAGIT® NE30D or EUDRAGIT® NE40D (Rohm America LLC) together with a hydrophilic pore former, the lubricant is present in an amount of from about 1% to about 30% by weight of the polymer dry weight; in other embodiments from about 2% to about 20%; and in still other embodiments at about 10% by weight of the micmoparticle taste masking coat dry weight. In another embodiment where the taste masking polymer is ethylcellulose (ETHOCEL™ PR100, PR45, PR20, PR10 or PR7 polymer, or a mixture thereof), the lubricant can be present in an amount of from about 10% to about 100% by weight of the iiiicroparticle taste masking coat dry weight; in another embodiment from about 20% to about 80%; and in still another embodiments at about 50% by weight of the microparticle taste masking coat dry weight. In other embodiments, the taste masking coat does not include a pore former.

Emulsifying agent(s) (also called emulsifiers or emulgents) can be included in the microparticle taste masking coat to facilitate actual emulsification during manufacture of the coat, and also to ensure emulsion stability during the shelf-life of the product. Emulsifying agents useful for the microparticle taste masking coat composition of certain embodiments include, but are not limited to naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Combinations of emulsifying agents are operable. In at least one embodiment, the emulsifying agent is TWEEN™ 80. The emulsifying agent(s) can be present in an amount of from about 0.01% to about 5% by weight of the microparticle taste masking polymer dry weight. For example, in certain embodiments the emulsifying agent is present in an amount of from about 0.05% to about 3%; in other embodiments from about 0.08% to about 1.5%, and in still other embodiments at about 0.1% by weight of the microparticle taste masking polymer dry weight.

Anti-foaming agent(s) can be included in the microparticle taste masking coat to reduce frothing or foaming during manufacture of the coat. Anti-foaming agents useful for the coat composition include, but are not limited to simethicone, polyglycol, silicon oil, and mixtures thereof. In at least one embodiment the anti-foaming agent is Simethicone C. The anti-foaming agent can be present in an amount of from about 0.1% to about 10% of the microparticle taste masking coat weight. For example, in certain embodiments the anti-foaming agent is present in an amount of from about 0.2% to about 5%; in other embodiments from about 0.3% to about 1%, and in still other embodiments at about 0.6% by weight of the microparticle taste masking polymer dry weight.

Plasticizer(s) can be included in the microparticle taste masking coat to provide increased flexibility and durability during manufacturing. Plasticizers that can be used in the microparticle taste masking coat of certain embodiments include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, and mixtures thereof. The plasticizer can be present in an amount of from about 1% to about 80% of the taste masking polymer dry weight. For example, in certain embodiments the plasticizer is present in an amount of from about 5% to about 50%, in other embodiments from about 10% to about 40%, and in still other embodiments at about 20% of the taste masking polymer dry weight.

The taste-masking coating can be present in an amount of from about 1% to about 90% by weight of the microparticle, depending upon the choice of polymer, the ratio of polymer:pore former, and the total surface area of the microparticle formulation. Since a certain thickness of taste masking coating has to be achieved in order to achieve effective taste masking, the amount of taste masking polymer coating used during manufacture is related to the total surface area of the batch of uncoated microparticles that requires a coating. The taste masking polymer surface area coverage can range from about 0.5 mg/cm2 to about 20 mg/cm2. For example, in certain embodiments the surface area coverage of the taste masking polymer is from about 0.6 mg/cm2 to about 10 mg/cm2, and in other embodiments is from about 1 mg/cm2 to about 5 mg/cm2. In at least one embodiment of the invention, EUDRAGIT® E is employed as the taste masking polymer at a surface area coverage of about 4 mg/cm2. One approach in estimating the total surface area of a multiparticulate batch is the permeability method according to Blaine (ASTM Des. C 205-55), which is based upon the mathematical model of laminar flow through capillaries arranged in parallel.

In the absence of an accurate determination of total surface area of a microparticle, the amount of taste masking polymer to be applied can be expressed as a percentage of the uncoated microparticle. For example, in certain embodiments the taste-masking coating is present in an amount of from about 5% to about 60%; in other embodiments from about 10% to about 40%; and in still other embodiments from about 15% to about 35% by weight of the microparticle. In at least one embodiment the taste-masking coating is present in an amount of about 30% by weight of tle microparticle.

In certain embodiments, the diameter of the micropar-ticles (with or without the taste-masking coating) range from about 50 μm to about 800 μm. For example, in certain embodiments the diameter of the microparticles range from about 100 μm to about 600 μm, and in other embodiments from about 150 μm to about 450 μm.

Microparticle Controlled Release Coat

The microparticles of the present invention can each be coated with at least one controlled release coat. As used herein, the term “microparticle controlled release coat” refers to the controlled release coat that substantially surrounds each microparticle. The microparticle controlled release coat is designed to achieve a controlled release of the tetrabenazine from the microparticle. For example, the microparticle controlled release coat can be an enteric coat with low solubility at a gastric pH to reduce or minimize the drug release in the lumen of the stomach, whilst possessing pH dependent solubility to facilitate drug release in the duodenum. In another embodiment, the controlled release coat can be a delayed release coating that provides a delayed release of the tetrabenazine with a predetermined lag time that is independent of, or alternatively dependent on, the pH of the dissolution medium. For example, by increasing the thickness of the microparticle controlled release coat using a pH independent diffusion polymer, lag times of about 1 hour, about 2 hours, about 3 hours, about 4 hours, about 5 hours, about 6 hours, about 7 hours, about 8 hours, about 9 hours, about 10 hours, about 11 hours, or about 12 hours can be achieved. Alternatively, controlled release polymers can be selected that become soluble above a certain pH. Drug release from such a system is reduced or minimized until the certain pH for the polymer of choice is exceeded. With either approach, following the predetermined lag, drug is released, for example within about 1 hour for an immediate release pulse, or alternatively over a prolonged period of time, for example from about 3 to about 24 hours. In other embodiments, the microparticle controlled release coat can provide a diffusion barrier that is independent of pH, thus facilitating a sustained release profile, with substantially full release of the tetrabenazine occurring in from about 3 to about 24 hours following administration. In at least one embodiment, the microparticle controlled release coat provides a delayed and sustained release of the tetrabenazine from the microparticle with substantially full release in about 24 hours following administration.

In certain embodiments, the microparticle controlled release coat can provide substantially full release of the tetrabenazine from the microparticle without requiring the use of any pore formers. Unnecessary pore formers that are not required in the microparticle controlled release coat include hydrophilic polymers such as hydroxypropyl methylcellulose.

The microparticle controlled release coat includes at least one polymer in an amount sufficient to achieve a controlled release of the tetrabenazine. In at least one embodiment of the invention the controlled release polymer is an acrylic polymer. Suitable acrylic polymers include but are not limited to acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid, methacrylic acid alkylamine copolymer, poly(methyl methacrylate), poly(methacrylic acid) (anhydride), glycidyl methacrylate copolymers, and mixtures thereof.

In at least one embodiment the controlled release coat includes polymerizable quaternary ammonium compounds, of which non-limiting examples include quaternized aminoalkyl esters and aminoalkyl amides of acrylic acid and methacrylic acid, for example β-methacryl-oxyethyl-trimethyl-ammonium methosulfate, β-acryloxy-propyl-trimethyl-ammonium chloride, trimethylaminomethyl-methacrylamide methosulfate and mixtures thereof. The quaternary ammonium atom can also be part of a heterocycle, as in methacryloxyethylmethyl-morpholiniom chloride or the corresponding piperidinium salt, or it can be joined to an acrylic acid group or a methacrylic acid group by way of a group containing hetero atoms, such as a polyglycol ether group. Further suitable polymerizable quaternary ammonium compounds include quaternized vinyl-substituted nitrogen heterocycles such as methyl-vinyl pyridinium salts, vinyl esters of quaternized amino carboxylic acids, and styryltrialkyl ammonium salts. Other polymerizable quaternary ammonium compounds useful in the present invention include acryl- and methacryl-oxyethyltrimethyl-ammonium chloride and methosulfate, benzyldimethylammoniumethyl-methlacrylate chloride, diethylmethylammoniumethyl-acrylate and -methacrylate methosulfate, N-trimethylammoniumpropylmethacrylamide chloride, N-trimethylammonium-2,2-dimethylpropyl-1-methacrylate chloride and mixtures thereof.

In at least one embodiment, the polymer of the controlled release coat is an acrylic polymer comprised of one or more ammonio methacrylate copolymers. Ammonio methacrylate copolymers (such as those sold under the trademark EUDRAGIT® RS and RL) are described in NF XVII as fully polymerized copolymers of acrylic and methacrylic acid esters with a low content of quaternary ammonium groups. In order to obtain a desirable dissolution profile for a given therapeutically active agent such as tetrabenazine, it may be helpful in some embodiments to incorporate two or more ammonio methacrylate copolymers having differing physical properties. For example, it is known that by changing the molar ratio of the quaternary ammonium groups to the neutral (meth)acrylic esters, the permeability properties of the resultant controlled release coat can be modified.

In other embodiments of the present invention, the acrylic polymer coating further includes a polymer whose permeability is pH dependent, such as anionic polymers synthesized from methacrylic acid and methacrylic acid methyl ester. Such polymers are commercially available, e.g., from Rohm Pharma GmbH under the trade name EUDRAGIT® L and EUDRAGIT® S, and the ratio of free carboxyl groups to the esters is said to be 1:1 in EUDRAGIT® L and 1:2 in EUDRAGIT® S. EUDRAGIT® L is insoluble in acids and pure water, but becomes increasingly permeable above pH 5.0. EUDRAGIT® S is similar, except that it becomes increasingly permeable above pH 7. The hydrophobic acrylic polymer coatings can also include a polymer which is cationic in character based on dimethylaminoethyl methacrylate and neutral methacrylic acid esters (such as EUDRAGIT® E, commercially available from Rohm Pharma). The hydrophobic acrylic polymer coatings of certain embodiments can further include a neutral copolymer based on poly (meth)acrylates, such as EUDRAGIT® NE (NE=neutral ester), commercially available from Rohm Pharma. EUDRAGIT® NE 30D lacquer films are insoluble in water and digestive fluids, but permeable and swellable.

In other embodiments of the invention the controlled release polymer is a dispersion of poly (ethylacrylate, methyl methacrylate) 2:1 (KOLLICOAT® EMM 30 D, BASF). In other embodiments the controlled release polymer can be a polyvinyl acetate stabilized with polyvinylpyrrolidone and sodium lauryl sulfate such as KOLLICOAT® SR30D (BASF). The dissolution profile can be altered by changing the relative amounts of different acrylic resin lacquers included in the coating. Also, by changing the molar ratio of polymerizable permeability-enhancing agent (e.g., the quaternary ammonium compounds) in certain embodiments to the neutral (meth)acrylic esters, the permeability properties (and thus the dissolution profile) of the resultant coating can be modified.

In at least one embodiment the controlled release polymer is ethylcellulose, which can be used as a dry polymer (such as ETHOCEL®, Dow Corning) solubilized in organic solvent prior to use, or as an aqueous dispersion. One commercially available aqueous dispersion of ethylcellulose is AQUACOAT® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the AQUACOAT® is intimately mixed with a suitable plasticizer prior to use. Another aqueous dispersion of ethylcellulose is commercially available as SURELEASE® (Colorcon, Inc., West Point, Pa., U.S.A.). This product can be prepared by incorporating a plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

Other examples of polymers that can be used in the microparticle controlled release coat include cellulose acetate phthalate, cellulose acetate trimaletate, hydroxy propyl methylcellulose phthalate, polyvinyl acetate phthalate, polyvinyl alcohol phthalate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, ethyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, pullulan, collagen, casein, agar, gum arabic, sodium carboxymethyl cellulose, (swellable hydrophilic polymers) poly(hydroxyalkyl methacrylate) (molecular weight from about 5 k to about 5000 k), polyvinylpyrrolidone (molecular weight from about 10 k to about 360 k), anionic and cationic hydrogels, zein, polyamides, polyvinyl alcohol having a low acetate residual, a swellable mixture of agar and carboxymethyl cellulose, copolymers of maleic anhydride and styrene, ethylene, propylene or isobutylene, pectin (molecular weight from about 30 k to about 300 k), polysaccharides such as agar, acacia, karaya, tragacanth, algins and guar, polyacrylamides, POLYOX® polyethylene oxides (molecular weight from about 100 k to about 5000 k), AQUAKEEP® acrylate polymers, diesters of polyglucan, crosslinked polyvinyl alcohol and poly N-vinyl-2-pyrrolidone, hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, methyl ethyl cellulose, ethylhydroxy ethylcellulose, cellulose acetate, cellulose butyrate, cellulose propionate, gelatin, starch, maltodextrin, pullulan, polyvinyl pyrrolidone, polyvinyl alcohol, polyvinyl acetate, glycerol fatty acid esters, polyacrylamide, polyacrylic acid, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

In at least one embodiment the controlled release coat of the microparticles includes polymers that can facilitate mucoadhsion within the gastrointestinal tract. Non-limiting examples of polymers that can be used for mucoadhesion include carboxymethylcellulose, polyacrylic acid, CARBOPOL™, POLYCARBOPHIL™, gelatin and other natural or synthetic polymers.

In at least one embodiment the microparticles are coated with a controlled release coat comprised of:

at least one film-forming polymer which is insoluble in the liquids of the digestive tract, present in an amount of from about 50% to about 90% (e.g. from about 50% to about 80%) by weight of dry matter of the controlled release coat composition, and including at least one non-hydrosoluble cellulose derivate, (e.g. ethylcellulose, cellulose acetate, or mixtures thereof);

at least one nitrogen-containing polymer, present in an amount of from about 2% to about 25% (e.,g. from about 5% to about 15%) by weight of dry matter of the controlled release coat composition, and including at least one polyacrylamide, poly-N-vinylaride, poly-N-vinyl-lactame, polyvinylpyrrolidone, or mixtures thereof,

optionally a plasticizer present in an amount of from about 2% to about 20% (e.g. from about 4% to about 15%) by weight of dry matter of the controlled release coat composition, and including at least one ofthe following compounds: glycerol esters, plitalates, citrates, sebacates, cetylalcolhol esters, castor oil, cutini, or mixtures thereof;

at least one surface-active and/or lubricating agent, present in an amount of from about 2% to about 20% (e.g. from about 4% to about 15%) by weight of dry matter of the controlled release coat composition, and chosen from anionic surfactants such as the alkali metal and alkaline-earth metal salts of fatty acids, (e.g. stearic acid, oleic acid, and mixtures thereof), and/or from nonionic surfactants such as polyoxyethylenated esters of sorbitan,

polyoxyethylenated esters of sorbitan, polyoxyethylenated derivatives of castor oil, and/or from lubricants such as stearates (e.g. calcium, magnesium, aluminium, zinc stearate and mixtures thereof), stearylfumarates (e.g. sodium stearylfumarate, glyceryl behenate and mixtures thereof); and mixtures thereof;

wherein the coated microparticles are designed so as to be able to remain in the small intestine for a period of at least about 5 hours; in certain embodiments at least about 7 hours; and in certain other embodiments for a period of from about 8 hours to about 24 hours; so as to allow absorption of the tetrabenazine during at least part of its time in the small intestine.

In a prophetic example of this embodiment of the invention, the microparticles are coated in a fluidized bead coater with the following coating solution:

| | |
|---|---|
| Ethylcellulose | about 44.7 g |
| PVP | about 4.8 g |
| Castor oil | about 4.8 g |
| Magnesium Stearate | about 6.1 g |
| Acetone | about 479 g |
| Isopropanol | about 53 g |

In other embodiments of the present invention, the release of the tetrabenazine from a controlled release formulation can be further influenced, i.e., adjusted to a desired rate, by the addition of one or more pore-formers to the controlled release coat, where the pore-formers can be inorganic or organic, and can include materials that can be dissolved, extracted or leached from the controlled release coat in the environment of use. Upon exposure to fluids in the environment of use, the pore-formers are, for example, dissolved, and channels and pores are formed that fill with the environmental fluid. For example, the pore-formers can include one or more water-soluble hydrophilic polymers in order to modify the release characteristics of the formulation. Non-limiting examples of suitable hydrophilic polymers include hydroxypropylmetlhylcellulose, cellulose ethers and protein-derived materials of these polymers, the cellulose ethers, (e.g. hydroxyalkylcelluloses and carboxyalkylcelluloses), and mixtures thereof. Also, synthetic water-soluble polymers can be used, such as polyvinylpyrrolidone, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, sorbitol, and mixtures thereof. In at least one embodiment the hydrophilic polymer(s) include hydroxypropyl-methylcellulose. Other examples of pore-formers include alkali metal salts such as lithium carbonate, sodium chloride, sodium bromide, potassium chloride, potassium sulfate, potassium phosphate, sodium acetate, sodium citrate, and mixtures thereof. The pore-forming solids can also be polymers which are soluble in the environment of use, such as CARBOWAX®, CARBOPOL®, and the like. The possible pore-formers embrace diols, polyols, polyhydric alcohols, polyalkylene glycols, polyglycols, poly(a-w)alkylenediols, and mixtures thereof. Other pore-formers which can be useful in the formulations of the present invention include starch, modified starch, and starch derivatives, gums, including but not limited to xanthan gum, alginic acid, other alginates, benitonite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambda-carrageenan, gum karaya, biosynthetic gum, and mixtures thereof. Other pore-formers include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In other embodiments a surfactant or an effervescent base can be included in the controlled release coat, which can reduce and in certain embodiments overcome surface tension effects. In addition, the controlled release coat of certain embodiments can include one or more osmagents (i.e., which can osmotically deliver the active agent from the device by providing an osmotic pressure gradient against the external fluid), swelling agents (i.e., which can include, but are not limited to hydrophilic pharmaceutically acceptable compounds with various swelling rates in water), or other pharmaceutically acceptable agents (i.e., provided in an amount sufficient to facilitate the entry of the environmental fluid without causing the disruption of the impermeable coating). The surfactants that can be used in the controlled release coat of certain embodiments can be anionic, cationic, nonionic, or amphoteric. Non-limiting examples of such surfactants include sodium lauryl sulfate, sodium dodecyl sulfate, sorbitan esters, polysorbates, pluronics, potassium laurate, and mixtures thereof. Non-limiting examples of effervescent bases that can be used in the controlled release coat of certain embodiments include sodium glycine carbonate, sodium carbonate, potassium carbonate, sodium bicarbonate, potassium bicarbonate, calcium bicarbonate, and mixtures thereof. Non-limiting examples of osmagents that can be used in the controlled release coat of certain embodiments include sodium chloride, calcium chloride, calcium lactate, sodium sulfate, lactose, glucose, sucrose, mannitol, urea, other organic and inorganic compounds known in the art, and mixtures thereof. The swelling agent can include, but is not limited to at least one pharmaceutically acceptable hydrophilic compound, having a swelling rate or swelling amount in water at about 25° C. that is: greater than or equal to at least about 10% by weight (wt/wt), greater than or equal to at least about 15% by weight (wt/wt), or greater than or equal to at least about 20% by weight (wt/wt). Non-limitinlg examples of swelling agents that can be used in the controlled release coat of certain embodiments of the present invention include crosslinked polyvinylpyrrolidones (e.g. polyplasdone, crospovidone and mixtures thereof), crosslinked carboxyalkylcelluloses, crosslinked carboxymethylcellulose (e.g. crosslinked sodium croscarmellose), hydrophilic polymers of high molar mass (i.e., which can be, but are not limited to being greater than or equal to 100,000 Daltons) which can include, but are not limited to: polyvinylpyrrolidone(s), polyalkylene oxides (e.g. polyethylene oxide, polypropylene oxide, and mixtures thereof), hydroxyalkylcelluloses (e.g. hydroxypropylcellulose, hydroxypropylmethylcellulose and mixtures thereof), carboxyalkylcellulose (e.g. carboxymethylcellulose), modified starch (e.g. sodium glycolate), starch or natural starch (e.g. corn, wheat, rice, potato and mixtures thereof), cellulose (i.e. which can be, but is not limited to being in powder form or microcrystalline form), sodium alginate, potassium polacriline, and corresponding blends or mixtures thereof. In other embodiments, non-limiting examples of the swelling agent include the following sub-set of compounds: crosslinked polyvinylpyrrolidone (e.g. polyplasdone, crospovidone or mixtures thereof), crosslinked carboxyalkylcelluloses (e.g. crosslinked carboxymethylcelluloses such as crosslinked sodium croscarmellose), and mixtures thereof. In other embodiments, the swelling agent can be a nitrogen containing polymer, non-limiting examples of which can include polyvinylpyrrolidone, crosslinked polyvinylpyrrolidone and mixtures thereof. The concentration of the swelling agent in the controlled release coat of certain embodiments of the present invention can be from about 3% to about 40% by weight of the microparticle. For example, in certain embodiments the concentration of the swelling agent in the controlled release coat is from about 4% to about 30%, and in other embodiments from about 5% to about 25% by weight of the microparticle.

In certain embodiments one or more pharmaceutically acceptable excipients consistent with the objects of the present invention can be used in the controlled release coat, such as a lubricant, an emulsifying agent, an anti-foaming agent, and/or a plasticizer.

Lubricants can be included in the controlled release coat to help reduce friction of coated microparticles during manufacturing. The lubricants that can be used in the controlled release coat of certain embodiments of the present invention include but are not limited to adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (e.g. COMPRITOL™), STEAR-O-WET™ and MYVATEX™ TL. In at least one embodiment, the lubricant is selected from magnesium stearate, talc and mixtures thereof. Combinations of these lubricants are operable. The lubricant can each be present in an amount of from about 1% to about 100% by weight of the controlled release coat dry weight. For example, in certain embodiments wherein the controlled release polymer is EUDRAGIT® NE30D or EUDRAGIT® NE40D (Rohm America LLC) together with a hydrophilic pore former, the lubricant is present in an amount of from about 1% to about 30% by weight of the controlled release coat dry weight; in other embodiments from about 2% to about 20%; and in still other embodiments at about 10% by weight of the microparticle controlled release coat dry weight. In another embodiments where the controlled release polymer is ethylcellulose (ETHOCEL™ PR100, PR45, PR20, PR10 or PR7 polymer, or a mixture thereof), the lubricant can be present in an amount of from about 10% to about 100% by weight of the microparticle control-releasing coat dry weight; in another embodiment from about 20% to about 80%; and in still another embodiments at about 50% by weight of the microparticle control-releasing coat dry weight.

Emulsifying agent(s) (also called emulsifiers or emulgents) can be included in the microparticle controlled release coat to facilitate actual emulsification during manufacture of the coat, and also to ensure emulsion stability during the shelf-life of the product. Emulsifying agents useful for the microparticle control-releasing coat composition include, but are not limited to naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Combinations of emulsifying agents are operable. In at least one embodiment, the emulsifying agent is TWEEN™ 80. The emulsifying agent(s) can be present in an amount of from about 0.01% to about 5% by weight of the microparticle controlled release coat dry weight. For example, in certain embodiments the emulsifying agent is present in an amount of from about 0.05% to about 3%; in other embodiments from about 0.08% to about 1.5%, and in still other embodiments at about 0.1% by weight of the microparticle controlled release coat dry weight.

Anti-foaming agent(s) can be included in the micropaiticle controlled release coat to reduce frothing or foaming during manufacture of the coat. Anti-foaminig agents useful for the coat composition include, but are not limited to simethicone, polyglycol and silicon oil. In at least one embodiment the anti-foaming agent is Simethicone C. The anti-foaming agent can be present in an amount of from about 0.1% to about 10% of the microparticle controlled release coat weight. For example, in certain embodiments the anti-foaming agent is present in an amount of from about 0.2% to about 5%; in other embodiments from about 0.3% to about 1%, and in still other embodiments at about 0.6% by weight of the micr-oparticle controlled release coat dry weight.

Plasticizer(s) can be included in the microparticle controlled release coat to modify the properties and characteristics of the polymers used in the coat for convenient processing during manufacturing (e.g. provide increased flexibility and durability during manufacturing). As used herein, the term “plasticizer” includes any compounds capable of plasticizing or softening a polymer or binder used in the present invention. Once the coat has been manufactured, certain plasticizers can function to increase the hydrophilicity of the coat in the environment of use. During manufacture of the coat, the plasticizer can lower the melting temperature or glass transition temperature (softening point temperature) of the polymer or binder. The addition of a plasticizer, such as low molecular weight PEG, generally broadens the average molecular weight of a polymer in which they are included thereby lowering its glass transition temperature or softening point. Plasticizers can also generally reduce the viscosity of a polymer. Non-limiting examples of plasticizers that can be used in the microparticle controlled release coat include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, and mixtures thereof. The plasticizer can be present in an amount of from about 1% to about 80% of the controlled release coat dry weight. For example, in certain embodiments the plasticizer is present in an amount of from about 5% to about 50%, in other embodiments from about 10% to about 40%, and in still other embodiments at about 20% of the controlled release coat dry weight.

The controlled release coat can be present in an amount of from about 1% to about 100% by weight of the micmoparticle, depending upon the choice of polymer, the ratio of polymer:pore former, and the total surface area of the microparticle formulation. Since a certain thickness of controlled release coating has to be achieved in order to achieve the desired dissolution profile, the amount of polymer coating required during manufacture is related to the total surface area of the batch of uncoated microparticles that requires a coating. The controlled release polymer surface area coverage can range from about 0.5 mg/cm2 to about 30mg/cm2. For example in certain embodiments the surface area coverage of the controlled release polymer is from about 0.6 mg/cm2 to about 20 mg/cm2, and in other embodiments from about 1 mg/cm2 to about 5 mg/cm2. In at least one embodiment of the invention, EUDRAGIT® NE30D is used as the controlled release polymer at a surface area coverage of about 10 mg/cm2. One approach to estimate the total surface area of a multiparticulate batch is the permeability method according to Blaine (ASTM Des. C 205-55), which is based upon the mathematical model of laminar flow through capillaries arranged in parallel. In the absence of an accurate determination of total surface area of a microparticle, the amount of controlled release polymer to be applied can be expressed as a percentage of the uncoated microparticle.

The controlled release polymer can be present in an amount of from about 1% to about 99% by weight of the coated microparticle, depending on the controlled release profile desired. For example, in certain embodiments the polymer is present in an amount of from about 5% to about 80%, and in other embodiments from about 10% to about 50% by weight of the coated microparticle. In at least one embodiment wherein the controlled release polymer is EUDRAGIT® NE30D, EUDRAGIT® NE40D (Rohm America LLC), KOLLICOAT® SR 30D, or a mixture thereof, the polymer is present in an amount of from about 1% to about 50%; in other embodiments from about 5% to about 30%; and in still other embodiments is about 15% by weight of the coated microparticle. In at least one embodiment wherein the controlled release polymer is ethylcellulose, the polymer is present in an amount of from about 1% to about 99% by weight of the coated microparticle; in other embodiments from about 5% to about 50%; and in still other embodiments at about 20% by weight of the coated microparticle. In at least one embodiment wherein the controlled release polymer is ETHOCEL™, an ethyl cellulose grade PR100, PR45, PR20, PR10, PR7 polymer, or a mixture thereof, the polymer is present in an amount of from about 5% to about 30% by weight of the coated microparticle; in other embodiments from about 10% to about 25%; and in still other embodiments at about 20% by weight of the coated microparticle.

In certain embodiments, the diameter of the micropar-ticles (with or without the controlled release coat) can range from about 50 μm to about 800 μm. For example, in certain embodiments the diameter of the microparticles range from about 100 μm to about 600 μm, and in other embodiments from about 150 μm to about 450 μm.

It is contemplated that in alternative embodiments, other excipients consistent with the objects of the present invention can also be used in the microparticle controlled release coat.

In at least one embodiment, the microparticle controlled release coat includes about 96% EUDRAGIT® NE30D, about 1.9% Magnesium stearate, about 1.9% Talc, about 0.04% TWEEN® 80, and about 0.19% Simethicone C, when expressed as percentage by weight of the dry controlled release coat composition. In another embodiment, the microparticle controlled release coat includes about 68% ethylcellulose, about 17% glyceryl monostearate and about 15% acetyl tributylcitrate when expressed as percentage by weight of the dry controlled release coat composition.

In certain embodiments the microparticle controlled release coat can be made according to any one of the methods described herein.

The manufacturing process for the microparticle controlled release coat can be as follows. Water is split into two portions of about 15% and about 85%. The anti-foaming agent and the emulsifying agent are then added to the 15% water portion, and mixed at about 300 rpm to forn portion A. In at least one embodiment, the anti-foaming agent is Simethicone C, and the emulsifying agent is TWEEN™ 80. A first lubricant is then added to the 85% water portion and mixed at about 9500 rpm to form portion B. In at least one embodiment, the first lubricant is talc. Then portion A is mixed with portion B, a second lubricant is slowly added, and mixed at about 700 rpm overnight. In at least one embodiment, the second lubricant is magnesium stearate. Finally, an aqueous dispersion of a neutral ester copolymer is added and mixed for about 30 minutes at about 500 rpm. In at least one embodiment, the aqueous dispersion of a neutral ester copolymer is EUDRAGIT® NE30D. The resultant controlled release coat solution can then be used to coat the microparticles to about a 35% weight gain with the following parameters: An inlet temperature of from about 10° C. to about 60° C., in certain embodiments from about 20° C. to about 40° C., and in at least one embodiment from about 25° C. to about 35° C.; an outlet temperature of from about 10° C. to about 60° C. in certain embodiments from about 20° C. to about 40° C., and in at least one embodiment from about 25° C. to about 35° C.; a product temperature of from about 10° C. to about 60° C., in certain embodiments from about 15° C. to about 35° C., and in at least one embodiment from about 22° C. to about 27° C.; an air flow of from about 10 cm/h to about 180 c.m/h, in certain embodiments from about 40 c.m/h to about 120 c.m/h, and in at least one embodiment from about 60 c.m/h to about 80 c.m/h; and an atomizing pressure of from about 0.5 bar to about 4.5 bar, in certain embodiments from about 1 bar to about 3 bar, and in at least one embodiment at about 2 bar. The resultant controlled release coated microparticles can then be discharged from the coating chamber and oven cured with the following parameters: A curing temperature of from about 20° C. to about 65° C., in certain embodiments from about 30° C. to about 55° C., and in at least one embodiment at about 40° C.; and a curing time of from about 2 hours to about 120 hours, in certain embodiments from about 10 hours to about 40 hours, and in at least one embodiment at about 24 hours. Any other technology resulting in the formulation of the microparticle controlled release coat consistent with the objects of the invention can also be used.

Microparticle Dosage Forms

Highly useful dosage forms result when microparticles made from compositions containing tetrabenazine, spheronization aids, and other excipient(s) are coated with controlled release polymer(s). The controlled release coated microparticles can then be combined with an excipient mass and/or other pharmaceutical excipients, and compressed into tablets. Conventional tablets can be manufactured by compressing the coated microparticles with suitable excipients using known compression techniques. The dissolution profile of the controlled release coated multiparticles is not substantially affected by the compression of the microparticles into a tablet. The resultant dosage forms enjoy the processing ease associated with the use of excipient masses and the release properties associated with controlled release coated microparticles. Alternatively, the coated microparticles can be filled into capsules.

The forms of administration according to the invention are suitable for oral administration. In certain embodiments the forms of administration are tablets and capsules. However, the composition of the invention can also take the form of pellets, beads or microtablets, which can then be packaged into capsules or compressed into a unitary solid dosage form. Other solid oral dosage forms as disclosed herein can be prepared by the skilled artisan, despite the fact that such other solid oral dosage forms may be more difficult to commercially manufacture.

The present invention also contemplates combinations of differently coated microparticles into a dosage form to provide a variety of different release profiles. For example, in certain embodiments, microparticles with a delayed release profile can be combined with other microparticles having a sustained release profile to provide a multiple component controlled release tetrabenazine formulation. In addition, other embodiments can include one or more further components of immediate release tetrabenazine. The immediate release tetrabenazine component can take the form of uncoated tetrabenazine microparticles or powders: tetrabenazine microparticles coated with a highly soluble immediate release coating, such as an OPADRY® type coating, as are known to those skilled in the art, or a combination of any of the foregoing. The multiple components can then be blended together in the desired ratio and placed in a capsule, or formed into a tablet. Examples of multiple component controlled release tetrabenazine formulations are described in U.S. Pat. No. 6,905,708.

Osmotic Dosage Forms

Osmotic dosage forms, osmotic delivery devices, modified release osmotic dosage forms, or osmosis-controlled extended-release systems are terms used interchangeably herein and are defined to mean dosage forms which forcibly dispense the tetrabenazine by pressure created by osmosis or by osmosis and diffusion of fluid into a material which expands and forces the tetrabenazine to be dispensed from the osmotic dosage form. Osmosis can be defined as the flow of solvent from a compartment with a low concentration of solute to a compartment with a high concentration of solute. The two compartments are separated by a membrane, wall, or coat, which allows flow of solvent (a liquid, aqueous media, or biological fluids) but not the solute. Examples of such membranes can for example be, a semipermeable membrane, microporous, asymmetric membrane, which asymmetric membrane can be permeable, semipermeable, perforated, or unperforated and can deliver the tetrabenazine by osmotic pumping, diffusion or the combined mechanisms of diffusion and osmotic pumping. Thus, in principle, osmosis controlled release of the tetrabenazine involves osmotic transport of an aqueous media into the osmotic dosage form followed by dissolution of the tetrabenazine and the subsequent transport of the saturated solution of the tetrabenazine by osmotic pumping of the solution through at least one passageway in the semipermeable membrane or by a combination of osmosis and diffusion through the semipermeable membrane.

It is well known to one of ordinary skill in the art that the desired in-vitro release rate and the in-vivo pharmacokinetic parameters can be influenced by several factors, such as for example, the amount of the tetrabenazine used to form the core, the amount of pharmaceutically acceptable excipient used to form the core, the type of pharmaceutically acceptable excipient used to form the core, the amount or type of any other materials used to form the core such as, for example, osmagents (the term osmagent, osmotically effective solutes, osmotically effective compound and osmotic agents are used interchangeably herein) osmopolymers, and any combination thereof. The release profile can also be influenced by the material used to form the semipermeable membrane covering the core or by the material used to form any coating, such as a controlled release coating (e.g. a delayed release coat) on the semipermeable membrane. With these factors in mind, an osmotic device can therefore be designed to exhibit an in-vitro release rate such that in certain embodiments, after about 2 hours from about 0 to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released, when measured for example by using a USP Type 1 apparatus (Rotating Basket Method) in 900 ml water, 0.1N HCl, 0.1N HCl+0.1% Cetrimilide, USP Buffer pH 1.5, Acetate Buffer pH 4.5, Phosphate Buffer, pH 6.5 or Phosphate Buffer pH 7.4 at 75 rpm at 37° C.±0.5° C. Alternatively dissolution may be effected in USP-3 media such as SGF pH 1.2, Acetate Buffer at pH 4.5 or phosphate buffer at pH 6.8.

Osmotic devices also may be designed to achieve an in-vitro release of no more than about 40% after about 2 hours, from about 40% to about 75% release after about 4 hours, at least about 75% after about 8 hours, and at least about 85% after about 16 hours when assayed using a dissolution medium such as identified above or known in the art.

In certain embodiments of the present invention, an osmotic dosage form is provided having a core including the tetrabenazine and one or more excipients. In at least one embodiment the osmotic dosage form includes an osmagent. The osmotic delivery system for example, can be in the form of a tablet or capsule containing microparticles.

In certain embodiments, the core of the osmotic dosage form includes a water swellable polymer, non-limiting examples of which include hydroxypropyl cellulose, alkylcellulose, hydroxyalkylcellulose, polyalkylene oxide, polyethylene oxide, and mixtures thereof. A binder can be included in the core of certain embodiments of the osmotic dosage form to increase the core's mechanical strength. Non-limiting examples of binders include polyvinyl pyrrolidine, carboxyvinyl polymer, methylcellulose, hydroxyethylcellulose, hydroxypropylcellulose, hydroxypropylmethylcellulose, a low molecular weight polyethylene oxide polymer, hydroxypropylmethylcellulose, dextrin, maltodextrin, gelatin, polyvinyl alcohol, xanthan gum, carbomers, carragheen, starch derivatives, and mixtures thereof. Lubricants can be included in certain embodiments of the osmotic dosage form to provide decreased friction between the solid and die wall during tablet manufacturing. Non-limiting examples of lubricants include stearic acid, magnesium stearate, glyceryl behenate, talc, mineral oil, sodium stearyl fumarate, hydrogenated vegetable oil, sodium benzoate, calcium stearate, and mixtures thereof. In other embodiments, additional inert excipients consistent with the objects of the invention can also be included in the core of the osmotic dosage form to facilitate the preparation and/or improve patient acceptability of the final osmotic dosage form as described herein. Suitable inert excipients are well known to the skilled artisan and can be found in the relevant literature, for example in the Handbook of Pharmaceutical Excipients (Rowe et. al., 4th Ed., Pharmaceutical Press, 2003).

In at least one embodiment, a modified release osmotic dosage form includes tetrabenazine in a therapeutically effective amount, which releases the tetrabenazine by forcibly dispensing the tetrabenazine from a core via a semipermeable membrane by diffusion and/or at least one passageway in the membrane by osmotic pumping (i) all or in part by pressure created in the core by osmosis i.e., positive hydrostatic pressure of a liquid, solvent, biological fluid or aqueous media and/or all or in part by the expansion of a swellable material which forces the tetrabenazine to be dispensed from the core of the dosage form, and (ii) is formulated such that the dosage form exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released.

In at least one embodiment, the modified release dosage form includes an osmotic delivery device including a homogenous solid core including substantially the tetrabenazine present in a therapeutically effective amount with at least one pharmaceutically acceptable excipient, said core surrounded by a semipermeable membrane which permits entry of an aqueous liquid into the core and delivery of the tetrabenazine from the core to the exterior of the dosage form through at least one passageway or by a combination of osmosis and diffusion such that the dosage form exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release rate of the tetrabenazine is such that after about 2 hours no more than about 40% is released, after about 4 hours from about 40% to about 75% is released, after about 8 hours at least about 75% is released, and after about 16 hours at least about 85% is released.

In at least one embodiment, the modified release dosage forn includes a multiparticulate dosage form, each micropaiticle including an osmotic delivery device, each microparticle including a homogenous solid core including substantially the tetrabenazine with at least one pharmaceutically acceptable excipient, said core of each microparticle surrounded by a semipermeable membrane which permits entry of an aqueous liquid into the core and delivery of the tetrabenazine from the core to the exterior of the dosage form through a plurality of pores formed in the semipermeable membrane by inclusion of a pore forming agent in the membrane or by a combination of osmosis and diffusion so as to allow communication of the core with the outside of the device for delivery of the tetrabenazine and is formulated such that the dosage form includes a therapeutically effective amount of the tetrabenazine and exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabeniazine is released. In at least one such eimbodimilent the in-vitro release rate of the tetrabenazine is such that after about 2 hours no more than about 40% is released, after about 4 hours from about 40% to about 75% is released, after about 8 hours at least about 75% is released and after about 16 hours at least about 85% is released.

In at least one embodiment, the modified release dosage form includes a inultiparticulate dosage form, each microparticle including an osmotic delivery device, each microparticle including a homogenous solid core including substantially the tetrabenazine in admixture with at least one pharmaceutically acceptable excipient, an osmagent and/or an osmopolymer, said core of each microparticle surrounded by a semipermeable membrane which permits entry of an aqueous liquid into the core and delivery of the tetrabenazine from the core to the exterior of the dosage form through a plurality of pores formed in the semipermeable membrane by inclusion of a pore forming agent in the membrane or by a combination of osmosis and by diffusion so as to allow communication of the core with the outside of the device for delivery of the tetrabenazine and is formulated such that the dosage form includes a therapeutically effective amount of the tetrabenazine and exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release rate of the tetrabenazine is such that after about 2 hours no more than about 40% is released, after about 4 hours from about 40% to about 75% is released, after about 8 hours at least about 75% is released and after about 16 hours at least about 85% is released.

In at least one embodiment, the modified release dosage forn includes a multiparticulate dosage form, each microparticle including a homogenous solid core including substantially the tetrabenazine with at least one pharmaceutically acceptable excipient in admixture with an osmagent, and/or an osmopolymer, and/or an absorption enhancer, said microparticles compressed into a tablet together with at least one pharmaceutically acceptable excipient, said tablet surrounded by a semipermeable membrane which permits entry of an aqueous liquid into the core and delivery of the tetrabenazine from the tablet interior to the exterior of the dosage form through at least one passageway in the semipermeable membrane and/or by diffusion through the semipermeable membrane so as to allow communication of the tablet interior with the exterior of the tablet for delivery of the tetrabenazine and is formulated such that the dosage form includes a therapeutically effective amount of the tetrabenazine and exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabeniazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released, and after about 16 hours at least about 85% is released.

In at least one embodiment, the modified release dosage form includes a niultiparticulate dosage form, each microparticle including a sugar sphere or nonpareil bead coated with at least one layer including substantially the tetrabenazine with at least one pharmaceutically acceptable excipient, said at least one layer surrounded by a semipermeable membrane which permits entry of an aqueous liquid into the layer and delivery of the tetrabenazine from the layer to the exterior of the dosage form through a plurality of pores formed in the semipermeable membrane by inclusion of a pore forming agent in the membrane and/or by diffusion so as to allow communication of the core with the outside of the device for delivery of the tetrabenazine and is formulated such that the dosage form includes a therapeutically effective amount of the tetrabenazine and exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

In at least one embodiment, the modified release dosage forn includes a multiparticulate dosage form, each microparticle including a sugar sphere or nonpareil bead coated with at least one layer including substantially the tetrabenazine in admixture with at least one pharmaceutically acceptable excipient, an osmagent and/or an osmopolymer, said at least one layer surrounded by a semipermeable membrane which permits entry of an aqueous liquid into the layer and delivery of the tetrabenazine from the layer to the exterior of the dosage form through a plurality of pores formed in the semipermeable membrane by inclusion of a pore forming agent in the membrane and/or by diffusion so as to allow communication of the core with the outside of the device for delivery of the tetrabenazine and is formulated such that the dosage form includes a therapeutically effective amount of the tetrabenazine and exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodinienlt the in-vitro release profile of tetrabenazine is such that after about 2 hours no more than about 40% is released, after about 4 hours from about 40% to about 75% is released. after about 8 hours at least about 75% is released and after about 16 hours at least about 85% is released.

In at least one embodiment, the modified release dosage forn includes a modified release osmotic dosage form including a homogenous core including a therapeutically effective amount of the tetrabeniazine in admixture with an osmagent, and/or an osmopolymer, and/or and absorption enhancer, said core surrounded by a nontoxic wall, membrane or coat, such as for example a semipermeable membrane which permits entry of an aqueous liquid into the core and delivery of the tetrabenazine from the core to the exterior of the dosage form through at least one passageway in the semipermeable membrane and/or by diffusion through the membrane so as to allow communication of the core with the outside of the dosage form for delivery of the tetrabenazine and is formulated such that the dosage form exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of tetrabenazine is such that after about 2 hours no more than about 40% is released, after about 4 hours from about 40% to about 75% is released, after about 8 hours at least about 75% is released and after about 16 hours at least about 85% is released.

In at least one embodiment the modified release dosage form includes an osmotic delivery device including the tetrabenazine present in a therapeutically effective amount in a layered, contacting arrangement with a swellable material composition to yield a solid core with two or more layers, which core is surrounded by a nontoxic wall, membrane or coat, such as for example a semipermeable membrane which permits entry of an aqueous liquid into the core and delivery of the tetrabenazine from the core to the exterior of the dosage form through at least one passageway in the semipermeable membrane or by osmosis and diffusion through the membrane so as to allow communication of the core with the outside of the dosage form for delivery of the tetrabenazine and is formulated such that the dosage form exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazilne is such that after about 2 hours no more than about 40% is released, after about 4 hours about 40% to about 75% is released, after about 8 hours at least about 75% is released and after about 16 hours at least about 85% is released.

In at least one embodiment, the modified release dosage fonn includes an osmotic delivery device including a core and a membrane surrounding said core, said core including a therapeutically effective amount of the tetrabenazine, and optionally at least one means for forcibly dispensing the tetrabenazine from the device, said membrane including at least one means for the exit of the tetrabenazine from the device, said device formulated such that when the device is in an aqueous medium, the tetrabenazine, and optionally the at least one means for forcibly dispensing the tetrabenazine from the device and the at least one means for the exit of the tetrabenazine from the device cooperatively function to exhibit an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenlazinie is such that after about 2 hours no more than about 40% is released, after about 4 hours from about 40% to about 75% is released, after about 8 hours at least about 75% is released and after about 16 hours at least about 85% is released.

In at least one embodiment, the modified release dosage forn includes an osmotic delivery device including a core and a membrane surrounding said core, said core including a therapeutically effective amount of the tetrabenazine, at least one means for increasing the hydrostatic pressure inside the membrane and optionally at least one means for forcibly dispensing the tetrabenazine from the device, said membrane including at least one means for the exit of the tetrabenazine from the device, said device formulated such that when the device is in an aqueous medium, the at least one means for increasing the hydrostatic pressure inside the membrane, and optionally the at least one means for forcibly dispensing the tetrabenazine from the device and the at least one means for the exit of the tetrabenazine cooperatively function to exhibit an in-vitro release rate such that after about 2 hours from about 0% to about 20% by weight of the tetrabenazine is released. after about 4 hours from about 15% to about 45% by weight of the tetrabeniazinle is released, after about 8 hours, from about 40% to about 90% by weight of the tetrabeniazine is released, and after about 16 hours, more than about 80% by weight of the tetrabenazine is released. In at least one such embodiment the in-vitro release profile of the tetrabenazilne is such that after about 2 hours no more than about 40% is released, after about 4 hours from about 40% to about 75% is released, after about 8 hours at least about 75% is released and after about 16 hours at least about 85% is released.

In at least one embodiment the invention is directed to the use of tetrabenazine, to produce once-daily administrable tablets or other dosage forms that are bioequivalent to Xenazine® (tetrabenazine) tablets, as defined by FDA criteria when administered once daily to a subject in need thereof. In particular at least one of the Tmax, Cmax, or AUC profile of certain embodiments of the present invention is within 80-125% of Xenazineg when administered once daily to a subject in need thereof. In at least one embodiment, the present invention encompasses once-daily 10 mg, 12.5 mg, 15 mg, 20 mg, 25 mg, 30 mg or 35 mg tetrabenazine formulations that are bioequivalent to Xenazine®.

In at least one embodiment, the invention is directed to a method of treating a condition including administering any one of the above described osmotic dosage forms to a patient in need of such administration once-daily.

The invention, in at least one embodiment, is directed to a method for administering tetrabenazine to the gastrointestinal tract of a human for the treatment or management of a condition, wherein the method includes: (a) admitting orally into the human a modified release dosage form including tetrabenazine, the modified release dosage form including an osmotic dosage form; and (b) administering the tetrabenazine from the osmotic dosage form in a therapeutically responsive dose to produce the treatment or management of the condition such that the osmotic dosage form exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

The invention, in at least one embodiment, is directed to a method for administering tetrabenazine to the gastrointestinal tract of a human for the treatment or management of a condition, wherein the method includes: (a) admitting orally into the human a modified release dosage form including a core and a membrane surrounding said core, said core including the tetrabenazine and optionally a means for forcibly dispensing the tetrabenazine from the device, said membrane including at least one means for the exit of the tetrabenazine from the dosage form, and (b) administering the tetrabenazine from the dosage form which is formulated such that when the dosage form is in an aqueous medium, the tetrabenazine and optionally the means for forcibly dispensing the tetrabenazine and the at least one means for the exit of the tetrabenazine cooperatively function to exhibit an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

The invention, in at least one embodiment, is directed to a method for administering tetrabenazine to the gastrointestinal tract of a human for the treatment or management of a condition, wherein the method includes: (a) admitting orally into the human a modified release dosage form including a core and a membrane surrounding said core, said core including the tetrabenazine, a means for increasing the hydrostatic pressure within the core and optionally a means for forcibly dispensing the tetrabenazine from the device, said membrane including at least one means for the exit of the tetrabenazine from the dosage form, and (b) administering the tetrabenazine from the dosage form which is formulated such that when the dosage form is in an aqueous medium, the tetrabenazine, the means for increasing the hydrostatic pressure within the core and optionally the means for forcibly dispensing the tetrabenazine and the at least one means for the exit of the tetrabenazine cooperatively function to exhibit an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

In at least one other embodiment. the osmotic dosage form fuither includes an immediate release coat for the immediate release of the tetrabenazine from the immediate release coat. In embodiments including the immediate release coat, the osmotic dosage form exhibits an in-vitro release rate such that after about 2 hours from about 0% to about 20% by weight of the tetrabenazine is released, after about 4 hours from about 1 5% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the teti-abenazine is such that after about 2 hours no more than about 40 % is released, after about 4 hours from about 40% to about 75% is released, after about 8 hours at least about 75% is released and after about 16 hours at least about 85% is released. In at least one other embodiment, the osmotic dosage forms further comprise an inert water-soluble coat covering the semipermeable membrane or coat. This inert water-soluble coat can be impermeable in a first external fluid, while being soluble in a second external fluid. In embodiments including the inert water-soluble coat, the osmotic dosage forin exhibits an in-vitro release rate such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

In at least one other embodiment. the osmotic dosage forms ftit-tler comprise an osmotic subcoat. In certain embodiments including the osmotic subcoat, the osmotic dosage form exhibits an in-vitro release rate such that after about 2 hours from about 0% to about 20% by weight of the tetrabenazinie is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In at least one such embodiment the in-vitro release profile of the tetrabenazine is such that after about 2 hours no more than about 40% is released, after about 4 hours from about 40% to about 75% is released, after about 8 hours at least about 75% is released and after about 16 hours at least about 85% is released.

In at least one other embodiment, the osmotic dosage forms further comprise a controlled release coat. The controlled release coat of the osmotic dosage formh can, for example, control, extend, and/or delay the release of the tetrabenazine. In certain embodiments including the controlled release coat, the osmotic dosage form exhibits an in-vitro release rate such that after about 2 hours from about 0% to about 20% by weight of the tetrabelnazine is released, after about 4 hours from about 15% to about 45% by weight of the tetrabenazine is released, after about 8 hours, from about 40% to about 90% by weight of the tetrabenlazinie is released, and after about 16 hours, more than about 80% by wveigilt of the tetrabenazine is released. In at least one such embodiment the in-vitio release profile ofthe tetrabeniazine is such that after about 2 hours no more than about 40% is released, after about 4 hours from about 40% to about 75% is released, after about 8 hours at least about 75% is released and after about 16 hours at least about 85% is released.

In at least one embodiment, the controlled release coat of the osmotic dosage form includes a material that is soluble or erodible in intestinal juices, substantially pH neutral or basic fluids of fluids having a pH higher than gastric fluid, but for the most part insoluble in gastric juices or acidic fluids.

In at least one embodiment. the controlled release coat of the osmotic dosage form includes at least one water-insoluble water-permeable film-forming polymer and at least one water-soluble polymer.

In at least one embodiment, the controlled release coat of the osmotic dosage form includes at least one water-insoluble water-permeable film-forming polymer and at least one water-soluble polymer and optionally at least one plasticizer.

In at least one embodiment, the controlled release coat of the osmotic dosage form includes at least one water-insoluble water-permeable film-forming polymer, at least one water-soluble polymer and at least one means for the exit of the tetrabenazine from the core of the osmotic dosage form.

In at least one embodiment, the controlled release coat of the osmotic dosage form includes at least one water-insoluble water-permeable film-forming polymer, at least one water-soluble polymer and at least one passageway.

In at least one embodiment, the controlled release coat of the osmotic dosage form includes at least one water-insoluble water-permeable film-forming polymer, at least one water-soluble polymer and at least one plasticizer.

In at least one embodiment, the controlled release coat of the osmotic dosage form includes at least one water-insoluble water-permeable film-forming polymer, at least one water-soluble polymer, optionally at least one plasticizer, and at least one means for the exit of the tetrabenazine from the core of the osmotic dosage form.

In at least one embodiment, the controlled release coat of the osmotic dosage form includes at least one water-insoluble water-permeable film-forming polymer, at least one water-soluble polymer, optionally at least one plasticizer, and at least one passageway.

In at least one embodiment, the controlled release coat of the osmotic dosage form includes an aqueous dispersion of a neutral ester copolymer without any functional groups; a poly glycol having a melting point greater than about 55° C., one or more pharmaceutically acceptable excipients, and optionally at least one means for the exit of the tetrabenazine form the core of the osmotic dosage form. This controlled release coat is cured at a temperature at least equal to or greater than the melting point of the polyglycol.

In at least one other embodiment, the controlled release coat of the osmotic dosage form includes at least one enteric polymers

In certain embodiments the membrane or wall is permeable to the passage of aqueous media but not to the passage of the tetrabenazine present in the core. The membrane can be, for example, a semipermeable membrane or an asymmetric membrane, which can be permeable, semipermeable, perforated, or unperforated and can deliver the tetrabenazine by osmotic pumping, or the combined mechanisms of diffusion and osmotic pumping. The structural integrity of such membranes preferably remains substantially intact during the period of delivery of the tetrabenazine. By “substantially intact” it is meant that the semipermeable property of the membrane is not compromised during the period of delivery of the tetrabenazine.

The semipermeable membrane of the osmotic dosage form of certain embodiments includes at least one pharmaceutically acceptable excipient, at least one polymer, wax, or combination thereof, although appropriately treated inorganic materials such as ceramics, metals or glasses can be used. When the semipermeable membrane includes at least one polymer, the molecular weight of the at least one polymer or combination of polymers are preferably such that the polymer or combination of polymers is solid at the temperature of use i.e., both in-vitro and in-vivo.

In certain embodiments, the at least one polymer included in the semipermeable membrane of the osmotic dosage form can be a cellulose ester, such as for example, cellulose acetate, cellulose acetate acetoacetate, cellulose acetate benzoate, cellulose acetate butylsulfonate, cellulose acetate butyrate, cellulose acetate butyrate sulfate, cellulose acetate butyrate valerate, cellulose acetate caprate, cellulose acetate caproate, cellulose acetate caprylate, cellulose acetate carboxymethoxypropionate, cellulose acetate chloroacetate, cellulose acetate dimethaminoacetate, cellulose acetate dimethylaminoacetate, cellulose acetate dimethylsulfamate, cellulose acetate dipalmitate, cellulose acetate dipropylsulfamate, cellulose acetate ethoxyacetate, cellulose acetate ethyl carbamate, cellulose acetate ethyl carbonate, cellulose acetate ethyl oxalate, cellulose acetate furoate, cellulose acetate heptanoate, cellulose acetate heptylate, cellulose acetate isobutyrate, cellulose acetate laurate, cellulose acetate methacrylate, cellulose acetate methoxyacetate, cellulose acetate methylcarbarnate, cellulose acetate methylsulfonate, cellulose acetate myristate, cellulose acetate octanoate, cellulose acetate palmitate, cellulose acetate phthalate, cellulose acetate propionate, cellulose acetate propionate sulfate, cellulose acetate propionate valerate, cellulose acetate p-toluene sulfonate, cellulose acetate succinate, cellulose acetate sulfate, cellulose acetate trimellitate, cellulose acetate tripropioniate, cellulose acetate valerate, cellulose benzoate, cellulose butyrate napthylate, cellulose butyrate, cellulose chlorobenzoate, cellulose cyanoacetates, cellulose dicaprylate, cellulose dioctanoate, cellulose dipentanate, cellulose dipentanlate, cellulose formate, cellulose methacrylates, cellulose methoxybenzoate, cellulose nitrate, cellulose nitrobenzoate, cellulose phosphate (sodium salt), cellulose phosphinates, cellulose phosphites, cellulose phosphonates, cellulose propionate, cellulose propionate crotonate, cellulose propionate isobutyrate, cellulose propionate succinate, cellulose stearate, cellulose sulfate (sodium salt), cellulose triacetate, cellulose tricaprylate, cellulose triformate, cellulose triheptanoate, cellulose triheptylate, cellulose trilaurate, cellulose trimyristate, cellulose trinitrate, cellulose trioctanoate, cellulose tripalmitate, cellulose tripropionate, cellulose trisuccinate, cellulose trivalerate, cellulose valerate palmitate; a cellulose ether, such as for example, 2-cyanoethyl cellulose, 2-hydroxybutyl methyl cellulose, 2-hydroxyethyl cellulose, 2-hydroxyethyl ethyl cellulose, 2-hydroxyethyl methyl cellulose, 2-hydroxypropyl cellulose, 2-hydroxypropyl methyl cellulose, dimethoxyethyl cellulose acetate, ethyl 2-hydroxylethyl cellulose, ethyl cellulose, ethyl cellulose sulfate, ethylcellulose dimethylsulfamate, methyl cellulose, methyl cellulose acetate, methylcyanoethyl cellulose, sodium carboxymethyl 2-hydroxyethyl cellulose, sodium carboxymethyl cellulose; a polysulfone, such as for example, polyethersulfones; a polycarbonate; a polyurethane; a polyvinyl acetate; a polyvinyl alcohol; a polyester; a polyalkene such as polyethylene, ethylene vinyl alcohol copolymer, polypropylene, poly(1,2-dimethyl-1-butenylene), poly(1-bromo-1-butenylene), poly(1, butene), poly(1-chloro-1-butenylene), poly(1-decyl-1-butenylene), poly(1-hexane), poly(1-isopropyl-1-butenylene), poly(1-pentene), poly(3-vinylpyrene), poly(4-methoxyl 1-butenylene), poly(ethylene-co-methyl styrene), poly vinyl-chloride, poly(ethylene-co-tetrafluoroethylene), poly(ethylene-terephthalate), poly(dodecafluorobutoxylethylene), poly(hexafluoroprolylene), poly(hexyloxyethylene), poly(isobutene), poly(isobutene-co-isoprene), poly(isoprene), poly-butadiene, poly[(pentafluoroethyl)ethylene], poly[2-ethylhexyloxy)ethylene], poly(butylethylene), poly(tertbutylethylene), poly(cylclohexylethy-lene), poly[(cyclohexylmethyl)ethylene], poly(cyclopentylethylene), poly(decylethylene), poly-(dodecy-lethylene), poly(neopentylethylene), poly(propylethylene); a polystyrene, such as for example, poly(2,4-dimethyl styrene), poly(3-methyl styrene), poly(4-methoxystyrene), poly(4-methoxystyrene-stat-styrene), poly(4-methyl styrene), poly(isopentyl styrene), poly(isopropyl styrene), polyvinyl esters or polyvinyl ethers, such as form example, poly(benzoylethylene), poly(butoxyethylene), poly(chloroprene), poly(cycloheXRoxyethylene), poly(decyloxyethylene), poly(dichloroethylene), poly(difluoroethylene), poly(vinyl acetate), poly(vinlyltrimethyilstyrene); a polysiloxane, such as for example, poly(dimethylsiloxane); a polyacrylic acid derivative, such as for example, polyacrylates, polymethyl methacrylate, poly(acrylic acid) higher alkyl esters, poly(ethylmethacrylate), poly(hexadecyl methacrylate-co-methylmethacrylate), poly-(methylacrylate-co-styrene), poly(n-butyl methacrylate), poly(n-butyl-acrylate), poly(cyclododecyl acrylate), poly(benzyl acrylate), poly(butylacrylate), poly(secbutylacrylate), poly(hexyl acrylate), poly(octyl acrylate), poly(decyl acrylate), poly(dodecyl acrylate), poly(2-methyl butyl acrylate), poly(adamantyl methacrylate), poly(benzyl methacrylate), poly(butyl methacrylate), poly(2-ethylhexyl methacrylate), poly(octyl methacrylate), acrylic resins; a polyamide, such as for example, poly(iminoadipoyliminododecamethylene), poly(iminoadipoyliminohexamethylene), polyethers, such as for example, poly(octyloxyethylene), poly(oxyphenylethylene), poly(oxypropylene), poly(pentyloxyethylene), poly(phenoxy styrene), poly(secbutroxylethylene), poly(tert-butoxyethylene); and combinations thereof.

In at least one embodiment, the at least one wax included in the semipermeable membrane of the osmotic dosage form can be, for example, insect and animal waxes, such as for example, Chinese insect wax, beeswax, spermaceti, fats and wool wax; vegetable waxes, such as for example, bamboo leaf wax, candelilla wax, carnauba wax, Japan wax, ouricury wax, Jojoba wax, bayberry wax, Douglas-Fir wax, cotton wax, cranberry wax, cape berry wax, rice-bran wax, castor wax, Indian corn wax, hydrogenated vegetable oils (e.g., castor, palm, cottonseed, soybean), sorghum grain wax, Spanish moss wax, sugarcane wax, caranda wax, bleached wax, Esparto wax, flax wax, Madagascar wax, orange peel wax, shellac wax, sisal hemp wax and rice wax; mineral waxes, such as for example, Montan wax, peat waxes, petroleum wax, petroleum ceresin, ozokerite wax, microcrystalline wax and paraffins; synthetic waxes, such as for example, polyethylene wax, Fischer-Tropsch wax, chemically modified hydrocarbon waxes, cetyl esters wax; and combinations thereof

In at least one embodiment, the semipermeable membrane of the osmotic dosage form can comprise a combination of at least one polymer, wax, or combinations thereof and optionally at least one excipient.

In embodiments where the tetrabenazine is released through the membrane or wall in a controlled manner by the combined mechanisms of diffusion and osmotic pumping, the membrane or wall can comprise at least one of the above described polymers and/or waxes or a combination of polymers, such as for example, cellulose esters, copolymers of methacrylate salts and optionally a plasticizer.

The poly(methacrylate) copolymer salts used in the manufacturing of the membrane for the osmotic dosage form can be, for example, insoluble in water and in digestive fluids, but are permeable to different degrees. Examples of such copolymers are poly(ammonium methacrylate) copolymer RL (EUDRAGIT®RL), poly(ammonium methacrylate) copolymer (type A-USP/NF), poly(aminoalkyl methacrylate) copolymer RL-JSP 1), and (ethyl acrylate)-(methyl methacrylate)-[(trimethylammonium)-ethylmethacrylate] (1:2:0.2) copolymer, MW 150,000. Other examples of such copolymers include those available from Rohm Pharma, Weiterstadt, such as for example, EUDRAGIT®RS 100: solid polymer, EUDRAGIT®RL 12.5:12.5% solution in solvent, EUDRAGIT®RL 30 D: 30% aqueous dispersion, and other equivalent products. The following poly (ammonium methiacrylate) copolymers can also be used: ammonium methacrylate copolymer RS (EUDRAGIT®RS), poly(ammonium methacrylate) copolymer (type B-USP/NF), poly(aminoalkyl methacrylate) copolymer (RSL-JSP 1), (ethyl acrylate)-(methyl methacrylate)-[(trimethylammonium)-ethyl methacrylate] (1:2:0.1) copolymer, PM 150,000. Specific polymers include (Rohm Pharma, Weiterstadt): EUDRAGIT®RS 100: solid polymer, EUDRAGIT®RS 12.5:12.5% solution in solvent, EUDRAGIT®RS 30 D: 30% aqueous dispersion and other equivalent products. RL is readily water permeable while EUDRAGIT®RS is hardly water permeable. By employing mixtures of both EUDRAGIT®RL and EUDRAGIT®RS, membranes having the desired degree of permeability to achieve the in-vitro dissolution rates and in-vivo pharmacokinetic parameters can be prepared.

The use of plasticizers is optional but can be included in the osmotic dosage forms of certain embodiments to modify the properties and characteristics of the polymers used in the coats or core of the osmotic dosage forms for convenient processing during manufacture of the coats and/or the core of the osmotic dosage forms if necessary. As used herein, the term “plasticizer” includes any compounds capable of plasticizing or softening a polymer or binder used in invention. Once the coat or membrane has been manufactured, certain plasticizers can function to increase the hydrophilicity of the coat(s) and/or the core of the osmotic dosage form in the environment of use. During manufacture of the coat, the plasticizer lowers the melting temperature or glass transition temperature (softening point temperature) of the polymer or binder. Plasticizers, such as low molecular weight PEG, can be included with a polymer and lower its glass transition temperature or softening point. Plasticizers also can reduce the viscosity of a polymer. The plasticizer can impart some particularly advantageous physical properties to the osmotic device of the invention.

Plasticizers useful in the osmotic dosage form of certain embodiments of the invention can include, for example, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol, glycerin, ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate, allyl glycolate and mixtures thereof. All such plasticizers are commercially available from sources such as Aldrich or Sigma Chemical Co. It is also contemplated and within the scope of the invention, that a combination of plasticizers can be used in the present formulation. The PEG based plasticizers are available commercially or can be made by a variety of methods, such as disclosed in Poly(ethylene glycol) Chemistry: Biochemical and Biomedical Applications (J. M. Harris, Ed.; Plenum Press, NY). Once the osmotic dosage form is manufactured, certain plasticizers can function to increase the hydrophilicity of the coat(s) and/or the core of the osmotic dosage form in the environment of use may it be in-vitro or in-vivo. Accordingly, certain plasticizers can function as flux enhancers.

The ratio of cellulose esters:copolymers of methacrylate salts:plasticizer of the osmotic dosage forms can be, for example, about 1% to about 99% of the cellulose ester by weight: about 0.5% to about 84% of the copolymers of methacrylate salt by weight: about 0.5% to about 15% of the plasticizer by weight. The total weight percent of all components including the wall is 100%.

Aside from the semipermeable membranes of the osmotic dosage forn described above, asymmetric membranes can also be used to surround the core of an osmotic dosage form for the controlled release of the tetrabenazine to provide the in-vitro release rates described above and the therapeutically beneficial in-vivo pharmacokinetic parameters for the treatment or management of a condition. Such asymmetric membranes can be permeable, semipermeable, perforated, or unperforated and can deliver the tetrabenazine by osmotic pumping, diffusion or the combined mechanisms of diffusion and osmotic pumping. The manufacture and use thereof of asymmetric membranes for the controlled-release of an active drug through one or more asymmetric membranes by osmosis or by a combination of diffusion osmotic pumping is known.

In certain embodiments of the osmotic dosage form, the semipermeable membrane can further comprise a flux enhancing, or channeling agent. “Flux enhancing agents” or “channeling agents” are any materials which function to increase the volume of fluid imbibed into the core to enable the osmotic dosage form to dispense substantially all of the tetrabenazine through at least one passageway in the semipermeable membrane by osmosis or by osmosis and by diffusion through the semipermeable membrane. The flux enhancing agent dissolves to form paths in the semipermeable membrane for the fluid to enter the core and dissolve the tetrabenazine in the core together with the osmagent, if one is present, but does not allow exit of the tetrabenazine. The flux enhancing agent can be any water soluble material or an enteric material which allows an increase in the volume of liquid imbibed into the core but does not allow for the exit of the tetrabenazine. Such materials can be, for example, sodium chloride, potassium chloride, sucrose, sorbitol, mannitol, polyethylene glycol, propylene glycol, hydroxypropyl cellulose, hydroxypropyl methylcellulose, hydroxypropyl methylcellulose phthalate, cellulose acetate phthalate, polyvinyl alcohols, methacrylic copolymers, and combinations thereof. Some plasticizers can also function as flux enhancers by increasing the hydrophilicity of the semipermeable membrane and/or the core of the osmotic dosage form. Flux enhancers or channeling agents can also function as a means for the exit of the tetrabenazine from the core if the flux enhancing or channeling agent is used in a sufficient amount.

The expression “passageway” as used herein includes means and methods suitable for the metered release of the tetrabenazine from the core of the osmotic dosage form. The means for the exit of the tetrabenazine includes at least one passageway, including orifice, bore, aperture, pore, porous element, hollow fiber, capillary tube, porous overlay, or porous element that provides for the osmotic controlled release of the tetrabenazine. The means for the exit can be linear or tortuous. The means for the exit includes a weakened area of the semipermeable membrane or a material that erodes or is leached from the wall in a fluid environment of use to produce at least one dimensioned passageway. The means for the exit of the tetrabenazine can comprise any leachable material, which when leaches out of the semipermeable membrane forms a passageway suitable for the exit of the tetrabenazine from the core of the osmotic dosage form. Such leachable materials can comprise, for example, a leachable poly(glycolic) acid or poly(lactic) acid polymer in the semipermeable membrane, a gelatinous filament, poly(vinyl alcohol), leachable polysaccharides, salts, oxides, sorbitol, sucrose or mixtures thereof. The means for exit can also comprise a flux enhancer or channeling agent if present in a sufficient amount. The means for the exit possesses controlled-release dimensions, such as round, triangular, square and elliptical, for the metered release of the tetrabenazine from the dosage form. The dimensions of the means of the exit for the tetrabenazine is sized such so as to allow the tetrabenazine to pass through the means for the exit. The dosage form can be constructed with one or more means for the exit in spaced apart relationship on a single surface or on more than one surface of the wall.

The expression “fluid environment” denotes an aqueous or biological fluid as in a human patient, including the gastrointestinal tract. The means for the exit can be preformed for example by mechanical means after the semipermeable membrane is applied to the core of the osmotic dosage form, such as for example by mechanical perforation, laser perforation, or by using a properly sized projection on the interior of a tablet punch to form the means for the exit of the tetrabenazine, such as for example a cylindrical or frustoconical pin which is integral with the inside surface of the upper punch of a punch used to form the osmotic dosage form. Alternatively, the means for the exit of the tetrabenazine can be formed by incorporating a leachable material or pore forming agent into the semipermeable composition before the semipermeable membrane is applied to the core of the osmotic dosage form. The means for the exit of the tetrabenazine can comprise a combination of the different exit means described above. The osmotic dosage form can comprise more than one means for the exit of the tetrabenazine including two, three, four, five, six seven, eight, nine ten or more exit means and can be formed in any place of the osmotic dosage form. The various positions of the means for the exit are disclosed. The type, number, and dimension(s) of the means for the exit of the tetrabenazine is such that the dosage form exhibits the desired in-vitro release rates described herein and can be determined by routine experimentation by those skilled in the pharmaceutical delivery arts. The means for the exit and equipment for forming the means for the exit are known.

The osmotic device can further comprise a controlled release coat surrounding the semipermeable membrane including an enteric or delayed release coat that is soluble or erodible in intestinal juices, substantially pH neutral or basic fluids of fluids having a pH higher than gastric fluid, but for the most part insoluble in gastric juices or acidic fluids. A wide variety of other polymeric materials are known to possess these various solubility properties. Such other polymeric materials include, for example, cellulose acetate phthalate (CAP), cellulose acetate trimelletate (CAT), poly(vinyl acetate) phthalate (PVAP), hydroxypropyl methylcellulose phthalate (HP), poly(methylacrylate ethylacrylate) (1:1) copolymer (MA-EA), poly(methacrylate methylmethacrylate) (1:1) copolymer (MA-MMA), poly(methacrylate methylmethacrylate) (1:2) copolymer, EUDRAGIT® L-30-D (MA-EA, 1:1), EUDRAGIT® L- 100-55 (MA-EA, 1:1), hyciroxypropyl methylcellulose acetate succinate (HPMCAS), COATERIC® (PVAP), AQUATERIC® (CAP), AQUACOAT) (HPMCAS) and combinations thereof. The enteric coat can also comprise dissolution aids, stability modifiers, and bioabsorption enhancers.

In at least one embodiment the controlled release coat of certain osmotic dosage forms include materials such as hydroxypropylcellulose, microcrystalline cellulose (MCC, AVICEL™ from FMC Corp.), poly (ethylene-vinyl acetate) (60:40) copolymer (EVAC from Aldrich Chemical Co.), 2-hydroxyethylmethacrylate (HEMA), MMA, terpolymers of HEMA: MMA:MA synthesized in the presence of N,N′-bis(methacryloyloxyethyloxycarbonylamino)-azobenzene, azopolymers, enteric coated timed release system (TIME CLOCKE® from Pharmaceutical Profiles, Ltd., UK), calcium pectinate, and mixtures thereof.

Polymers that can be used in the controlled release coat of osmotic dosage forms of certain embodiments can be, for example, enteric materials that resist the action of gastric fluid avoiding permeation through the semipermeable wall while one or more of the materials in the core of the dosage form are solubilized in the intestinal tract thereby allowing delivery of the tetrabenazine in the core by osmotic pumping in the osmotic dosage form to begin. A material that adapts to this kind of requirement can be, for example, a poly(vinylpyriolidone)-vinyl acetate copolymer, such as the material supplied by BASF under its KOLLIDON® VA64 trademark, mixed with magnesium stearate and other similar excipients. The coat can also comprise povidone, which is supplied by BASF under its KOLLIDON® K 30 trademark, and hydroxypropyl methylcellulose, which is supplied by Dow under its METHOCEL® E-15 trademark. The materials can be prepared in solutions having different concentrations of polymer according to the desired solution viscosity. For example, a 10% P/V aqueous solution of KOLLIDON® K 30 has a viscosity of about 5.5 to about 8.5 cps at 20° C., and a 2% P/V aqueous solution of METHOCEL® E- 15 has a viscosity of about 13 to about 18 cps at 20° C.

The controlled release coat of osmotic dosage forms of certain embodiments can comprise one or more materials that do not dissolve, disintegrate, or change their structural integrity in the stomach and during the period of the that the tablet resides in the stomach, such as for example a member chosen from the group (a) keratin, keratin saridarac-tolu, salol (phenyl salicylate), salol beta-naphthylbenzoate and acetotannin, salol with balsam of Peru, salol with tolu, salol with gum mastic, salol and stearic acid, and salol and shellac; (b) a member chosen from the group of formalized protein, formalized gelatin, and formalized cross-linked gelatin and exchange resins; (c) a member chosen from the group of myristic acid-hydrogenated castor oil-cholesterol, stearic acid-mutton tallow, stearic acid-balsam of tolu, and stearic acid-castor oil; (d) a member chosen from the group of shellac, ammoniated shellac, ammoniated shellac-salol, shellac-wool fat, shellac-acetyl alcohol, shellac-stearic acid-balsam of tolu, and shellac n-butyl stearate; (e) a member chosen from the group of abietic acid, methyl abictate, benzoin, balsam of tolu, sandarac, mastic with tolu, and mastic with tolu, and mastic with acetyl alcohol; (f) acrylic resins represented by anionic polymers synthesized from methacrylate acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methylmethacrylate copolymer of about 150,000 molecular weight, methacrylic acid-methylmethacrylate 50:50 copolymer of about 135,000 molecular weight, methacrylic acid-methylmethacrylate-30:70-copolymer of about 135,000 mol. wt., methacrylic acid-dimethylaminoethyl-methacrylate-ethylacrylate of about 750,000 mol. wt., methacrylic acid-methylmethacrylate-ethylacrylate of about 1,000,000 mol. wt., and ethylacrylate-methylmethacrylate-ethylacrylate of about 550,000 mol. wt; and, (g) an enteric composition chosen from the group of cellulose acetyl phthalate, cellulose diacetyl phthalate, cellulose triacetyl phthalate, cellulose acetate phthalate, hydroxypropyl methylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl includes from about 1 to about 7 straight and branched alkyl groups, aryl phthalates, and other materials known to one or ordinary skill in the art. Combinations thereof are operable.

Accordingly, in at least one other embodiment, the controlled release coat of osmotic dosage forms of certain embodiments includes a water-insoluble water-permeable film-forming polymer, water-soluble polymer, and optionally a plasticizer and/or a pore-forming agent. The water-insoluble, water-permeable film-forming polymers useful for the manufacture of the controlled release coat can be cellulose ethers, such as for example, ethyl celluloses chosen from the group of ethyl cellulose grade PR100, ethyl cellulose grade PR20, cellulose esters, polyvinyl alcohol, and any combination thereof. The water-soluble polymers useful for the controlled release coat can be, for example, polyvinylpyrrolidone, hydroxypropyl methylcellulose, hydroxypropyl cellulose, and any combination thereof.

The skilled artisan will appreciate that that the desired in-vitro release rates described herein for the tetrabenazine can be achieved by controlling the permeability and/or the amount of coating applied to the core of the osmotic dosage form. The permeability of the controlled release coat, can be altered by varying the ratio of the water-insoluble, water-permeable film-forming polymer:water-soluble polymer:optionally the plasticizer and/or the quantity of coating applied to the core of the osmotic dosage form. A more extended release is generally obtained with a higher amount of water-insoluble, water-permeable film forming polymer. The addition of other excipients to the core of the osmotic dosage form can also alter the permeability of the controlled release coat. For example, if the core of the osmotic dosage form includes a swellable polymer, the amount of plasticizer in the controlled release coat can be increased to make the coat more pliable as the pressure exerted on a less pliable coat by the swellable polymer could rupture the coat. Further, the proportion of the water-insoluble water-permeable film forming polymer and water-soluble polymer can also be altered depending on whether a faster or slower in-vitro dissolution is desired.

In at least one other embodiment, the controlled release coat of the osmotic dosage form includes an aqueous dispersion of a neutral ester copolymer without any functional groups; a poly (glycol having a melting point greater than about 55° C., and one or more pharmaceutically acceptable excipients and cured at a temperature at least equal to or greater than the melting point of the poly glycol. The manufacture and use of such coating formulations are known. In brief, examples of neutral ester copolymers without any functional groups including the coat can be EUDRAGIT® NE30D, EUDRAGIT® NE40D (Röhm America LLC), or mixtures thereof. This coat can comprise hydrophilic agents to promote wetting of the coat when in contact with gastrointestinal fluids. Such hydrophilic agents Include, for example, hydrophilic water-soluble polymers such as hydroxypropyl methylcellulose (HPMC), hydroxypropyl cellulose (HPC) and combinations thereof. The poly glycol can be, for example, chosen from the group of polyethylene glycol 6000, polyethylene glycol 8000, polyethylene glycol 10000, polyethylene glycol 20000, Poloxamer 188, Poloxamer 338, Poloxamer 407, Polyethylene Oxides, Polyoxyethylene Alkyl Ethers, and Polyoxyethylene Stearates, and combinations thereof. This controlled release coat of the osmotic dosage form can further comprise a pore-forming agent. In at least one embodiment the pore former is sufficiently insoluble in the aqueous dispersion, and is sufficiently soluble in the environment of use. Methods for producing such coats are known.

The controlled release coat of certain embodiments of the osmotic dosage form of certain embodiments of the present invention includes at least one polymer in an amount sufficient to achieve a controlled release of the tetrabenazine. Examples of polymers that can be used in the controlled release coat of these embodiments include cellulose acetate phthalate, cellulose acetate trimaletate, hydroxy propyl i methylcellulose phthalate, polyvinyl acetate phthalate, ammonio methacrylate copolymers such as those sold under the trademark EUDRAGIT® RS and RL, poly acrylic acid and poly acrylate and methacrylate copolymers such as those sold under the trademark EUDRAGIT® S and L, polyvinyl acetaldiethylamino acetate, hydroxypropyl methylcellulose acetate Succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (EUDRAGIT® RS-PM, Rohm & Haas), pullulan, collagen, casein, agar, gum arabic, sodium carboxymethyl cellulose, (swellable hydrophilic polymers) poly(hydroxyalkyl methacrylate) (molecular weight from about 5K to about 5000K), polyvinylpyrrolidone (molecular weight from about 10K to about 360K), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (molecular weight from about 30K to about 300K), polysaccharides such as agar, acacia, karaya, tragacanth, algins and guar, polyacrylamides, POLYOX® polyethylene oxides (molecular weight from about 100K to about 5000K), AQUAKEEP® acrylate polymers, diesters of polyglucan, crosslinked polyvinyl alcohol and poly N-vinyl-2-pyrrolidone, sodium starch glycolate (e.g. EXPLOTAB®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. POLYOX, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. EUDRAGIT®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. In at least one embodiment of the osmotic dosage form of the present invention, the polymer is an acrylate dispersion such as EUDRAGIT® NE30D, EUDRAGIT® NE40D (Rohm America LLC), KOLLICOAT® SR 30D, SURELEASE®, or a mixture thereof. The polymer can be present in an amount of from about 20% to about 90% by weight of the controlled release coat, depending on the controlled release profile desired. For example, in certain embodiments of the osmotic dosage form, the polymer is present in an amount of from about 50% to about 95%, in other embodiments from about 60% to about 90%, and in still other embodiments about 75% of the controlled release coat weight.

The controlled release coat of certain embodiments of the osmotic dosage form of the present invention can also include one or more pharmaceutically acceptable excipients such as lubricants, emulsifiers, anti-foaming agents, plasticisers, solvents and the like.

Lubricants can be included in the controlled release coat of certain embodiments of the osmotic dosage form of the present invention to help reduce friction of coated microparticles during manufacturing. The lubricants that can be used in the controlled release coat include but are not limited to adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (i.e. COMPRITOL™), STEAR-O-WET™ and MYVATEX™ TL. Combinations of these lubricants are operable. In at least one embodiment, the lubricant is selected from magnesium stearate, talc and a mixture thereof. The lubricant(s) can each be present in an amount of from about 0.1% to about 80% of the controlled release coat weight. For example, in certain embodiments the lubricant is present in an amount of from about 0.5% to about 20%, in other embodiments from about 0.8% to about 10%, and in still other embodiments about 1.5% of the controlled release coat weight.

Emulsifying agent(s) (also called emulsifiers or emulgents) can be included in the controlled release coat of the osmotic dosage forms of certain embodiments of the present invention to facilitate actual emulsification during manufacture of the coat, and also to increase or ensure emulsion stability during the shelf-life of the product. Emulsifying agents useful for the controlled release coat composition of the osmotic dosage form include, but are not limited to naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Combinations of emulsifying agents are operable. The emulsifying agent(s) can be present in an amount of from about 0.01% to about 0.25% of the controlled release coat weight. For example, in certain embodiments the emulsifying agent is present in an amount offrom about 0.01% to about 0.15%, in other embodiments from about 0.01% to about 0.07%, and in still other embodiments about 0.03% of the controlled release coat weight.

Anti-foaming agent(s) can be included in the controlled release coat of the osmotic dosage form of certain embodiments of the present invention to reduce frothing or foaming during manufacture of the coat. Anti-foaming agents useful for the controlled release coat composition of the osmotic dosage form include, but are not limited to simethicone, polyglycol, silicon oil and mixtures thereof. In at least one embodiment the anti-foaming agent is Simethicone C. The anti-foaming agent can be present in an amount of from about 0.01% to about 10% of the controlled release coat weight. For example, in certain embodiments the anti-foaming agent is present in an amount of from about 0.05% to about 1%, in other embodiments from about 0.1% to about 0.3%, and in still other embodiments about 0.15% of the controlled release coat weight.

It is contemplated that in certain embodiments, other excipients consistent with the objects of the present invention can also be used in the controlled release coat of the osmotic dosage form.

In at least one embodiment, the controlled release coat of the osmotic dosage form includes about 75% EUDRAGIT® NE30D, about 1.5% Magnesium stearate, about 1.5% Talc, about 0.03% TWEEN™ 80, about 0.15% Simethicone C, and about 21.82% water, by weight of the controlled release coat composition.

The osmotic dosage forn of certain embodiments can be made according to any one of the methods described herein. In a prophetic example of certain embodiments of osmotic dosage forms of the present invention, the manufacturing process for the controlled release coat of the osmotic dosage form can hypothetically be as follows: Water is split into two portions of about 15% and about 85%. The anti-foaming agent and the emulsifying agent are then added to the 15% water portion, and mixed at about 300 rpm to form portion A. In at least one embodiment, the anti-foaming agent is Simethicone C, and the emulsifying agent is TWEEN™ 80. A first lubricant is then added to the 85% water portion and mixed at about 9500 rpm to form portion B. In at least one embodiment, the first lubricant is talc. Then portion A is mixed with portion B, a second lubricant is slowly added, and mixed at about 700 rpm overnight. In at least one embodiment, the second lubricant is magnesium stearate. Finally, an aqueous dispersion of a neutral ester copolymer is added and mixed for about 30 minutes at about 500 rpm. In at least one embodiment, the aqueous dispersion of a neutral ester copolymer is EUDRAGIT® NE30D. The resultant coat solution can then be used to coat the osmotic subcoated microparticles to about a 35% weight gain with the following parameters: An inlet temperature of from about 10° C. to about 60° C., in certain embodiments from about 20° C. to about 40° C., and in at least one embodiment from about 25° C. to about 35° C.; an outlet temperature of from about 10° C. to about 60° C., in certain embodiments from about 20° C. to about 40° C., and in at least one embodiment from about 25° C. to about 35° C.; a product temperature of from about 10° C. to about 60° C., in certain embodiments from about 15° C. to about 35° C., and in at least one embodiment from about 22° C. to about 27° C.; an air flow of from about 10 cm/h to about 180 cm/h, in certain embodiments from about 40 cm/h to about 120 cm/h, and in at least one embodiment from about 60 cm/h to about 80 cm/h; and an atomizing pressure of from about 0.5 bar to about 4.5 bar, in certain embodiments from about 1 bar to about 3 bar, and in at least one embodiment at about 2 bar. The resultant coated microparticles can then be discharged from the coating chamber and overcured with the following parameters: A curing temperature of from about 20° C. to about 65° C., in certain embodiments from about 30° C. to about 55° C., and in at least one embodiment at about 40° C.; and a curing time of from about 2 hours to about 120 hours, in certain embodiments from about 10 hours to about 40 hours, and in at least one embodiment at about 24 hours. Any other technology resulting in the coating formulation of the controlled release coat of the osmotic dosage form that is consistent with the objects of the invention can also be used.

In at least one other embodiment, the osmotic dosage forms comprise a water-soluble or rapidly dissolving coat between the semipermeable membrane and the controlled release coat. The rapidly dissolving coat can be soluble in the buccal cavity and/or upper GI tract, such as the stomach, duodenum, jejunum or upper small intestines. Materials suitable for the manufacture of the water-soluble coat are known. In certain embodiments, the rapidly dissolving coat can be soluble in saliva, gastric juices, or acidic fluids. Materials which are suitable for making the water soluble coat or layer can comprise, for example, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as, for example, hydroxymethylcellulose, hydroxyethlylcellulose, and hydroxypropylcellulose; synthetic water-soluble cellulose-based lamina formers such as, for example, methyl cellulose and its hydroxyalkyl methylcellulose cellulose derivatives such as a member chosen from the group of hydroxyethyl methylcellulose, hydroxypropyl methylcellulose, and hydroxybutyl methylcellulose; croscarmellose sodium; other cellulose polymers such as sodium carboxymethylcellulose; and mixtures thereof. Other lamina forming materials that can be used for this purpose include, for example, poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer and mixtures thereof. The water soluble coating can comprise other pharmaceutical excipients that in certain embodiments can alter the way in which the water soluble coating behaves. The artisan of ordinary skill will recognize that the above-noted materials include film-forming polymers. The inert water-soluble coat covering the semipermeable wall and blocking the passageway of osmotic dosage forms of the present invention, is made of synthetic or natural material which, through selective dissolution or erosion can allow the passageway to be unblocked thus allowing the process of osmotic delivery to start. This water-soluble coat can be impermeable to a first external fluid, while being soluble in a second external fluid. This property can help to achieve a controlled and selective release of the tetrabenazine from the osmotic dosage form so as to achieve the desired in-vitro release rates.

In embodiments where the core of the osmotic dosage form does not comprise an osmagent, the osmotic dosage forms can comprise an osmotic subcoat, which can surround the core of the osmotic dosage form. The osmotic subcoat includes at least one osmotic agent and at least one hydrophilic polymer. The osmotic subcoat of these embodiments provides for the substantial separation of the tetrabenazine from the osmotic agent into substantially separate compartments/layers. This separation can potentially increase the stability of the tetrabenazine by reducing possible unfavorable interactions between the tetrabenazine and the osmagent, and/or between the tetrabenazine and the components of the controlled release coat. For example, the osmagent can be hygroscopic in nature, and can attract water that can lead to the degradation of the tetrabenazine. Since the osmotic agent of these embodiments can be substantially separated from the tetrabenazine, the tetrabenazine can be less prone to degradation from the water drawn in by the osmagent. The controlled release coat includes at least one controlled release polymer and optionally a plasticizer. The coated cores of the osmotic dosage form can be filled into capsules, or alternatively can be compressed into tablets using suitable excipients. In these embodiments the osmotic dosage form can utilize both diffusion and osmosis to control drug release, and can be incorporated into sustained release and/or delayed release dosage forms. In addition, in certain embodiments the osmotic pressure gradient and rate of release of the tetrabenazine can be controlled by varying the level of the osmotic agent and/or the level of the hydrophilic polymer in the osmotic subcoat, without the need for a seal coat around the osmotic subcoat.

The hydrophilic polymer used in an osmotic subcoat of certain embodiments of the present invention functions as a carrier for the osmotic agent. In certain embodiments the hydrophilic polymer in the osmotic subcoat does not substantially affect the drug release. In at least one embodiment, the hydrophilic polymer used in the osmotic subcoat does not act as a diffusion barrier to the release of the tetrabenazine. In at least one embodiment the release profile of the osmotic agent is substantially the same as the release profile of the tetrabenazine. Such hydrophilic polymers useful in an osmotic subcoat of certain embodiments of the present invention include by way of example, polyvinyl pyrrolidone, hydroxyethyl cellulose, hydroxypropyl cellulose, low molecular weight hydroxypropyl methylcellulose (HPMC), polymethacrylate, ethyl cellulose, and mixtures thereof. In at least one embodiment, the hydrophilic polymer of the osmotic subcoat is a low molecular weight and a low viscosity hydrophilic polymer. A wide variety of low molecular weight and low viscosity hydrophilic polymers can be used in the osmotic subcoat. Examples of HPMC polymers that can be used in the osmotic subcoat include PHARMACOAT® 606, PHARMACOAT® 606G, PHARMACOAT® 603, METHOCEL® E3, METHOCEL® E5, METHOCEL® E6, and mixtures thereof. The hydrophilic polymer of the osmotic subcoat can be present in an amount of from about 1% to about 30% by weight of the osmotic subcoat composition. For example, in certain embodiments the hydrophilic polymer is present in an amount of from about 1% to about 20%, in other embodiments from about 3% to about 10%, and in still other embodiments about 7% by weight of the osmotic subcoat composition.

In at least one embodiment, the osmotic subcoat includes about 7% PHARMACOAT® 606, about 1% sodium chloride. and about 92% water, by weight of the osmotic subcoat compositiont.

One method for producing the osmotic subcoat can be as follows. The at least one osmotic agent, for example sodium chloride, is dissolved in water. The solution of osmotic agent and water is then heated to about 60° C. The hydrophilic polymer is then added gradually to the solution. A magnetic stirrer can be used to aid in the mixing of the hydrophilic polymer to the solution of osmotic agent and water. The resultant osmotic subcoating solution can then be used to coat the core of the osmotic dosage form in a fluidized bed granulator, such as a granulator manufactured by Glatt (Germany) or Aeromatic (Switzerland) to the desired weight gain. An inlet temperature of from about 10° C. to about 70° C., in certain embodiments from about 30° C. to about 55° C., and in at least one embodiment from about 40° C. to about 45° C.; an outlet temperature of from about 10° C. to about 70° C. in certain embodiments from about 20° C. to about 45° C., and in at least one embodiment from about 30° C. to about 35° C.; a product temperature of from about 10° C. to about 70° C., in certain embodiments from about 20° C. to about 45° C., and in at least one embodiment from about 30° C. to about 35° C.; an air flow of from about 10 cm/h to about 180 cm/h; in certain embodiments from about 40 cm/h to about 120 cm/h; and in at least one embodiment from about 60 cm/h to about 80 cm/h; an atomizing pressure of from about 0.5 bar to about 4.5 bar, in certain embodiments from about 1 bar to about 3 bar, and in at least one embodiment at about 2 bar; a curing temperature of from about 10° C. to about 70° C., in certain embodiments from about 20° C. to about 50° C., and in at least one embodiment from about 30° C. to about 40° C.; and a curing time of from about 5 minutes to about 720 minutes; in certain embodiments from about 10 minutes to about 120 minutes, and in at least one embodiment at about 30 minutes. Any other technology resulting in the coating formulation of the osmotic subcoat consistent with the objects of the invention can also be used.

The ratio of the components in the core, semipermeable membrane and/or water-soluble membrane and/or at least one controlled release coat and/or osmotic subcoat as well as the amount of the various membranes or coats applied can be varied to control delivery of the tetrabenazine either predominantly by diffusion across the surface of the semipermeable membrane to predominantly by osmotic pumping through the at least one passageway in the semipermeable membrane, and combinations thereof such that the dosage form can exhibit a modified-release, controlled-release, sustained-release, extended-release, prolonged-release, bi-phasic release, delayed-release profile or a combination of release profiles whereby the in-vitro release rates of the tetrabenazine is such that after about 2 hours from about 0% to about 20% by weight of the tetrabenazine is released, after about 4 hours from about 15% to about 45% by weight of the tetrabenazine is released, after about 8 hours, from about 40% to about 90% by weight of the tetrabenazine is released, and after about 16 hours, more than about 80% by weight of the tetrabenazine is released. In embodiments where the mode of exit of the tetrabenazine includes a plurality of pores, the amount of pore forming agent employed to achieve the desired in-vitro dissolution rates can be readily determined by those skilled in the drug delivery art.

In at least one embodiment of the osmotic dosage form, the core includes tetrabenazine in an amount of from about 40% to about 99% of the core dry weight. For example in certain embodiments the core includes tetrabenazine in an amount of about 40%, about 45%, about 50%, about 55%, about 60%, about 65%, about 70%, about 75%, about 80%, about 85%, about 90%, about 95% or about 99% of the core dry weight.

In certain embodiments, the core of the osmotic dosage form includes at least one means for increasing the hydrostatic pressure inside the membrane or coat. The membrane or coat can be a semipermeable membrane, a controlled release coat, a water-soluble coat, an osmotic subcoat, or any combination thereof. The core of the osmotic dosage form has an effective osmotic pressure greater than that of the surrounding fluid in the environment of use so that there is a net driving force for water to enter the core. The at least one means for increasing the hydrostatic pressure inside the membrane or coat can be any material that increases the osmotic pressure of the core of the osmotic dosage form. The at least one means for increasing the hydrostatic pressure inside the membrane or coat can be, for example, the tetrabenazine, an osmagent, any material which can interact with water and/or an aqueous biological fluid, swell and retain water within their structure, such as for example an osmopolymer, and any combination thereof. The osmagent can be soluble or swellable. Examples of osmotically effective solutes are inorganic and organic salts and sugars. The tetrabenazine can itself be an osmagent or can be combined with one or more other osmagents, such as for example, magnesium sulfate, magnesium chloride, sodium chloride, lithium chloride, potassium sulfate, sodium carbonate, sodium sulfite, lithium sulfate, potassium chloride, calcium carbonate, sodium sulfate, calcium sulfate, potassium acid phosphate, calcium lactate, d-mannitol, urea, inositol, magnesium succinate, tartaric acid, water soluble acids, alcohols, surfactants, and carbohydrates such as raffinose, sucrose, glucose, lactose, fructose, algin, sodium alginate, potassium alginate, carrageenan, fucoridan, furcellaran, laminaran, hypnea, gum arabic, gum ghatti, gum karaya, locust bean gum, pectin, starch and mixtures thereof. In certain embodiments the core includes osmagent in an amount of about 15%, about 20%, about 25%, about 30%, about 35%, about 40%, about 45%, about 50%, about 55%, about 60%, about 65%, about 70%, about 75%, about 80%, about 85%, about 90%, or about 95% of the core dry weight.

The osmagent useful in certain embodiments of the present invention can be any agent that can generate an osmotic pressure gradient for the transport of water from the external environment of use into the osmotic dosage form. Osmagenits are also known as osmotically effective compounds, osmotic solutes, and osmotic fluid imbibing agents. Osmagenlts useful in certain embodiments of the present invention are soluble in aqueous and biological fluids, such as ionizing compounds, inherently polar compounds, inorganic acids, organic acids, bases and salts. In at least one embodiment the osmagent is a solid and dissolves to form a solution with fluids imbibed into the osmotic dosage form. A wide variety of osagents can be used to provide the osmotic pressure gradient used to drive the tetrabenazine from the core of the osmotic dosage form. Examples of inorganic salts useful as osmagents include lithium chloride, lithium sulfate, lithium phosphate, magnesium chloride, magnesium sulfate, potassium chloride, potassium sulfate, potassium phosphate, potassium acid phosphate, sodium chloride, sodium sulfate, sodium phosphate, sodium sulfite, sodium nitrate, sodium nitrite, and mixtures thereof. Examples of salts of organic acids useful as osagents include sodium citrate, potassium acid tartrate, potassium bitartrate, sodium bitartrate, and mixtures thereof. Examples of ionizable solid acids useful as osmagents include tartaric, citric, maleic, malic, fumaric, tartronic, itaconic, adipic, succinic, mesaconic acid, and mixtures thereof. Examples of other compounds useful as osmagents include potassium carbonate, sodium carbonate, ammonium carbonate, calcium lactate, mannitol, urea, inositol, magnesium succinate, sorbitol, and carbohydrates such as raffinose, sucrose, glucose, lactose, lactose monohydrate, a blend of fructose glucose and mixtures thereof. In at least one embodiment the osmagent is selected from sodium chloride, sodium bromide, sodium bisulfate, potassium acid tartrate, citric acid, mannitol, sucrose and mixtures thereof. Combinations of these osmagents is permissible. The osmagent can be present in an amount of from about 0.1% to about 50% of the dosage form weight. For example, in certain embodiments the osmagent is present in an amount of from about 1% to about 40%, and in other embodiments from about 1% to about 20% of the dosage form weight.

In certain embodiments, the at least one means for increasing the hydrostatic pressure can comprise, in addition to an osmagent, any material which can interact with water and/or an aqueous biological fluid, swell and retain water within their structure. In certain embodiments where the at least one means for increasing the hydrostatic pressure is an osmopolymer, which can be slightly cross-linked or uncross-linked. The uncross-linked polymers to be used as osmopolymers, when in contact with water and/or aqueous biological fluid, preferably do not dissolve in water, hence maintaining their physical integrity. Such polymers can be, for example, chosen from the group of polyacrylic acid derivatives (e.g., polyacrylates, poly-methyl methacrylate, poly(acrylic acid) higher alkyl esters, poly(ethylmethacrylate), poly(hexadecyl methacrylate-co-methylmethacrylate), poly(methylacrylate-co-styrene), poly(n-butyl methacrylate), poly(n-butyl-acrylate), poly(cyclododecyl acrylate), poly(benzyl acrylate), poly(butylacrylate), poly(secbutylacrylate), poly(hexyl acrylate), poly(octyl acrylate), poly(decyl acrylate), poly(dodecyl acrylate), poly(2-methyl butyl acrylate), poly(adamantyl methacrylate), poly(benzyl methacrylate), poly(butyl methacrylate), poly(2-ethylhexyl methacrylate), poly(octyl methacrylate), acrylic resins), polyacrylamides, poly(hydroxy ethyl methacrylate), poly(vinyl alcohol), poly(ethylene oxide), poly N-vinyl-2-pyrrolidone, naturally occurring resins such as polysaccharides (e.g., dextrans, water-soluble gums, starches, chemically modified starches), cellulose derivatives (e.g., cellulose esters, cellulose ethers, chemically modified cellulose, microcrystalline cellulose, sodium carboxymethylcellulose and methylcellulose), starches, CARBOPOL™, acidic carboxy polymer, CYANAMER™, polyacrylamides, cross-linked water-swellable idene-maleic anhydride polymers, GOOD-RITE™, polyacrylic acid, polyethylene oxide, starch graft copolymers, AQUA-KEEPS™, acrylate polymer, diester cross-linked polyglucan, and any combination thereof.

In certain embodiments, the core of the osmotic dosage form further includes a means for forcibly dispensing the tetrabenazine from the core to the exterior of the dosage form. The at least one means for forcibly dispensing the tetrabenazine can be any material which can swell in water and/or aqueous biological fluid and retain a significant fraction of water within its structure, and will not dissolve in water and/or aqueous biological fluid, a means for generating a gas, an osmotically effective solute or any combination thereof which can optionally be surrounded by a membrane or coat depending on the particular means used. The membrane or coat can be, for example, a membrane or coat that is essentially impermeable to the passage of the tetrabenazine, gas and compounds, and is permeable to the passage of water and/or aqueous biological fluids. Such a coat or membrane includes, for example, a semipermeable membrane, microporous membrane, asymmetric membrane, which asymmetric membrane can be permeable, semipermeable, perforated, or unperforated. In at least one embodiment, the at least one means for forcibly dispensing the tetrabenazine from the core of the osmotic dosage form includes a means for generating gas, which means for generating gas is surrounded by, for example, a semipermeable membrane. In operation, when the gas generating means imbibes water and/or aqueous biological fluids, the means for generating gas reacts and generates gas, thereby enlarging and expanding the at least one means for forcibly dispensing the tetrabenazine unidirectionally or multidirectionally. The means for generating a gas includes any compound or compounds, which can produce effervescence, such as for example, at least one solid acid compound and at least one solid basic compound, which in the presence of a fluid can react to form a gas, such as for example, carbon dioxide. Examples of acid compounds include, organic acids such as malic, fumaric, tartaric, itaconic, maleic, citric, adipic, succinic and mesaconic, and inorganic acids such as sulfamic or phosphoric, also acid salts such as monosodium citrate, potassium acid tartrate and potassium bitartrate. The basic compounds include, for example, metal carbonates and bicarbonates salts, such as alkali metal carbonates and bicarbonates. The acid and base materials can be used in any convenient proportion from about 1 to about 200 parts of the at least one acid compound to the at least one basic compound or from about 1 to about 200 parts of the at least one basic compound to the at least one acid compound. The means for generating gas is known.

In at least one embodiment, the at least one means for forcibly dispensing the tetrabenazine form the core of the osmotic dosage form includes any material which can swell in water and/or aqueous biological fluid and retain a significant fraction of water within its structure, and will not dissolve in water and/or aqueous biological fluid, such as for example, a hydrogel. Hydrogels include, for example, lightly cross-linked hydrophilic polymers, which swell in the presence of fluid to a high degree without dissolution, usually exhibiting a 5-fold to a 50-fold volume increase. Non-limiting examples of hydrogels include poly(hydroxyalkyl methacrylates), poly(acrylamide), poly(methacrylamide), poly(N-vinyl-2-pyrrolidone), anionic and cationic hydrogels, polyelectrolyte complexes, a water-insoluble, water-swellable copolymer produced by forming a dispersion of finely divided copolymers of maleic anhydride with styrene, ethylene, propylene butylene or isobutylene cross-linked with from about 0.001 to about 0.5 moles of a polyunsaturated cross-linking agent per mole of maleic anhydride in a copolymer, water-swellable polymers or N-vinyl lactams, semi-solid cross-linked poly(vinyl pyrrolidone), diester cross-linked polyglucan hydrogels, anionic hydrogels of heterocyclic N-vinyl monomers, ionogenic hydrophilic gels, and mixtures thereof. Some of the osmopolymers and hydrogels are interchangeable. Such means can optionally be covered by a membrane or coat impermeable to the passage of the tetrabenazine, and compounds, and is permeable to the passage of water and/or aqueous biological fluids. Such a coat or membrane includes, for example, a semipermeable membrane, microporous membrane, asymmetric membrane, which asymmetric membrane can be permeable, semipermeable, perforated, or unperforated.

In at least one other embodiment, the at least one means for forcibly dispensing the tetrabenazine from the core of the osmotic dosage form includes at least one osmotically effective solute surrounded by a membrane or coat impermeable to the passage of the tetrabenazine, and compounds, and is permeable to the passage of water and/or aqueous biological fluids such that the osmotically effective solute exhibits an osmotic pressure gradient across a membrane or coat. Such coat or membrane includes, for example, a semipermeable membrane, microporous membrane, asymmetric membrane, which asymmetric membrane can be permeable, semipermeable, perforated, or unperforated. The osmotically effective solutes include, for example, the osmagents described above.

In embodiments of the osmotic dosage font where the means for forcibly dispensing the tetrabenazine is surrounded by a membrane or coat, at least one plasticizer can be added to the membrane composition to impart flexibility and stretchability to the membrane or coat. In embodiments where the means for forcibly dispensing the tetrabenazine includes a means for generating a gas, the membrane or coat preferably is stretchable so as to prevent rupturing of the membrane or coat during the period of delivery of the tetrabenazine. Methods of manufacturing such a membrane or coat is known. Plasticizers, which can be used in these embodiments include, for example, cyclic and acyclic plasticizers, phthalates, phosphates, citrates, adipates, tartrates, sebacates, succinates, glycolates, glycerolates, benzoates, myristates, sulfonamides halogenated phenyls, poly(alkylene glycols), poly(alkylenediols), polyesters of alkylene glycols, dialkyl phthalates, dicycloalkyl phthalates, diaryl phthalates and mixed alkyl-aryl phthalates, such as for example, dimethyl phthalate, dipropyl phthalate, di(2-ethylhexyl)phthalate, di-isopropyl phthalate, diamyl phthalate and dicapryl phthalate; alkyl and aryl phosphates, such as for example, tributyl phosphate, trioctyl phosphate, tricresyl phosphate, trioctyl phosphate, tricresyl phosphate and triphenyl phosphate; alkyl citrate and citrates esters such as tributyl citrate, triethyl citrate, and acetyl triethyl citrate; alkyl adipates, such as for example, dioctyl adipate, diethyl adipate and di(2-methoxyethyl)adipate; dialkyl tartrates, such as for example, diethyl tartrates and dibutyl tartrate; alkyl sebacates, such as for example, diethyl sebacate, dipropyl sebacate and dinonyl sebacate; alkyl succinates, such as for example, diethyl succinate and dibutyl succinate; alkyl glycolates, alkyl glycerolates, glycol esters and glycerol esters, such as for example, glycerol diacetate, glycerol triacetate, glycerol monolactate diacetate, methyl phthalyl ethyl glycolate, butyl phthalyl butyl glycolate, ethylene glycol diacetate, ethylene glycol dibutyrate, triethylene glycol diacetate, triethylene glycol dibutyrate, triethylene glycol dipropionate and mixtures thereof. Other plasticizers include camphor, N-ethyl (o- and p-toulene) sulfonamide, chlorinated biphenyl, benzophenone, N-cyclohexyl-p-toluene sulfonamide, substituted epoxides and mixtures thereof.

The at least one means for forcibly dispensing the tetrabenazine from the core of certain embodiments of the osmotic dosage form can be located such that it is approximately centrally located within the core of the osmotic dosage form and is surrounded by a layer including the tetrabenazine. Alternatively, the core of the osmotic dosage form includes at least two layers in which the first layer includes the tetrabenazine salt, osmagent and/or osmopolymer and optionally at least one pharmaceutically acceptable excipient adjacent to a second layer including the means for forcibly dispensing the tetrabenazine. Alternatively, the core of the osmotic dosage form includes a multilayered structure in which the layer including the tetrabenazine is sandwiched between two layers of the means for forcibly dispensing the tetrabenazine from the osmotic dosage form.

AQ Controlled Release Coat

In certain embodiments of the present invention, there is provided a controlled release oral dosage form including a core that is surrounded by a controlled release coating (“AQ Controlled Release Coat”), wherein the AQ Controlled Release Coat includes a neutral ester copolymer without any functional groups, a poly glycol having a melting point of at least about 55° C., and one or more pharmaceutically acceptable excipients. The AQ Controlled Release Coat is formed by a process that includes coating the core with a coating composition that includes an aqueous dispersion of a neutral ester copolymer without any functional groups, a poly glycol having a melting point greater than about 55° C., and one or more pharmaceutically acceptable excipients, to form a coated core; and curing the coated core at a temperature at least equal to or greater than the melting point of the poly glycol, to form a stable controlled release monolithic coating. The coating formulation of the AQ Controlled Release Coat is quite versatile in that it can be used to coat a variety of drug cores and can be easily manipulated to obtain the desired drug release profile.

In at least one embodiment, the AQ Controlled Release Coat is formed by a process that excludes usage of an organic solvent.

In at least one embodiment, the AQ Controlled Release Coat hydrates when placed in an aqueous environment (e.g. water).

In at least one embodiment the controlled release dosage form coated with the AQ Controlled Release Coat expands in a dimensionally restricted manner when placed in an aqueous environment.

In at least one embodiment the controlled release dosage form coated with the AQ Controlled Release Coat floats when placed in an aqueous environment.

In at least one embodiment, the controlled release dosage form, upon oral administration to a patient, provides controlled release of an effective amount of the active drug to at least one region of the patient's upper gastrointestinal tract (e.g. the stomach).

In at least one embodiment the AQ Controlled Release Coat is further surrounded by a non-functional overcoat.

In at least one embodiment the core includes at least one therapeutically active agent and one or more first pharmaceutically acceptable excipients. In at least one embodiment the one or more first pharmaceutically acceptable excipients includes a superdisintegrant. Non-limiting examples of the superdisintegrant include crospovidone, crosscarmelose sodium (e.g. Ac-Di-Sol®), sodium starch glycolate (e.g. Explotab®), low-substituted hydroxypropylcellulose (L-HPC), and mixtures thereof.

In at least one embodiment, the curing is conducted for a time period of from about 1 to about 24 hours. In at least one embodiment, the curing is conducted for a time period of from about 1 to about 16 hours. In at least one embodiment, the curing is conducted for a time period of from about 1 to about 7 hours. In at least one embodiment, the Curing is conducted for a time period of from about 1 to about 3 hours.

The coating composition used to form the AQ Controlled Release Coat includes an aqueous dispersion of a neutral ester copolymer without any functional groups. The aqueous dispersion of a neutral ester copolymer without any functional groups can be an ethyl acrylate and methyl methacrylate copolymer dispersion. Non-limiting examples of ethyl acrylate and methyl methacrylate copolymer dispersions include a 30% aqueous dispersion of a neutral copolymer based on ethyl acrylate and methyl methacrylate (e.g. Eudragit® NE30D), a 40% aqueous dispersion of a neutral copolymer based on ethyl acrylate and methyl methacrylate (e.g. Eudragit® NE40D), Eudragit® NM30D, Kollicoat® EMM30D, and combinations thereof. In at least one embodiment the polymer is Eudragit® NE30D, which can be present in an amount of from about 1% to about 35% by weight of the coating composition, including all values and ranges therebetween, depending on the controlled release profile desired. In certain embodiments the neutral ester copolymer without any functional groups is present in an amount from about 20% to about 99.5% by dry weight of the coat, including all values and subranges therebetween. In other embodiments the neutral ester copolymiier without any functional groups is present in an amount from about 25% to about 60% by dry weight of the coat, including all values and subranges therebetween. In still other embodiments the neutral ester copolymer without any functional groups is present in an amount from about 37% to about 50% by dry weight of the coat, including all values and subranges therebetween. In certain embodiments the neutral ester copolymer without any functional groups is present in the coating composition in an amount of from about 0.4% to about 39.8% by dry weight of the tablet including all values and subranges therebetween; in other embodiments in an amount of from about 0.8% to about 24.0% by dry weight of the tablet, including all values and subranges therebetween; and in still other embodiments in an amount of from about 2.0% to about 5.5% by dry weight of the tablet, including all values and subranges therebetween.

In certain embodiments, the coating composition used to form the AQ Controlled Release Coat includes an aqueous dispersion of an ethylcellulose, a poly glycol having a melting point of at least about 55° C., and one or more pharmaceutically acceptable excipients; wherein said coating composition is coated onto the dosage form and cured at a temperature at least equal to or greater than the melting point of the poly glycol. Non limiting examples of aqueous dispersions of an ethylcellulose include SURELEASE® (Colorcoll, Inc., West Point, Pa., U.S.A.), and AQUACOAT® (FMC Corp., Philadelphia, Pa., U.S.A.). Combinations are operable.

The coating composition used to form the AQ Controlled Release Coat also includes a poly glycol with a melting point of at least about 55° C. Non-limiting examples of a poly gycol with a melting point of at least about 55° C. that can be used with the AQ Controlled Release Coat include polyethylene glycol 4000, polyethylene glycol 4600, polyethylene glycol 6000, polyethylene glycol 8000, polyethylene glycol 10000, polyethylene glycol 12000, polyethylene glycol 20000, polyethylene glycol 35000, and mixtures thereof. In at least one embodiment, the poly glycol is polyethylene glycol 8000. The poly glycol can be present in an amount of from about 0.1% to about 10% by weight of the coating composition, including all values and ranges therebetween. In certain embodiments the poly glycol is present in an amount of from about 0.5% to about 28% by dry weight of the coat, including all values and subranges therebetween. In other embodimenits the poly glycol is present in an amount from about 4% to about 17% by dry weight of the coat, including all values and subranges therebetween. In still other embodiments the poly glycol is present in an amount from about 7.2% to about 15.2% by dry weight of the coat, including all values and subranges therebetween. In certain embodiments the poly glycol is present in the coating composition in an amount of from about 0.1% to about 11.2% by dry weight of the tablet, including all values and subranges therebetween; in other embodiments in an amount of from about 0.1% to about 8.0% by dry weight of the tablet, including all values and subranges therebetween; and in still other embodiments in an amount of from about 0.2% to about 2.8% by dry weight of the tablet, including all values and subranges therebetween.

In addition to the copolymers and the poly glycol, the AQ Controlled Release Coat formulation includes at least one pharmaceutically acceptable excipient. The excipients can include but are not limited to anti-tacking agents, emulsifying agents, antifoaming agents, hydrophilic agents, flavourants, colourants, and mixtures thereof. It is known in the art that depending on the intended main function, excipients can affect the properties of the coat in a series of ways, and many substances used in coat formulations can thus be described as multifunctional. A skilled worker will know, based on his technical knowledge, which pharmaceutically acceptable excipients are suitable for the desired AQ Controlled Release Coat.

Hydrophilic agents can also be included in the AQ Controlled Release Coat to promote wetting of the coat when in contact with gastrointestinal fluids. Non-limiting examples of such hydrophilic agents include hydrophilic water soluble polymers such as hydroxypropyl methylcellulose (HPMC), hydroxypropyl cellulose (HPC) and combinations thereof. In at least one embodiment, HPMC is the hydrophilic water soluble polymer. If hydrophilic agents are to be included in the coat composition, the agents can be present in an amount from about 0.1% to about 10% by weight of the coating composition, including all values and ranges therebetween. For example, in certain embodiments the hydrophilic agents are present in an amount of from about 0.1% to about 5%, and in other embodiments from about 0.1% to about 3% by weight of the coating composition.

The tackiness of polymeric films is a factor for the coating of solid dosage forms and for the subsequent curing step (post coating thermal treatment). During coating with either cellulosic or acrylic polymers, sometimes an unwanted agglomeration of several granules or beads can occur, for example at higher product processing temperatures. Accordingly, the addition of anti-tacking agents to coating formulations can be desirable in certain embodiments. The anti-tacking agents which can be used in certain embodiments include but are not limited to adipic acid, magnesium stearate, calcium stearate, zinc stearate, hydrogenated vegetable oils, sterotex, glyceryl monostearate, talc, sodium benzoate, sodium lauryl sulfate, magnesium lauryl sulfate, and mixtures thereof. In at least one embodiment, talc is the anti-tacking agent. Talc can also function as a wetting agent. Mixtures of the anti-tacking agents are operable. The amount of anti-tacking agent in the coating composition can range from about 1% to about 15% by weight of the coating composition, including all values and ranges therebetween. For example, in certain embodiments the anti-tacking agent is present in an amount of from about 1% to about 7% by weight of the coating composition.

Certain embodiments can include anti-foaminig agents in the AQ Controlled Release Coat. Non-limiting examples of useful anti-foaming agents include silicon oil, simethicone, and mixtures thereof. In at least one embodiment, simethicone is the anti-foaming agent used in the coat composition. The anti-foaming agent can be present in an amount of up to about 0.5% by weight of the coating composition. For example, in certain embodiment the anti-foaming agent is present in an amount of from about 0.1% to about 0.4% by weight of the coating composition, including all values and ranges therebetween.

Certain embodiments can include emulsifying agents (also called emulsifiers or emulgents) in the AQ Controlled Release Coat. Emulsifying agents can facilitate emulsification during manufacture of the AQ Controlled Release Coat, and also provide emulsion stability during the shelf-life of the product. Non-limiting examples of emulsifying agents include naturally occurring materials and their semi synthetic derivatives, such as the polysaccharides, as well as glycerol esters, cellulose ethers, sorbitan esters and polysorbates. Mixtures are operable. In at least one embodiment the emulsifying agent is Polysorbate 80 (polyoxyethylene sorbitan mono-oleate) (TWEEN™ 80). The emulsifying agent can be present in an amount of up to about 0.5% by weight of the coating composition. For example, in certain embodiments the emulsifying agent is present in an amount of from about 0.1% to about 0.3% by weight of the coating composition, including all values and ranges therebetween.

Certain embodiments can include colorants in the film coat formula. Such colorants can be water-insoluble colors (pigments). Pigments have certain advantages over water-soluble colors in that they tend to be more chemically stable towards light, provide better opacity and covering power, and optimize the impermeability of a given film to water vapor. Non-limiting examples of suitable colorants include iron oxide pigments, titanium dioxide, and aluminum Lakes. Mixtures are operable. In at least one embodiment the pigment is titanium dioxide. The pigment or colorant can be present in an amount of from about 0.1% to about 10% by weight of the coating composition, including all values and ranges therebetween. For example, in certain embodiments the pigment or colorant is present in an amount of from about 0.1% to about 5%, and in other embodiments from about 0.1% to about 2% by weight of the coating composition.

In certain embodiments the AQ Controlled Release Coat of the dosage form can be made according to any one of the methods described herein.

The AQ Controlled Release Coat can be applied onto a core that includes an effective amount of the drug (e.g. tetrabenazine) by a process which involves the atomization (spraying) of the coating solution or suspension onto a bed of the tablet cores. Some examples of equipment suitable for film coating include: ACCELA COTA® (Manesty Machines, Liverpool, UK). HI-COATER® (Freund Company, Japan), DRIACOATER™ (Driam Metallprodukt GmbH, Germany), HTF/150 (GS, Italy), and IDA™ (Dumoulin, France). Examples of units that function on a fluidized-bed principle include: AEROMATIC™ (Fielder, Switzerland and UK) and GLATT™ AG (Switzerland). In at least one embodiment, the apparatus used for film coating is the ACCELA COTA®.

The coating fluid can be delivered to the coating apparatus from a peristaltic pump at the desired rate and sprayed onto the rotating or fluidizing tablet cores. The cores are pre-warned to about 30° C. During the coating process, the product temperature range is maintained at from about 25° C. to about 35° C. by adjusting the flow rate of the inlet and outlet air, temperature of the inlet air and spray rate. A single layer of coat is applied and once spraying is complete, the coated tablet cores are dried from about 30° C. to about 40° C. for a time period of from about 3 to about 5 minutes at a low pan speed and low air flow. The pan is readjusted to jog speed, and drying continues for a time period of from about 12 to about 15 minutes.

The coated cores are placed onto a tray and cured (post coating thermal treatment) in an electrical or steam oven at a temperature above the temperature of the melting point of the polyethylene glycol or derivative thereof. In certain embodiments the curing temperature is greater than the melting point of the polyethylene glycol or derivative thereof. In certain embodiments the curing time is from about 2 to about 7 hours. The cured coated dosage forms are subsequently cooled to room temperature.

The length and time for the delay in the release of drug from the dosage form coated with the AQ Controlled Release Coat can be controlled by rate of hydration and the thickness of the coat. The drug release rate subsequent to the delay can be determined by the thickness and permeability of the hydrated coat. Thus, it is possible to regulate the rate of hydration and permeability of the AQ Controlled Release Coat so that the desired controlled-release drug profile can be achieved. There is no preferred coat thickness, as this will depend on the controlled release profile desired. Other parameters in combination with the thickness of the coat include varying the concentrations of some of the ingredients of the stable coat composition of the invention described and/or varying the curing temperature and length of curing the coated tablet cores. The skilled artisan will know which parameters or combination of parameters to change for a desired controlled release profile.

As will be seen from the non-limiting examples described herein, the AQ Controlled Release Coat used in certain embodiments of the present invention are quite versatile. For example, the length and time for the lag time can be controlled by the rate of hydration and the thickness of the controlled release coat. Other parameters in combination with the thickness of the coatings include varying the concentrations of some of the ingredients of the coating compositions of certain embodiments described and/or varying the curing temperature and length of curing the coated cores. The skilled artisan will know which parameters or combination of parameters to change for a desired controlled release profile.

Methods of Use

The compositions described herein can be used in a variety of therapeutic methods, including methods of treating any disease, disorder or condition currently treated with tetrabenazine. In general, the tetrabenazine compositions described herein are useful for treating hyperkinetic movement disorders such, e.g., as Huntington's disease, hemiballismus, senile chorea, tic, tardive dyskinesia, myoclonus, dystonia and Tourette's syndrome, see for example Ondo et al., Am. J. Psychiatry. (1999) August; 156(8):1279-81 and Jankovic et al., Neurology (1997) February; 48(2):358-62. In some embodiments, the compositions described herein are combined with other therapeutic agents, for example, to optimize treatment of such diseases, disorders and conditions.

Thus, some embodiments involve a method of treating a disease, disorder or condition in an individual in need of such treatment that includes administering a therapeutically effective amount of tetrabenazine, wherein the tetrabenazine is formulated in any manner described herein, for example, with a a release-retarding agent. The method can involve treating a hyperkinetic movement disorder such as Huntington's disease, hemiballismus, senile chorea, tic, tardive dyskinesia, myoclonus, dystonia and/or Tourette's syndrome.

Tourette's disorder is a neuropsychiatric disorder characterised clinically by motor and vocal tics, which may be associated to conductual disorders such as obsessive-compulsive disorder (OCD) and attention-deficit hyperactivity disorder (ADHD). Although the neurochemistry of Tourette's disorder is not well known, a number of therapeutic agents may beneficially be combined with tetrabenazine in the compositions described herein to treat Tourette's disorder, tics, OCD and/or ADHD.

Examples of therapeutic agents that can be used in the tetrabenazine compositions described herein include antipsychotics (e.g., pimozide, haloperidol, clonidine, risperidonie, olanzapinie, clozapine, ziprasidonle), other dopamleingic drugs (flupllienazilne, sulpiride, tiapride, metoclopramide, piquindone, tetrabenazine), clonazepam, calcium channel antagonists, botulinum toxin, dopamine agonists, and/or selegiline. Many of the agents listed in the foregoing sentence are useful for treating tics and Tourette's disorder.

Some patients may suffer from obsessive-compulsive disorder as well as Tourette's disorder. Therapeutic agents that can be combined with tetrabenazine for treatment of obsessive-compulsive disorder and/or Tourette's disorder include selective serotonin reuptake inhibitors (SSRIs), the tricyclic antidepressant clomiplamine, which inhibits both serotonin and noradrenaline uptake.

For treatment of ADHD and/or Tourette's disorder, the tetrabenazine compositions described herein can include psychostimulants (e.g., methylphenidate), clonidine, guanfacine, selegiline, some tricyclic antidepressants, sertraline, pimozide and clonazepam.

Huntington's disease is an inherited neurodegenerative disorder that worsens as brain cells known as medium spiny neurons are killed off by a mutant protein. The disease brings with it an array of other difficulties as well, including cognitive problems, changes in personality, and psychiatric problems like depression. As many as one-quarter of patients with the disease attempt suicide, and many suffer from progressive cognitive decline. Unlike Alzheimer's disease, where patients usually lose their memory and insight into their disease at some point, most Huntington's patients understand exactly what is happening to them throughout most of their illness.

The disease usually strikes people in their 30s and 40s, though some patients are affected as early as childhood, while others aren't affected until their older years. Virtually everyone with the disease had a parent with the disease, and children of a person with Huntington's have a 50-percent chance of inheriting the disease. Thirteen years ago the gene that causes the disease was identified by scientists, and now a simple blood test can tell people whether they will develop the disease or not. But since there is no way known to prevent the disease or slow its progression, and for other reasons as well, many patients decline the test, instead waiting to see if they develop symptoms like the ones they witnessed in a parent. Patients usually live for 15 to 20 years after the onset of symptoms.

Viewed simply, in some ways Huntington's disease is the opposite of Parkinson's disease, where damage to the neurons that produce dopamine hinders a person's ability to move and cause other symptoms. In Huntington's, too many dopamine signals result in random, uncontrollable movements. Tetrabenazine inhibits a molecule known as vesicular monoamine transporter 2 (VMAT2), an action that ultimately blocks the release of dopamine.

Therapeutic agents that can be combined with tetrabenazine in the compositions described herein to effectively treat Huntington's disease include antipsychotics (e.g., haloperidol).

Hemiballismus is a type of movement disorder considered over a hundred times rarer compared to the more common Parkinson's disease. People who are afflicted with Hemiballismus are subject to severe movement-related symptoms that render them unable to go about their day-to-day activities. This disease is linked to people who have suffered structural lesions in the brain, but it sometimes accompanies some metabolic abnormalities. Therapeutic agents that can be used with tetrabenazine in the compositions and treatment methods described herein include dopamine receptor blocking agents, neuroleptics such as haloperidol and perphenazine, antipsychotics Such as risperidone and clozapine, and/or catecholamine-depleting agents such as reserpine.

Tardive dyskinesia is characterized by repetitive, involuntary, purposeless movements. Features of the disorder may include grimacing, tongue protrusion, lip smacking, puckering and pursing of the lips, and rapid eye blinking. Rapid movements of the extremities may also occur. Impaired movements of the fingers may also appear. Tetrabenazine can be combined with a variety of therapeutic agents for treatment of tardive dyskinesia. For example, the tetrabenazine compositions described herein can include a neuroleptic, cannibis, Aricept, Baclofen, Requip, Mirapex, Clonidine and/or Botox. Botox injections can be for more advanced tardive dyskinesia.

Myoclonus involves brief, involuntary twitching of a muscle or a group of muscles. Treatment of myoclonus focuses on medications that may help reduce symptoms. Therapeutic agents that can be combined with tetrabenazine for the treatment of Myoclonus include benzodiazepines such as clonazepam, antiepileptics, barbiturates, phenytoin, primidone, 5-hydroxytryptophan (5-HTP), sodium valproate, and piracetam.

Dystonia is a neurological movement disorder in which sustained muscle contractions can cause twisting and repetitive movements or abnormal postures. The disorder may be inherited or caused by other factors such as birth-related or other physical trauma, infection, poisoning (e.g. lead poisoning) or reaction to drugs, particularly neuroleptics.

Therapeutic agents that can be used with tetrabenazine for the treatment of dystonia include anti-Parkinsons agents (Trihexyphenidyl, Trihexyphenidyl-Hydrochloride (PAKISONAL)), muscle relaxers (Valium), keppra, beta-blockers (including some blood pressure medications), anticholinergics, clonazepam (an anti-seizure medicine). Botulinum toxin injections into affected muscles have proved quite successful in providing some relief for around 3-6 months, depending on the kind of dystonia. Botox injections have the advantage of ready availability (the same form is used for cosmetic surgery) and the effects are not permanent. There is a risk of temporary paralysis of the muscles being injected or the leaking of the toxin into adjacent muscle groups causing weakness or paralysis in them. The injections have to be repeated as the effects wear off and around 15% of recipients will develop immunity to the toxin. There is a Type A and Type B toxin approved for treatment of dystonia; often those that develop resistance to Type A may be able to use Type B. One type of dystonia, dopa-responsive dystonia, can be treated with regular doses of L-dopa in a form such as Sinemet (carbidopa/levodopa). In the case of Oculogyric crisis, benadryl may be administered. A baclofen pump has been used to treat patients of all ages exhibiting muscle spasticity along with dystonia. The pump delivers baclofen via a catheter to the thecal space surrounding the spinal cord. The pump itself is placed in the abdomen. It can be refilled periodically by access through the skin. Diphenhydramine (Benadryl) 25-50 mg IV push is often used because it possesses some anticholinergic properties.

Thus, the tetrabenazine compositions and methods described herein can involve a composition that includes tetrabenazine with antidepressants, anticholinergics, antiepileptics, anti-Parkinsons agents, antipsychotics, aricept, baclofen, barbiturates, benzodiazepines, beta-blockers, botulinum toxin (Botox), calcium channel antagonists, catecholamine-depleting agents, clomiplamine, clonidine, clonazepam, clozapine, diphenhydramine, dopaminergic drugs, dopamine agonists, fluphenazine, guanfacime, haloperidol, 5-hydroxytryptophan (5-HTP), keppra, L-dopa, methylphenidate, metoclopramide, mirapex, muscle relaxers (e.g., Valium), neuroleptics, olanzapine, perphenazine, phenytoin, pimozide, piquindone, piracetam, primidone, psychostimulants, requip, risperidone, selegiline, serotonin reuptake inhibitors (SSRIs), sertraline, sodium valproate, sulpiride, tiapride, tricyclic antidepressants, trihexyphenidyl, trihexyphenidyl-hydrochloride (Pakisonal), ziprasidone and combinations thereof.

The examples below are non-limiting and are representative of various aspects of certain embodiments of the present invention.

### Examples

### Example 1

Tetrabenazine 50 mg Tablets

Tetrabenazine tablets of total individual weights of 250 mg and containing 50 mg of tetrabenazine were prepared according to the dry granulation method set out below. The tablets all contained tetrabenazine and other excipients in a matrix containing the release-retarding agent hydroxypropylmethylcellulose.

Three different formulations were employed, each differing only with respect to the grade of hydroxypropylmethylcellulose used. The three grades were (a) HPMC (K4M), (b) HPMC (K100 LV) and (c) HPMC (E15LV), the properties of each of which are set out above.

| Ingredient | Function | 250 mg tablet |
|---|---|---|
| Tetrabenazine | Active agent | 50 mg, 20% (w/w) |
| Lactose | Diluent | 78.9 mg, 31.6% (w/w) |
| Starch | Binder/Disintegrant | 40.5 mg, 16.2% (w/w) |
| (a) HPMC (K4M); or | Controlled-release | 75 mg, 30% (w/w) |
| (b) HPMC (K100 LV); or | agent | |
| (c) HPMC (E15LV) | | |
| Talc | Glidant | 4 mg, 1.6% (w/w) |
| Magnesium stearate | Lubricant | 1.6 mg, 0.6% (w/w) |

Tetrabenazine, lactose, starch and the chosen grade of HPMC were sifted through a 30 mesh hand sieve into a suitable container. The powders were then mixed in a Hobart mixer for 10 minutes with the kneader forward on slow speed.

The talc was transferred through a 30 mesh hand sieve and into a suitable container and the magnesium stearate was transferred and sifted through a 60 mesh hand sieve into a suitable container.

The sifted talc and magnesium stearate were added to the tetrabenazine, lactose, starch and HPMC in the Hobart mixer and all ingredients were mixed for 2 minutes with the kneader forward on slow speed to form the granulate.

The granulate blend was then sealed in polyethylene containers that have been double lined with polyethylene bags.

The 250 mg tablets were formed by compression using an 8 mm round, flat, beveled edge punch with a single break line for both the upper and lower punches.

The compressed 250 mg tablets were packed into 85 ml HDPE bottles with inner polypropylene caps containing a liner consisting of Suryln/aluminum/polyethylene/bleached kraft membrane.

### Example 2

### Investigation of the Pharmacokinetics of a 50 ml Controlled Release Tetrabenazine Tablet Containing Hydroxypropylmethylcellulose (K100 LV Grade) as the Release-Retarding Agent

In an initial study (results not shown), the pharmacokinetics of the three formulations described in Example 1 were compared. It was found that when formulated using HPMC (K100 LV) as the release-retarding agent, the half life for tetrabenazine (measured as the concentrations of α and β dihydrotetrabenazine metabolites) was approximately 13 hours whereas when the K4M and E15LV grades of HPMC were used, the half lives were approximately 9 hours in each case.

The formulation containing the K100 LV grade of HPMC was therefore selected for further study.

Accordingly, the steady state pharmacokinetics of the 50 mg controlled-release tablet formulation containing the K100 LV grade of HPMC were assessed. In addition, the safety and tolerability of tetrabenazine administered as a controlled-release formulation was assessed.

The study included 9 healthy male and female volunteer subjects. Each subject received a daily dose of a 50 mg controlled-release tetrabenazine tablet for 7 days in Period 1 and a single dose of 2×50 mg controlled-release tetrabenazine tablets in Period 2. The subjects were resident in the clinic for 11 days during Period 1, and 5 days during Period 2. There was at least a seven day washout period between the last dose in Period 1 and the first dose of Period 2.

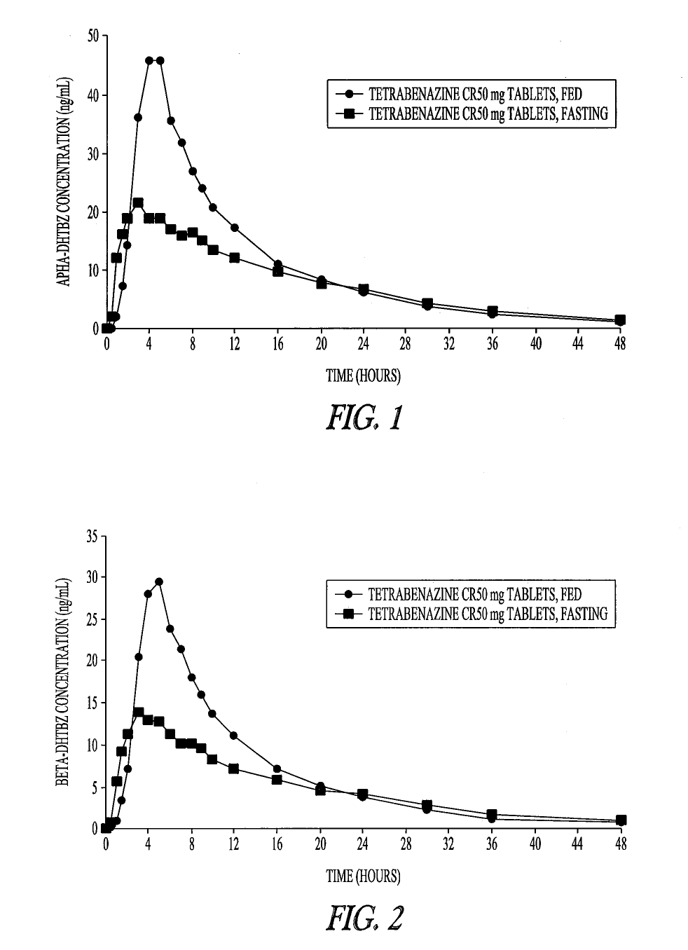

The concentration of tetrabenazine and its metabolites (α- and β-dihydrotetrabenazine (HTBZ)) was determined by taking blood samples from the subjects. In this regard, during Period 1 blood samples were drawn before each dose and at 0.5, 1, 2, 3, 4, 5, 6, 8, 12 and 16 hours before the first dose and at 0.5, 1, 2, 3, 4, 5, 6, 8, 12, 16, 24, 48 and 72 hours after the last (seventh) dose. During Period 2, blood samples were drawn before the single dose and at 0.5, 1, 2, 3, 4, 5, 6, 8, 12, 16, 24, 48 and 72 hours. After oral administration of the controlled-release tetrabenazine tablets, tetrabenazine was rapidly transformed into metabolites α-HTBZ and β-HTBZ with little parent compound detected in plasma (FIG. 1).

Steady state was achieved for both metabolites α-HTBZ and β-HTBZ at day 7 after a daily dose of 50 mg controlled-release tetrabenazine tablets for seven days. Peak plasma concentration at steady state was reached at 2 hours for α-HTBZ (median Tₘₐₓ) and at 1 hour (median Tₘₐₓ) for β-HTBZ. The elimination half-life, calculated from steady state plasma α-HTBZ and β-HTBZ concentration, was 13.53 hours for α-HTBZ and 12.48 hours for β-HTBZ.

Dose accumulation was observed by comparing day 1 and day 7 Cₘₐₓ and AUC data. The α-HTBZ and β-HTBZ absorption (Cₘₐₓ and AUC) calculated from the blood samples collected before the last dose on day 7 was much higher than that on day 1.

In summary, both a daily dose of 50 mg tetrabenazine controlled-release tablets for seven days and a single dose of 2×50 mg controlled-release tetrabenazine tablet were well tolerated. After oral administration of controlled-release tetrabenazine tablets, tetrabenazine was rapidly transformed into α-HTBZ and β-HTBZ with little parent compound detected in the plasma. Steady state was achieved for both α-HTBZ and β-HTBZ at day 7 after a daily dose of 50 mg controlled-release tetrabenazine tablets for seven days. Dose accumulation was observed by comparing day 1 and day 7 Cₘₐₓ and AUC data. The α-HTBZ and β-HTBZ absorption (Cₘₐₓ and AUC) calculated from the blood samples collected after the last dose on day 7 was much higher than that on day 1.

### Comparative Example 1

Tetrabenazine Solubility

The following example employs immediate-release tablets of tetrabenazine, in contrast to the controlled-release tablets of the present invention, to determine the solubility of tetrabenazine across the pH range 2-12.

The dissolution of tetrabenazine 12.5 mg and 25 mg immediate-release tablets was conducted in 0.1 M hydrochloric acid solution (pH 1.5).

The solubility of tetrabenazine was determined across the pH range 2-12 in water, adjusted with hydrochloric acid/sodium hydroxide as necessary and if feasible complete dissolutions at pH 7.0 and pH 12.

It was found that tetrabenazine was soluble in pH 2 hydrochloric acid solution at approximately 850.0 mg/100 ml (i.e. 1 in 117—categorized as slightly soluble).

The solubility decreased significantly between pH 2 and pH 3, such that at pH 3 it was only soluble at approximately 4.0 mg/100 ml (i.e. 1 in 25,000—categorized as practically insoluble). The solubility remained relatively constant between pH 4 and pH 12 at approximately 3.0 mg/100 ml (i.e. 1 in 33,333—categorized as practically insoluble).

It was not feasible to complete tablet dissolution at pH 7 and pH 12 because of the lack of solubility.

All samples were protected from light throughout the experiment.

In summary, tetrabenazine was found to be practically insoluble at the pH range of 3-12 and slightly soluble at approximately 850 mg/100 ml at pH 2 (i.e. 1 in approximately 117).

### Example 3

### Preparation of Tablets Containing 50 mg Tetrabenazine in a Matrix Including Polyethylene Oxide and Hydroxypropylmethylcellulose and Polyoxyalkylene Block Copolymer

For the manufacture of a 4 kg batch of 50 mg tetrabenazine tablets, half the required amount of microcrystalline cellulose, half the required amount of lactose, half the required amount of polyethylene oxide (PEO), half the required amount of hydroxypropylmethylcellulose (HPMC) and half the required amount of polyoxyalkylene block copolymer (Plurollic®) are filled into a Pharmatech AB-050 V Shell blender. Subsequently, the tetrabenazine, with the remaining microcrystalline cellulose, lactose, PEO, H1PMC and Pluronic® are added to the Blender. The blend is then mixed at 25 rpm for 10 minutes without the use of an intensifier bar. Following the 10 minutes blending, the magnesium stearate is added to the blend, and the blend further tumbled in the V Blender for one minute at 25 rpm without the use of the intensifier. The tablet blend is discharged from the V Blender and compressed into tablets using a Riva Picolla Rotary tablet press model B/10 fitted with 17 mm×9 mm caplet tooling. Compression parameters are adjusted in order to achieve a tablet weight of 650 mg and hardness of 80-120N.

### Example 4

### Preparation of Tablets Containing Tetrabenazine in a Matrix Including Polyethylene Oxide and Hydroxypropylmethylcellulose and Polyoxyalkylene Block Copolymer—PVA Granulation Method

4A. Preparation of Tetrabenazine Granules

In an alternative to the procedure described in Example 3, tetrabenazine is granulated prior to mixing with other tablet excipients, in order to improve powder flow during compression. Granulation can be achieved through either wet or dry granulation. In one embodiment of the invention, in order to manufacture a 30 kg batch of 50 mg tetrabenazine tablets, tetrabenazine is first wet granulated with lactose and polyvinyl alcohol (PVA) as a binder in an Aeromatic Fielder MP3/2/3 fluidized bed granulator. In brief, the granulation binder solution is prepared by dispersing the PVA in cold water which is subsequently heated to approximately 60° C. to solubilize the PVA. The solution is then allowed to cool for at least 2 hours. The granulation solution is then top-sprayed onto an 18 kg fluidized bed of tetrabenazine and lactose (58.41:41.59 ratio of lactose:tetrabenazine), fluidized in an Aeromatic Fielder MP3/2/3 fluidized bed granulator with the following process conditions:

| Process Parameter | Setting |
|---|---|
| Product Temperature | 25-26° C. |
| Inlet Air Temperature | 65° C. |
| Air velocity | 250 m³/h |
| Atomising Air Pressure | 1 bar |
| Spray Rate | 70 g/min |

Following application of 252 g of PVA to the fluidized bed, spraying is stopped and the granules further fluidized to dry the granulates to a moisture content of approximately 1.5% w/w.

4B. Preparation of Tablets Containing Tetrabenazine

To blend the tetrabenazine granules with the other tablet excipients, half the required amount of microcrystalline cellulose, half the required amount of lactose, half the required amount of PEO, half the required amount of HPMC and half the required amount of the Pluronic® are filled into a Pharmatech AB-400 V Shell blender. Subsequently, the tetrabenazine granules, with the remaining microcrystalline cellulose, lactose, PEO, HPMC and Pluronic® are added to the Blender. The 30 kg blend is then mixed at 25 rpm for 10 minutes without the use of an intensifier bar. Following the 10 minutes blending, the magnesium stearate is added to the blend, and the blend further tumbled in the V Blender for one minute at 25 rpm without the use of the intensifier. The tablet blend is discharged from the V Blender and compressed into tablets using a Fette 1200 tablet press fitted with 17 mm×9 mm caplet tooling. Compression parameters are adjusted in order to achieve a tablet weight of 650 mg and hardness of 80-120N.

### Example 5

### Preparation of Tablets Containing a Tetrabenazine: Eudragit® E Extrudate

5A. Manufacture of 30:70 Tetrabenazine:Eudragit® E Extrudate

Each heating zone of an APV Baker 19 mm twin-screw extruder is heated to a target temperature of 70° C., 140° C., 140° C., 130° C., and 100° C. for each of heating zones 1,2,3,4 and 5 respectively. The extruder twin screws are then rotated at 140 rpm and a 4.6 kg blend of tetrabenazine and Eudragit® E, preblended in a Pharmatech AB50 V blender for 5 minutes, is fed into the extruder hopper until all five heating zone temperatures are within 5° C. of the target temperature. Extrusion of the blend is continued at 140 rpm and milled extrudate is collected on a stainless steel tray.

4B. Preparation of Tablets Containing the Extrudate

In order to manufacture a 4 kg batch of 50 mg tetrabenazine tablets including the melt extrusion of Example 5A, half the required amount of microcrystalline cellulose, half the required amount of lactose, half the required amount of PEO, half the required amount of HPMC and half the required amount of Pluronic® are filled into a Pharmatech AB-050 V Shell blender. Subsequently, the tetrabenazine extrudate, with the remaining microcrystalline cellulose, lactose, PEO, HPMC and Pluronic® are added to the blender. The blend is then mixed at 25 rpm for 10 minutes without the use of an intensifier bar. Following the 10 minutes blending, the magnesium stearate is added to the blend, and the blend is further tumbled in the V Blender for one minute at 25 rpm without the use of the intensifier. The tablet blend is discharged from the V Blender and compressed into tablets using a Riva Picolla Rotary tablet press model B/10 fitted with 17 mm×9 mm caplet tooling. Compression parameters are adjusted in order to achieve a tablet weight of 650 mg and hardness of 80-120N.

### Example 6

The formulations of Examples 6A to 6C in the table below ma)y be prepared by the method described in Example 3.

Example 6A is a 650 mg 17 mm×9 mm tablet matrix formulation (hardness 60-80N) including 50 mg tetrabenazine, 10% w/w 5,000,000 MW Polyethylene oxide (PEO WSR Coag.), 10% w/w 4,000 cps HPMC (Methocel K4M) together with 20% polyoxyalkylene block copolymer (Pluronic® F127) as a drug release modifier.

Example 6B is a tablet identical in size and shape and hardness to 1A, has the same levels of K4M and PEO WSR Coag., but differs in that the Pluronic® F127 is replaced with lactose as a drug release modifier.

Example 6C is a tablet identical in size and shape and hardness to 1A, has the same levels of Methocel K4M and PEO WSR Coag., but differs from both 1A and 1B in that both Pluronic® FP127 and lactose are present in the formulation.

The ingredients of the formulations of each of Examples 6A to 6C are set out in the table below.

| | Tetrabenazine example | | |
|---|---|---|---|
| | formulations | | |
| Components of Tablet | Example 6A | Example 6B | Example 6C |
| Formulation (%) | (%) | (%) | (%) |
| Tetrabenazine | 7.7 | 7.7 | 7.7 |
| PEO WSR Coagulant | 10 | 10 | 10 |
| HPMC K4M | 10 | 10 | 10 |
| Lactose monohydrate | — | 35.7 | 25.65 |
| Microcrystalline | 51.3 | 35.7 | 25.65 |
| Cellulose | | | |
| Magnesium Stearate | 1 | 1 | 1 |
| Pluronic ® F127 | 20 | — | 20 |

### Example 7

Examples 7A and 7B are similar to those presented in Example 6, but use a higher viscosity grade of HPMC (100,000 cps)

Tetrabenazine example
formulations

| Components of Tablet Formulation | Example 7A | Example 7B |
|---|---|---|
| (%) | (%) | (%) |
| Tetrabenazine | 7.7 | 7.7 |
| PEO WSR Coagulant | 10 | 10 |
| HPMC K100M | 10 | 10 |
| Lactose monohydrate | 25.65 | — |
| Microcrystalline Cellulose | 25.65 | 71.3 |
| Magnesium Stearate | 1 | 1 |
| Pluronic ® F127 | 20 | — |

### Example 8

The following tables provide examples of formulations of different drug potency including tetrabenazine and Pluronic®. The formulations shown below may be prepared by first granulating the drug with a binder (in this case polyvinyl alcohol) to aid powder flow during compression.

8A. Tablets Containing 6.25 mg or 12.5 mg or 25 mg Tetrabenazine

| Component | Composition (mg/Tablet and % w/w) | | | | | |
|---|---|---|---|---|---|---|
| Compendial | 6.25 mg | | 12.5 mg | | 25 mg | |
| Name | mg | % | mg | % | mg | % |
| Tetrabenazine | 6.25 | 0.96 | 12.50 | 1.92 | 25.00 | 3.85 |
| Polyethylene | 65.00 | 10.00 | 65.00 | 10.00 | 65.00 | 10.00 |
| oxide | | | | | | |
| Hypromellose | 65.00 | 10.00 | 65.00 | 10.00 | 65.00 | 10.00 |
| Pluronic ® | 130.00 | 20.00 | 130.00 | 20.00 | 130.00 | 20.00 |
| F127 | | | | | | |
| Micro- | 188.24 | 28.96 | 184.79 | 28.43 | 177.19 | 27.26 |
| crystalline | | | | | | |
| cellulose | | | | | | |
| Lactose | 188.30 | 28.97 | 184.79 | 28.43 | 178.46 | 27.46 |
| Monohydrate | | | | | | |
| Polyvinyl | 0.71 | 0.11 | 1.42 | 0.22 | 2.85 | 0.44 |
| Alcohol | | | | | | |
| Magnesium | 6.50 | 1.00 | 6.50 | 1.00 | 6.50 | 1.00 |
| Stearate | | | | | | |
| Total | 650.00 | 100.00 | 650.00 | 100.00 | 650.00 | 100.01 |

8B. Tablets Containing 50 mg or 75 mg or 100 mg Tetrabenazine

| Component | Composition (mg/Tablet and % w/w) | | | | | |
|---|---|---|---|---|---|---|
| Compendial | 50 mg | | 75 mg | | 100 mg | |
| Name | mg | % | mg | % | mg | % |
| Tetrabenazine | 50.00 | 7.69 | 75.00 | 11.54 | 100.01 | 14.29 |
| Polyethylene | 65.00 | 10.00 | 65.00 | 10.00 | 70.00 | 10.00 |
| oxide | | | | | | |
| Hypromellose | 65.00 | 10.00 | 65.00 | 10.00 | 70.00 | 10.00 |
| Pluronic ® | 130.00 | 20.00 | 130.00 | 20.00 | 140.00 | 20.00 |
| F127 | | | | | | |
| Micro- | 165.88 | 25.52 | 153.01 | 23.54 | 154.84 | 22.12 |
| crystalline | | | | | | |
| cellulose | | | | | | |
| Lactose | 165.94 | 25.53 | 152.97 | 23.53 | 154.79 | 22.11 |
| Monohydrate | | | | | | |
| Polyvinyl | 1.68 | 0.26 | 2.52 | 0.39 | 3.36 | 0.48 |
| Alcohol | | | | | | |
| Magnesium | 6.50 | 1.00 | 6.50 | 1.00 | 7.00 | 1.00 |
| Stearate | | | | | | |
| Total | 650.00 | 100.00 | 650.00 | 100.00 | 700.00 | 100.00 |

### Example 9

Gastric Retentive Formulations T

he following table sets out some examples of gastric retentive formulations according to the present invention. The following formulations are of different drug potency and may be made by direct compression, i.e. in the absence of polyvinyl alcohol. The skilled person will appreciate that the formulations set out below will demonstrate that the rate and extent of drug dissolution is independent of drug potency in the formulation.

9A. Tablets Containing 6.25 mg or 12.5 mg or 25 mg Tetrabenazine

| | Composition (mg/Tablet and % w/w) | | | | | |
|---|---|---|---|---|---|---|
| Compendial | 6.25 mg | | 12.5 mg | | 25 mg | |
| Name | mg | % | mg | % | mg | % |
| Tetrabenazine | 6.25 | 0.96 | 12.50 | 1.92 | 25.00 | 3.85 |
| PEO Coagulant | 65.00 | 10.00 | 65.00 | 10.00 | 65.00 | 10.00 |
| HPMC K15M | 65.00 | 10.00 | 65.00 | 10.00 | 65.00 | 10.00 |
| Pluronic ® | 130.00 | 20.00 | 130.00 | 20.00 | 130.00 | 20.00 |
| F127 | | | | | | |
| Micro- | 188.95 | 29.07 | 186.21 | 28.65 | 180.04 | 27.7 |
| crystalline | | | | | | |
| cellulose | | | | | | |
| Lactose | 188.30 | 28.97 | 184.79 | 28.43 | 178.46 | 27.46 |
| Monohydrate | | | | | | |
| Magnesium | 6.50 | 1.00 | 6.50 | 1.00 | 6.50 | 1.00 |
| Stearate | | | | | | |
| Total | 650.00 | 100.00 | 650.00 | 100.00 | 650.00 | 100.01 |
| | Composition (mg/Tablet and % w/w) | | | | | |
| Compendial | 50 mg | | 75 mg | | 100 mg | |
| Name | mg | % | mg | % | mg | % |
| Tetrabenazine | 50.00 | 7.69 | 75.00 | 11.54 | 100.01 | 14.29 |
| PEO Coagulant | 65.00 | 10.00 | 65.00 | 10.00 | 70.00 | 10.00 |
| HPMC K15M | 65.00 | 10.00 | 65.00 | 10.00 | 70.00 | 10.00 |
| Pluronic ® | 130.00 | 20.00 | 130.00 | 20.00 | 140.00 | 20.00 |
| F127 | | | | | | |
| Micro- | 167.56 | 25.78 | 155.53 | 23.93 | 158.2 | 22.6 |
| crystalline | | | | | | |
| cellulose | | | | | | |
| Lactose | 165.94 | 25.53 | 152.97 | 23.53 | 154.79 | 22.11 |
| Monohydrate | | | | | | |
| Magnesium | 6.50 | 1.00 | 6.50 | 1.00 | 7.00 | 1.00 |
| Stearate | | | | | | |
| Total | 650.00 | 100.00 | 650.00 | 100.00 | 700.00 | 100.00 |

### Example 10

The following table sets out some examples of formulations containing various combinations of tetrabenazine, PEO, HPMC and Poloxamer.

| Wt/mg based on | 10A | 10B | 10C | 10D | 10E | 10F | 10G |
|---|---|---|---|---|---|---|---|
| 650 mg tablet | % w/w | % w/w | % w/w | % w/w | % w/w | % w/w | % w/w |
| Tetrabenazine | 7.7 | 7.7 | 7.7 | 7.7 | 7.7 | 7.7 | 7.7 |
| PEO WSR N-60K | — | 20 | — | — | — | 15 | — |
| PEO WSR Coagulant | 10 | — | 15 | 10 | 10 | 10 | 30 |
| Methocel K100M | — | — | 15 | 15 | 10 | — | — |
| Methocel K15M | — | — | — | — | — | — | 10 |
| Methocel K4M | 10 | 20 | — | — | — | 15 | — |
| Pluronic ® F68 | 20 | — | — | 7.7 | 20.5 | — | — |
| Pluronic ® F127 | — | 20 | 20 | — | — | 10 | 20 |
| Avicel ® pH 101 | 51.3 | 15.65 | 20.65 | 58.6 | 63.5 | 41.3 | 15.65 |
| Lactose Monohydrate | — | 15.65 | 20.65 | — | — | | 15.65 |
| Magnesium Stearate | 1.0 | 1.0 | 1.0 | 1.0 | 1.0 | 1.0 | 1.0 |

### Example 11

Tablet Formulation Containing 30:70 Tetrabenazine:Eudragit® E Melt Extrudate

| Components of Tablet Formulation (%) | (%) |
|---|---|
| Tetrabenazine | — |
| Tetrabenazine/Eudragit ® E (30:70) extrudate | 25.6 |
| PEO WSR Coagulant | 10 |
| HPMC K4M | 10 |
| Lactose monohydrate | 26.7 |
| Microcrystalline Cellulose | 26.7 |
| Magnesium Stearate | 1 |

### Example 12

Tablet Formulation Containing 20:80 Tetrabenazine: Eudragit® E Melt Extrudate

The following example is similar to Example 11, but uses a Tetrabenazine:Eudragit® E ratio of 20:80 in the formation of the solid dispersion.

| Components of Tablet Formulation (%) | (%) |
|---|---|
| Tetrabenazine | — |
| Tetrabenazine/Eudragit ® 20:80 extrudate | 38.5 |
| PEO WSR Coagulant | 10 |
| HPMC K4M | 10 |
| Pluronic ® F127 | 10 |
| Microcrystalline Cellulose | 30.5 |
| Magnesium Stearate | 1 |

### Example 13

### Tablet Formulation Containing 40:60 Tetrabenazine:Eudragit® E Melt Extrudate

The following example is similar to Example 11, but uses a Tetrabenazine:Eudragit® E ratio of 40:60 in the formation of the solid dispersion.

| Components of Tablet Formulation (%) | (%) |
|---|---|
| Tetrabenazine | — |
| Tetrabenazine/Eudragit ® 40:60 extrudate | 19.25 |
| PEO WSR Coagulant | 10 |
| HPMC K4M | 10 |
| Lactose monohydrate | 29.9 |
| Microcrystalline Cellulose | 29.9 |
| Magnesium Stearate | 1 |

### Example 14

### Tablet Formulation Containing Granules Including Tetrabenazine and Hydroxymethyl Cellulose and Hydroxyethylcellulose

### Example 14A

| Components of tablet | % by weight |
|---|---|
| Tetrabenazine | 25% |
| Methocel K100LV CR Premium | 7.5% |
| (Hydroxypropylmethylcellulose) | |
| Methocel K15M Premium | 8.0% |
| (Hydroxypropylmethylcellulose) | |
| Natrosol 250 HHX | 3.5% |
| (Hydroxyethylcellulose) | |
| Flowlac 100 | 50% |
| (Lactose) | |
| Ethocel 100FP Premium | 5% |
| (Ethylcellulose) | |
| Magnesium Stearate | 1% |

Tetrabenazine is blended with Methocel K100LV CR Premium, Methocel K15M Premium, Natrosol 250HHX and Flowlac in a Diosna P1-6 high shear mixer for approximately 5 minutes with the chopper motor set at approximately 600 rpm and the mixer motor set at approximately 400 rpm. The blend is granulated with 2-propanol for approximately 5 minutes and the granules are dried in a Casburt laminar flow drying oven at a temperature of 40° C. for 18 h and screened through a 800 μm screen. The granules and the Ethocel 100FP are blended in a V-type PK Blendmaster with a mixing time of approximately 5 minutes with set speeds for the blender shell and intensifier bar. Magnesium stearate is added to the blend and the mixture is further blended for approximately 1.5 min with set speed for the blender shell and the intensifier bar turned off. The blend is compressed into tablets.

### Examples 14B to 14D

Using a similar procedure to that described in Example 14A, tablets with the following compositions may be prepared.

| | Formulation | Formulation | Formulation |
|---|---|---|---|
| | 14B % by | 14C % by | 9D % by |
| Component | weight | weight | weight |
| Tetrabenazine | 25% | 25% | 25% |
| Methocel K100LV CR | 15% | 0% | 0% |
| Premium | | | |
| (Hydroxypropylmethyl- | | | |
| cellulose) | | | |
| Methocel K15M Premium | 0% | 15% | 15% |
| (Hydroxypropylmethyl- | | | |
| cellulose) | | | |
| Natrosol 250 HHX | 3.5% | 3.5% | 3.5% |
| (Hydroxyethylcellulose) | | | |
| Flowlac 100 | 50.5% | 50.5% | 35.5% |
| (Lactose) | | | |
| Poloxamer F127 | 0% | 0% | 15% |
| (Surfactant) | | | |
| Ethocel 100FP Premium | 5% | 5% | 5% |
| (Ethylcellulose) | | | |
| Magnesium Stearate | 1% | 1% | 1% |
| (Lubricant) | | | |

### Example 15

### Comparison of Controlled Release Tetrabenazine Tablets with Immediate Release Tetrabenazine Tablets

A single dose, 4 way crossover pilot study was carried out to compare the three 50 mg formulations of controlled release tetrabenazine (Example 1) with 2×25 mg immediate release tetrabenazine.

The purpose of the study was to delineate the pharmacokinetics of three prototypes of a novel once daily tetrabenazine 50 mg controlled release (CR) formulation and evaluate the systemic bioavailability relative to a 2×25 mg immediate release (IR) formulation.

The formulations used in the study were the formulations described in Example 1, which differed only with regard to the grade and molecular weight of the hydroxypropylmethylcellulose used.

Study Design:

The study followed a four-period, four-treatment, non-randomized, open-label, crossover design under fasting conditions with a sample size of 8 subjects.

Subjects received the following treatments after a 10-hour overnight fast. The treatments were not randomized. The study periods were separated by a 7-day washout:

Treatment A: Oral dose of 2×25 mg immediate release (IR) tetrabenazine tablets: Reference

Treatment B: Oral dose of 1×50 mg controlled release (CR) tetrabenazine tablets:

Test 1—Formulation of Example 1, HPMC (E15LV)

Treatment C: Oral dose of 1×50 mg controlled release (CR) tetrabenazine tablets:

Test 2—Formulation of Example 1, HPMC (K100 LV)

Treatment D: Oral dose of 1×50 mg controlled release (CR) tetrabenazine tablets:

Test 3—Formulation of Example 1, HPMC (K4M)

Serial blood samples were collected from 0-36 hours for all treatments. Plasma concentrations of tetrabenazine and the metabolites, α-dihydrotetrabenazine and β-dihydrotetrabenazine were quantified using a LCMS/MS assay.

Results and Discussion:

A total of 10 subjects were enrolled into the study at Simbec Research and 7 subjects completed the study.

Three subjects (#1, #4 and #5) withdrew after period 1 due to personal reasons.

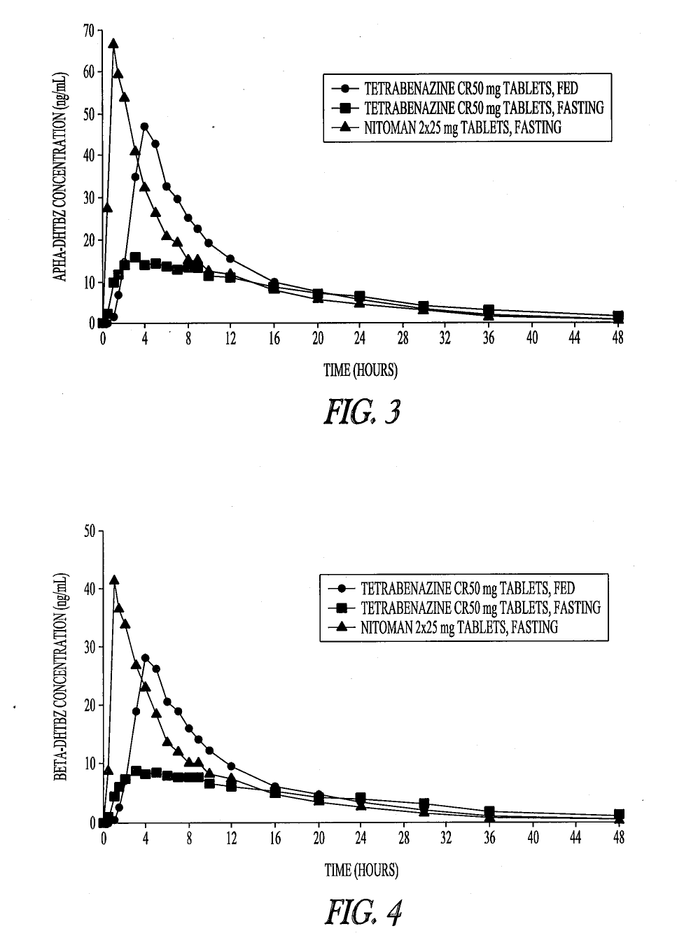

Pharmacokinetic and statistical analyses were carried out on plasma tetrabenazine, α-dihydrotetrabenazine (α-DHBZ) and β-dihydrotetrabenazine (β-DHBZ) from 7 subjects. The mean plasma concentration-time plots are shown in FIGS. 2 to 4. Mean pharmacokinetic parameters for each analyte are shown in Table 1.

Summary statistics are presented in Table 2. A listing of the α-HTBZ/β-HTBZ ratios for each treatment is presented in Table 3.

TABLE 1
Mean Pharmacokinetic Parameters (Mean ± SD) for Tetrabenazine and Metabolites (n = 7)

| | | 2 × 25 mg | 50 mg CR | 50 mg CR | 50 mg CR |
|---|---|---|---|---|---|
| | | IR | (1) | (2) | (3) |
| Tetrabenazine | AUC₀₋ₜ (ng * hr/mL) | 0.90 | 0.19 | 0.21 | 0.64 |
| (Very Few | AUC_{0-∞} (ng * hr/mL) | 5.96 | NC | NC | 4.47 |
| Datapoints) | Cₘₐₓ (ng/mL) | 1.05 | 0.18 | 0.18 | 0.72 |
| | Tₘₐₓ (hr)* | 0.5 | 1.0 | 4.25 | 1.0 |
| | t_{1/2} (hr) | NC | NC | NC | NC |
| α-HTBZ | AUC₀₋ₜ (ng * hr/mL) | 317 ± 128 | 190 ± 87 | 253 ± 129 | 193 ± 103 |
| | AUC_{0-∞} (ng * hr/mL) | 325 ± 129 | 204 ± 90 | 308 ± 175 | 213 ± 136 |
| | Cₘₐₓ (ng/mL) | 64.1 ± 26.0 | 19.2 ± 10.6 | 17.9 ± 9.6 | 11.9 ± 6.3 |
| | Tₘₐₓ (hr)* | 1.0 (0.5, 2.0) | 2.0 (1.0, 6.0) | 3.0 (1.0, 8.0) | 3.0 (1.0, 24.0) |
| | t_{1/2} (hr) | 5.7 ± 1.8 | 9.8 ± 1.6 | 14.0 ± 4.5 | 8.5 ± 2.0 |
| β-HTBZ | AUC₀₋ₜ (ng * hr/mL) | 104 ± 45 | 51 ± 37 | 78 ± 55 | 52 ± 40 |
| | AUC_{0-∞} (ng * hr/mL) | 110 ± 50 | 67 ± 34 | 95 ± 69 | 103 ± 90 |
| | Cₘₐₓ (ng/mL) | 28.5 ± 12.6 | 7.9 ± 5.5 | 7.8 ± 5.0 | 4.1 ± 2.6 |
| | Tₘₐₓ (hr)* | 1.0 (1.0, 2.0) | 3.0 (1.0, 6.0) | 3.0 (1.0, 8.0) | 3.0 (1.0, 16.0) |
| | t_{1/2} (hr) | 2.9 ± 0.6 | 8.2 ± 5.8 | 9.3 ± 4.4 | 11.9 ± 7.1 |

*Median Tₘₐₓ (Min, Max)

TABLE 2
Comparisons of Three Formulations of CR Tablets Against IR Tablets

| | | CR (1) | CR (2) | CR (3) |
|---|---|---|---|---|
| | % Ratio | vs IR | vs IR | vs IR |
| α-HTBZ | AUC₀₋ₜ | 58.89 | 75.71 | 56.58 |
| | AUC_{0-∞} | 61.78 | 87.15 | 78.52 |
| | Cₘₐₓ | 27.58 | 26.13 | 17.27 |
| β-HTBZ | AUC₀₋ₜ | 39.67 | 60.26 | 37.27 |
| | AUC_{0-∞} | 57.73 | 71.68 | 64.00 |
| | Cₘₐₓ | 23.16 | 24.31 | 12.20 |

TABLE 3
α-HTBZ/β-HTBZ Ratios Based on AUC

| α/β | 2 × 25 mg | 50 mg CR | 50 mg CR | 50 mg CR |
|---|---|---|---|---|
| Ratio | IR | (1) | (2) | (3) |
| AUC₀₋ₜ | 3.11 ± 0.39 | 5.02 ± 2.41 | 4.12 ± 1.56 | 5.35 ± 3.51 |
| AUC_{0-∞} | 3.04 ± 0.37 | 3.50 ± 0.95 | 3.80 ± 1.01 | 3.52 ± 1.89 |

Tetrabenazine

As shown in FIG. 2, plasma tetrabenazine concentrations were mostly undetectable from the IR and CR formulations due to significant first pass metabolism. Very few subjects had detectable concentration data above the analytical limit of quantitation (LLoQ=0.2 ng/mL).

Tetrabenazine 2×25 mg IR Tablets

α-Dihydrotetrabenazine (α-HTBZ)

The concentration of α-HTBZ rose sharply and reached a peak concentration of 64.1±26.0 ng/mL at a median Tₘₐₓ of 1 hour (FIG. 2). The concentration then decreased gradually during the elimination phase and eventually reached the analytical limit of quantitation by 36-hour post-dose (LLoQ=0.5 ng/mL). The mean apparent half-life based on non-compartmental analysis was 5.7±1.8 hours. The mean AUC₀₋₁ and AUC_{0-∞}were 317±128 ng*hr/mL and 325±129 ng*hr/mL, respectively (Table A).

β-Dihydrotetrabenazine

Similar to α-HTBZ, β-dihydrotetrabenazine (β-HTBZ) appeared readily in the bloodstream after drug administration and reached a peak concentration (Cₘₐₓ) of 28.5±12.6 ng/mL at a median Tₘₐₓ of 1.0 hour.

After Cₘₐₓ, the concentration decreased and fell below the analytical limit of quantitation by 36-hour post-dose (LLoQ=0.5 ng/mL). The mean apparent half-life was 2.9±0.6 hours. The mean AUC₀₋ₜ and AUC_{0-∞} were 104±45 ng*hr/mL and 110±50 ng*hr/mL, respectively (Table 1).

α-HTBZ was found to be the major metabolite in the bloodstream. Based on comparison of AUC, α-HTBZ was about 3-fold higher than that of β-HTBZ (Table 3).

Three Formulations of Tetrabenazine 50 mg CR Tablets

α-Dihydrotetrabenazine

All three CR formulations demonstrated a broader plasma concentration-time profile of α-HTBZ with a short lag time (FIG. 1). Unlike the IR tablets, the ER plasma concentrations rose gradually and reached peak concentration (Cmax) at a later time. The decrease in concentration during the elimination phase was very slow and continuous. The concentration at 24-hour post dose, especially from Test 2 and 3 was relatively higher than that of the IR tablets.

The mean pharmacokinetic parameters are presented in Table A. All three test CR formulations demonstrated significantly lower Cₘₐₓ and smaller AUCs of α-HTBZ when compared to the IR tablets.

Comparisons of mean Cₘₐₓ from Tests 1, 2 and 3 to the IR tablets resulted in ratios of 27.58%, 26.13% and 17.27%, respectively. The mean AUC_{0-∞} ratios were 58.89%, 75.71% and 56.58%; the mean AUC_{0-∞} ratios were 61.78%, 87.15% and 78.52% (Table 2). The median Tₘₐₓ values (Test 1=2.0 hours; Test 2=2 hours, Test 3=3 hours) were significantly longer than that of the IR tablet. The mean apparent half-life of α-HTBZ from all three formulations (Test 1=9.8 hours, Test=14.0 hours, Test 3=27.3 hours) were significantly longer than the half-life of 5.7 hours from the IR tablets (p=0.0002), indicating flip-flop kinetics with the CR formulation.

β-Dihydrotetrabenazine

The β-HTBZ began to appear in the bloodstream after a short lag time, and rose gradually until peak concentration at a median Tₘₐₓ of 3 hours before drug elimination. The decease in concentration during elimination was very slow, reaching the analytical limit of quantitation at 36-hour post-dose. The concentrations at 24-hour post-dose, in particular from Tests 2 and 3, were relative higher than that of the IR tablets.

The mean pharmacokinetic parameters for β-HTBZ are presented in Table A. All three test CR formulations demonstrated significantly lower Cₘₐₓ and smaller AUCs of β-HTBZ when compared to the IR tablets.

Comparisons of mean Cₘₐₓ from Tests 1, 2 and 3 to the IR tablets resulted in ratios of 23.16%, 24.31% and 12.20%, respectively. The mean AUC₀₋ₜ ratios were 39.67%, 60.26% and 37.27%; the mean AUC_{0-∞} ratios were 57.73%, 71.68%, 64.00% (Table B). All three CR formulations showed a median Tₘₐₓ of 3.0 hours and were significantly longer than that of the IR tablet. The mean apparent half-life of β-HTBZ (Test 1=8.2 hours, Test 2=9.3 hours, Test 3=11.9 hours) were significantly longer relative to the half-live of 2.9 hours from the IR tablets (p=0.0229), indicating flip-flop kinetics with the ER formulation.

Rank Order Relationship Between Test Formulations

For both α-HTBZ and β-HTBZ, the three CR formulations demonstrated a rank order relationship for Cₘₐₓ (Table B). The rank order for Cₘₐₓ was Test 1>Test 2>Test 3. The half-life of each metabolite also showed a rank order of Test 3>Test 2>Test 1. AUC₀₋ₜ did not demonstrate a rank order since Test 2 had the largest value when compared to the other two test formulations. The results suggested that Test 1 has the fastest rate of tetrabenazine drug release in-vivo, followed by Test 2 and Test 3. Based on this finding, it appeared that the rate of formation of the two metabolites could be controlled by adjusting the input rate of tetrabenazine such that slowing down the rate of parent drug input would result in greater systemic exposure (AUC) of the metabolites. However, as indicated by the results of Test 3, significant reduction in the parent drug input rate would result in smaller AUC of the metabolite due to incomplete absorption.

α-Dihydrotetrabenazine vs β-Dihydrotetrabenazine

The plasma concentration of α-HTBZ was about 4- to 5-fold higher than that of β-HTBZ for all three CR formulations (Table C). These differences however were not significantly different than the value observed from the IR tablets (p=0.2777).

### CONCLUSIONS

The results from the three controlled release formulations of tetrabenazine showed characteristics of a once daily controlled release product with respect to the two metabolites: Prolonged rate of metabolite formation, lower Cₘₐₓ, longer Tₘₐₓ, longer half-life, adequate blood level coverage over 24 hours.

### Examples 16-29

### Prophetic Examples

### Example 16

### Unitary Osmotic System

| Tablet Core Ingredients | % of Tablet |
|---|---|
| Tetrabenazine | 22.0 |
| Lactose | 42.0 |
| Colloidal Silicon Dioxide | 0.74 |
| Polyvinyl alcohol | 5.48 |
| D-Mannitol | 29.04 |
| Sodium Stearyl Fumarate | 0.74 |
| Semipermeable Membrane Ingredients | % of Coating |
| Cellulose Acetate | 45.0 |
| Hydroxypropyl Cellulose | 40.0 |
| Acetyl Triethyl Citrate | 5.00 |
| Sodium Chloride | 10.00 |
| Organic Solvents (evaporated in process) | — |

Procedure
Granulate all tablet ingredients except D-mannitol and lubricant. Add D-mannitol and lubricant and compress using conventional means. Coat core with solution using vented pan coating process, to form a semipermeable membrane around core.

### Example 17

### Multiparticulate Osmotic System

| Microsphere Ingredients | % of Sphere |
|---|---|
| Tetrabenazine | 22 |
| Compritol ATO 888 | 35 |
| Fumaric acid (fine powder) | 8 |
| Gelucire 50/13 | 35 |
| Sustained Release Coating Ingredients | % of Coating |
| Ethyl Cellulose Prem. Std. 45 cps/10 cps 1:1 | 56.0 |
| Hydroxypropyl cellulose | 32.0 |
| Talc-micronized | 12.0 |
| Isopropranol/Acetone (evaporated in process) | — |

Procedure
Blend tetrabenazine microsphere ingredients under high shear and process using Ceform ™ processing technology. Place microspheres in Wurster-based fluidized bed coater and apply sustained release coating.

### Example 18

### Hydrophobic Core Controlled Release System (Lipid)

| Mini-Tablet Core Ingredients | % of Tablet |
|---|---|
| Tetrabenazine | 25.0 |
| Hydrogenated Vegetable Oil (Lubritab) | 32.5 |
| Hyprocellulose K100LV | 18.5 |
| Hydroxypropyl cellulose | 18.5 |
| Fumaric Acid | 5.00 |
| Magnesium Stearate | 0.50 |
| Tablet Coating Ingredients | % of Coating |
| Opadry (Clear) 5% solution | 100 |
| Purified Water (evaporated in process) | — |

Procedure
Melt granulate the drug, Lubritab, Fumaric Acid, HPMC and HPC above 80 degrees C in jacketed high shear mixer. Congeal and screen/mill/size granulate. Add lubricant and compress. Apply cosmetic coat to tablets using vented coating pan.

### Example 19

### Hydrophobic Core Controlled Release System (Wax)

| Tablet Core Ingredients | % of Tablet |
|---|---|
| Tetrabenazine | 29.35 |
| Carnauba Wax | 35.50 |
| Stearyl alcohol | 24.65 |
| Citric Acid | 10.00 |
| Magnesium Stearate | 0.50 |
| Tablet Coating Ingredients | % of Coating |
| Opadry (Clear) 5% solution | 100 |
| Purified Water (evaporated) | — |

Procedure
Melt granulate the drug, carnauba wax, citric acid and stearyl alcohol at 95-100 degrees C in jacketed high shear mixer. Congeal and screen/mill/size the granulate. Add lubricant and compress into tablets. Apply cosmetic coat to tablets using vented coating pan.

### Example 20

### Hydrophobic Core Controlled-Release System (Insoluble Polymer)

| Tablet Core Ingredients | % of Tablet |
|---|---|
| Tetrabenazine | 44.0 |
| Colloidal Silicon Dioxide | 0.74 |
| Polyvinyl alcohol | 19.48 |
| Ethyl Cellulose | 27.00 |
| Fumaric Acid | 5.00 |
| Ludipress | 3.04 |
| Sodium Stearyl Fumarate | 0.74 |
| Tablet Coating Ingredients | % of Coating |
| Opadry (Clear) 5% solution | 100 |
| Purified Water (evaporated) | — |

Procedure
Granulate tetrabenazine and silicon dioxide using PVA solution in fluid bed granulator using top-spray method. Compress granulate, ethyl cellulose, Ludipress, citric acid, and lubricant into tablets using rotary compression. Coat with cosmetic coating using vented coating pan spray technology.

### Example 21

### Hydrophobic Coat (Lipid)

| Mini-Tablet Core Ingredients | % of Tablet |
|---|---|
| Tetrabenazine | 38.00 |
| Lactose | 55.16 |
| Colloidal Silicon Dioxide | 0.96 |
| Polyvinyl alcohol | 4.92 |
| Citric Acid | 5.00 |
| Sodium Stearyl Fumarate | 0.96 |
| Mini-Tablet Coating Ingredients | % of Coating |
| Glyceryl monostearate | 75.25 |
| Polyethylene Glycol 8000 | 24.75 |

Procedure
Granulate the tetrabenazine, citric acid and lactose with colloidal silicon dioxide using PVA solution, under top-spray fluid bed process. Add lubricant to granulate and compress using conventional rotary process. Coat mini-tablets with molten lipid-based coating in Wurster fluid-bed processor outfitted with hot melt coating apparatus.

### Example 22

### Hydrophobic Coat (Wax)

| Mini-Tablet Core Ingredients | % of Tablet |
|---|---|
| Tetrabenazine | 53.00 |
| Lactose | 40.16 |
| Colloidal Silicon Dioxide | 0.96 |
| Polyvinyl alcohol | 4.92 |
| Sodium Stearyl Fumarate | 0.96 |
| Mini-Tablet Coating Ingredients | % of Coating |
| Hydrogenated Castor Oil (Castorwax) | 55.25 |
| Polyethylene Glycol 8000 | 24.75 |

Procedure
Granulate the tetrabenazine and lactose with colloidal silicon dioxide using PVA solution, under top-spray fluid bed process. Add lubricant to granulate and compress using conventional rotary process. Coat mini-tablets with molten wax-based coating in Wurster fluid-bed processor outfitted with hot melt coating apparatus.

### Example 23

### Hydrophobic Coat (Insoluble Polymer)

| Tablet Core Ingredients | % of Tablet |
|---|---|
| Tetrabenazine | 53.0 |
| Lactose | 40.16 |
| Colloidal Silicon Dioxide | 0.96 |
| Polyvinyl alcohol | 4.92 |
| Sodium Stearyl Fumarate | 0.96 |
| Tablet Coating Ingredients | % of Coating |
| Ethylcellulose | 64.09 |
| Hydroxypropyl Cellulose | 26.82 |
| Dibutyl Sebacate | 9.09 |
| Isopropanol/Acetone (evaporated) | — |

Procedure
Granulate the tetrabenazine and lactose with colloidal silicon dioxide using PVA solution, under top-spray fluid bed process. Add lubricant to granulate and compress using conventional rotary process. Coat with solvent coating in conventional vented coating pan.

### Example 24

### Hydrophilic Core (Swellable)

| Tablet Core Ingredients | % of Tablet |
|---|---|
| Tetrabenazine | 30.12 |
| Colloidal Silicon Dioxide | 0.66 |
| Polyvinyl alcohol | 4.00 |
| Hypromellose K100LV | 20.00 |
| Eudragit RL © powder | 44.26 |
| Sodium Stearyl Fumarate | 0.96 |
| Tablet Coating Ingredients | % of Coating |
| Opadry (Clear) 5% solution | 100 |
| Purified Water (evaporated in process) | — |

Procedure
Granulate all tablet ingredients except Eudragit E © and lubricant in top spray fluid bed granulator. Add Eudragit E © and lubricant and compress into tablet using conventional means. Apply cosmetic coat to tablets using vented coating pan.

### Example 25

### Hydrophilic Core (Soluble Polymer)

| Tablet Core Ingredients | % of Tablet |
|---|---|
| Tetrabenazine | 30.00 |
| Colloidal Silicon Dioxide | 0.66 |
| Polyvinyl alcohol | 1.00 |
| Hydroxypropyl Methylcellulose | 57.38 |
| Ethyl cellulose | 10.00 |
| Sodium Stearyl Fumarate | 0.96 |
| Tablet Coating Ingredients | % of Coating |
| Opadry (Clear) 5% solution | 100 |
| Purified Water (evaporated in process) | — |

Procedure
Granulate all tablet ingredients except HPMC and lubricant in top spray fluid bed granulator. Add HPMC and lubricant and compress using conventional means. Apply cosmetic coat to tablets using vented coating pan.

### Example 26

### Hydrophilic Coat (Swellable)

| Tablet Core Ingredients | % of Tablet |
|---|---|
| Tetrabenazine | 40.15 |
| Lactose | 48.01 |
| Colloidal Silicon Dioxide | 0.96 |
| Fumaric Acid | 5.00 |
| Polyvinyl alcohol | 4.92 |
| Sodium Stearyl Fumarate | 0.96 |
| Tablet Coating Ingredients | % of Coating |
| Eudragit RS © | 14.0 |
| Eudragit RL © | 56.0 |
| Acetyl Triethyl Citrate | 15.0 |
| Talc | 15.0 |
| Alcoholic/Acetone Solvents (evaporates) | — |

Procedure
Granulate the tetrabenazine and fumaric acid with colloidal silicon dioxide using PVA solution, under top-spray fluid bed process. Add lubricant to granulate and compress using conventional rotary process. Apply coating to tablets using vented coating pan..

### Example 27

### Hydrophilic Coat (Soluble Polymer)

| Tablet Core Ingredients | % of Tablet |
|---|---|
| Tetrabenazine | 36.16 |
| Lactose | 60.00 |
| Colloidal Silicon Dioxide | 0.96 |
| Polyvinyl alcohol | 1.92 |
| Sodium Stearyl Fumarate | 0.96 |
| Tablet Coating Ingredients | % of Coating |
| Hydroxymethyl Cellulose | 62.0 |
| Hydroxyethyl Cellulose | 38.0 |
| Water (evaporated) | — |

Procedure
Granulate the tetrabenazine and lactose with colloidal silicon dioxide using PVA solution, under top-spray fluid bed process. Add lubricant to granulate and compress using conventional rotary process. Coat with sufficient aqueous coating in conventional vented coating pan to sustain drug release.

### Example 28

### Tetrabenazine AQ Coated Tablet

| Tablet Core Ingredients | % of Tablet |
|---|---|
| Tetrabenazine | 23.00 |
| Lactose | 57.16 |
| Colloidal Silicon Dioxide | 0.96 |
| Polyvinyl alcohol | 4.92 |
| Kollidon CL | 8.00 |
| Citric Acid | 5.00 |
| Sodium Stearyl Fumarate | 0.96 |
| Tablet Coating Ingredients | % of Coating |
| Eudragit NE30D | 40.03 (as dry) |
| Hydroxypropyl Methylcellulose 6 cps | 23.01 |
| Polyethylene Glycol 8000 | 11.26 |
| Talc 400 | 20.26 |
| Titanium dioxide | 4.31 |
| Simethicone | 1.13 |

Procedure
Granulate the tetrabenazine, lactose, and citric acid with colloidal silicon dioxide using PVA solution, under top-spray fluid bed process. Add lubricant to granulate and compress using conventional rotary process. Coat with aqueous-based coating dispersion/suspension in conventional vented coating pan.

### Example 29

### Delayed Release System (Reverse Enteric Coat, Hydrophilic Core)

| Tablet Core Ingredients | % of Tablet |
|---|---|
| Tetrabenazine | 60.0 |
| Colloidal Silicon Dioxide | 0.74 |
| Polyvinyl alcohol | 5.00 |
| Hypromellose | 30.00 |
| Ludipress | 3.52 |
| Sodium Stearyl Fumarate | 0.74 |
| Tablet Coating Ingredients | % of Coating |
| Eudragit E100 | 66.9 |
| Acetyl Triethyl Citrate | 10.0 |
| Talc 400 | 23.1 |

Procedure
Granulate the tetrabenazine with colloidal silicon dioxide using PVA solution, under top-spray fluid bed process. Add hypromellose, Ludipress, and lubricant to granulate and compress using conventional rotary process. Coat with reverse-enteric coating in conventional vented coating pan using an alcohol-based solution.

### Example 30

### Pharmacokinetic Parameters

A single dose, 4 way crossover pilot study was performed to compare three 50 mg formulations of controlled release tetrabenazine with administration of 2×25 mg of an immediate release formulation.

TABLE 11.4-2
Summary of Pharmacokinetic Parameters

| | | | | | AUC_{τ} | AUC₁ | | | |
|---|---|---|---|---|---|---|---|---|---|
| | | | Cₘₐₓ | Tₘₐₓ | (ng · mL⁻¹ · | (ng · mL⁻¹ · | Kel | t_{1/2} | CL/F |
| Analyte | Period | Subject | (ng · mL⁻¹) | (h) | h) | h) | (h⁻¹) | (h) | (mL/h) |
| α-HTBZ | 1 | N | 10 | 10 | 10 | 10 | 10 | 10 | NA |
| | | Mean | 66.028 | 0.95 | 277.408 | 307.787 | 0.188 | 4.409 | NA |
| | | SD | 21.609 | 0.44 | 130.125 | 123.459 | 0.091 | 1.845 | NA |
| | | Min | 27.620 | 0.50 | 103.105 | 131.781 | 0.090 | 1.805 | NA |
| | | Median | 63.300 | 1.00 | 276.534 | 288.611 | 0.152 | 4.559 | NA |
| | | Max | 100.400 | 2.00 | 469.200 | 475.584 | 0.384 | 7.690 | NA |
| | | Geometric | 62.451 | NA | 247.965 | 284.023 | 0.171 | 4.044 | NA |
| | | Mean | | | | | | | |
| | 2 | N | 7 | 7 | 7 | 7 | 7 | 7 | NA |
| | | Mean | 19.224 | 2.57 | 190.179 | 203.432 | 0.072 | 9.748 | NA |
| | | SD | 10.553 | 1.72 | 87.120 | 90.486 | 0.009 | 1.094 | NA |
| | | Min | 7.006 | 1.00 | 93.884 | 101.424 | 0.063 | 7.647 | NA |
| | | Median | 24.190 | 2.00 | 203.310 | 213.199 | 0.070 | 9.890 | NA |
| | | Max | 33.150 | 6.00 | 289.648 | 312.373 | 0.091 | 11.025 | NA |
| | | Geometric | 16.356 | NA | 171.544 | 184.847 | 0.072 | 9.691 | NA |
| | | Mean | | | | | | | |
| | 3 | N | 7 | 7 | 7 | 7 | 7 | 7 | NA |
| | | Mean | 17.864 | 3.57 | 252.616 | 303.019 | 0.055 | 13.398 | NA |
| | | SD | 9.585 | 2.57 | 129.205 | 167.972 | 0.015 | 3.475 | NA |
| | | Min | 6.442 | 1.00 | 99.483 | 111.697 | 0.038 | 8.447 | NA |
| | | Median | 14.200 | 3.00 | 244.631 | 274.538 | 0.053 | 13.162 | NA |
| | | Max | 32.120 | 8.00 | 401.578 | 510.444 | 0.082 | 18.062 | NA |
| | | Geometric | 15.496 | NA | 220.639 | 259.033 | 0.053 | 13.003 | NA |
| | | Mean | | | | | | | |
| | 4 | N | 7 | 7 | 7 | 5 | 5 | 5 | NA |
| | | Mean | 11.885 | 8.00 | 193.256 | 214.647 | 0.078 | 9.464 | NA |
| | | SD | 6.334 | 8.50 | 103.335 | 134.835 | 0.021 | 2.564 | NA |
| | | Min | 3.081 | 1.00 | 49.516 | 68.103 | 0.052 | 6.425 | NA |
| | | Median | 11.620 | 3.00 | 184.269 | 183.895 | 0.073 | 9.521 | NA |
| | | Max | 23.070 | 24.00 | 346.867 | 392.441 | 0.108 | 13.333 | NA |
| | | Geometric | 10.241 | NA | 164.917 | 178.258 | 0.075 | 9.194 | NA |
| | | Mean | | | | | | | |
| β-HTBZ | 1 | N | 10 | 10 | 10 | 10 | 10 | 10 | NA |
| | | Mean | 29.631 | 1.10 | 99.905 | 118.848 | 0.262 | 2.776 | NA |
| | | SD | 12.083 | 0.32 | 49.233 | 60.875 | 0.059 | 0.657 | NA |
| | | Min | 9.618 | 1.00 | 34.324 | 38.491 | 0.165 | 1.874 | NA |
| | | Median | 30.590 | 1.00 | 94.439 | 108.822 | 0.250 | 2.779 | NA |
| | | Max | 44.420 | 2.00 | 171.531 | 228.010 | 0.370 | 4.212 | NA |
| | | Geometric | 26.914 | NA | 88.014 | 103.744 | 0.256 | 2.710 | NA |
| | | Mean | | | | | | | |
| | 2 | N | 7 | 7 | 7 | 7 | 7 | 7 | NA |
| | | Mean | 7.850 | 2.86 | 50.753 | 59.727 | 0.122 | 7.429 | NA |
| | | SD | 5.481 | 1.57 | 36.690 | 38.599 | 0.073 | 4.238 | NA |
| | | Min | 2.178 | 1.00 | 12.467 | 15.886 | 0.044 | 2.553 | NA |
| | | Median | 9.219 | 3.00 | 61.039 | 65.908 | 0.114 | 6.058 | NA |
| | | Max | 15.970 | 6.00 | 104.015 | 120.928 | 0.272 | 15.643 | NA |
| | | Geometric | 5.936 | NA | 37.377 | 47.545 | 0.107 | 6.502 | NA |
| | | Mean | | | | | | | |
| | 3 | N | 7 | 7 | 7 | 7 | 7 | 7 | NA |
| | | Mean | 7.827 | 3.86 | 77.860 | 94.921 | 0.104 | 9.113 | NA |
| | | SD | 5.037 | 3.02 | 55.258 | 69.226 | 0.080 | 4.290 | NA |
| | | Min | 1.946 | 1.00 | 15.020 | 21.207 | 0.043 | 2.495 | NA |
| | | Median | 5.946 | 3.00 | 78.459 | 86.649 | 0.074 | 9.328 | NA |
| | | Max | 14.050 | 8.00 | 151.935 | 200.607 | 0.278 | 16.052 | NA |
| | | Geometric | 6.231 | NA | 56.803 | 70.729 | 0.086 | 8.024 | NA |
| | | Mean | | | | | | | |
| β-HTBZ | 4 | N | 7 | 7 | 7 | 5 | 5 | 5 | NA |
| | | Mean | 4.068 | 6.00 | 52.420 | 103.229 | 0.089 | 12.215 | NA |
| | | SD | 2.639 | 5.80 | 39.656 | 89.523 | 0.080 | 6.862 | NA |
| | | Min | 0.619 | 1.00 | 3.808 | 21.286 | 0.034 | 3.040 | NA |
| | | Median | 2.955 | 3.00 | 47.570 | 68.502 | 0.052 | 13.387 | NA |
| | | Max | 7.273 | 16.00 | 110.467 | 236.480 | 0.228 | 20.446 | NA |
| | | Geometric | 3.130 | NA | 35.139 | 72.704 | 0.068 | 10.142 | NA |
| | | Mean | | | | | | | |
| Tetra- | 1 | N | 10 | 8 | 10 | 1 | 1 | 1 | 1 |
| benazine | | Mean | 1.019 | 0.75 | 0.758 | 5.956 | 0.971 | 0.714 | 8394772.950 |
| | | SD | 1.875 | 0.53 | 1.746 | NC | NC | NC | NC |
| | | Min | 0.000 | 0.50 | 0.000 | 5.956 | 0.971 | 0.714 | 8394772.950 |
| | | Median | 0.278 | 0.50 | 0.082 | 5.956 | 0.971 | 0.714 | 8394772.950 |
| | | Max | 6.041 | 2.00 | 5.639 | 5.956 | 0.971 | 0.714 | 8394772.950 |
| | | Geometric | NC | NA | NC | 5.956 | 0.971 | 0.714 | 8394772.950 |
| | | Mean | | | | | | | |
| | 2 | N | 7 | 1 | 7 | 1 | 1 | 1 | 1 |
| | | Mean | 0.184 | 1.00 | 0.191 | 1.864 | 0.520 | 1.333 | 26817201.981 |
| | | SD | 0.486 | NC | 0.505 | NC | NC | NC | NC |
| | | Min | 0.000 | 1.00 | 0.000 | 1.864 | 0.520 | 1.333 | 26817201.981 |
| | | Median | 0.000 | 1.00 | 0.000 | 1.864 | 0.520 | 1.333 | 26817201.981 |
| | | Max | 1.287 | 1.00 | 1.335 | 1.864 | 0.520 | 1.333 | 26817201.981 |
| | | Geometric | NC | NA | NC | 1.864 | 0.520 | 1.333 | 26817201.981 |
| | | Mean | | | | | | | |
| | 3 | N | 7 | 2 | 7 | 0 | 0 | 0 | 0 |
| | | Mean | 0.179 | 4.25 | 0.213 | NC | NC | NC | NC |
| | | SD | 0.351 | 5.30 | 0.527 | NC | NC | NC | NC |
| | | Min | 0.000 | 0.50 | 0.000 | NC | NC | NC | NC |
| | | Median | 0.000 | 4.25 | 0.000 | NC | NC | NC | NC |
| | | Max | 0.923 | 8.00 | 1.405 | NC | NC | NC | NC |
| | | Geometric | NC | NA | NC | NC | NC | NC | NC |
| | | Mean | | | | | | | |
| | 4 | N | 7 | 2 | 7 | 1 | 1 | 1 | 1 |
| | | Mean | 0.718 | 1.00 | 0.643 | 4.474 | 1.408 | 0.492 | 11174642.155 |
| | | SD | 1.768 | 0.00 | 1.603 | NC | NC | NC | NC |
| | | Min | 0.000 | 1.00 | 0.000 | 4.474 | 1.408 | 0.492 | 11174642.155 |
| | | Median | 0.000 | 1.00 | 0.000 | 4.474 | 1.408 | 0.492 | 11174642.155 |
| | | Max | 4.719 | 1.00 | 4.274 | 4.474 | 1.408 | 0.492 | 11174642.155 |
| | | Geometric | NC | NA | NC | 4.474 | 1.408 | 0.492 | 11174642.155 |
| | | Mean | | | | | | | |

NC: Not calculated
NA: Not applicable
1 = Period 1 (Reference: Tetrabenazine 2 × 25 mg immediate release formulation)
2 = Period 2 (Test formulation 1: Tetrabenazine 50 mg controlled release formulation)
3 = Period 3 (Test formulation 2: Tetrabenazine 50 mg controlled release formulation)
4 = Period 4 (Test formulation 3: Tetrabenazine 50 mg controlled release formulation)

TABLE 11.4-3
Summary of Statistical Analysis Data

| | | Test 1 | Reference | Ratio (%) | |
|---|---|---|---|---|---|
| Type | Parameter | Geometric LSmeans | | Test 1/Reference | 90% C.I.s |
| Tetrabenazine | Cₘₐₓ | 0.19 | 0.54 | 35.34 | 6.33-197.31 |
| | (ng/ml) | | | | |
| | AUC_{τ} | 0.10 | 0.26 | 38.18 | 3.83-380.89 |
| | (ng · h/ml) | | | | |
| Alpha- | Cₘₐₓ | 16.36 | 59.31 | 27.58 | 19.11-39.79 |
| dihydrotetrabenazine | (ng/ml) | | | | |
| | AUC_{τ} | 171.54 | 291.46 | 58.86 | 50.21-68.99 |
| | (ng · h/ml) | | | | |
| | AUC₁ | 184.85 | 298.52 | 61.92 | 53.45-71.74 |
| | (ng · h/ml) | | | | |
| Beta- | Cₘₐₓ | 5.94 | 25.64 | 23.15 | 14.83-36.13 |
| dihydrotetrabenazine | (ng/ml) | | | | |
| | AUC_{τ} | 37.38 | 94.27 | 39.65 | 27.53-57.10 |
| | (ng · h/ml) | | | | |
| | AUC₁ | 47.55 | 99.41 | 47.83 | 35.07-65.23 |
| | (ng · h/ml) | | | | |
| | | Test 2 | Reference | Ratio (%) | |
| Type | Parameter | Geometric LSmeans | | Test 2/Reference | 90% C.I.s |
| Tetrabenazine | Cₘₐₓ | 0.18 | 0.54 | 33.45 | 9.03-123.91 |
| | (ng/ml) | | | | |
| | AUC_{τ} | 0.09 | 0.26 | 33.44 | 5.80-192.73 |
| | (ng · h/ml) | | | | |
| Alpha- | Cₘₐₓ | 15.50 | 59.31 | 26.13 | 18.10-37.70 |
| dihydrotetrabenazine | (ng/ml) | | | | |
| | AUC_{τ} | 220.64 | 291.46 | 75.70 | 64.58-88.74 |
| | (ng · h/ml) | | | | |
| | AUC₁ | 259.03 | 298.52 | 86.77 | 74.90-100.53 |
| | (ng · h/ml) | | | | |
| Beta- | Cₘₐₓ | 6.23 | 25.64 | 24.30 | 15.57-37.92 |
| dihydrotetrabenazine | (ng/ml) | | | | |
| | AUC_{τ} | 56.80 | 94.27 | 60.26 | 41.84-86.78 |
| | (ng · h/ml) | | | | |
| | AUC₁ | 70.73 | 99.41 | 71.15 | 52.17-97.04 |
| | (ng · h/ml) | | | | |
| | | Test 3 | Reference | Ratio (%) | |
| Type | Parameter | Geometric LSmeans | | Test 3/Reference | 90% C.I.s |
| Tetrabenazine | Cₘₐₓ | 0.70 | 0.54 | 129.18 | 34.87-478.49 |
| | (ng/ml) | | | | |
| | AUC_{τ} | 0.46 | 0.26 | 176.09 | 30.56-1014.80 |
| | (ng · h/ml) | | | | |
| Alpha-dihydro- | Cₘₐₓ | 10.24 | 59.31 | 17.27 | 11.96-24.91 |
| tetrabenazine | (ng/ml) | | | | |
| | AUC_{τ} | 164.92 | 291.46 | 56.58 | 48.27-66.33 |
| | (ng · h/ml) | | | | |
| | AUC₁ | 198.92 | 298.52 | 66.64 | 56.47-78.63 |
| | (ng · h/ml) | | | | |
| Beta-dihydro- | Cₘₐₓ | 3.13 | 25.64 | 12.21 | 7.82-19.05 |
| tetrabenazine | (ng/ml) | | | | |
| | AUC_{τ} | 35.14 | 94.27 | 37.28 | 25.88-53.68 |
| | (ng · h/ml) | | | | |
| | AUC₁ | 66.11 | 99.41 | 66.51 | 46.93-94.25 |
| | (ng · h/ml) | | | | |

### Example 31

### Tetrabenazine Sustained-Release (SR) Formulations, 25 mg and 50 mg

Sustained-release (SR) formulation that uses multiparticulate to improve solubility/delivery of the drug, and these drug-loaded particles are incorporated and released from a matrix tablet system by a combination of gelation and erosion of tablet.

Drug Loaded Particles can be Ceforim, Shearform, extrusion-spheronization beads, layered beads, or other multiparticulate technology)

Examples Using Ceform Microspheres:

| | |
|---|---|
| Tetrabenazine | 24% |
| Precirol ATO 5 (glycerol palmitostearate) | 38% |
| Milled Gelucire 50/13 pellets (stearyl macrogoglycerides) | 38% |
| | 100% |

Tablet Excipients:

| | |
|---|---|
| Drug-loaded CEFORM Microspheres | 30% |
| Polyox (polyethyleneoxide) WSR NF750 | 20% |
| Encompress (dibasic calcium phosphate dihydrate) | 49% |
| Magnesium Stearate | 1% |
| | 100% |

Blend drug and microsphere excipients, process the multiparticulates to encapsulate the drug. Blend multiparticulates with other tablet excipients and compress by standard means into a tablet. For strengths of Tetrabenazine at 25 mg (375 mg total tablet weight) & 50 mg (750 mg total tablet weight), tablet sizes were formulated to be dose-proportional.

### Example 32

### Tetrabenazine Controlled Release Formulations (15 mg, 25 mg, 30 mg and 50 mg)

The following table shows the 25 mg and 50 mg tetrabenazine formulations that have been made and tested and the proposed lower dosage 15 mg and 30 mg tetrabenazine formulations.

| | Tetrabenazine CR | | | | |
|---|---|---|---|---|---|
| | 50 mg | 25 mg | 30 mg | 15 mg | Range |
| Ingredients | % w/w | % w/w | % w/w | % w/w | % w/w |
| Tetrabenazine | 20 | 10 | 12 | 6 | 6-20% |
| Lactose | 30.96 | 31.56 | 39.16 | 35.66 | 25-45% |
| Monohydrate DC | | | | | |
| Starch 1500 | 16.2 | 25.9 | 16.2 | 25.9 | 15-30% |
| Methocel K100LV | 30 | 30 | 30 | 30 | 25-35% |
| Aerosil 200 | 0.6 | 0.3 | 0.4 | 0.2 | 0.2-0.6% |
| Talc | 1.6 | 1.6 | 1.6 | 1.6 | 1-3% |
| Magnesium stearate | 0.64 | 0.64 | 0.64 | 0.64 | 0.5-1.0% |
| Total: | 100 | 100 | 100 | 100 | |

One Example of Manufacturing Procedure:
1. Tetrabenazine, Lactose DC, Starch 1500 & HPMC (K I0OLV) are sieved via a 30 mesh screen (approximately 600 Micron) into suitable containers.
2. The sieved powders are then blended in a suitable Mixer for 10 minutes at slow speed.
3. The Talc is sieved through a 30 mesh screen (approximately 600 Micron) and the Magnesium Stearate sieved through a 60 mesh screen (approximately 250 Micron).
4. The Talc and Magnesium Stearate were added to the Mixer and blended for 2 minutes at slow speed.
5. The powder blend was compressed on a rotary tabletting machine, using Flat Bevelled Edge punches.

### Example 33

### A Pilot 3-Way Single-Dose Food-Effect Study on Tetrabenazine 50 mg Controlled Release (CR) Tablets

Tetrabenazine CR 50 mg Tablets demonstrated a significant food-effect (n=13); the substantial food-effect was observed in 10 out of 13 subjects. For both alpha- and beta-dihydrotetrabenazine, the peak concentration (Cₘₐₓ) was more than doubled (238% & 263%; α & β, respectively) and the systemic exposure (AUC) was increased by about half (144% & 153%; α & β, respectively) when the CR tablet was given with food relative to fasting. In addition, the half life, an indicator of controlled-release characteristics, was shortened in the presence of the high-fat meal for both analytes and approaching those observed for the IR.

The Tmax's in the presence of food appeared to be similar to or longer than those in the fasting state; this apparent unchanged Tmax was due to the longer lag time observed in the fed state. When corrected for lag time, the Tmax's in the fed state approach those of the IR.

In conclusion, the CR formulation appears to lose its extended-release characteristics when taken with a high-fat meal relative to fasting. However, the substantial food-effect may not result in significant safety concerns as the observed Cmax's of both analytes in the fed state were lower than those of the IR in the fasting state in 5 out of 10 volunteers (mean ratio approx 70%). Moreover, significant differences in AE's between fasted and fed states were not observed in adults in this study.

The α:β dihydrotetrabenazine AUC ratios were similar across all three treatments. The differences in this ratio between IR and CR formulations observed in the previous pilot study may have been due to inter-subject variation and the smaller sample size (n=7).

TABLE 1
Mean Pharmacokinetic Parameters (Mean ± SD)
for α-DHBZ and β-DHBZ

| | | | | Nitoman ® |
|---|---|---|---|---|
| | | CR 50 mg, | CR 50 mg, | 2 × 25 mg |
| | | Fed | Fasting | Fasting |
| | | (n = 13) | (n = 13) | (n = 10) |
| α-DHTBZ | AUC₀₋ₜ | 501 ± 297 | 372 ± 261 | 444 ± 306 |
| | (ng * hr/mL) | | | |
| | AUC_{0-∞} | 531 ± 325 | 414 ± 307 | 468 ± 328 |
| | (ng * hr/mL) | | | |
| | Cₘₐₓ (ng/mL) | 53.7 ± 16.4 | 26.2 ± 17.8 | 72.7 ± 35.7 |
| | Tₘₐₓ (hr)* | 4.0 (3.0, 5.0) | 3.0 (1.0, 10.0) | 1.0 (0.5, 3.0) |
| | t_{1/2} (hr) | 9.3 ± 2.7 | 11.3 ± 3.0 | 8.7 ± 1.8 |
| | MRT (hr) | 13.0 ± 4.0 | 17.8 ± 4.6 | 10.1 ± 3.6 |
| β-DHTBZ | AUC₀₋ₜ | 316 ± 374 | 226 ± 289 | 280 ± 386 |
| | (ng * hr/mL) | | | |
| | AUC_{0-∞} | 330 ± 395 | 264 ± 339 | 298 ± 400 |
| | (ng * hr/mL) | | | |
| | Cₘₐₓ (ng/mL) | 34.8 ± 20.3 | 16.9 ± 16.3 | 46.2 ± 30.7 |
| | Tₘₐₓ (hr)* | 4.0 (3.0, 6.0) | 4.0 (1.0, 10.0) | 1.0 (1.0, 4.0) |
| | t_{1/2} (hr) | 8.0 ± 3.1 | 13.6 ± 5.2 | 8.3 ± 2.6 |
| | MRT (hr) | 11.1 ± 3.4 | 19.7 ± 6.7 | 9.1 ± 3.5 |

*Median Tₘₐₓ (Min, Max)

TABLE 2
Summary Statistics for α-DHBZ and β-DHBZ

| | | CR Tablets | CR (Fed) vs | CR (Fasting) vs |
|---|---|---|---|---|
| | | Fed vs | Nitoman ® | Nitoman ® |
| | | Fasting | (Fasting) | (Fasting) |
| | % Ratio | (n = 13) | (n = 10) | (n = 10) |
| α-DHTBZ | AUC₀₋ₜ | 144.71% | 102.67% | 67.17% |
| | AUC_{0-∞} | 138.55% | 102.40% | 70.91% |
| | Cₘₐₓ | 238.71% | 73.03% | 25.30% |
| β-DHTBZ | AUC₀₋ₜ | 153.44% | 94.50% | 58.27% |
| | AUC_{0-∞} | 133.45% | 93.89% | 68.82% |
| | Cₘₐₓ | 263.46% | 69.29% | 21.24% |

TABLE 3
α-DHTBZ/β-DHTBZ Ratios Based on AUC

| α/β | CR 50 mg | CR 50 mg | Nitoman ® 2 × 25 mg |
|---|---|---|---|
| Ratio | Fed (n = 13) | Fasting (n = 13) | Fasting (n = 10) |
| AUC₀₋ₜ | 2.25 | 2.39 | 2.15 |
| AUC_{0-∞} | 2.17 | 2.08 | 2.06 |

### Example 34

Dissolution Profile

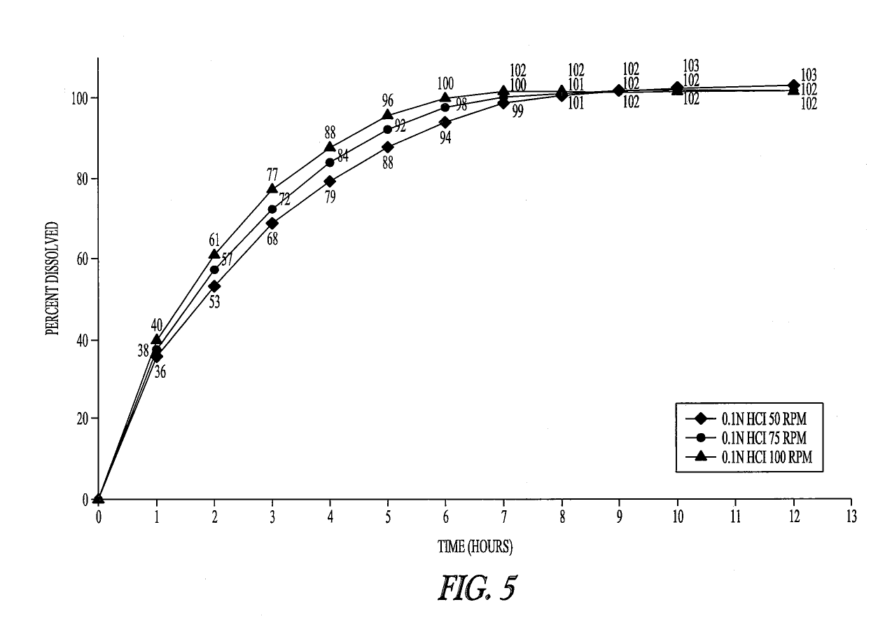

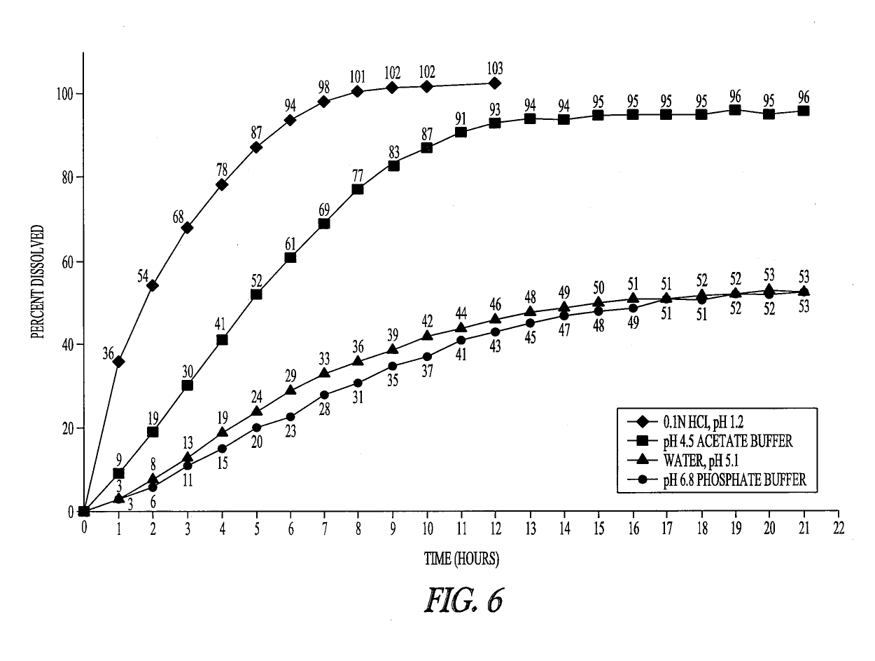

The dissolution of the 50 mg tablet with the formulation described in Example 32 was tested using a variety of different dissolution media (FIG. 6) and a dissolution apparatus employing paddles with sinkers. Three different mixing speeds were also tested (FIG. 5). The results of these dissolution tests are shown in FIGS. 5 and 6.

### Example 35

### Tetrabenazine (1TBZ) Controlled-Release (CR) Drug Layered Bead (Multiparticulate) Examples, Solvent and Aqueous-Based

This Example illustrates several types of tetrabenazine formulations that may be made and employed for delivery of tetrabenazine.

1. Tetrabenazine Sustained Release Capsules

A. Tetrabenazine-Loaded Beads

| | % |
|---|---|
| Tetrabenazine | 10.0 |
| Hypromellose 2910 (6 cps) USP | 2.00 |
| Triacetin USP | 0.40 |
| Citric Acid | 0.60 |
| Sodium Lauryl Sulfate (SLS) | 0.40 |
| Sugar Spheres USP (20-25 mesh) | 86.4 |
| Water USP (evaporated) | — |
| Total | 100.0% |

The coating composition is prepared as a 10% aqueous suspension. The suspension is applied to Sugar Spheres using standard Wurster-based air suspension coating using conditions suitable for Hypromellose-based coating (inlet target 50-70° C.).

B. Sustained Release (SR) Tetrabenazine Beads

Second-coated Sustained Release Beads can be prepared from drug spheres having the following composition:

| | % |
|---|---|
| Tetrabezaine Loaded Sugar Spheres | 84.75 |
| Ethylcellulose Std 45 Premium NF | 6.58 |
| Ethylcellulose Std 10 Premium NF | 2.19 |
| Hydroxypropyl Cellulose NF | 4.38 |
| Triethyl Citrate NF | 2.10 |
| Ethanol/Acetone 40:60 (evaporated) | — |
| Total | 100.0 |

The coating composition is prepared as a 15% alcohol/acetonle solution that includes the two types of ethylcellulose, the hydroxypropyl cellulose, and the triethyl citrate. The solution is applied to Tetrabenazine Loaded Sugar Spheres using standard Wurster-based air suspension coating using conditions suitable for Ethocel-based coatings (inlet target 45-65° C.).

The functional coating polymers for SR coating can be solvent or aqueous-based, cellulosics, methacrylics, pH independent, or pH dependent in nature. In addition to polymer application on drug layered beads, tetrabenazine beads manufactured by extrusion/spheronization can also be used as a substrate.

C. Immediate Release Overcoated SR Tetrabenzaine Beads

A final immediate release (IR) coating (identical to first coating in the bead composition described under 1.A. above, but applied as a different coating percentage) is optionally applied to SR Tetrabenazine Spheres to provide a pulsed immediate release drug component. The percentage of tetrabenazine dose from IR portion could be from 0-70%, or 5-50%, or 10-30%. The tetrabenazine-loaded beads could also be supplied in a capsule containing both IR and SR beads in selected dosage fractions.

D. Capsule Filling of Tetrabenazine-containing beads (SR, SR/IR, IR)

The coated beads can be filled into hard gelatin capsules of a suitable size. The capsule shell can be any pharmaceutically acceptable capsule shell but is preferably a hard gelatin capsule shell and is of suitable size for containing from about 10 mg to about 60 mg of Tetrabenazine. Conventional machinery and techniques are used in filling the capsule shells.

Compression of beads into tablets (either immediate release or matrix type) is also contemplated.

2. Tetrabenazine Aqueous-Based Sustained Release Capsules

A. Tetrabenazine-Loaded Beads

| | % |
|---|---|
| Tetrabenazine | 10.0 |
| Hypromellose 2910 (6 cps) USP | 2.00 |
| Triacetin USP | 0.40 |
| Citric Acid | 0.60 |
| Sodium Lauryl Sulfate (SLS) | 0.40 |
| Sugar Spheres USP (20-25 mesh) | 86.4 |
| Water USP (evaporated) | — |
| Total | 100.0% |

The coating composition containing the hypromellose, triacetin, citric acid, and sodium lauryl sulfate is prepared as a 10% aqueous suspension. The suspension is applied to Sugar Spheres using standard Wurster-based air suspension coating and conditions suitable for Hypromellose-based coating (inlet target 50-70° C.).

Compression of beads into tablets (either immediate release or SR matrix type tablets) is contemplated. Tetrabenazine-loaded Beads made by using layering technique on Sugar Spheres are preferred, but one can use drug-loaded granules, floatable particles, extruded/spheronized pellets, Ceform microspheres, or other multiparticulates for drug core component as well. The typical bead size is from about 2 millimeters to about 0.1 mm in diameter or longest dimension before coating. Solubizers and acids (or absence thereof) can also be used in the core or in the coating component of the drug-loaded beads.

B. Sustained Release (SR) Tetrabenazine Beads

Second-coated Sustained Release Beads having the following composition can be prepared from drug spheres having the following composition:

| | % |
|---|---|
| Tetrabezaine Loaded Sugar Spheres | 82.0 |
| Eudragit NE30D (as dry weight) | 6.40 |
| Hypromellose 2910 6 cps NF | 2.60 |
| Talc | 9.00 |
| Purified Water (evaporated) | — |
| Total | 100.0 |

The aqueous-based coating composition containing the eudragit, hypomellose and talc can be prepared as a 20% aqueous dispersion. The dispersion can then applied to Tetrabenazine Loaded Sugar Spheres using standard Wurster-based air suspension coating and conditions suitable for Eudragit NE 30D-based coatings (product temperature target 25-35 ° C.).

The functional coating polymers for SR coating can be solvent or aqueous-based, cellulosics, methacrylics, pH independent, or pH dependent in nature.

C. Immediate Release Overcoated SR Tetrabenazine Beads (Optional)

A final immediate release (IR) coating (identical to first coating described in 2.A. above but employed at a different coating percentage) is optionally applied to SR Tetrabenazine Spheres to provide a pulsed immediate release drug component. Percentage of dose from IR portion could be from 0-70%, 5-50%, or 10-30%. The tetrabeniazine-loaded beads could also be supplied in a capsule containing both IR and SR beads in selected dosage fractions.

D. Capsule Filling of Tetrabenazine-Containing Beads (SR, SR/IR, IR)

The aqueous-based coated beads can then be filled into hard gelatin capsules of a suitable size. The capsule shell can be any pharmaceutically acceptable capsule shell but is preferably a hard gelatin capsule shell and is of suitable size for containing from about 10 mg to about 60 mg of Tetrabenazine. Conventional machinery and technique are used in filling the capsule shells.

Compression of beads into tablets (either immediate release or SR matrix type tablets) is also contemplated. Tetrabenazine-loaded Beads using layering technique on Sugar Spheres are preferred, but one can use drug-loaded granules, floatable particles, extruded/spheronized pellets, Ceform microspheres, or other multiparticulates for drug core component as well. Typical bead size is from about 2 millimeters to about 0.1 mm in diameter or longest dimension before coating. Other solubizers and acids (or absence thereof) can also be used in the core or coating component of the drug-loaded beads.

### Prophetic Examples 35-36

### Example 35

This example granulates the drug and excipients with Eudragit NE30D dispersion. The granulate is then dried, sized and compressed into matrix controlled release tablets by conventional means.

| Tetrabenazine example formulations | Example |
|---|---|
| Components of Tablet Formulation (%) | (%) |
| Tetrabenazine | 20 |
| Eudragit NE30D | 10 |
| HPMC K100LV | 20 |
| PEO WSR Coagulant | 15 |
| Lactose monohydrate | 34 |
| Magnesium Stearate | 1 |

### Example 36

This example incorporates a reverse enteric, a swellable, and a hydrophilic polymer into a tablet matrix by dry blending or granulation to control the release of tetrabenazine.

| Tetrabenazine example formulations | Example |
|---|---|
| Components of Tablet Formulation (%) | (%) |
| Tetrabenazine | 20 |
| Eudragit EPO or E100 (fine powder) | 15 |
| HPMC K100LV | 20 |
| PEO WSR Coagulant | 15 |
| Lactose monohydrate | 29 |
| Magnesium Stearate | 1 |

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A pharmaceutical composition comprising tetrabenazine and a release-retarding agent.

2. The pharmaceutical composition of claim 1, in an oral unit dosage form.

3. The pharmaceutical composition of claim 1 or claim 2, wherein the tetrabenazine is the sole therapeutic agent.

4. The pharmaceutical composition of any one of claims 1 to 3, wherein the tetrabenazine is combined with a second therapeutic agent.

5. The pharmaceutical composition of claim 4, wherein the second therapeutic agent is an antidepressant, anticholinergic, antiepileptic, anti-Parkinsons agent, antipsychotic, aricept, baclofen, barbiturate, benzodiazepine, beta-blocker, botulinum toxin, calcium channel antagonist, catecholamimine-depleting agent, clomiplamine, clonidine, clonazepam, clozapine, diphenhydramine, dopaminergic drug, dopamine agonist, fluphenazine, guanfacine, haloperidol, 5-hydroxytryptophan, keppra, L-dopa, methylphenidate, metoclopramide, mirapex, muscle relaxant, neuroleptics, olanzapine, perphenazine, phenytoin, pimozide, piquindone, piracetam, primidone, psychostimulant, requip, risperidone, selegiline, serotonin reuptake inhibitor, sertraline, sodium valproate, sulpiride, tiapride, tricyclic antidepressants, trihexyphenidyl, trihexyphenidyl-hydrochloride (Pakisonal)), ziprasidone, or a combination thereof.

6. The pharmaceutical composition of any one of claims 1 to 5, which is a tablet, powder, capsule, sachet, troche or lozenge.

7. The pharmaceutical composition of any one of claims 1 to 6, further comprising at least one of a diluent, disintegrant, glidant and lubricant.

8. The pharmaceutical composition of claim 7, wherein the diluent is a sugar.

9. The pharmaceutical composition of claim 8, wherein the sugar is lactose.

10. The pharmaceutical composition of any one of claims 7 to 9, wherein the diluent comprises about 30% (w/w) to about 40% (w/w) of the composition.

11. The pharmaceutical composition of any one of claims 7 to 10, wherein the disintegrant is starch.

12. The pharmaceutical composition of any one of claims 7 to 11, wherein the disintegrant comprises about 15% (w/w) to about 30% (w/w) of the composition.

13. The pharmaceutical composition of any one of claims 7 to 12, wherein the glidant is talc, colloidal silicon dioxide, or a combination thereof

14. The pharmaceutical composition of any one of claims 7 to 13, wherein the glidant comprises about 1% (w/w) to about 2% (w/w) of the composition.

15. The pharmaceutical composition of any one of claims 7 to 14, wherein the lubricant is magnesium stearate.

16. The pharmaceutical composition of any one of claims 7 to 15, wherein the lubricant comprises about 0.1 (w/w) to about 2% (w/w) of the composition.

17. The pharmaceutical composition of any one of claims 1 to 16, wherein the tetrabenazine comprises about 5% (w/w) to about 20% (w/w) of the composition.

18. The pharmaceutical composition of claim 2, wherein the unit dosage form:
(i) contains about 10 mg of tetrabenazine; or
(ii) contains about 12.5 mg of tetrabenazine; or
(iii) contains about 15 mg of tetrabenazine; or
(iv) contains about 20 mg of tetrabenazine; or
(v) contains about 25 mg of tetrabenazine; or
(vi) contains about 30 mg of tetrabenazine; or
(vii) contains about 50 mg of tetrabenazine.

19. The pharmaceutical composition of any one of claims 1 to 18 that exhibits a food effect.

20. The pharmaceutical composition of any one of claims 1 to 19, wherein the release-retarding agent comprises an agent selected from a cellulose derivative, a polyoxyalkylene block co-polymer, and mixtures thereof.

21. The pharmaceutical composition of claim 20, wherein:
(i) the release-retarding agent comprises a cellulose derivative: or
(ii) the release-retarding agent is a cellulose derivative.

22. The pharmaceutical composition of claim 21, wherein the release-retarding agent comprises hydroxypropyl methyl cellulose (HPMC).

23. The pharmaceutical composition of any one of claims 1 to 22, wherein the release-retarding agent comprises about 20% (w/w) to about 40% (w/w) of the composition.

24. The pharmaceutical composition of any one of claims 1 to 23, which is a modified-release dosage unit form, a controlled-release dosage unit form, an extended release dosage unit form, a prolonged-release dosage unit form, a delayed release dosage unit form, an enhanced absorption dosage unit form, a pulsatile release dosage unit form, a gastro-retention unit dosage form, or a sustained-release dosage unit form.

25. A method of treating a hyperkinetic movement disorder, the method comprising administering an effective amount of the pharmaceutical composition of any one of claims 1 to 24, for a period of time effective to treat the hyperkinetic movement disorder.

26. The method of claim 25, wherein the hyperkinetic movement disorder comprises at least one of Huntington's disease, chorea associated with Huntington's disease, hemiballismus, senile chorea, tic disorders, tardive dyskinesia, myoclonus, dystonia and Tourette's syndrome.

27. The method of claim 25, wherein the pharmaceutical composition comprises a second therapeutic agent.

28. The method of claim 27, wherein the second therapeutic agent is an antidepressant, anticholinergic, antiepileptic, anti-Parkinsons agent, antipsychotic, aricept, baclofen, barbiturate, benzodiazepine, beta-blocker, botulinum toxin, calcium channel antagonist, catecholamine-depleting agent, clomiplamine, clonidine, clonazepam, clozapine, diphenhydramine, dopaminergic drug, dopamine agonist, fluphenazine, guanfacine, haloperidol, 5-hydroxytryptophan, keppra, L-dopa, methylphenidate, metoclopramide, mirapex, muscle relaxant, neuroleptics, olanzapine, perphenazine, phenytoin, pimozide, piquindone, piracetam, primidone, psychostimulant, requip, risperidone, selegiline, serotonin reuptake inhibitor, sertraline, sodium valproate, sulpiride, tiapride, tricyclic antidepressants, trihexyphenidyl, trihexyphenidyl-hydrochloride (Pakisonal), ziprasidone, or a combination thereof.

29. The method of claim 25, wherein the pharmaceutical composition is administered within about 1 hour, before or after, of ingesting food.

30. The method of claim 25, wherein the pharmaceutical composition is administered within about 1 hour, before or after, of ingesting a high-fat food or a high-fat beverage.

31. The method of claim 29 or 30, wherein the Fed/Fast ratio of the systemic exposure (AUC) of each of the active metabolites alpha- and beta-dihydrotetrabenazine is at least about 140%.

32. The method of claim 29 or 30, wherein the Fed/Fast ratio of the peak concentration (Cmax) of each of the active metabolites alpha- and beta-dihydrotetrabenazine is at least about 220%.

33. The method of claim 29 or 30, wherein the Cmax of each of the active metabolites alpha- and beta-dihydrotetrabenazine in the blood is obtained between about 3 hours and about 6 hours after administration of the composition.

34. The method of claim 25, wherein the pharmaceutical composition is administered about once a day (q.d.).

35. The method of claim 25, wherein the pharmaceutical composition is administered about twice a day (b.i.d.).

36. The method of claim 25, wherein the method of treating the hyperkinetic movement disorder in a patient in need thereof reduces the incidence of hyperkinetic movement in the patient.

37. The method of claim 25, wherein the method of treating the hyperkinetic movement disorder in a patient in need thereof reduces the severity of hyperkinetic movement in the patient.

38. The method of claim 25, wherein the patient experiences a lower incidence of adverse effects, as compared to an immediate release composition that contains tetrabenazine.

39. The method of claim 25, wherein the patient experiences a lower severity of adverse effects, as compared to an immediate release composition that contains tetrabenazine.

40. The method of claim 38 or 39, wherein the adverse effects comprise at least one of akathisia, depression, suicidal thoughts, suicidal behavior (suicidality), dizziness, drowsiness, sedation, somnolence, insomnia, fatigue, nervousness, anxiety, nausea and Parkinisonism.

41. A pharmaceutical composition according to any one of claims 1 to 24 for use in a method as defined in any one of claims 25 to 40.

42. The use of tetrabenazine and a release-retarding agent, and optionally a second therapeutic agent, as defined in any one of claims 1 to 24, for the manufacture of a medicament for use in a method as defined in any one of claims 25 to 40.
